(19) United States
(12) Patent Application Publication
(10) Pub. No.: US 2026/0000669 A1
(43) Pub. Date: Jan. 1, 2026

(54) TREATMENT REGIMEN FOR AUTOIMMUNE DISEASES AND INFLAMMATORY DISEASES

(71) Applicant: Merck Patent GmbH, Darmstadt (DE)

(72) Inventor: Yann Hyvert, Darmstadt (DE)

(73) Assignee: Merck Patent GmbH, Darmstadt (DE)

(21) Appl. No.: 18/878,328

(22) PCT Filed: Jun. 23, 2023
(86) PCT No.: PCT/EP2023/067166

§ 371 Date: Dec. 23, 2024

Related U.S. Application Data
(60) Provisional application No. 63/355,428, filed on Jun. 24, 2022.
(60) Provisional application No. 63/486,336, filed on Feb. 22, 2023.

(51) Int. Cl. A61K 31/506; A61K 39/145; A61K 39/215; A61P 31/14; A61P 31/16; A61P 37/06

(52) U.S. Cl. CPC A61K 31/506; A61K 39/145; A61K 39/215; A61P 31/14; A61P 31/16; A61P 37/06

(57) ABSTRACT

The present disclosure relates to a novel treatment regimen for the treatment of autoimmune diseases and other inflammatory diseases with BTK (Bruton's tyrosine kinase) inhibitors. In particular, the present disclosure relates to a treatment that combines BTK inhibitor treatment with vaccination to avoid, reduce the risk of contracting or ameliorate an infection. Moreover, the present disclosure relates to vaccination in the presence of a BTK inhibitor.

### FIELD OF THE INVENTION

The present disclosure relates to a novel treatment regimen for the treatment of autoimmune diseases and other inflammatory diseases with BTK (Bruton's tyrosine kinase) inhibitors. In particular, the present disclosure relates to a treatment that combines BTK inhibitor treatment with vaccination to avoid, reduce the risk of contracting or ameliorate an infection. Moreover, the present disclosure relates to vaccination in the presence of a BTK inhibitor.

### BACKGROUND OF THE INVENTION

Autoimmune disorders, such as multiple sclerosis (MS), rheumatoid arthritis (RA), systemic lupus erythematosus (SLE), neuromyelitis optica spectrum disorders (NMOSD) and myasthenia gravis (MG), chronic spontaneous urticaria (CSU) and idiopathic thrombocytopenia (ITP) and certain other inflammatory diseases, such as graft versus host disease (GVHD), large vessel vasculitis (LVV) and asthma, result from an over-activation the body's own immune system. Therefore, a suppression or modulation of at least a part of the immune system can lead to improvements of the clinical picture of the respective diseases.

Accordingly, a variety of directly or indirectly immunosuppressant and immunomodulatory drugs are widely used in the treatment of autoimmune disorders and inflammatory diseases, including, but not limited to, corticosteroids, such as cortisol, glucocorticoids, such as prednisone, dexamethasone and hydrocortisone, cytostatics, such as nitrogen mustards (e.g. cyclophosphamide), nitrosoureas, platinum compounds, anti-metabolites, such as methotrexate, azathioprine, mercaptopurine, fluorouracil and cladribine, B and/or T cell receptor directed antibodies, such as obinutuzumab, rituximab, ocaratuzumab, ocrelizumab and muromonab, drugs acting on immunophilins, such as ciclosporin, tacrolimus, sirolimus and everolimus, and other drugs targeting the immune system, such as interferons, opioids, TNF binding proteins, such as infliximab, etanercept and adalimumab, and mycophenolate.

Similarly, Bruton's tyrosine kinase (BTK) inhibitors are a class of immunomodulatory drugs that has shown great potential for the clinical treatment of autoimmune diseases and other inflammatory diseases.

Bruton's tyrosine kinase (BTK) is a member of the Tec family of tyrosine kinases and is expressed in B cells, macrophages, and monocytes, but not in T cells. Mutation of BTK in humans leads to the condition X-linked agammaglobulinemia (XLA) (reviewed in Rosen et al., New Eng. J. Med. (1995), vol. 333, p. 431 and Lindvall et al., Immunol. Rev. (2005), vol. 203, p. 200). These patients are immunocompromised and show impaired maturation of B cells, decreased immunoglobulin and peripheral B cell levels, diminished T cell-independent immune responses as well as attenuated calcium mobilization following BCR stimulation.

Evidence for a role for BTK in autoimmune and inflammatory diseases has also been provided by BTK-deficient mouse models. In preclinical murine models of SLE, BTK-deficient mice show marked amelioration of disease progression. In addition, BTK-deficient mice are resistant to collagen-induced arthritis (Jansson and Holmdahl, Clin. Exp. Immunol. (1993), vol. 94, p. 459). A selective BTK inhibitor (BTKi) has demonstrated dose-dependent efficacy in a mouse arthritis model (Pan et al., Chem. Med Chem. (2007), vol. 2, p. 58-61).

BTK is also expressed by cells other than B cells that may be involved in disease processes. BTK is a key component of Fc-gamma signaling in myeloid cells. For example, BTK is expressed by mast cells and BTK-deficient bone marrow-derived mast cells demonstrate impaired antigen induced degranulation (Iwaki et al., J. Biol. Chem. (2005), vol. 280, p. 40261). This shows BTK could be useful to treat pathological mast cells responses such as allergy and asthma. Also, monocytes from XLA patients, in which BTK activity is absent, show decreased TNF alpha production following stimulation (Horwood et al., J. Exp. Med. (2003), vol. 197, p. 1603). Therefore, TNF alpha-mediated inflammation could be modulated by small molecular BTK inhibitors.

Such findings suggest that BTK inhibitors might be useful in the treatment of autoimmune diseases and other inflammatory diseases involving several B cell subtypes and/or macrophage activation.

Indeed, several BTK inhibitors that have already shown great potential for the treatment of autoimmune diseases and other inflammatory diseases or are currently in clinical trials for such diseases (reviewed in Ringheim et al., Frontiers in Immunology (2021), vol. 66, p. 2223). Specifically, at least thirteen compounds targeting BTK in nine indications have attained clinical trial status of phase II or beyond. These studies relate to indications including rheumatoid arthritis, systemic lupus erythematosus, multiple sclerosis, including relapsing MS (RMS) and progressive forms (PMS), pemphigus vulgaris (PV), Sjogren's syndrome (SJ), chronic spontaneous urticaria, idiopathic thrombocytopenia, graft versus host disease, and asthma. Eleven of these BTK inhibitor clinical drug candidates are covalent alkylating agents, forming an irreversible bond with cysteine 481 in the ATP binding pocket. Of the thirteen BTK inhibitors tested in the clinic, only two have noncovalent inhibition mechanisms of action, BMS-986142 and fenebrutinib. A new trial in myasthenia gravis (MG) with tolebrutinib has been announced recently (NCT05132569). Two BTK inhibitors, evobrutinib and tolebrutinib, have shown positive results in RMS phase II trials (Montalban et al., New Engl. J. of Medicine (2019), vol. 380, p. 2406; Reich et al., Lancet Neurology (2021), vol. 20, p. 729).

During treatment, patients treated for an autoimmune disease or inflammatory disease are particular vulnerable to infection, in particular if the disease or the medical drug used for treatment interferes with the normal immune response. Hence, it is highly desirable that infections of such patients are avoided or the risk of contracting such an infection is reduced and, if an infection is indeed contracted, that the course of the infection is as much as possible ameliorated.

One of the most powerful approaches to prevent or ameliorate infections is vaccination against the agent causing the infection. However, also vaccination generally requires a proper immune response of the respective body.

A successful vaccine response resulting in vaccine-mediated protection is a complex immunological process. Most antigens trigger B cell (humoral immunity) and T cell responses (cellular immunity). Early protective efficacy is primarily conferred by the induction of immune effectors produced by B cells and antigen-specific antibodies capable of binding to a pathogen or toxin. Long-term protection requires the persistence of vaccine antibodies above a protective threshold and/or the maintenance of immune memory capable of rapid and effective reactivation.

Memory B cells are generated during primary responses to T cell-dependent vaccines. They persist in the absence of antigens, and re-exposure to antigen drives their differentiation into antibody-producing plasma cells. This reactivation is rapid such that booster responses are characterized by fast increase to higher titers of antibodies that have a higher affinity for antigens than do antibodies generated during primary responses (Siegrist, Vaccines—Vaccine Immunology, book chapter in: “Vaccines”, editors: Plotkin, Orenstein and Offit, published by Saunders/Elsevier, 5th ed. (2008), p. 16-34; Papp et al., J. of Cutaneous Medicine and Surgery (2019), vol. 32, p. 50-74). Thus, a vaccine response also depends on the type of response (recall response versus response to a novel antigen).

Moreover, responses to vaccines also depend on the vaccine type. For example, for the following vaccines protective efficacy is mostly mediated by antibodies for the following vaccines: cholera, diphtheria toxoid, hepatitis A and B, Haemophilus influenzae type b, influenza, Japanese encephalitis, meningococcal polysaccharide (PS) and conjugates, papillomavirus, pneumococcal PS and conjugates, polio (Sabin and Salk), rabies, rotavirus, rubella, tetanus toxoid, typhoid PS, and yellow fever (YF). Effects are solely born by T cells for tuberculosis (BCG), or by a combination of antibodies and T for measles and intranasal influenza vaccination (Zrzavry et al., Frontiers in Immunology (2019), vol. 10, p. 1883).

Moreover, responses to vaccines also depend on the impact of the individual disease-modifying therapy on humoral and cellular immunity, since immunosuppressant and immunomodulatory treatment often interferes with a proper vaccination response.

For example, from scientific considerations there is reason to believe that BTK inhibitors may not be compatible with vaccination, most notably because their effects on B cell receptor signalling, B/T cell interaction and antigen presentation, T follicular helper cell (Tfh)/B cell interactions in the germinal center resulting in altered B cell maturation, decreased class-switching and immunoglobulin synthesis, This mode of action may translate into a reduced primary and secondary humoral response and lower levels of antibodies generated against both novel and recall target antigens (Rijvers et al., AAN (2021), Abstract; Bogers et al., ECTRIMS (2022), Abstract; Rijvers et al., JCI Insight (2022, in press).

In line with this assumption, Li et al. (Acta Neuropathologica (2022), vol. 143, p. 505) showed through an autologous antigen-specific B cell:T cell co-culture system that a BTK inhibitor significantly diminished the proliferation of B cells and T cells and limited expression of the T cell pro-inflammatory cytokines IFN gamma and TNF alpha in response to the recall vaccine-associated tetanus toxoid.

In addition, in a recent study by Parry et al. (J. of Hematology & Oncology (2022), vol. 15, p. 3), antibody responses after dual COVID-19 vaccination were tested in 279 non-treated CLL patients under monitoring and 208 patients having received therapy (20% of which were currently taking BTKi therapy). Spike-specific seroconversion post-vaccine was highest (79%) amongst non-treated CLL patients on monitoring. The overall antibody response rate in CLL patients on BTKi therapy was significantly lower (32%) compared to the untreated patients, but also to the overall antibody response in all patients who had previously received therapy (43%).

The study Douglas et al., Haematologica (2017), vol. 102, p. e398, tested for serological responses before and after influenza vaccination in patients of chronic lymphocytic leukemia (CLL) and Waldenstrom macroglobulinemia (WM) under treatment with the BTK inhibitor ibrutinib. The authors conclude that their results demonstrate an absence of a serological response to influenza vaccination in patients on treatment with this BTK inhibitor.

These results seemed to be at odds with the finding reported by Sun et al. (JAMA Oncology (2016), vol. 2(12), p. 1656-1657), after studying a similar question. However, as Douglas et al. point out, the study of Sun et al. suffers from multiple flaws, such as an unclear extent of prior treatment in their population, an unclear proportion of patients who received a high-dose vs. low-dose influenza vaccine, and in particular a lack of a control arm, which makes the results of Sun et al. difficult to interpret and raises doubts whether their conclusions are justified.

In Pleyer et al., Blood (2021), vol. 137(2), p. 185-189, vaccination with recombinant adjuvanted hepatitis B vaccine (HepB-CpG) or with recombinant adjuvanted shingles vaccine (RZV, recombinant zoster vaccine) was studied in patients with CLL on BTK inhibitor therapy with the BTK inhibitors ibrutinib or acalabrutinib. With regard to the hepatitis vaccination, the authors conclude that de novo immune response to hepatitis B vaccine was “nearly absent” in CLL patients on BTKi and impaired in treatment-naïve patients. While the effects of BTK inhibitor therapy on the rate of response to RZV was much weaker (the authors of Pleyer et al. conclude that the responses were overall “not significantly different”), seroconversion was detected in patients on BTK inhibitor treatment at a significantly lower rate than in patients not treated with BTK inhibitors.

In a second, more recent study, Pleyer et al. (Blood Advances (2022), vol. 6, p. 1733-1740) show that recombinant zoster vaccine (RZV) induced both humoral and cellular immune responses in treated and untreated CLL patients, albeit with lower response rates in patients on BTKi therapy compared with treatment naïve CLL patients. The antibody response rate was significantly higher in the treatment naïve (TN) cohort (76.8%) than in patients receiving a BTKi (40.0%). The cellular response rate was also significantly higher in the TN cohort (70.0%) compared with the BTKi group (41.3%).

Thus, the current in vitro and in vivo data on the mode of action of evobrutinib and other BTK inhibitors and the vaccination data obtained with selected BTK inhibitors in CLL patients, suggest that de novo and recall vaccination outcomes might be blunted for BTK inhibitors in general, and also in other situations where administration of BTK inhibitors may be advantageous, such as in autoimmune diseases or other inflammatory diseases.

In situations where a proper immune response to a vaccine cannot be expected, it is generally recommended to carry out vaccination well in advance of the start and/or well after the end of immunosuppressive or immunomodulatory treatment. Normally, a gap or an interval with a duration of a few weeks to several months between a vaccination and the beginning and/or end of an immunosuppressive treatment is applied in order to have a safety margin. Typical recommendations for vaccination in patients who will undergo immunosuppressive/immunomodulatory treatment are that vaccination should be given at least two weeks for inactivated and ≥4 weeks for live vaccines. Importantly, the refractory period after immunosuppression has to be considered as well, which may be up to a full year depending on the type of medication (e.g., rituximab or alemtuzumab). Except for a few treatments which only lead to mild immunosuppression, live vaccines are strongly contraindicated under immunosuppressive treatment (Zrzavry et al., Frontiers in Immunology (2019), vol. 10, p. 1883).

These recommendations are in line with Papp et al., Journal of Cutaneous Medicine and Surgery (2019), vol. 32, p. 50-74. The authors of this publication recommend that immunization status be assessed and age- and condition-appropriate vaccines be administered prior to initiation of immunosuppressive treatment in patients newly diagnosed with immune-mediated diseases. To optimize the immunogenicity of inactivated vaccines or live attenuated herpes zoster vaccine in treatment in treatment-naive patients with immune-mediated conditions, they suggest that immunization be performed at least 2 to 4 weeks prior to initiation of immunosuppressive therapy.

However, in many therapeutic situations, immunosuppressive or immunomodulatory treatment is already ongoing at the time when vaccination is desired. Due to the severity and unpredictability of many autoimmune diseases and immune-related inflammatory diseases that can impose serious outbreaks, flares or relapses of disease activity basically at any given time, treatment of such diseases can often not be interrupted to provide a gap or interval as safety margin for vaccination. Moreover, as many of these diseases are typically not curable, but rather are chronic lifelong diseases, sustained administration of immunosuppressive/immunomodulatory drugs over the years, if not decades, is often necessary. In these situations, vaccination before the start or after the end of treatment is not possible.

It would therefore be highly desirable to find ways how patients suffering from autoimmune diseases and other inflammatory diseases can be vaccinated while being continuously treated with an immunosuppressant/immunomodulatory drug, i.e., how such patients can be continuously treated with an immunosuppressant or immunomodulatory drug, but still be successfully vaccinated.

Accordingly, there is a need in the art for improved ways to treat autoimmune diseases and inflammatory diseases. Moreover, there is a need in the art for improved ways to treat autoimmune diseases, such as SLE and MS, in particular MS. Moreover, there is a need in the art for improved ways to treat autoimmune diseases with BTK inhibitors. Moreover, there is a need in the art for improved ways to treat autoimmune or inflammatory diseases that allow for successful vaccination during treatment of such diseases. Moreover, there is a need in the art for improved ways to vaccinate patients with autoimmune diseases or inflammatory diseases. Moreover, there is a need in the art for improved ways to vaccinate patients with autoimmune diseases that are treated with immunomodulatory drugs.

The present disclosure overcomes the above-described problems and addresses the above-described needs.

### SUMMARY

The present disclosure is, in part, based on the surprising observation that patients under treatment with BTK inhibitors, in particular under treatment with the BTK inhibitor evobrutinib, can upon vaccination against seasonal influenza or COVID-19, mount a humoral response comparable to untreated patients. It was further observed that a humoral response comparable to untreated patients was elicited in different autoimmune diseases, such as SLE and MS, implying that successful vaccination under evobrutinib treatment (and likely under treatment with closely related BTK inhibitors) may occur for other autoimmune diseases as well, and feasibly also for other inflammatory diseases. Moreover, our analysis indicates that a humoral response under BTK inhibitor treatment, in particular under treatment with evobrutinib, can be obtained for vaccination with a recall antigen as well as with a novel antigen.

These surprising findings open the door for novel ways of treatment that combine BTK inhibitor administration with vaccination that allow to address the above-described needs by the different aspects and embodiments of the present disclosure:

In an aspect, the present disclosure relates to a method for treating a disease in a patient in need thereof, the method comprising:
(a) administering a BTK inhibitor (Bruton's tyrosine kinase inhibitor) to said patient, and
(b) vaccinating the patient against an infection.

In an aspect, the present disclosure relates to a method for treating a disease in a patient in need thereof, the method comprising:
(a) identifying a patient in need of treatment of said disease who is at risk of contracting an infection,
(b) vaccinating said patient who is at risk of contracting said infection against said infection, and
(c) administering a BTK inhibitor to said patient.

In another aspect, the present disclosure relates to a method for treating a disease in a patient in need thereof, the method comprising:
(a) identifying a patient in need of treatment of said disease who is at risk of contracting an infection,
(b) vaccinating said patient who is at risk of contracting said infection against said infection, and
(c) treating said disease by administering a BTK inhibitor to said patient.

In another aspect, the present disclosure relates to a method for treating a disease in a patient in need thereof, the method comprising:
(a) administering a BTK inhibitor to said patient,
(b) identifying that said patient is at risk of contracting an infection, and
(c) vaccinating said patient against said infection.

In another aspect, the present disclosure relates to a method for treating a disease in a patient in need thereof, the method comprising:
(a) administering said BTK inhibitor recurrently to a patient, thereby treating said patient for said disease,
(b) identifying that said patient is at risk of contracting an infection while on recurrent treatment with said BTK inhibitor, and
(c) vaccinating said patient against said infection while on recurrent treatment with said BTK inhibitor, and optionally
(d) continuing said recurrent treatment with said BTK inhibitor.

In another aspect, the present disclosure relates to a method for treating a disease in a patient in need thereof, the method comprising:
(a) administering a BTK inhibitor to said patient,
(b) vaccinating said patient against an infection, and
(c) continuing to administer said BTK inhibitor to said patient.

In another aspect, the present disclosure relates to a method for treating a disease in a patient in need thereof, the method comprising:
(a) optionally administering a BTK inhibitor to said patient,
(b) identifying that said patient is at risk of contracting an infection,
(c) vaccinating said patient against said infection, and
(d) optionally administering said BTK inhibitor to said patient, with the proviso that said method comprises one or more treatment periods in which said BTK inhibitor is administered,
thereby treating said patient against said disease and reducing the risk of said patient of contracting said infection.

In another aspect, the present disclosure relates to a method for treating a disease in a patient in need thereof, said treatment comprising administering a BTK inhibitor to said patient, wherein, while said patient is under ongoing treatment with said BTK inhibitor, said patient is vaccinated against an infection.

In another aspect, the present disclosure relates to a method for reducing the risk of a subject to contract an infection, the method comprising:
(a) administering a BTK inhibitor to said subject, and
(b) vaccinating said subject against said infection.

In another aspect, the present disclosure relates to a method for reducing the risk of a subject to contract an infection, the method comprising vaccinating said subject against said infection, wherein said subject is vaccinated while under ongoing treatment with a BTK inhibitor.

In another aspect, the present disclosure relates to a method for treating a disease in a patient in need thereof and reducing the risk of an infection in said patient, said method comprising
(a) administering a BTK inhibitor to said patient, and
(b) vaccinating said patient against said infection.

In another aspect, the present disclosure relates to a BTK inhibitor for use in the treatment of a disease by a method comprising:
(a) administering said BTK inhibitor to a patient having said disease, and
(b) vaccinating the patient against an infection.

In another aspect, the present disclosure relates to a BTK inhibitor for use in the treatment of a disease by a method comprising:
(a) identifying a patient who is at risk of contracting an infection,
(b) vaccinating said patient who is at risk of contracting said infection against said infection, and
(c) administering said BTK inhibitor to said patient.

In another aspect, the present disclosure relates to a BTK inhibitor for use in the treatment of a disease, wherein said BTK inhibitor is administered as follows:
(a) administering said BTK inhibitor to a patient,
(b) identifying that said patient is at risk of contracting an infection, and
(c) vaccinating said patient against said infection.

In another aspect, the present disclosure relates to a BTK inhibitor for use in the treatment of a disease, wherein said BTK inhibitor is administered as follows:
(a) administering said BTK inhibitor recurrently to a patient, thereby treating said patient for said disease,
(b) identifying that said patient is at risk of contracting an infection while on recurrent treatment with said BTK inhibitor, and
(c) vaccinating said patient against said infection while on recurrent treatment with said BTK inhibitor, and optionally
(d) continuing said recurrent treatment with said BTK inhibitor.

In another aspect, the present disclosure relates to a BTK inhibitor for use in the treatment of a disease, wherein said BTK inhibitor is administered as follows:
(a) administering said BTK inhibitor to a patient,
(b) vaccinating said patient against an infection, and
(c) continuing to administer said BTK inhibitor to said patient.

In another aspect, the present disclosure relates to a BTK inhibitor for use in the treatment of a disease, wherein said BTK inhibitor is administered by the following method:
(a) optionally administering said BTK inhibitor to a patient,
(b) identifying that said patient is at risk of contracting an infection,
(c) vaccinating said patient against said infection, and
(d) optionally administering said BTK inhibitor to said patient, with the proviso that said method comprises one or more treatment periods in which said BTK inhibitor is administered.

In another aspect, the present disclosure relates to a BTK inhibitor for use in the treatment of a disease by a method that involves administering said BTK inhibitor to a patient having said disease, said method involving vaccinating said patient against an infection while said patient is under ongoing treatment with said BTK inhibitor.

In another aspect, the present disclosure relates to a BTK inhibitor for use in the treatment of a disease, wherein, while a patient having said disease is under ongoing treatment with said BTK inhibitor, said patient is vaccinated against an infection.

In another aspect, the present disclosure relates to a BTK inhibitor for use in the treatment of a disease, wherein said BTK inhibitor is administered in combination with a vaccine.

In another aspect, the present disclosure relates to a vaccine for use in the prevention of an infection by a method comprising:
(a) administering a BTK inhibitor to a subject, and
(b) vaccinating the subject against said infection.

In another aspect, the present disclosure relates to a vaccine for use in the prevention of an infection by a method comprising vaccinating said subject against said infection, wherein said subject is vaccinated while under ongoing treatment with a BTK inhibitor.

In another aspect, the present disclosure relates to a vaccine for use in the prevention of an infection by a method comprising:
(a) identifying a subject who is at risk of contracting said infection,
(b) vaccinating said subject who is at risk of contracting said infection with said vaccine against said infection, and
(c) administering a BTK inhibitor to said subject.

In another aspect, the present disclosure relates to a vaccine for use in the prevention of an infection by a method comprising:
(a) administering a BTK inhibitor to a subject,
(b) vaccinating said subject with said vaccine against said infection, and
(c) continuing to administer said BTK inhibitor to said subject.

In another aspect, the present disclosure relates to the use of a BTK inhibitor for the manufacture of a medicament for the treatment of a disease by a method comprising:
(a) administering said BTK inhibitor to a patient having said disease, and
(b) vaccinating the patient against an infection.

In another aspect, the present disclosure relates to the use of a BTK inhibitor for the manufacture of a medicament for the treatment of a disease by a method comprising:
(a) identifying a patient who is at risk of contracting an infection,
(b) vaccinating said patient who is at risk of contracting said infection against said infection, and
(c) administering said BTK inhibitor to said patient.

In another aspect, the present disclosure relates to the use of a BTK inhibitor for the manufacture of a medicament for the treatment of a disease, wherein said BTK inhibitor is administered as follows:
(a) administering said BTK inhibitor to a patient,
(b) identifying that said patient is at risk of contracting an infection, and
(c) vaccinating said patient against said infection.

In another aspect, the present disclosure relates to the use of a BTK inhibitor for the manufacture of a medicament for the treatment of a disease, wherein said BTK inhibitor is administered as follows:
(a) administering said BTK inhibitor recurrently to a patient, thereby treating said patient for said disease,
(b) identifying that said patient is at risk of contracting an infection while on recurrent treatment with said BTK inhibitor, and
(c) vaccinating said patient against said infection while on recurrent treatment with said BTK inhibitor, and optionally
(d) continuing said recurrent treatment with said BTK inhibitor.

In another aspect, the present disclosure relates to the use of a BTK inhibitor for the manufacture of a medicament for the treatment of a disease, wherein said BTK inhibitor is administered as follows:
(a) administering said BTK inhibitor to a patient,
(b) vaccinating said patient against an infection, and
(c) continuing to administer said BTK inhibitor to said patient.

In another aspect, the present disclosure relates to the use of a BTK inhibitor for the manufacture of a medicament for the treatment of a disease, wherein said BTK inhibitor is administered by the following method:
(a) optionally administering said BTK inhibitor to a patient,
(b) identifying that said patient is at risk of contracting an infection,
(c) vaccinating said patient against said infection, and
(d) optionally administering said BTK inhibitor to said patient,
with the proviso that said method comprises one or more treatment periods in which said BTK inhibitor is administered.

In another aspect, the present disclosure relates to the use of a BTK inhibitor for the manufacture of a medicament for the treatment of a disease by a method wherein, while a patient having said disease is under ongoing treatment with said BTK inhibitor, said patient is vaccinated against an infection.

In another aspect, the present disclosure relates to the use of a vaccine for the manufacture of a medicament for the prevention of an infection by a method comprising:
(a) administering a BTK inhibitor to a subject, and
(b) vaccinating the subject against said infection.

In another aspect, the present disclosure relates to the use of a vaccine for the manufacture of a medicament for the prevention of an infection by a method comprising vaccinating said subject against said infection, wherein said subject is vaccinated while under ongoing treatment with a BTK inhibitor.

In another aspect, the present disclosure relates to the use of a vaccine for the manufacture of a medicament for the prevention of an infection by a method comprising:
(a) identifying a subject who is at risk of contracting an infection,
(b) vaccinating said subject who is at risk of contracting said infection with said vaccine against said infection, and
(c) administering a BTK inhibitor to said subject.

In another aspect, the present disclosure relates to the use of a vaccine for the manufacture of a medicament for the prevention of an infection by a method comprising:
(a) administering a BTK inhibitor to a subject,
(b) vaccinating said subject with said vaccine against said infection, and
(c) continuing to administer said BTK inhibitor to said subject.

### BRIEF DESCRIPTION OF THE FIGURES

In the following, reference is made to the figures.

FIG. 1 shows the study design of the study NCT02975336 (NCT number; EudraCT Number: 2016-002950-19) and relevant information about the study and vaccination post hoc analysis as carried out. *As available, before the vaccine (could be before treatment start); tAs indicated (not by trial design); $As available, 3-17 weeks after the vaccine. ACR, American College of Rheumatology; BID, twice daily; dsDNA, anti-double stranded DNA; HI, haemagglutination inhibition; QD, once daily; R, randomization; SLE, systemic lupus erythematosus; SLEDAI-2K, Systemic Lupus Erythematosus Disease Activity Index 2000; SLICC, Systemic Lupus International Collaborating Clinics.

FIGS. 2 and 3 summarize the baseline characteristics of the patients included in the post-hoc influenza vaccination analysis. BID, twice daily; BILAG, British Isles Lupus Assessment Group; QD, once daily; SD, standard deviation; SLE, systemic lupus erythematosus.

FIG. 4 provides an overview of patient vaccination in this study and indicates the proportion of subjects vaccinated using a specific strain category and type.

FIG. 5 summarizes the haemagglutination inhibition serum antibody titer measurements obtained in Example 1, for pre- and post-vaccination (GM Pre/Post) and the ratio of post-vaccination vs pre-vaccination titer levels (GM Ratio). BID, twice daily; GM, geometric mean; HI, haemagglutination inhibition; QD, once daily FIG. 6 is a depiction of the seroconversion rate data obtained for the different patient groups in the analysis. BID, twice daily; CI, confidence interval; QD, once daily.

FIG. 7 provides a tabular overview of the seroconversion rates determined in Example 1.

FIG. 8 shows the seroprotection rate data obtained for the different patient groups in the analysis. For each patient group, the pre-vaccination seroprotection rate value is depicted by the left bar, the post-vaccination value by the right bar. BID, twice daily; CI, confidence interval; QD, once daily.

FIG. 9 provides a tabular overview of the seroprotection rates determined in Example 1 (9A: Part 1; 9B: Part 2).

FIG. 10 shows the study design of the study NCT02975349 (NCT number; EudraCT Number: 2016-001448-21) and relevant information about the study and mRNA vaccine against COVID post-hoc analysis as carried out. *As available, prior to vaccine; ‡As indicated (not by trial design); § As available, at least 4 weeks after the second dose of vaccine. BID, twice daily; DBP, double-blind period; DMF, dimethyl fumarate; Evo, evobrutinib; OLE, open-label extension; QD, once daily; R, randomization.

FIG. 11 shows a box plot of the S1/S2 IgG antibody levels (measured as a single value) of pre- and post-vaccination plasma samples determined in Example 2 (for all patients who received two doses of an mRNA vaccine against COVID while on treatment with 75 mg evobrutinib twice daily).

FIG. 12 shows box plots for the S1/S2 IgG antibody levels of pre- and post-vaccination plasma samples determined in Example 2 for all patients who received two doses of an mRNA vaccine against COVID while on treatment with 75 mg evobrutinib twice daily, split up into (A) the sub-group of seronegative patients and (B) the sub-group of seropositive patients.

FIG. 13 shows a table summarizing the measurements of S1/S2 IgG antibody levels of pre- and post-vaccination samples determined for patients who received two doses of an mRNA vaccine against COVID while on treatment with 75 mg evobrutinib twice daily. SD, Standard deviation; CI, confidence interval; Q1, 25% quantile; Q3, 75% quantile; Min, minimum; Max, maximum.

FIG. 14 shows frequencies and summary statistics of fold changes of S1/S2 IgG antibody levels pre- and post-vaccination samples for patients who received two doses of an mRNA vaccine against COVID while on treatment with 75 mg evobrutinib twice daily. SD, Standard deviation; CI, confidence interval; Q1, 25% quantile; Q3, 75% quantile; Min, minimum; Max, maximum.

FIG. 15 shows a table summarizing the frequencies and summary statistics of the time between the second dose of mRNA vaccine against COVID while on treatment with 75 mg evobrutinib twice daily and the post-vaccination S1/S2 IgG antibody level assessment in this study. SD, Standard deviation; Q1, 25% quantile; Q3, 75% quantile; Min, minimum; Max, maximum.

FIG. 16 shows box plots for the S1/S2 IgG antibody levels of pre- and post-vaccination samples for patients who received two doses of an mRNA vaccine against COVID while on treatment split up into categories defined by the time between the second dose of mRNA COVID-19 vaccine and the post-vaccination S1/S2 IgG antibody levels assessment (all patients on evobrutinib 75 mg BID treatment). CI, confidence interval.

FIG. 17 shows a table summarizing the frequencies within different categories of fold changes of S1/S2 IgG antibody levels of pre- and post-vaccination samples in dependence of time between the second dose of mRNA vaccine against COVID and the post-vaccination S1/S2 IgG antibody level assessment (all patients on evobrutinib 75 mg BID treatment).

FIG. 18 shows a table summarizing S1/S2 IgG antibody levels (AU/ml) in pre- and post-vaccination samples and the fold-change for participants (N=45) in the VAC-OLE II study who received a complete cycle of an mRNA or a non-mRNA vaccine against COVID-19 while on treatment with 75 mg evobrutinib twice daily. a) Pre-vaccination timepoint sample collection date occurred before the first vaccination date. b) Post-vaccination timepoint sample collection date occurred≥complete vaccination cycle date plus 28 days. c) Fold change to post-vaccination is the ratio between the post-vaccination S1/S2 IgG antibody geometric mean and the pre-vaccination S1/S2 IgG antibody geometric mean. AU, arbitrary units; CI, confidence interval; Min, minimum; Max, maximum; SD, standard deviation.

FIG. 19 shows boxplots for the S1/S2 IgG antibody levels of pre-vaccination and post-vaccination timepoints for participants (N=45) in the VAC-OLE II study.

FIG. 20 shows a table summarizing the frequencies within different categories of fold changes of S1/S2 IgG antibody levels from pre-vaccination to post-vaccination for participants (N=45) in the VAC-OLE II study. The fold change to post-vaccination is the ratio between the post-vaccination S1/S2 IgG antibody geometric mean and the pre-vaccination S1/S2 IgG antibody geometric mean as determined for each individual participant.

FIG. 21 shows a table summarizing S1/S2 IgG antibody levels of pre-vaccination and post-vaccination samples, in participants in the VAC-OLE II study when sub-grouped based upon their pre-vaccination serostatus; seropositive=S1/S2 antibody levels ≥15 AU/mL and seronegative=S1/S2 antibody levels <15 AU/mL. a) Pre-vaccination timepoint sample collection date occurred before the first vaccination date. b) Post-vaccination timepoint sample collection date occurred≥complete vaccination cycle date plus 28 days. AU, arbitrary units; CI, confidence interval; Min, minimum; Max, maximum; SD, standard deviation.

FIG. 22 shows boxplots for the S1/S2 IgG antibody levels of pre-vaccination and post-vaccination timepoints for participants in the VAC-OLE II study sub-grouped based upon their pre-vaccination serostatus (seropositive at pre-vaccination N=13; seronegative at pre-vaccination N=32).

FIG. 23 shows a table summarizing the frequencies and summary statistics for participants sub-grouped based upon the elapse time between receiving a complete vaccination cycle of an mRNA or a non-mRNA COVID-19 vaccine and analysis of their post-vaccination sample (i.e. S1/S2 antibody levels (AU/mL) and fold change by time between completed vaccination cycle and post-vaccination sampling). a) Pre-vaccination timepoint sample collection date occurred before the first vaccination date. b) Post-vaccination timepoint sample collection date occurred≥complete vaccination cycle date plus 28 days. c) Fold change to post-vaccination is the ratio between the post-vaccination S1/S2 IgG antibody geometric mean and the pre-vaccination S1/S2 IgG antibody geometric mean. AU, arbitrary units; CI, confidence interval; Min, minimum; Max, maximum; SD, standard deviation.

FIG. 24 shows boxplots for the S1/S2 IgG antibody levels of pre-vaccination and post-vaccination timepoints for participants in the VAC-OLE II study sub-grouped based upon the elapse time between receiving a complete cycle of an mRNA or a non-mRNA COVID-19 vaccine and analysis of their post-vaccination sample.

FIG. 25 shows a table summarizing the level of fold changes in S1/S2 antibody levels in the VAC-OLE II study for participants sub-grouped based upon the elapse time between receiving a complete cycle of an mRNA or a non-mRNA COVID-19 vaccine and analysis of their post-vaccination sample. a) Fold change to post-vaccination is the ratio between the post-vaccination S1/S2 IgG antibody geometric mean and the pre-vaccination S1/S2 IgG antibody geometric mean as determined for each individual participant.

FIG. 26 shows boxplots for the S1/S2 IgG antibody levels at pre-vaccination, post-vaccination and post-booster vaccination timepoints in the sub-group of participants (N=14) that received booster vaccinations in the VAC-OLE II study.

FIG. 27 shows a table listing the S1/S2 IgG antibody levels in a sub-group of participants (N=14) in the VAC-OLE II study after receiving a booster vaccination, the fold-change from post-vaccination to post-booster and pre-vaccination to post-booster. a) Post-booster sample collection date occurred 7 or more days after the booster vaccination date. b) Fold change from post-vaccination to post-booster vaccination is the ratio between the post-booster vaccination S1/S2 IgG antibody geometric mean and the post-vaccination S1/S2 IgG antibody geometric mean. c) Fold change from pre-vaccination to the post-booster vaccination is the ratio between the post-booster vaccination S1/S2 IgG antibody geometric mean and the pre-vaccination S1/S2 IgG antibody geometric mean. d) Summary statistics are based on participants receiving a booster vaccine. AU, arbitrary units; CI, confidence interval; Min, minimum; Max, maximum; SD, standard deviation.

### DETAILED DESCRIPTION

Although the present disclosure is described in detail above and below, it is to be understood that this disclosure is not limited to the particular methodologies, protocols and reagents described by the present disclosure, as these may vary. It is also to be understood that the terminology used herein is for the purpose of describing particular embodiments only and is not intended to limit the scope of the present disclosure which will be limited only by the appended claims. Unless defined otherwise, all technical and scientific terms used herein have the same meanings as commonly understood by one of ordinary skill in the art.

In the following, certain elements of the present disclosure will be described in more detail, including the description of specific embodiments. However, the variously described examples and preferred embodiments should not be construed to limit the present disclosure to only the explicitly described embodiments. This description should be understood to support and encompass embodiments which combine the explicitly described embodiments with any number of the disclosed and/or preferred elements and in any manner. Furthermore, any permutations and combinations of all described elements in this application should be considered disclosed by the description of the present application except for where this leads to logical contradictions or the context indicates otherwise.

Unless defined otherwise herein, scientific and technical terms used in connection with the present disclosure shall have the meanings that are commonly understood by those of ordinary skill in the art. Generally, nomenclatures and techniques referred to in the present disclosure, e.g. nomenclatures and techniques of organic chemistry, chemical synthesis, biology, medicinal and pharmaceutical chemistry, medicine, pharmacology or toxicology, are those well-known and commonly used in the art. The methods and techniques of the present disclosure are generally performed according to conventional methods well-known in the art and as described in the references cited and discussed throughout the present disclosure unless otherwise indicated.

### Autoimmune Diseases

The immune system defends the body against infections. When the immune system malfunctions, it can attack healthy cells of the body. An autoimmune disease is a condition in which the body's immune system mistakes its own healthy tissues as foreign and attacks it causing inflammation that can affect many parts of the body depending on the particular type of autoimmune disease a person has.

An overview of autoimmune diseases, the current scientific understanding regarding the underlying mechanisms and details about their treatment in clinical practice are available from common textbooks in the field, such as “The Autoimmune Diseases Book” (2014), 5th edition, editors: Rose and Mackay, published by Elsevier Inc., ISBN: 978-0-12-384929-8, https://doi.org/10.1016/C2009-0-64586-4; or “Autoimmune Diseases, Acute and Complex Situations” (2011), 1st edition; editors: Khamashta and Ramos-Casals, published by Springer London, ISBN: 978-0-85729-358-9, https://doi.org/10.1007/978-0-85729-358-9.

There are ˜100 different types of autoimmune diseases. Some are more common, such as Type 1 diabetes, multiple sclerosis, lupus, and rheumatoid arthritis, while others are rare. For most autoimmune diseases there is no cure, with some of them requiring lifelong treatment to address the symptoms or prevent further damage of healthy tissues (Lleo et al., Autoimmunity Reviews (2010), vol. 9, p. A259-266).

Research into the treatment of autoimmune diseases has made great strides toward improving our understanding of disease mechanisms driving pathology. Dysregulation of B lymphocytes plays a major role in many of the rheumatic and neuroinflammatory diseases, but T cells and innate immune cells also play crucial roles in autoimmune diseases.

### Systemic Lupus Erythematosus (SLE)

SLE is a potentially fatal, chronic, multisystem autoimmune disorder that typically affects women between puberty and menopause. SLE can affect connective tissues, the joints, skin, brain, lungs, kidneys, and blood vessels. There is no cure for lupus and the disease severity can range from mild to life-threatening. The causes of SLE are unknown, but are believed to be a combination of environmental, genetic, and hormonal factors. Some of the strongest genetic links to SLE are the rare complement component C1Q and C4 single-gene defects. In most patients, SLE is a quantitative trait with several genes contributing to the risk of developing the disease; genome-wide association studies implicate several candidate loci including interferon (IFN) regulatory factor 5 (IRF5), mutations in which are associated with increases in the levels of the type 1 IFN family of molecules in patients with SLE. For review, see Kaul et al., Nature Reviews Disease Primers (2016), vol. 2(1), art. 16039.

### Rheumatoid Arthritis (RA)

RA is a chronic disease and the most common form of autoimmune arthritis. About 75% of RA patients are women with more than one million Americans affected. Although affected less, symptoms in men tend to be more severe. RA causes joint pain, stiffness, swelling and results in decreased movement of the joints and in long term damage of cartilage tissue and surrounding bones. Small joints in the hands and feet are most commonly affected in most cases, to a lesser degree other organs, such as eyes, skin or lungs can also be affected in some patients over time. What causes RA is unclear with infections, smoking and genetics being risk factors that can influence RA. Current treatments include medications, balanced exercise and targeted rest for the affected joint and surgery to address joint and bone damage (Bullock et al., Med. Princ. Pract. (2018), vol. 27, p. 501-507).

### Pemphigus Vulgaris (PV)

PV presents itself with blistering and sores of the skin and mucous membranes that are caused by autoantibodies against specific proteins in these tissues. The exact cause of the disease is unclear, but some medications including penicillamine, ACE (angiotensin converting enzyme) inhibitors and nonsteroidal anti-inflammatory drugs (NSAIDs) can be triggers of the disease. If untreated, PV can be dangerous, and in some cases complications including severe infections can be fatal. Treatment typically involves the use of corticosteroids to suppress the immune system, wound care similar to severe burns and treatments aimed to prevent infections (see Scully & Challacombe, Crit. Rev. Oral. Biol. Med. (2002), vol. 13, p. 397).

### Sjogren's Syndrome (SJ)

In Sjogren's syndrome, the body's immune cells attack glands in the body that produce tears, saliva and other body fluids. The amount of moisture produced by glands is reduced causing dryness of the mouth, eyes, nose, throat, and skin, but the disease can also affect other parts of the body, including joints, lungs, kidneys, blood vessels, digestive organs, and nerves. Most people affected by Sjogren's syndrome are women. The disease is sometimes linked to other autoimmune conditions such as rheumatoid arthritis and lupus. Treatments currently mostly address individual symptom relief depending on the tissues affected. Interventions may include eye drops that keep the eyes wet, sprays, medicated sweets and gels that keep the mouth wet, and medicine that support the body in producing more tears and saliva. For review, see Venables, Best. Pract. Res. Clin. Rheumatol. (2004), vol. 18(3), p. 313-329.

### Multiple Sclerosis (MS)

MS is a chronic, inflammatory, demyelinating and neuro-degenerative disease of the central nervous system (CNS). MS is a multifactorial, immune-mediated disease that is caused by complex gene-environment interactions. The pathological hallmark of MS is the accumulation of demyelinating lesions that occur in the white matter and the grey matter of the brain and spinal cord. The clinical manifestations and course of MS are heterogeneous; in most patients, reversible episodes of neurological deficits (known as relapses) that usually last for days or weeks characterize the initial phases of the disease (clinically isolated syndrome (CIS) and relapsing-remitting MS (RRMS)). Over time, the development of permanent neurological deficits and the progression of clinical disability become prominent (known as secondary progressive MS (SPMS)). A minority of patients have a progressive disease course from onset, which is referred to as primary progressive MS (PPMS)). Each subtype of MS can be classified as active or not active on the basis of clinical assessment of relapse occurrence or lesion activity detected using MRI; moreover, patients with PPMS or SPMS, can be classified according to whether disability has progressed over a given time. MS typically affects young adults, with an onset between 20 years and 40 years of age and has a higher prevalence in women, although some patients experience their initial demyelinating event during childhood or adolescence, typically with an RRMS (Filippi et al., Nature Reviews Disease Primers (2018), vol. 4, art. 43).

### Idiopathic Thrombocytopenia (ITP)

ITP is a hematological condition which is characterized by a low platelet count of less than 100,000 per microliter. This platelet deficit can be caused by decreased production, immune-mediated destruction or increased splenic sequestration of platelets, but typically involves autoantibodies to glycoproteins expressed on megakaryocytes, the precursor cells to platelets. Symptoms of ITP can vary, but tend to be symptoms of thrombocytopenia in general, such as petechiae, purpura, mucosal bleeding such as epistaxis, and in the most severe cases, fatal intracranial hemorrhage. ITP is idiopathic in 80% of cases, and primary ITP is often thought of as an autoimmune condition. However, in 20% of cases of ITP can arise secondary to coexisting illnesses. For example, ITP is often seen after infection. For review, see Samson et al., Cureus (2019), vol. 11(10), p. e5849.

### Chronic Urticaria (CU)

CU is characterized by wheals, angioedema or both for longer than 6 weeks. CU can be further divided into chronic spontaneous urticaria (CSU) and chronic inducible urticaria (CIndU). CSU is defined by the absence of a specific trigger to skin lesions, whereas in CIndU lesions are activated by a specific stimulus (e.g., symptomatic dermographism, cold urticaria, delayed pressure urticaria, solarurticaria, heat urticaria, vibratory angioedema, cholinergic urticaria, contact urticaria or aquagenic urticarial).

The underlying pathophysiology of CSU involves mast cell and basophil degranulation with release of histamine, leukotrienes, prostaglandins (PGs) and different inflammatory mediators. The wheal is characterized by edema, mast cell degranulation and a perivascular mixed infiltrate composed of predominantly CD4+ lymphocytes, monocytes, neutrophils, eosinophils and basophils, similar to allergen-mediated late-phase reactions. The cytokine profile is characterized by increases in interleukin (IL)-4, IL-5 and interferon-gamma, which is suggestive of a mixed type 1 T helper (Th1)/type 2 T helper (Th2) response (Choi et al., Immunol. Allergy Clin. North Am. (2010), vol. 30, p. 75-101; Min et al., Allergy Asthma Immunol. Res. (2019), vol. 11, p. 470).

### Myastenia Gravis (MG)

Myasthenia gravis (MG) is an autoimmune disease that affects the postsynaptic membrane at the neuromuscular junction. Its predominant manifestation is muscle weakness. Respiratory muscle weakness can occur infrequently, leading to a life-threatening condition that requires intensive care and respiratory support. However, with adequate treatment, most patients with MG are in a stable condition with only mild muscle weakness and are fully capable of their daily functions.

Eighty percent of patients with MG have detectable antibodies against the acetylcholine (ACh) receptor (AChR), whereas a small minority instead has antibodies against muscle-specific kinase (MuSK) or lipoprotein-receptor-related protein 4 (LRP4). The anti-LRP4 antibodies may be less MG-specific than those against AChR and MuSK, and this MG subgroup is less well established than the others. Antibodies are not detected in 10-15% of patients with generalized MG, usually because the sensitivity of the assay used is too low.

MG is classified into subgroups according to clinical manifestations, age at onset, the autoantibody pattern and thymus pathology. These subgroups reflect differences in epidemiology, disease mechanisms, severity and therapeutic response and help guide personalized treatment. Ocular MG and MG with anti-LRP4 antibodies tend to be milder, whereas MuSK MG and probably also thymoma MG tend to be more severe. The thymus has a key role in AChR-mediated MG, and thymectomy is a treatment option for patients with this subtype. MG is induced by a thymoma in 10% of patients, and thymectomy is a treatment option for patients with thymoma or thymic hyperplasia (Gilhus et al., Nature Reviews Disease Primers (2019), vol. 5, art. 30).

### Neuromyelitis Optica (NMO)/Neuromyelitis Optica Spectrum Disorders (NMOSD)

Neuromyelitis optica (NMO) is characterized by simultaneous or consecutive attacks of acute optic neuritis (ON) and transverse myelitis (TM). In ≥80% of cases, NMO is caused by pathogenetic IgG autoantibodies to aquaporin 4 (AQP4-IgG), the most abundant water channel protein in the central nervous system (CNS). Around 10-40% of individuals with NMO who lack AQP4-IgG have IgG autoantibodies to myelin oligodendrocyte glycoprotein (MOG-IgG); of note, MOG-IgG is also present in a subset of patients (mostly children) with acute disseminated encephalomyelitis (ADEM).

AQP4-IgG-positive NMO is primarily an autoimmune astrocytopathy, although secondary damage to oligodendrocytes and neurons occurs as a result of astrocyte dysfunction and loss and, probably, bystander inflammation. By contrast, primary demyelination is found in those with MOG-IgG. NMO has also been described in patients with sarcoidosis, infectious disease, connective tissue disorders (CTDs) and paraneoplastic neurological disorders, although cases are rare. In a small subgroup of patients, the cause remains unknown (known as idiopathic NMO).

AQP4-IgG-positive and MOG-IgG-positive NMO usually have a relapsing disease course with no major disease progression between attacks, although cases of monophasic MOG-IgG-positive disease have been reported. Untreated NMO can lead to severe, persisting visual and motor dysfunction owing to incomplete recovery from acute attacks (Jarius et al., Nature Reviews Disease Primers (2020), vol 6, art. 85).

In 2015 the word “spectrum disorder” was added to the formal name NMO, when physicians defined a broad continuum of clinical presentations as NMOSD that include spinal cord and/or optic nerve involvement as well as other indications, because they are all due to the same immunological mechanisms. NMOSD is a term used to encompass NMO (with both optic neuritis and myelitis) and limited phenotypes such as recurrent optic neuritis or myelitis. It is an autoimmune disorder, mediated in most cases by antibodies to the aquaporin-4 (AQP4) water channel and for the majority of patients serum AQP4 antibodies can be detected with an immunoassay. Making a definitive diagnosis of antibody-negative NMOSD can be challenging because the more prevalent relapsing-remitting multiple sclerosis (RRMS) can present similarly. For review, see Matthews et al., Neurology (2013), vol. 80, p. 1330.

In 2015 the word spectrum disorder was added to the formal name NMO when physicians defined a broad continuum of clinical presentations as NMOSD that include spinal cord and/or optic nerve involvement as well as other indications, because they are all due to the same immunological mechanisms.

### Other Inflammatory Diseases

There are further inflammatory diseases that, even though not autoimmune diseases, show similarities to autoimmune diseases with regard to misdirected inflammation and their clinical manifestations and underlying inflammatory reactions. Examples of such inflammatory diseases that are particularly relevant with regard to the present disclosure are discussed below.

### Large-Vessel Vasculitis (LVV)

LVV manifests as inflammation of the aorta and its major branches and is the most common primary vasculitis in adults. LVV comprises two distinct conditions, giant cell arteritis (GCA) and Takayasu arteritis (TAK), although the phenotypic spectrum of primary LVV is complex. Non-specific symptoms often predominate and so patients with LVV present to a range of health-care providers and settings. Rapid diagnosis, specialist referral and early treatment are key to good patient outcomes. Unfortunately, disease relapse remains common and chronic vascular complications are a source of considerable morbidity. Although accurate monitoring of disease activity is challenging, progress in vascular imaging techniques and the measurement of laboratory biomarkers may facilitate better matching of treatment intensity with disease activity.

GCA is an idiopathic inflammatory condition characterized by granulomatous arteritis in temporal artery biopsy (TAB) specimens. It is now recognized that GCA encompasses a broad phenotypic spectrum of medium and large artery inflammation. Nomenclature has evolved to reflect this, with the terms large-vessel GCA (LV-GCA), cranial GCA (C-GCA) and LV-GCA with cranial involvement now suggested depending on the site of inflammation.

TAK was first described in 1908 as a series of retinal vascular abnormalities. Its association with absent or diminished peripheral pulses led to the term ‘pulseless disease’, and autopsy studies demonstrated a pan-arteritis involving the aorta and its major branches. Although early descriptions of the disease involved individuals of Japanese origin, TAK is now recognized to occur worldwide. Further, advances in our understanding of disease pathophysiology have paved the way for novel biologic treatments that target important mediators of disease in both giant cell arteritis and Takayasu arteritis (Pugh et al., Nature Reviews Disease Primers (2021), vol. 7(1), art. 93).

### Graft Versus Host Disease (GVHD)

Allogeneic hematopoietic cell transplantation (HCT) is an important therapeutic option for a variety of malignant and nonmalignant conditions. As the number of allogeneic HCT continues to increase, greater attention to improvements in supportive care, infectious prophylaxis, immunosuppressive medications and DNA-based tissue typing are continuously being made. However, graft versus host disease (GVHD) remains the most frequent and serious complication following allogeneic HCT.

GVHD has an acute form and a chronic form. Acute GVHD is characterized by damage to the skin, liver and the gastrointestinal tract, whereas chronic GVHD has more diverse manifestations and can resemble autoimmune syndromes with, for example, eosinophilic fasciitis, scleroderma-like skin disease and salivary and lacrimal gland involvement. For the purpose of epidemiological studies, acute GVHD occurs within the first 100 days after transplantation, and chronic GVHD occurs after day 100 post-transplantation. However, it is now recognized that clinical features of chronic GVHD can occur within 100 days of transplant and that features of acute GVHD and chronic GVHD may coexist, leading to new definitions in which diagnosis focuses on the constellation of symptoms rather than their time of onset (Choi et al., Immunol. Allergy Clin. North Am. (2010), vol. 30(1), p. 75-101).

### Asthma

Asthma is as a heterogeneous disease characterized by chronic airway inflammation and variable remodelling that results in a range of clinical presentations, treatment responses and natural history across the life course of the patient. Asthma involves a history of respiratory symptoms including wheeze, shortness of breath, chest tightness and cough that vary over time and in intensity, as well as variable expiratory airflow limitation and airway hyper-responsiveness to a range of stimuli, such as exercise and inhaled irritants. At the population level, a subset of individuals with asthma exhibit an accelerated decline in lung function over their life time, which, in severe chronic disease, manifests as fixed airflow obstruction. This decline is especially prominent in late-onset asthma. The origin and severity of asthma are driven by strong genetic and environmental factors. Although most cases of asthma begin in childhood in association with IgE-dependent sensitization to common environmental allergens, asthma can also emerge later in life. Adult-onset asthma often occurs in the absence of allergy, but can be accompanied by intolerance to NSAIDs, rhinosinusitis and nasal polyps. Intolerance to NSAIDs most likely results from reduced production of the anti-bronchoconstrictor prostaglandin E2 under conditions of inflammation. For review, see Holgate et al., Nature Reviews Disease Primers (2015), vol. 25(1), art. 15025.

In clinical practice, there is a considerable need for medical treatments for autoimmune diseases or other inflammatory diseases with immunosuppressive or immunomodulatory agents. At the same time, however, such patients are in danger due to infections, especially viral and/or bacterial infections.

### Bacterial Infections

Multiple types of bacteria, especially multiple types of pathogenic bacteria, that potentially can infect, more specifically pathologically infect, one or more host species, including warm-blooded animals, mammals and humans, are known. Generally, an infection by (pathologic) bacteria often leads to bacterial disease or infectious bacterial disease in said host, when an organism's body (i.e. the host's body) is invaded by one or more of said pathogenic bacteria, and the host body's cannot control or not sufficiently control the replication and spread of said pathogenic bacteria within the host's body, or parts or organs thereof, and thus cause a bacterial disease or disorder.

Other bacteria are characterized as opportunistic pathogens and cause disease mainly in people suffering from immunosuppression or cystic fibrosis. Examples of these opportunistic pathogens include Pseudomonas aeruginosa (causing diseases such as hospital-acquired infections, ventilator-associated pneumonia, a type of lung infection that occurs in people who are on mechanical ventilation breathing machines in hospitals, and various sepsis syndromes), Burkholderia cenocepacia, and Mycobacterium avium (found in fresh and salt water, in household dust and in soil, is causing a disease in humans called Mycobacterium avium-intracellular infection or Mycobacterium avium complex infection, normally only in subjects who are immunocompromised or those with severe lung disease.

Preferably, bacterial infections, diseases or disorders include, but are preferably not limited to, Anthrax, Cholera, Diphtheria, Haemophilus influenzae, Meningococcal Meningitis, Pertussis, Plague, Pneumococcal Disease, Streptococcus pneumoniae, Tetanus, Tuberculosis, and Typhus.

Antibacterial vaccines that may be used in the present disclosure include, without limitation, one or more of the below given vaccines, but are preferably not limited to below given vaccines: pneumococcal 23-polyvalent vaccine, e.g. Pneumovax 23 or Pneumovax 23 (Pro), pneumococcal 13-valent vaccine, e.g. Prevnar 13 or Prevnar 13 (Pro), meningococcal conjugate vaccine, e.g. Menactra or Menactra (Pro), Haemophilus b conjugate (prp-t) vaccine, e.g. ActHIB or ActHIB (Pro), meningococcal group B vaccine, e.g. Bexsero or Bexsero (Pro), anthrax vaccine adsorbed, e.g. Biothrax or Biothrax (Pro), Haemophilus b conjugate (prp-t) vaccine, e.g. Hiberix or Hiberix (Pro), Haemophilus b conjugate (prp-omp) vaccine, e.g. Liquid PedvaxHIB or Liquid PedvaxHIB (Pro), Haemophilus b conjugate (prp-t) vaccine/meningococcal conjugate vaccine, e.g. MenHibrix, meningococcal polysaccharide vaccine, e.g. Menomune A/C/Y/W-135, meningococcal conjugate vaccine, e.g. Menveo or Menveo (Pro), pneumococcal 7-valent vaccine, e.g. Prevnar or Prevnar (Pro), tetanus toxoid, e.g. Te Anatoxal Berna, tetanus toxoid, e.g. Tetanus Toxoid Adsorbed, meningococcal group B vaccine, e.g. Trumenba or Trumenba (Pro) typhoid vaccine, inactivated, e.g. Typhim Vi or Typhim Vi (Pro) cholera vaccine, live, e.g. Vaxchora or Vaxchora (Pro), and/or typhoid vaccine, live, e.g. Vivotif Berna.

### Viral Infections

Multiple types of viruses that potentially can infect, more specifically pathologically infect, one or more host species, including warm-blooded animals, mammals and humans, are known. Generally, a viral infection, often leading to a viral disease or infectious viral disease, in said host occurs when an organism's body (i.e. the host's body) is invaded by pathogenic viruses, and infectious virus particulates (such as virions) attach to and enter susceptible cells of said body of said host or subject.

Basic structural characteristics, such as genome type, virion shape and replication site, generally share the same features among virus species within the same family:
Double-stranded DNA families: three are non-enveloped (Adenoviridae, Papillomaviridae and Polyomaviridae) and two are enveloped (Herpesviridae and Poxviridae). All of the non-enveloped families have icosahedral capsids.
Partly double-stranded DNA viruses: Hepadnaviridae. These viruses are enveloped.
One family of single-stranded DNA viruses infects humans: Parvoviridae. These viruses are non-enveloped.
Positive single-stranded RNA families: three non-enveloped (Astroviridae, Caliciviridae and Picornaviridae) and four enveloped (Coronaviridae, Flaviviridae, Retroviridae and Togaviridae). All the non-enveloped families have icosahedral nucleocapsids.
Negative single-stranded RNA families: Arenaviridae, Bunyaviridae, Filoviridae, Orthomyxoviridae, Paramyxoviridae and Rhabdoviridae. All are enveloped with helical nucleocapsids.
Double-stranded RNA genome: Reoviridae.
The Hepatitis D virus has not yet been assigned to a family, but is clearly distinct from the other families infecting humans.
Viruses known to infect humans that have not been associated with disease: the family Anelloviridae and the genus Dependovirus. Both of these taxa are non-enveloped single-stranded DNA viruses.

Human-infecting virus families offer rules that may assist physicians and medical microbiologists/virologists.

As a general rule, DNA viruses replicate within the cell nucleus while RNA viruses replicate within the cytoplasm. Exceptions are known to this rule: poxviruses replicate within the cytoplasm and orthomyxoviruses and hepatitis D virus (RNA viruses) replicate within the nucleus.

Some types of viruses have segmented genomes, such as Bunyaviridae, Orthomyxoviridae, Arenaviridae, and Reoviridae (acronym BOAR). All of these are RNA viruses.

Some viruses are transmitted almost exclusively by arthropods: Bunyavirus, Flavivirus, and Togavirus. Some Reoviruses are transmitted from arthropod vectors. All of these are RNA viruses.

One family of enveloped viruses causes gastroenteritis (Coronaviridae). All other viruses associated with gastroenteritis are non-enveloped.

Table 1 below summarizes some of the most clinically important pathogenic viruses that are known to afflict a plurality of hosts, including humans, and the type, family, exemplary transmission routes and infections/diseases commonly attributed to the respective viruses. However, the clinical characteristics of viruses may differ substantially among species within the same family.

TABLE 1

| Type | Family | Transmission | Infections/Diseases |
|---|---|---|---|
| Adenovirus | Adenoviridae | droplet contact | gastroenteritis |
| | | fecal-oral | keratoconjunctivitis |
| | | venereal | pharyngitis |
| | | direct eye | croup |
| | | contact | pharyngoconjunctival fever |
| | | | pneumonia |
| | | | pneumonia |
| | | | cystitis |
| Coxsackievirus | Picornaviridae | fecal-oral | Hand, foot and mouth disease |
| | | respiratory | pleurodynia |
| | | droplet contact | aseptic meningitis |
| | | | pericarditis |
| | | | myocarditis |
| Epstein-Barr | Herpesviridae | saliva | infectious mononucleosis |
| virus | | | Burkitt's lymphoma |
| | | | Hodgkin's lymphoma |
| | | | nasopharyngeal carcinoma |
| Hepatitis A | Picornaviridae | fecal-oral | acute hepatitis |
| virus | | | |
| Hepatitis B | Hepadnaviridae | bodily fluids | acute hepatitis |
| virus | | Vertical and | chronic hepatitis |
| | | sexual | hepatic cirrhosis |
| | | | hepatocellular carcinoma |
| Hepatitis C | Flaviviridae | blood | acute hepatitis |
| virus | | sexual contact | chronic hepatitis |
| | | | hepatic cirrhosis |
| | | | hepatocellular carcinoma |
| Herpes | Herpesviridae | direct contact | herpes labialis, cold sores - |
| simplex | | saliva | can recur by latency |
| virus, type 1 | | | gingivostomatitis in children |
| | | | tonsillitis & pharyngitis |
| | | | in adults |
| | | | keratoconjunctivitis |
| Herpes | Herpesviridae | sexual contact | Skin vesicles, mucosal ulcers, |
| simplex | | vertical | Oral and/or genital |
| virus, type 2 | | transmission | Can be latent |
| | | | Aseptic meningitis |
| Cytomegalovirus | Herpesviridae | vertical | infectious mononucleosis |
| | | transmission | Cytomegalic inclusion disease |
| | | bodily fluids | Premature birth |
| | | | liver, lung and spleen diseases |
| | | | in the newborn |
| | | | Small size at birth |
| | | | Small head size |
| | | | congenital seizures in the newborn |
| Human | Herpesviridae | Saliva | Kaposi sarcoma |
| herpesvirus, | | Sexual | multicentric Castleman disease |
| type 8 | | | primary effusion lymphoma |
| HIV | Retroviridae | sexual contact | AIDS |
| | | blood | |
| | | breast milk | |
| | | vertical | |
| | | transmission | |
| Influenza | Orthomyxoviridae | droplet contact | influenza |
| virus | | | (Reye syndrome) |
| Measles virus | Paramyxoviridae | droplet contact | measles |
| | | | postinfectious encephalomyelitis |
| Mumps virus | Paramyxoviridae | droplet contact | mumps |
| Human | Papillomaviridae | direct contact | hyperplastic epithelial |
| papillomavirus | | sexual contact | lesions (common, flat, plantar |
| | | vertical | and anogenital warts, laryngeal |
| | | transmission | papillomas, epidermodysplasia |
| | | | verruciformis) |
| | | | Malignancies for some species |
| | | | (cervical carcinoma, squamous |
| | | | cell carcinomas) |
| Parainfluenza | Paramyxoviridae | droplet contact | croup |
| virus | | | pneumonia |
| | | | bronchiolitis |
| | | | common cold |
| Poliovirus | Picornaviridae | fecal-oral | poliomyelitis |
| Rabies virus | Rhabdoviridae | animal bite | rabies (fatal encephalitis) |
| | | droplet contact | |
| Respiratory | Paramyxoviridae | droplet contact | bronchiolitis |
| syncytial | | hand to mouth | pneumonia |
| virus | | | influenza-like syndrome |
| | | | severe bronchiolitis with |
| | | | pneumonia |
| Rubella virus | Togaviridae | Respiratory droplet | congenital rubella |
| | | contact | German measles |
| Varicella- | Herpesviridae | droplet contact | chickenpox |
| zoster virus | | direct contact | herpes zoster |
| | | | Congenital varicella syndrome |
| SARS-CoV-2 | Coronaviridae | droplet contact | COVID-19 |
| | | fecal-oral | |

Preferably, all of the above given viruses and/or viral disorders are to be regarded as clinically relevant viruses and/or viral disorders in the context of the uses according to the instant invention and/or methods of treatment according to the instant invention. More preferably, e.g. in MS, a number of vaccine-preventable infectious diseases are relevant as the risk of infection or poor outcomes is increased by the MS itself or its therapy. These primarily include hepatitis B virus (HBV) and varicella zoster virus (VZV). Other infectious diseases might include Measles, Influenza, Poliovirus, Pneumococci, Diphtheria, Tetanus and Bordetella pertussis, COVID-2 and/or COVID-19. Vaccines against COVID-2 and/or COVID-19 are now available, with further alternatives to become available soon.

Viral infections and/or the resulting diseases can typically be detected by clinical presentation, signs and/or symptoms, including, but not limited to severe muscle and joint pains preceding fever, or skin rash and swollen lymph glands. Standard laboratory investigation may help detecting viral infections. Laboratory investigation may also be useful in diagnosing bacterial infections associated with a viral infection. Viral infections are commonly of limited duration, and treatment according to the prior art typically consists of reducing the symptoms, and antipyretic and analgesic drugs are commonly prescribed.

However, the best protection of subjects against the potentially serious and harmful outcome of infections is the prevention of amelioration of the infection as such. The most advantageous way of preventing or ameliorating inventions is currently regarded the vaccination of subjects that are endangered by said infections.

However, precautionary measures against infections, like vaccinations, generally require a proper immune response of the respective body and are thus generally recommended to be done well in advance of serious medical interventions, i.e. surgery, chemotherapy, radiotherapy or of the start of an immunosuppressive treatment, and/or well after the end of the respective serious medical intervention.

Accordingly, precautionary measures against infections, like vaccinations, which require a proper immune response of the respective body are generally recommended to be done well in advance of the start of the respective immunosuppressive treatment, and/or well after the end of the respective immunosuppressive treatment, if any.

In this context, a vaccination is preferably the administration of a vaccine to help the immune system develop protection from a disease, preferably an infectious disease. Vaccines typically contain the pathogen of the respective disease, preferably a microorganism or virus, in a weakened, live or killed/dead state, or proteins or toxins from the respective organism. In stimulating the body's adaptive immunity, they help prevent sickness from an infectious disease. Vaccination is the most effective method of preventing infectious diseases, thus preventing and infectious diseases in people with reduced immunity due to a immunosuppressive treatment are generally regarded as important and advantageous/desirous.

Infectious disorders for which vaccines are available or at least desirous, include, but are not limited to influenza and influenza-like diseases, such as Parainfluenza virus (causing croup, pneumonia, bronchiolitis and/or common cold), and coronavirus-induced infections.

Antiviral vaccines that may be used in the present disclosure include, without limitation, one or more of the below given vaccines, but are preferably not limited to below given vaccines: influenza virus vaccine, inactivated, e.g. Afluria or Afluria (Pro), influenza virus vaccine, inactivated, e.g. Fluarix Quadrivalent or Fluarix Quadrivalent (Pro), influenza virus vaccine, inactivated, e.g. Flublok Quadrivalent or Flublok Quadrivalent (Pro), influenza virus vaccine, inactivated, e.g. Fluvirin or Fluvirin (Pro), hepatitis b adult vaccine, e.g. Engerix-B, influenza virus vaccine, inactivated, e.g. Flublok or Flublok (Pro), zoster vaccine live, e.g. Zostavax or Zostavax (Pro), human papillomavirus vaccine, e.g. Gardasil 9 or Gardasil 9 (Pro), influenza virus vaccine, inactivated, e.g. Flucelvax Quadrivalent or Flucelvax Quadrivalent (Pro), zoster vaccine, inactivated, e.g. Shingrix or Shingrix (Pro), influenza virus vaccine, live, trivalent, e.g. FluMist or FluMist (Pro), influenza virus vaccine, inactivated, e.g. Fluzone, influenza virus vaccine, inactivated, e.g. Fluzone High-Dose or Fluzone High-Dose (Pro), influenza virus vaccine, inactivated, e.g. Fluad or Fluad (Pro), influenza virus vaccine, live, trivalent, e.g. FluMist Quadrivalent, yellow fever vaccine, e.g. Stamaril or Stamaril (Pro), smallpox vaccine, e.g. ACAM2000 or ACAM2000 (Pro), influenza virus vaccine, inactivated, e.g. Afluria Quadrivalent or Afluria Quadrivalent (Pro), influenza virus vaccine, inactivated, e.g. Agriflu, measles virus vaccine, e.g. Attenuvax, human papillomavirus vaccine, e.g. Cervarix or Cervarix (Pro), smallpox vaccine, e.g. Dryvax, hepatitis b pediatric vaccine, e.g. Engerix-B Pediatric, influenza virus vaccine, inactivated, e.g. Fluarix or Fluarix (Pro), influenza virus vaccine, inactivated, e.g. Flucelvax, influenza virus vaccine, inactivated, e.g. FluLaval or FluLaval (Pro), influenza virus vaccine, inactivated, e.g. FluLaval Preservative-Free Quadrivalent, influenza virus vaccine, inactivated, e.g. FluLaval Quadrivalent or FluLaval Quadrivalent (Pro), influenza virus vaccine, inactivated, e.g. Fluzone High-Dose Quadrivalent, influenza virus vaccine, inactivated, e.g. Fluzone Intradermal Quadrivalent, influenza virus vaccine, inactivated, e.g. Fluzone Quadrivalent or Fluzone Quadrivalent (Pro), hepatitis a adult vaccine, e.g. Havrix or Havrix (Pro), hepatitis a pediatric vaccine, e.g. Havrix Pediatric, rabies vaccine, human diploid cell, e.g. Imovax Rabies or Imovax Rabies (Pro), poliovirus vaccine, inactivated, e.g. Ipol or Ipol (Pro), japanese enceph vacc sa14-14-2, inactivated, e.g. Ixiaro or Ixiaro (Pro), rubella virus vaccine, e.g. Meruvax II, mumps virus vaccine, e.g. Mumpsvax, rabies vaccine, purified chick embryo cell, e.g. RabAvert or RabAvert (Pro), hepatitis b adult vaccine, e.g. Recombivax HB Adult, hepatitis b adult vaccine, e.g. Recombivax HB Dialysis Formulation, hepatitis b pediatric vaccine, e.g. Recombivax HB Pediatric/Adolescent, rotavirus vaccine, e.g. Rotarix or Rotarix (Pro), rotavirus vaccine, e.g. RotaTeq or RotaTeq (Pro), hepatitis a adult vaccine, e.g. Vaqta or Vaqta (Pro), hepatitis a pediatric vaccine, e.g. Vaqta Pediatric, varicella virus vaccine, e.g. Varivax or Varivax (Pro), and/or yellow fever vaccine, e.g. YF-Vax or YF-Vax (Pro).

### Coronavirus

Coronavirus-induced infections include the epidemics or pandemics caused by SARS-CoV, HCoV NL63, HCoV HKU1, MERS-CoV, and SARS-CoV-2/COVID-19, and/or its mutants or successors.

Coronaviruses preferably constitute the subfamily Orthocoronavirinae, in the family Coronaviridae, order Nidovirales, and realm Riboviria. They are regarded as enveloped viruses with a positive-sense single-stranded RNA genome and/or a nucleocapsid of helical symmetry. The latter is typically wrapped in a icosahedral protein shell. The genome size of coronaviruses is believed to range from approximately 26 to 32 kilobases, one of the largest among RNA viruses. Moreover, they typically have characteristic club-shaped spikes that project from their surface, which in electron micrographs create an image reminiscent of the solar corona, from which their name is believed to be derived.

Human coronaviruses were discovered in the 1960s, including viruses tagged B814, 229E, IBV (infectious bronchitis virus), and OC43. This new group of viruses came to be known as coronaviruses after their distinctive morphological appearance. Since then, a plurality of other human coronaviruses have been identified, including SARS-CoV in 2003, HCoV NL63 in 2004, HCoV HKU1 in 2005, MERS-CoV in 2012, and SARS-CoV-2 in 2019. However, there have also been a large number of animal coronaviruses identified since the 1960s.

The COVID-19 pandemic has caused unprecedented disruption to normal social and economic life worldwide. As of June 2022, over 500 million cases have occurred worldwide with more than 6 million deaths (https://covid19.who.int/). The disease is caused by SARS-CoV-2, a novel coronavirus, which infects cells via the angiotensin-converting enzyme receptor type 2 expressed on cells of the respiratory tract and also key tissues such as the brain.

Vaccines against COVID-19 and/or mutants thereof are highly desirable for use according to the instant disclosure. Several vaccines directed against the virus that causes COVID-19 (i.e. SARS-COV-2) are now available and further vaccines are under development close to Health Authority approval. Actual and/or candidate approaches include subunit vaccines containing viral proteins, particularly the surface-membrane spike protein that mediates receptor attachment; nucleic acid-based vaccines that enable host cells to produce the viral antigen i.e. the spike protein or its immunogenic portions; and whole-microbe approach such as live—attenuated vaccine, inactivated vaccine or viral vector vaccine.

Especially preferred for use as a vaccine in the context of the present invention are Corona vaccines, in particular COVID-19 vaccines, including, but not limited to
mRNA-technology based vaccines, such as:
   mRNA-1273 (Moderna/NIAID vaccine),
   BNT162b2 (Pfizer/BioNTech vaccine);
Vector-based vaccines, preferably Adenovirus/Vector-based vaccines, such as:
   Ad26.COV2.S (Johnson & Johnson/Janssen Pharmaceuticals vaccine),
   AZD1222 (ChAdOx1-S) (AstraZeneca/University of Oxford vaccine),
   Gam-COVID-Vac/Sputnik V (Gamaleya Institute),
   Ad5-nCoV (CanSino Biologics);
Virus-Like Particles (VLPs) based vaccines, such as:
   CVnCoV (CureVac),
   CoVLP (Medicago);
DNA-based vaccines, such as:
   INO-4800 (Inovio Pharmaceuticals/International Vaccine Institute);
Protein-based vaccines, such as:
   ZF2001 (Anhui Zhifei Longcom Biopharmaceutical), preferably plus adjuvants,
   NVX-CoV2373 (Novavax), preferably plus adjuvants;
Inactivated Virus-based or Dead Virus-based vaccines, such as:
Covaxin/BBV152 (Bharat Biotech/Indian Council of Medical Research (ICMR)/National Institute for Virology (NIV),
   CoronaVac (Sinovac),
   Sinopharm/Wuhan Institute of Biological Products,
   BBIBP-CorV (Sinopharm/Beijing Institute of Biological Products);
   and the like.

The same preferably applies to further Corona vaccines, in particular COVID-19 vaccines.

mRNA-based COVID-19 vaccines (mRNA vaccines against COVID-19), such as the mRNA vaccine against COVID-19 of BioNTech/Pfizer and of Moderna are especially preferred. mRNA-based vaccines contain mRNA that is introduced into cells in the body of the vaccinated individual and instructs these cells to make a protein (or even just a piece of a protein) that triggers an immune response inside the body.

In contrast, non-mRNA vaccines do not contain such mRNA and instead may for example contain a viral vector (which causes production of a protein or piece of a protein inside the cells of the vaccinated individual) or a protein or protein subunit. The protein, in turn, causes, possibly in collaboration with adjuvants, an immune response inside the body of the vaccinated individual.

Examples for mRNA vaccines are the mRNA-based COVID-19 vaccines Comirnaty® by BioNTech/Pfizer or Spikevax® by Moderna. Examples for non-mRNA vaccines are the non-mRNA COVID-19 vaccines Vaxzevria®/Covishield® (AstraZeneca) or Jcovden® (Janssen/Johnson & Johnson).

### Influenza

Influenza, commonly known as “the flu”, is an infectious disease caused by an influenza virus. Symptoms can be mild to severe. The most common symptoms include: high fever, runny nose, sore throat, muscle and joint pain, headache, coughing, and feeling tired. These symptoms typically begin two days after exposure to the virus and most last less than a week. The cough, however, may last for more than two weeks. In children, there may be diarrhea and vomiting, but these are not common in adults. Complications of influenza may include viral pneumonia, secondary bacterial pneumonia, sinus infections, and worsening of previous health problems such as asthma or heart failure.

There are four types of influenza virus: type A, B, C and D. Typically, three of the four types affect humans, namely the types A, B, and C. Type D has not been known to infect humans to date, but is believed to have the potential to do so. Only influenza A and B cause seasonal epidemics of respiratory illness that occur every year. Influenza A viruses are further divided into different subgroups based on two proteins found on the surface of the virus—hemagglutinin (HA) and Neuaminidase (NA). There are 18 different subtypes of HA (H1-18) and 11 different subtypes of NA (N1-11).

Each year the annual flu vaccine protects against 3 or 4 strains (the number depending upon what research predicts will be most common for the upcoming flu season) of flu. H1N1, H1N2, and H3N2 are the only known Influenza A virus subtypes currently circulating among humans and are included in the trivalent vaccines, while an extra influenza B strain is included in the quadrivalent vaccine.

H1N1 flu is also known as swine flu. The term swine flu derives from the past, when the people who caught it had direct contact with pigs. That changed several years ago, when a new virus emerged that spread among people who had not been near pigs. In 2009, HINI was spreading fast around the world, so the World Health Organization called it a pandemic.

The H3N2 virus continues to circulate worldwide as a seasonal influenza A virus. Seasonal H3N2 viruses, which are associated with severe illness in older people, undergo regular antigenic drift. In years in which H3N2 is the predominant strain, there are more hospitalizations, particular among high risk groups such as older adults and younger children.

The trivalent 2018-2019 northern hemisphere influenza vaccines contained: A/Michigan/45/2015-like (H1N1) pdm09 virus, A/Singapore/INFIMH-16-0019/2016 A(H3N2)-like virus and a B/Colorado/06/2017-like Victoria lineage virus. Quadrivalent vaccines contained an additional B/Phuket/3073/2013-like Yamagata lineage virus. Genetic characterization of viruses indicated that the majority of circulating A (H3N2) viruses in the US were antigenically distinct, or drifted from the 2018-19 Northern hemisphere vaccine strain, while the circulating A(H1N1)pdm9 viruses were well matched.

Influenza B virus is known only to infect humans and seals. This limited host range is apparently responsible for the lack of associated influenza pandemics in contrast with those caused by the morphologically similar influenza A virus as both mutate by both antigenic drift and reassortment. There are two known circulating lineages of Influenza B virus based on the antigenic properties of the surface glycoprotein hemagglutinin. The lineages are termed B/Yamagata/16/88-like and B/Victoria/2/87-like viruses. The quadrivalent influenza vaccine licensed by the CDC is currently designed to protect against both co-circulating lineages and has been shown to have greater effectiveness in prevention of influenza caused by Influenza B virus than the previous trivalent vaccine. Further diminishing the impact of this virus, “in humans, influenza B viruses evolve slower than A viruses and faster than C viruses”. Influenza virus B mutates at a rate 2 to 3 times slower than type A.

Injected trivalent inactivated influenza vaccines are most commonly used throughout the world.

Quadrivalent inactivated influenza vaccines available from the 2014/2015 season, were used in the Magnify study.

In 2011, a live attenuated influenza vaccine (LAIV) for intranasal use was approved in the EU/EEA for children and adolescents (2-17 years of age). All live attenuated influenza vaccines currently available are quadrivalent combination vaccines containing two influenza A strains (H1N1 and H3N2 subtypes) and two influenza B strains (Victoria and Yamagata lineages) as per WHO recommendations.

Since seasonal influenza vaccines are usually recommended for several vulnerable populations who are poorer immune responders due to age or disease, several attempts to improve the vaccines have been explored over the last 10-15 years such as: increasing the antigen dose administered, intradermal administration to activate other arms of the immune system, and adding immunostimulating compounds such as adjuvants.

Products utilizing these new techniques are now authorized and available in some EU/EEA countries. See table below. For human influenza vaccines, MF59 (squalene) and AS03 (squalene and α-tocopherol) have been approved as adjuvants by regulatory agencies in the EU, Canada and the United States. Data from several observational studies indicating improved performance and reductions in hospitalizations in older adults against influenza disease and hospitalizations have been included in the label for the adjuvanted trivalent influenza vaccine. Most influenza vaccines, both inactivated and live attenuated, are based on production of influenza viruses/antigens in fertilized hens' eggs. These vaccines can therefore not be given to egg-allergic individuals developing severe symptoms upon exposure to egg proteins. Hence, a few manufacturers have developed cell-based influenza vaccines.

Influenza A virus subtype H1N2 (A/H1N2) is a subtype of the species Influenza A virus (sometimes called bird flu virus). It is currently endemic in both human and pig populations. H1N1, H1N2, and H3N2 are the only known Influenza A virus subtypes currently circulating among humans. The virus generally does not cause more severe illness than other influenza viruses, and no unusual increases in influenza activity have been associated with it.

The H3N2 virus continues to circulate worldwide as a seasonal influenza A virus. Seasonal H3N2 viruses, which are associated with severe illness in older people, undergo regular antigenic drift.

Injected trivalent inactivated influenza vaccines have in the past been most commonly used throughout the world. Influenza antigen preparation varies between manufacturers. The inactivated influenza vaccines available in the EU/EEA may contain either split virion influenza virus products or subunit influenza products. Adjuvanted inactivated subunit influenza vaccines for older people are available in some EU/EEA Member States.

Injected quadrivalent inactivated influenza vaccines, available from the 2014/2015 season in some EU/EEA countries, are expected to replace the trivalent vaccines over time. Although available in the private market in some European countries, vaccine authorization, vaccine availability, observed vaccine effectiveness and the cost may influence the speed of this replacement.

In 2011, a live attenuated influenza vaccine (LAIV) for intranasal use was approved in the EU/EEA for children and adolescents (2-17 years of age). All live attenuated influenza vaccines currently available are quadrivalent combination vaccines containing two influenza A strains (H1N1 and H3N2 subtypes) and two influenza B strains (Victoria and Yamagata lineages) as per WHO recommendations.

The universal paediatric programmes organised in Finland and the United Kingdom offer quadrivalent LAIV to either young children 2-3 or 2-11 years of age, respectively.

TABLE 2
Overview of available seasonal influenza vaccines in the EU/EEA (2019/20 season)

| Name of | | Vaccine | | Administration | Produced | Age |
|---|---|---|---|---|---|---|
| product | Manufacturer | type | Adjuvant | route | in | recommended |
| 3Fluart | Fluart Innovative | Inactivated | Aluminium | intramuscular | Egg | From 3 years |
| | Vaccines Kft | | phosphate | | | |
| | | | gel | | | |
| Afluria | Pfizer/Seqirus | Inactivated | None | intramuscular | Egg | From 5 years |
| Agrippal | Seqirus | Inactivated/ | None | intramuscular | Egg | From 6 months |
| | | subunit | | | | |
| Fluad | Seqirus | Inactivated/ | Squalene | intramuscular | Egg | From 65 years |
| | | subunit | (MF59) | | | |
| Fluarix | GlaxoSmithKline | Inactivated/ | None | intramuscular/ | Egg | From 6 months |
| | | split | | subcutaneous | | |
| Fluarix Tetra | GlaxoSmithKline | Inactivated/ | None | intramuscular | Egg | From 6 months |
| | | split | | | | |
| Flucelvax Tetra | Seqirus | Inactivated | None | intramuscular | Cell | From 9 years |
| Fluenz Tetra | AstraZeneca | Live | None | intranasal | Egg | From 24 months |
| | | attenuated | | | | to 17 years |
| Imuvac | Abbot | Inactivated | None | intramuscular | Egg | From 6 months |
| | Biologicals/Mylan | | | | | |
| | Products Ltd. | | | | | |
| | (Marketing | | | | | |
| | Authorisation | | | | | |
| | Holder) | | | | | |
| Influvac Xanaflu | Mylan Products Ltd. | Inactivated | None | intramuscular | Egg | From 6 months |
| Influvac Tetra | Mylan Products Ltd | Inactivated | None | intramuscular/ | Egg | From 3 years |
| | | | | subcutaneous | | |
| Trivalent | Sanofi Pasteur | split virion, | None | intramuscular | Egg | From 65 years |
| Influenza Vaccine | | inactivated | | | | |
| High Dose | | | | | | |
| Vaxigrip Istivac | Sanofi Pasteur | Inactivated | None | intramuscular | Egg | From 6 months |
| Mutagrip | | | | subcutaneous | | |
| Vaxigrip Tetra | Sanofi Pasteur | Inactivated | None | intramuscular/ | Egg | From 6 months |
| | | | | subcutaneous | | |
| Xanaflu Tetra | Abbot | Inactivated | None | intramuscular/ | Egg | From 3 years |
| Fluvaccinol | Biologicals/Mylan | | | deeply | | |
| Subunit Tetra | Products Ltd. | | | subcutaneous | | |
| Batrevac Tetra | (Marketing | | | | | |
| Influenza vaccine | Authorisation | | | | | |
| Tetra MYL | Holder) | | | | | |

TABLE 3
available seasonal influenza vaccines in the UK (2020/2021 season)
All influenza vaccines available in the UK for the 2020 to 2021 season

| | | | Age | Ovalbumin content |
|---|---|---|---|---|
| Supplier | Name of product | Vaccine type | indications | micrograms/dose |
| AstraZeneca | Fluenz ® Tetra | Quadrivalent LAIV (live | From 24 | Less than 0.024 |
| UK Ltd | | attenuated influenza | months to less | micrograms per |
| | | vaccine) supplied as | than 18 years | 0.2 ml dose |
| | | nasal spray suspension | of age | |
| GSK | Fluarix Tetra | QIVe (standard egg- | From 6 | Equal to or less than |
| | | grown quadrivalent | months | 0.05 micrograms per |
| | | influenza vaccine), | | 0.5 ml dose |
| | | split virion, | | |
| | | inactivated | | |
| MASTA | Quadrivalent Influenza | QIVe (standard egg- | From 6 | Equal to or less than |
| | vaccine ▾ | grown quadrivalent | months | 0.05 micrograms per |
| | | influenza vaccine), | | 0.5 ml dose |
| | | split virion, | | |
| | | inactivated | | |
| Sanofi | Quadrivalent Influenza | QIVe (standard egg- | From 6 | Equal to or less than |
| Pasteur | vaccine ▾ | grown quadrivalent | months | 0.05 micrograms per |
| Vaccines | | influenza vaccine), | | 0.5 ml dose |
| | | split virion, | | |
| | | inactivated | | |
| Mylan | Quadrivalent Influvac ® | QIVe (standard egg- | From 3 years | Equal to or less than |
| | sub-unit Tetra ▾ | grown quadrivalent | | 0.1 micrograms per |
| | | influenza vaccine), | | 0.5 ml dose |
| | | surface antigen, | | |
| | | inactivated | | |
| Seqirus UK | Flucelvax ® Tetra ▾ | QIVc (cell-grown | From 9 years | Egg-free |
| Ltd | | quadrivalent influenza | | |
| | | vaccine), surface | | |
| | | antigen, inactivated | | |
| Seqirus UK | Adjuvanted Trivalent | aTIV (egg-grown | From 65 years | Equal to or less than |
| Ltd | Influenza Vaccine | trivalent influenza | | 0.2 micrograms per |
| | | vaccine), surface | | 0.5 ml dose |
| | | antigen, inactivated, | | |
| | | adjuvanted with | | |
| | | MF59C.1 | | |

Suitable influenza (virus) vaccines include, but are not limited to: Mutagrip may contain traces of egg, such as ovalbumin, and of neomycin, formaldehyde and octoxynol-9, used during the manufacturing process (as per Spanish label).

Quadrivalent inactivated influenza vaccine (VaxigripTetra™): VaxigripTetra™ (IIV4; Sanofi Pasteur) is a quadrivalent split-virion influenza vaccine approved in Europe in 2016 for individuals ≥3 years of age. IIV4 builds on the well-established record of the trivalent split-virion influenza vaccine (Vaxigrip®). IIV4 is immunogenic and well tolerated. Adding a second B strain to the trivalent split-virion influenza vaccine provides a superior immune response for the additional strain but does not reduce the immune response for the three other strains or negatively affect the safety profile. By offering broader protection against co-circulating influenza B lineages, IIV4 has the potential to further reduce influenza-related morbidity and mortality beyond that achieved with trivalent vaccines.

VaxigripTetra™ contain a buffer solution of sodium chloride, potassium chloride, disodium phosphate dihydrate, potassium dihydrogen phosphate, and water for injections. Some components such as eggs (ovalbumin, chicken proteins), neomycin, formaldehyde or octoxinol-9 may be present in very small amounts.

Alfuria quad—A single 0.5 mL dose of AFLURIA QUADRIVALENT contains sodium chloride (4.1 mg), monobasic sodium phosphate (80 mcg), dibasic sodium phosphate (300 mcg), monobasic potassium phosphate (20 mcg), potassium chloride (20 mcg), and calcium chloride (0.5 mcg). From the manufacturing process, each 0.5 mL dose may also contain residual amounts of sodium taurodeoxycholate (≤10 ppm), ovalbumin (<1 mcg), sucrose (<10 mcg), neomycin sulfate (≤81.8 nanograms [ng]), polymyxin B (≤14 ng), beta-propiolactone (≤1.5 ng) and hydrocortisone (≤0.56 ng).

Trivalent inactivated influenza virus vaccines and Quadrivalent inactivated influenza vaccines vaccines are preferred influenza (virus) vaccines for use according to the instant invention.

Especially preferred for use as a vaccine in the context of the present invention is Quadrivalent Influenza Vaccine (split virion, inactivated), which is indicated for the prevention of influenza disease caused by the two influenza A virus subtypes and the two influenza B virus types contained in the vaccine. Quadrivalent Influenza Vaccine (split virion, inactivated) is a vaccine administered to protect subjects or patients against influenza (flu). Quadrivalent Influenza Vaccine (split virion, inactivated), or similar types of vaccines, is indicated for the prevention of influenza disease caused by the two influenza A virus subtypes and the two influenza B virus types contained in the vaccine. Typically, such influenza vaccines are only administered once a year, preferably prior to or during flu-season. Preferably, influenza vaccines are administered once per year or in connection with the flu-season, but preferably in accordance with the vaccination periods of the instant invention.

However, such flu vaccines can also be administered more than once per year or per flu-season, preferably 2 or 3 times, more preferably 2 times, e.g. in order to boost or optimize the immunization achieved or achievable by said vaccine. If such a vaccine is to be administered 2 times, a first dose is preferably administered at any time within the vaccination periods described herein, preferably within the early vaccination periods described herein, followed by a second dose administered either within 1-3 weeks after said first administration, preferably within 1-3 weeks of the same early vaccination period, or within 4 weeks to 3 months later than the first dose, preferably within a later or subsequent vaccination period as described herein.

### Varicella Zoster Virus

Herpes zoster is caused by the same virus (varicella) that causes chickenpox in children. When this virus becomes active again in an adult, it can cause herpes zoster, or shingles. Zostavax® (zoster vaccine live) is a live vaccine that helps prevent shingles, as preferably inactivated or recombinant VZV vaccines, such as Shingrix® or Shingrix (Pro)® also do.

Herpes zoster (HZ) often occurs following reactivation of latent varicella-zoster virus (VZV) in sensory and autonomic neurons. Incidence of HZ varies from 6-8 cases/1000 person-years at age 50-59 years of age to >11 cases/1000 person-years at 70 years of age. The severity of HZ and its complications also increase with age, closely corresponding to the age-related decline in VZV-specific T-cell-mediated immunity (CMI) that is considered important in preventing the reactivation of latent VZV and preventing the propagation of the reactivated virus. HZ vaccines are believed to boost VZV-specific memory T cells, preventing their decline below the presently unknown threshold required for protection against HZ.

Suitable varicella zoster vaccines are known in the art.

A live attenuated VZV vaccine (Zostavax, Merck Sharpe & Dohme Corp, hereafter referred to as Zoster Vaccine Live [ZVL]), is available to prevent HZ in individuals ≥50 years of age. However, ZVL has some limitations. Clinical trials indicate that vaccine efficacy against HZ is 70% in adults 50-59 years of age and declines with age from 64% in persons 60-69 years to 18% in those ≥80 years. Moreover, efficacy of ZVL against HZ decreases over time, from 62% in the first year after vaccination to approximately 40% by the fifth year postvaccination.

A recombinant glycoprotein E (gE) subunit vaccine (HZ/su) was developed to overcome the unmet medical need for a better vaccine. HZ/su consists of the recombinant VZV gE and the AS01B Adjuvant System. gE was selected as the vaccine antigen because it is the most abundant glycoprotein expressed by VZV-infected cells and it induces both neutralizing antibody and CD4 T-cell responses. AS01B contains Quillaja saponaria Molina, fraction 21 (QS-21; licensed by GSK from Antigenics LLC, a wholly owned subsidiary of Agenus Inc., a Delaware, US corporation) and 3-O-desacyl-4′-monophosphoryl lipid A (MPL). AS01B stimulates a local and transient activation of the innate response leading to the recruitment and activation of antigen-presenting dendritic cells. QS-21 is an adjuvant that induces transient local cytokine responses and activation of dendritic cells and macrophages in muscle and draining lymph nodes in animal models. The toll-like receptor type 4 agonist MPL synergizes with QS-21 to enhance the immune response to the coadministered antigen through the production of interferon-gamma (IFN-7).

Phase I and II trials demonstrated that a single HZ/su dose elicits substantial humoral and CMI responses, which further increase after a second dose.

Especially preferred for use as a vaccine in the context of the present invention is an inactivated and/or recombinant herpes zoster vaccine or varicella zoster vaccine, preferably one that has European Medicines Agency (EMA) product number EMEA/H/C/004336, in which the active substance comprises recombinant varicella zoster virus glycoprotein E, having the International non-proprietary name (INN) or common name “Herpes zoster vaccine” or “Herpes zoster vaccine (recombinant, adjuvanted)”, for which the therapeutic areas (MeSH) comprise Herpes Zoster, and/or for which the Anatomical therapeutic chemical (ATC) code is J07BK03. This vaccine is known under the tradename SHINGRIX®. Preferably, said inactivated and/or recombinant herpes zoster vaccine or varicella zoster vaccine is available as a powder and a suspension, preferably to be mixed together, e.g. by a doctor or a nurse, before being administered to a said patient, preferably by injection, e.g. by injection into the upper arm muscle of a said patient. The vaccination in this regard preferably comprises 2 separate vaccinations, 2 separate injections or 2 separate shots, preferably given about 3 weeks to about 8 months apart, preferably about 4 weeks to about 6 months apart.

Typically, in this regard, the two separate vaccinations, two separate shots or two separate injections, are administered to a said patient or subject
(i) about 3 to 5 weeks or about one month apart
(ii) about 8 to 10 weeks or about 2 months apart,
(iii) about 24 to 28 weeks or about 6 months apart, or
(iv) about 4 to 28 weeks apart, about 6 to 26 weeks apart, or about 8 weeks to 25 weeks apart, or basically up to 6 months apart.

Accordingly, if desired or necessary in this regard, the second vaccination, dose, injection or shot can be given later than 3 to 10 weeks apart, but preferably within 6 months after the first vaccination, dose injection or shot. People whose immune system does not work properly, who are under immunosuppressive therapy, and/or are treated or are to be treated according to the methods of treatment of the instant invention, and thus would benefit from a shorter vaccination schedule can have the second dose one to two months after the first dose, preferably within the vaccination periods as outlined herein. This is especially preferred with regard to inactivated or recombinant zoster vaccines, herpes zoster vaccines, varicella zoster vaccines and/or VZV vaccines, especially such vaccines that are generally administered 2 times to said subject or said patient in order to achieve optimum protection or immunity for said subject or patient. Administration is typically done by 2 separate vaccinations, 2 separate injections or 2 separate shots, preferably as it is known in the art, and especially as described herein. A preferred example of such a vaccine is the Zoster Vaccine Recombinant, Adjuvanted, e.g. the one commercially available under the tradename SHINGRIX®. Such vaccines are preferably indicated for prevention of herpes zoster (shingles), more preferably for prevention of herpes zoster (shingles) in adults aged 50 years and older. Such vaccines are also preferred for use as a vaccine according to the invention. The two doses of said vaccines are preferably administered via injection, preferably intramuscularly, and preferably according to the following schedules: A first dose at any time within the vaccination periods described herein, preferably within the early vaccination periods described herein, followed by a second dose administered a) at any time between 1 and 6 months later than the first dose, or b) at any time between 2 and 6 months later than the first dose, preferably within the later vaccination periods described herein.

The vaccine Zoster Vaccine Recombinant, Adjuvanted, e.g. SHINGRIX®, was shown to boost VZV-specific immune responses, preferably if used in accordance with the methods of the instant invention, which boost is believed to be the mechanism by which it protects said subjects or patients against said varicella zoster and/or herpes zoster disease.

Especially preferred in this regard is a vaccine comprising recombinant varicella zoster virus glycoprotein E, preferably a vaccine having the International non-proprietary name (INN) or common name “Herpes zoster vaccine” or “Herpes zoster vaccine (recombinant, adjuvanted)”, e.g. SHINGRIX®.

Preferred with regards to varicella zoster virus vaccines are live vaccines or live-attenuated vaccines, such as Zostavax® (zoster vaccine live), which can be used to prevent varicella zoster virus (shingles) in people age 50 and older. Also preferred in this regard non-live varicella zoster virus (VZV) vaccines, preferably including, but not limited to inactivated VZV vaccines and recombinant VZV vaccines, such as Shingrix® or Shingrix (Pro)®,

Also especially preferred for use as a vaccine in the context of the present disclosure is Zoster Vaccine Live, which is preferably a live attenuated virus vaccine, e.g. ZOSTAVAX© (Zoster Vaccine Live), and is preferably indicated for prevention of herpes zoster (shingles), more preferably indicated for prevention of herpes zoster (shingles) in individuals 50 years of age and older. Zoster Vaccine Live, e.g. ZOSTAVAX©, is preferably a lyophilized preparation of a live, attenuated varicella-zoster virus (VZV) strain, e.g. the Oka/Merck strain of live, attenuated varicella-zoster virus (VZV). ZOSTAVAX©, when reconstituted as directed, is a sterile suspension for subcutaneous administration. Typically, such Zoster Vaccine Live is for single administration, thus it is typically administered only once (i.e as a single dose) to a subject or patient in accordance with the instant invention. However, once repeated administration can be recommended or prescribed by the treating physician. In any case, it is typically administered subcutaneously, preferably in the deltoid region of the upper arm of said subject or patient. Herpes zoster (HZ), commonly known as shingles or zoster, is preferably a manifestation of the reactivation of varicella zoster virus (VZV), which, as a primary infection, produces chickenpox (varicella). Following initial infection, the virus remains latent in the dorsal root or cranial sensory ganglia until it reactivates, producing zoster. Zoster is characterized by a unilateral, painful, vesicular cutaneous eruption with a dermatomal distribution. The risk of developing zoster and/or Shingles appears to be related to a decline in VZV-specific immunity. Varicella Zoster Virus Vaccine (or Varicella Zoster Vaccine), preferably Zoster Vaccine Live and especially ZOSTAVAX® was shown to boost VZV-specific immunity, which is thought to be the mechanism by which it protects against zoster and its complications.

Other Diseases for which Vaccines are Available or Desirous

Further infectious diseases for which vaccines are available or at least desirous, include, but are preferably not limited to hepatitis B virus (HBV) and varicella zoster virus (VZV). Other infection diseases might include Hepatitis B, Measles, Influenza vaccines, Poliovirus, Pneumococci, Diphtheria, Tetanus and Bordetella pertussis and SARS-Covid 2. Vaccines against SARS Covid-2 are now available.

Other diseases, including smallpox, chickenpox, polio, tetanus, measles, mumps, rubella, whooping cough, and/or influenza-like disorders, are also relevant.

### Vaccination

Vaccination with vaccines is a way of artificially activating the immune system to protect against infectious disease. The activation occurs through priming the immune system with an immunogen. Stimulating immune responses with an infectious agent is known as immunization. Vaccination includes various ways of administering immunogens.

Various routes for administration of vaccines are known to the skilled artisan. Typically, vaccine administration may be oral, by injection (intramuscular, intradermal, subcutaneous), by puncture, transdermal or intranasal. Several recent clinical trials have aimed to deliver the vaccines via mucosal surfaces to be taken up by the common mucosal immunity system, thus avoiding the need for injections.

Most vaccines are administered before a patient has contracted a disease to help increase future protection. However, some vaccines are administered after the patient already has contracted a disease. Vaccines given after exposure to smallpox are reported to offer some protection from disease or may reduce the severity of disease.

Most vaccines are given by injection as they are not absorbed reliably through the intestines. Live attenuated polio, rotavirus, some typhoid, and some cholera vaccines are given orally to produce immunity in the bowel. While vaccination provides a lasting effect, it usually takes several weeks to develop and requires an immune system that is able to mount suitable immune response.

### Vaccine Types

There are several different types of vaccines. Each type is designed to teach your immune system how to fight off certain kinds of germs and the serious diseases they cause. When scientists create vaccines, they consider: How your immune system responds to the germ, who needs to be vaccinated against the germ, the best technology or approach to create the vaccine. Based on a number of these factors, scientists decide which type of vaccine they will make. There are several main types of vaccines, e.g., live-attenuated vaccines, inactivated vaccines, subunit, recombinant, polysaccharide, and conjugate vaccines, toxoid vaccines.

The most relevant types of vaccines are described in more detail below:
Live-attenuated vaccines: Live vaccines use a weakened (or attenuated) form of the germ that causes a disease. Because these vaccines are so similar to the natural infection that they help prevent, they create a strong and long-lasting immune response. Just 1 or 2 doses of most live vaccines can give you a lifetime of protection against a germ and the disease it causes. But live vaccines also have some limitations. For example: Because they contain a small amount of the weakened live virus, some people should talk to their health care provider before receiving them, such as people with weakened immune systems, long-term health problems, or people who've had an organ transplant. They need to be kept cool, so they don't travel well. That means they can't be used in countries with limited access to refrigerators. Live vaccines are used to protect against: Measles, mumps, rubella (MMR combined vaccine), Rotavirus, Smallpox, Chickenpox, Yellow fever, nasal Influenza vaccines.
Inactivated vaccines: Inactivated vaccines use the killed version of the germ that causes a disease. Inactivated vaccines usually don't provide immunity (protection) that's as strong as live vaccines. So you may need several doses over time in order to get ongoing immunity against diseases. Inactivated vaccines are used to protect against: Hepatitis A, Flu (shot only), Polio (shot only), Rabies.
Subunit, recombinant, polysaccharide, and conjugate vaccines: Theses vaccines use specific pieces of the germ—like its protein, sugar, or capsid (a casing around the germ). Because these vaccines use only specific pieces of the germ, they give a very strong immune response that's targeted to key parts of the germ. They can also be used on almost everyone who needs them, including people with weakened immune systems and long-term health problems. One limitation of these vaccines is that you may need booster shots to get ongoing protection against diseases. These vaccines are used to protect against: Hib (Haemophilus influenzae type b) disease, Hepatitis B, HPV (Human papillomavirus), Whooping cough (part of the DTaP combined vaccine, Pneumococcal disease, Meningococcal disease, Shingles.
Toxoid vaccines: Toxoid vaccines use a toxin (harmful product) made by the germ that causes a disease. They create immunity to the parts of the germ that cause a disease instead of the germ itself. That means the immune response is targeted to the toxin instead of the whole germ. Like some other types of vaccines, you may need booster shots to get ongoing protection against diseases. Toxoid vaccines are used to protect against: Diphtheria, Tetanus.
DNA plasmid vaccines: comprise a small circular piece of DNA called a plasmid that carries genes encoding proteins from the pathogen of interest. The manufacturing process for DNA plasmid vaccines is well-established, allowing experimental vaccines to be quickly developed to address emerging or re-emerging infectious diseases. NIAID's Vaccine Research Center has developed candidate DNA vaccines to address several viral disease threats during outbreaks, including SARS coronavirus (SARS-CoV) in 2003, H5N1 avian influenza in 2005, H1N1 pandemic influenza in 2009, and Zika virus in 2016. The time from selection of the viral genes to be included in the vaccine to initiation of clinical studies in humans was shortened from 20 months with SARS-CoV to slightly longer than three months with Zika virus.
mRNA Vaccines: Vaccines based on messenger RNA (mRNA), an intermediary between DNA and protein, also are being developed. Recent technological advances have largely overcome issues with the instability of mRNA and the difficulty of delivering it into cells, and some mRNA vaccines have demonstrated encouraging early results. For example, NIAID-supported researchers developed an experimental mRNA vaccine that protected mice and monkeys against Zika virus infection after a single dose.
Recombinant Vector vaccines (platform-based vaccines): Rather than delivering DNA or mRNA directly to cells, some vaccines use a harmless virus or bacterium as a vector, or carrier, to introduce genetic material into cells. Several such recombinant vector vaccines are approved to protect animals from infectious diseases, including rabies and distemper. Many of these veterinary vaccines are based on a technology developed by NIAID researchers in the 1980s that uses weakened versions of a poxvirus to deliver the pathogen's genetic material. Today, NIAID-supported scientists are developing and evaluating recombinant vectored vaccines to protect humans from viruses such as HIV, Zika virus and Ebola virus.

However, vaccinations can lead to severe clinical crisis in the vaccinated body, e.g. by vaccine failure. Typically, vaccine failure is implied when an organism contracts a disease in spite of being vaccinated against it. Primary vaccine failure occurs when an organism's immune system does not produce antibodies when first vaccinated. Vaccines can fail when several series are given and fail to produce an immune response. The term “vaccine failure” does not necessarily imply that the vaccine is defective. Most vaccine failures are simply from individual variations in immune response, but also occur in immunosuppressed bodies, e.g. under immunosuppressive treatment of patients with autoimmune disease.

### Vaccine Response

### Effector Mechanisms

Vaccines are found to induce effector mechanisms (cells or molecules) capable of rapidly controlling replicating pathogens or inactivating their toxic components. Early protective efficacy is primarily conferred by the induction of immune effectors produced by B cells, antigen specific antibodies, capable of binding to a pathogen or toxin. Long term protection requires the persistence of vaccine antibodies above protective threshold and/or the maintenance of immune memory capable of rapid and effective reactivation. Antigen-specific antibodies have been formally demonstrated as conferring vaccine induced protection against many diseases. The predominant role of B cells in the efficacy of current vaccines should not overshadow the importance of T cell responses. Most antigens and vaccines trigger B (humoral immunity) and T cell responses (cellular immunity).

CD4+ T cells are required for most antibody responses and antibodies exert significant influences on T cell responses to intracellular pathogens. T cells are essential to the induction of high affinity antibodies and immune memory. Cytotoxic CD8+T lymphocytes are additional effectors that would limit the spread of infectious agents by recognizing and killing infected cells or secreting specific anti-viral cytokines. CD4+T helper cells (Th) provide support to the generation and maintenance of B cell and CD8 responses and contribute to protection by cytokine production (example: TH1 cells expressing IFN-gamma). Other additional Th subtype effector cells are follicular Th cells (Tfh) supporting potent B cell activation and differentiation in antibody secreting cells or TH17 cells.

TABLE 4
Effector Mechanisms Triggered by Vaccines

Antibodies prevent or reduce infections by clearing extracellular
pathogens through:
Binding to the enzymatic active sites of toxins or preventing
their diffusion
Neutralizing viral replication (e.g., preventing viral binding
and entry into cells)
Promoting opsonophagocytosis of extracellular bacteria (i.e.,
enhancing their clearance by macrophages and neutrophils)
Activating the complement cascade
CD8⁺ T cells do not prevent infection but reduce, control,
and clear intracellular pathogens by:
Directly killing infected cells (release of perforin, granzyme,
etc.)
Indirectly killing infected cells through antimicrobial
cytokine release
CD4⁺ T cells do not prevent infection but participate in
the reduction, control, and clearance of extracellular and
intracellular pathogens by their homing and cytokine-
production capacities. Their main subsets include:
Follicular T-helper (Tfh) cells producing mainly interleukin
(IL)-21 and providing B cell help
T-helper 1 (Th1) effector cells producing interferon (IFN)-gamma,
tumour necrosis factor (TNF)-alpha/TNF-beta, IL-2, and mainly
involved in protection against intracellular pathogens (viruses,
Mycobacterium tuberculosis)
Th2 effector cells producing IL-4, IL-5, IL-13, and responding
to extracellular pathogens (bacteria and helminths)
Th9 effector cells producing IL-9 and also responding to
extracellular pathogens
Th17 effector cells producing IL-17, IL-22, and IL-26 and
contributing to mucosal defense (Streptococcus pneumoniae,
Bordetella pertussis, Mycobacterium tuberculosis)

The Nature of the Vaccine Exerts a Direct Influence on the Type of the Immune Effectors Elicited that Mediate Protective Activity

A hall mark of T dependent responses (elicited by Toxoid, protein, inactivated or live attenuated viral vaccines) is to induce higher affinity antibodies and immune memory. In addition, live attenuated vaccines/vectors usually generate CD8 cytotoxic T cells. Most current vaccines mediate their protective efficacy through the induction of vaccine antibodies, whereas vaccine induced CD4+ T cells contribute to macrophage activation and control of Mycobacterium tuberculosis. Current vaccines mostly mediate protection through the induction of highly specific IgG serum antibodies. Live vaccines induce serum IgA and secretory IgA levels which help to limit viral shedding at mucosal surfaces. There is conclusive evidence that that T cells are the main effectors for BCG in BCG immunized infants or in zoster immunized adults. There is indirect evidence that vaccine induced T cells contribute to the protection conferred by other vaccines.

TABLE 5
T-Cell Responses to Vaccines

Type

| | | |
|---|---|---|
| CD4⁺ T-helper cells | | |
| Th1 | IFN-gamma production | Extrafollicular B-cell help |
| Th1 | Cell contact, IFN-gamma | Activation of CD8⁺ T cells |
| Th1/Th2 | Cell contact, CD40L | Dendritic cell activation |
| Th2 | IL-4, IL-5, IL-13 | Extrafollicular B-cell help |
| Th2 | Cell contact, IL-4 | Suppression of CD8⁺ T cells |
| Th17 | IL-17, IL-21, IL-22 | Mucosal inflammation |
| CD4⁺ follicular T-helper cells | | |
| Tfh1 | IFN-gamma | Germinal center B-cell help |
| Tfh2 | IL-4, IL-5, IL-13 | Germinal center B-cell help |
| CD4⁺ regulatory T cells | Multiple mechanisms | Suppression of CD4⁺/CD8⁺ |
| | | responses |
| CD8⁺ T cells | IFN-gamma, TNF-alpha | Killing of infected cells |
| Effector memory T cells | Th1/Th2 cytokines, perforin, | Rapid secondary effector- |
| | granzyme | responses in periphery |
| Memory T cells | | |
| Effector memory T cells | Th1/Th2 cytokines, perforin, | Rapid secondary effectors |
| | granzyme | responses in periphery |
| Central memory T cells | IL-2, IL-10, CD40L | Delayed activation/ |
| | | proliferation in lymph nodes |
| Tissue-resident memory | Th1/Th2 cytokines, perforin, | Tissue localisation enabling |
| T cells | granzyme | immediate-early reactivation |

IFN; interferon;
IL; interleukin;
Th, T-helper;
TNF, tumor necrosis factor

### Innate Immunity and Vaccination

When exposed to pathogens in the tissues or the sites of injections immature dendritic cells maturate and migrate to secondary lymph nodes where the induction of B and T cell responses are to occur. The central role of mature DCs (dendritic cells) in the induction of a vaccine response is to provide sufficient danger signals through vaccine antigens or adjuvants to trigger an inflammatory action. DCs, monocytes and neutrophils express pattern recognition receptors (ex. Toll like receptors, TLRs) sensing the potential danger when they encounter a pathogen and get activated. They modulate the expression of cell surface proteins and produce proinflammatory cytokines and chemokines. The inflammatory milieux drives monocyte differentiation into macrophages and DC maturation. In absence of a danger signal DCs remain immature and on contact with naïve cells, CD4+ cells do not differentiate into effector cells. Life vaccines trigger most efficiently the innate immune system in time and space via TLRs. Non-live vaccines (ex. proteins, glycoconjugates, inactivated microorganisms) may still contain pathogen recognition receptors, however vaccine induced activation stays more limited at the site of injection. After activation, DCs change their homing receptors and migrate to draining lymph nodes, where B and T cell reactions occur.

### Primary Antibody Responses

In response to a protein antigen, B cells capable of binding to this antigen with their surface immunoglobulins undergo brisk activation. The initial antigen exposure elicits an extrafollicular response where B cells rapidly differentiate in plasma cells that produce low affinity antibodies (IgM+/−IgG isotypes) that results in a rapid appearance of low Ig titers in the serum within a few days after immunization. Antigen specific T helper cells that have been activated by antigen bearing DCs trigger antigen specific B cells to migrate toward follicular DCs, initiating the germinal center (GC) reaction. In GCs B cells receive additional signals from T follicular helper (Tfh) cells, undergo massive clonal proliferation and switch from IgM toward IgG, Ig A or IgE. As B cell proliferate in Germinal Centers IgG antibody titers increase up to peak values, usually reached at 4 weeks after immunization. The short lifespan of these plasma cells results in rapid decrease of antibody titers. A minimal interval of 3 weeks between 2 primary doses allows development of successive waves of Ag specific primary responses without interference.

### Secondary Antibody Response

In the secondary immune response booster exposure to antigen reactivates immune memory and results in a rapid increase of IgG titers. Short lived plasma cells maintain peak levels for several weeks until long-lived plasma cells that have reached the survival niches in the bone marrow continue to produce antigen specific antibodies which then decline with slower kinetics. A minimal interval of 4 month between priming and boosting allow affinity maturation of memory B cells and thus higher secondary responses. This generic pattern may not apply to live vaccines triggering long term IgG antibodies for extended periods.

TABLE 6
Determinants of the Duration of Vaccine
Antibody Responses in Healthy People

| Determinants | Mechanisms (Presumed) |
|---|---|
| VACCINE TYPE | |
| Live vs inactivated | Life vaccines generally induce more |
| | sustained Ab responses, presumably |
| | through Ag persistence within the |
| | host. |
| Polysaccharide | Failure to generate Tfh cells and |
| antigens | GCs limits the induction of memory |
| | responses and of high affinity long- |
| | lived plasma cells. |
| VACCINE SCHEDULE | |
| Interval between | A minimal interval of three weeks |
| primary doses | between primary doses allows development |
| | of successive waves of Ag-specific |
| | primary responses without interference. |
| Interval before | A minimal interval of 4 months between |
| boosting | priming and boosting allows affinity |
| | maturation of memory B cells and thus |
| | higher secondary responses. |
| Age at | Early life immune immaturity and age- |
| immunization | associated immunosenescence limit the |
| | induction/persistence of long-lived |
| | plasma cells. |
| Environmental | Mostly and identified |
| factors | |

Ab: antibody;
Ag: antigen;
GC: germinal center;
Tfh: follicular T-helper cells

### Vaccine Induced B Cell Memory

Memory B cells are generated during primary responses to T-dependent vaccines. They persist in the absence of antigens—re-exposure to antigen drives their differentiation into antibody-producing plasma cells. This reactivation is rapid, does not need T cell help, such that booster responses are characterized by rapid increase to higher titers of antibodies that have a higher affinity for antigens than do antibodies generated during primary responses.

TABLE 7
Hallmarks of Memory B-Cell Responses
Memory B cells:

Are generated only during T -dependent responses inducing follicular
T-helper cells and thus germinal center responses
Are resting cells that do not produce antibodies
Rapidly (days) differentiate into antibody-secreting plasma cells on
reexposure to antigen primary plasma cells
Differentiate into plasma cells that produce high(er)-affinity
antibodies than do primary plasma cells

### Vaccine Induced T Cell Memory

TABLE 8
Determinants of Memory T-Cell Responses

| Main Factors | Determinants |
|---|---|
| Frequency of memory T cells | Magnitude of T-cell expansion (initial |
| | antigen load, antigen persistence) |
| Phenotype of memory T cells | |
| Effector memory | Induction favored by prolonged antigen |
| | persistence |
| Tissue-resident memory | |
| Central memory | Induction favored by rapid antigen |
| | clearance |
| Persistence of memory T cells | Supported by interleukin (IL)-15, IL-7 |

TABLE 9
In Elderly People

| | |
|---|---|
| Limited magnitude of Ab | Low reservoir of IgM+ memory B cells; |
| responses to PS | weaker differentiation into plasma cells |
| Limited magnitude of Ab | Limited GC responses: suboptimal CD4⁺ |
| responses to proteins | helper responses, suboptimal B-cell |
| | activation, limited FDC network |
| | development; changes in B-/T-cell repertoire |
| Limited quality (affinity, | Limited GC responses; changes in B-/T-cell |
| isotype) of antibodies | repertoire |
| Short persistence of Ab | Limited plasma cell survival |
| responses to proteins | |
| Limited induction of CD4⁺/ | Decline in naive T-cell reservoir |
| CD8⁺ responses | (accumulation of effector memory and CD8⁺ |
| | T cell clones) |
| Limited persistence of | Limited induction of new effector memory T |
| CD4⁺ responses | cells (IL-2, IL-7) |

Ab, antibody;
FDC, follicular dendritic cell;
GC: germinal center;
IFN: interferon;
Ig: immunoglobulin;
IL: interleukin;
PS: polysaccharide.

Problems with Vaccination Under Treatment with Immunosuppressant/Immunomodulatory Drugs

Vaccination is a powerful tool to prevent infections, to reduce the risk of contracting an infection and, if an infection is indeed contracted, increase the chance that the course of the infection is mild. However, as laid out in detail in the Background section, many immunosuppressant and immunomodulatory drugs affect the immune system in such a way that a full vaccination response does not occur. Thus, in these cases vaccination will not result in the desired immunization.

In view of this situation, there are strong recommendations to avoid vaccinations while on treatment with medications that have such a modifying effect on the immune response or in close timely proximity to such a treatment. Instead, it is recommended to always leave a gap or an interval of a few weeks to several months between a vaccination and the beginning and/or end of a treatment with such an immunosuppressive or immunomodulatory drug (see Background section above).

Based on their mode of action, it would seem likely that such problems will also occur for BTK inhibitors. This assumption is in line with the vaccination data obtained with selected BTK inhibitors, which suggests that vaccination outcomes might be blunted under treatment with BTK inhibitors (see Background section above). According to the general recommendation, this means that vaccination in close temporal proximity to treatment with an immunomodulatory drug should also be avoided in the case of the BTK inhibitors.

### BTK Inhibitors

Bruton's tyrosine kinase (BTK) is a cytoplasmatic kinase of the Tec family that is expressed by hematopoietic and plasma cells that regulate different signals, including the PI3K, MAPK, and NF-kappa B pathways.

As discussed in the Background section, BTK is expressed in almost all hematopoietic cells, in particular in B cells, but also in macrophages and monocytes, but not in T cells. In fact, it is an essential component of different B cell receptor (BCR) signal pathways that regulate the differentiation of antibody-producing plasma cells, as well as cell activation, proliferation, and survival. In addition, BTK plays a crucial role in the secretion of pro-inflammatory cytokines, as well as degranulation and histamine release.

Therefore, BTK inhibition causes a block of different downstream cell signalling pathways related to the development of B-cell malignancies as well as autoimmune and inflammatory diseases.

BTK is made up of 659 amino acids. The protein comprises five domains. From the N-terminus to the C-terminus, these are: the pleckstrin homology (PH) domain, the proline-rich TEC homology (TH) domain, the SRC homology (SH) domains (named SH3 and SH2), and finally the catalytic domain. The function of the PH domain is to mediate protein-phospholipid and protein-protein interactions. The TH domain, in contrast, contains a zinc finger motif important for protein activity and stability. In the SH2 and SH3 domains, the autophosphorylation site Tyr223 is located. The catalytic domain contains two phosphorylation sites (Tyr551 and Cys481).

Based on their mechanism of action and binding mode, BTK inhibitors can be classified into two types: (i) irreversible inhibitors that form a covalent bond with the conserved cysteine 481 residue in the ATP binding site, and (ii) reversible inhibitors that bind to a specific pocket in the SH3 domain through weak, reversible interactions (e.g., hydrogen bonds or hydrophobic interactions), causing an inactive conformation of the enzyme.

Examples of irreversible BTK inhibitors are evobrutinib, spebrutinib, remibrutinib, tolebrutinib, olmutinib, branebrutinib, TAK-020 (by Takeda), elsubrutinib or rilzabrutinib. Examples of reversible BTK inhibitors are vecabrutinib, BMS-935177 (Bristol Meyers Squibb), BMS-986142 (Bristol Meyers Squibb) or fenebrutinib,

BTK inhibitors are reviewed e.g. in Tasso et al., Molecules (2021), vol. 26, p. 7411 or Ringheim et al., Frontiers in Immunology (2021), vol. 66, p. 2223, the contents of which are incorporated herein by reference. See also the discussion and citations in the Background section, the contents of which are also herewith incorporated by reference.

BTK inhibitors have shown great potential in the clinical treatment of diseases.

Ibrutinib was the first BTK inhibitor approved by the FDA in 2013 for the treatment of chronic lymphocytic leukemia (CLL), small lymphocytic lymphoma (SLL), Waldenstrom's macroglobulinemia (WM), marginal zone lymphoma (MZL), and relapsed/refractory mantle cell lymphoma (MCL). Subsequently, it was also approved in 2017 for chronic graft-versus-host disease (cGVHD) patients. More recently, acalabrutinib and zanubrutinib were launched in the market in 2017 and 2019, respectively, for CLL, SLL, and MCL. In addition, tirabrutinib is currently registered in Japan for the treatment of recurrent or refractory primary central nervous system lymphoma and has also received supplemental approval for WM and lymphoplasmacytic lymphoma. In December 2020, orelabrutinib received its first approval in China for the treatment of patients with MCL, CLL, and SLL who have received at least one treatment in the past. The clinical development of orelabrutinib for various indications is underway in the USA and China. See e.g. Tasso et al. for review of such compounds and treatments.

As laid out in detail in the Background section, various BTK inhibitors have also shown great potential in the treatment of autoimmune diseases and other inflammatory diseases, or are currently in clinical trials for such diseases (reviewed in Ringheim et al., Frontiers in Immunology (2021), vol. 66, p. 2223). This includes the treatment of autoimmune diseases like rheumatoid arthritis, systemic lupus erythematosus, multiple sclerosis, including relapsing MS (RMS) and progressive forms (PMS), pemphigus vulgaris, Sjogren's syndrome, chronic spontaneous urticaria and idiopathic thrombocytopenia, as well as other inflammatory diseases like graft versus host disease, and asthma. The BTK inhibitors evobrutinib and tolebrutinib have shown positive results in phase II trials for RMS treatment (Montalban et al., New Engl. J. of Medicine (2019), vol. 380, p. 2406; Reich et al., Lancet Neurology (2021), vol. 20, p. 729). Moreover, recently a trial with tolebrutinib in myasthenia gravis (MG) has been announced recently (NCT05132569).

Thus, BTK inhibitors can be successfully administered to treat different diseases, including various autoimmune diseases and other inflammatory diseases. For details about these treatments, see e.g. in Ringheim et al., Tasso et al., the references cited therein, the study postings at www.clinicaltrials.gov as well as the corresponding study publications.

Therefore, such BTK inhibitors are considered for the practice of the present disclosure.

### Evobrutinib

Evobrutinib is a BTK inhibitor with the structure shown below in formula (I):

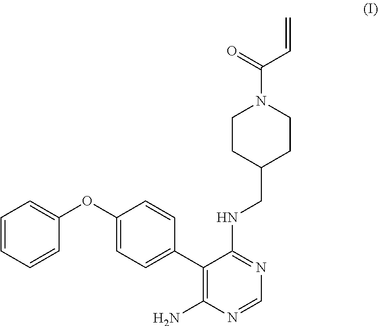 [SMILES: C=CC(=O)N1CCC(CNc2ncnc(N)c2-c2ccc(Oc3ccccc3)cc2)CC1]

The chemical name of evobrutinib is 1-[4-({[6-amino-5-(4-phenoxyphenyl)pyrimidin-4-yl]amino}methyl)piperidin-1-yl]prop-2-en-1-one, or (synonymously)N-[(1-acryloylpiperidin-4-yl)methyl]-5-(4-phenoxyphenyl)pyrimidine-4,6-diamine.

Preferably, the naming of an active ingredient, active principle (API), medicament or international nonproprietary name (INN) thereof includes all prodrugs, salts and solvates thereof, especially those that are functionally equivalent and/or are deemed a suitable substitute from a clinical point of view.

Evobrutinib and its use in medical treatment is described e.g. in published international patent applications WO 2012/170976 A2, WO 2017/087445 A1 and WO 2020/016850 A1 or in publications on clinical trials with evobrutinib, such as Montalban et al., the contents of which are incorporated herein by reference.

A related BTK inhibitor is 1-(4-(((6-amino-5-(4-phenoxyphenyl)pyrimidin-4-yl)amino)methyl)-4-fluoropiperidin-1-yl)prop-2-en-1-one, described e.g. in WO 2012/170976 A2, WO 2017/087445 A1 and WO 2017/079542 A1 (compound A225).

Evobrutinib is an irreversible BTK inhibitor. It binds covalently to cysteine 481 in the active site of BTK, causing strong and sustained inhibition of BTK enzymatic activity.

Functional changes induced by BTK inhibition through evobrutinib include the inhibition of B cell proliferation, activation, maturation, antigen presentation, cytokine/antibody production, and CNS homing. Due to the inhibition of T cell and B cell interactions, evobrutinib also affects indirectly pathological T cell responses. In myeloid cells (monocyte, macrophages, dendric cells) and CNS Glia (microglia, astrocytes), evobrutinib acts downstream of various receptors (eg. GM-CSF receptor, FcR) decreasing proinflammatory while increasing anti-inflammatory signals, thus potentially promoting a more anti-inflammatory state in the periphery and the CNS compartment.

Evobrutinib is under clinical investigation for autoimmune disorders and, when orally administered, it showed good efficacy in mouse models of RA and SLE, as demonstrated by a reduction of disease severity and histological damage (Caldwell et al., J. Med. Chem. (2019), vol. 62, p. 7643-7655; Haselmayer et al., J. Immunol. (2019), vol. 202, p. 2888-2906.).

Moreover, evobrutinib has shown effects in the treatment of patients with relapsing forms of multiple sclerosis (Montalban et al.).

Evobrutinib (and similarly the related BTK inhibitor 1-(4-(((6-amino-5-(4-phenoxyphenyl)pyrimidin-4-yl)amino)methyl)-4-fluoropiperidin-1-yl)prop-2-en-1-one) may be considered for use in the practice of the teachings of the present disclosure.

BTK inhibitors, and in particular evobrutinib can be formulated in any pharmaceutical preparation, wherein preferably such a pharmaceutical preparation is suitable for oral administration. Representative oral formulations of BTK inhibitors are described in the product characteristics of BTK inhibitors that have obtained marketing authorization by the FDA or EMA (see respective United States Prescribing Information (USPI) approved by the US Food and Drug Administration (FDA) or Summary of Product Characteristics (SmPC) approved by the European Health Authority (EMA)). Examples of ingredients for oral formulations are provided below.

Processes for preparing evobrutinib are known in the art. For example, the preparation of evobrutinib is described in published international patent application WO 2012/170976 A2.

A BTK inhibitor may be used in the form of a free base, pharmaceutically acceptable salt, solvate, or solvate of a salt. As used herein, the term “pharmaceutically acceptable salt” refers to salts prepared from pharmaceutically acceptable non-toxic bases or acids, including inorganic bases or acids and organic bases or acids. Accordingly, a BTK inhibitor which contains one or more basic groups (such as evobrutinib), i.e., groups which can be protonated, can be present in salt form, and can be used according to the invention in the form of an addition salt with inorganic or organic acid. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. Similarly, a BTK inhibitor which contains one or more acidic groups can be present in salt form, and can be used according to the present disclosure, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids.

Oral administration of evobrutinib may be in capsule, tablet, oral suspension, or syrup form. The tablet or capsules may contain from about 10 to 500 mg of said BTK inhibitor (such as evobrutinib). Preferably they may contain 30 to about 300 mg of evobrutinib, more preferably about 40 to 150 mg of evobrutinib. Tablets may contain the foregoing amount of the compound and a binder, which may be a gelatin solution, a starch paste in water, polyvinyl polyvinyl alcohol in water, etc. with a typical sugar coating.

Preferably, the term “evobrutinib”, as used in the context of the instant invention, if not explicitly defined to be otherwise, preferably means “oral evobrutinib”, i.e. the active ingredient evobrutinib to be orally administered according to the invention. Preferably, the evobrutinib to be orally administered according to the invention is administered in the form of an oral dosage form, such as a capsule, e.g. a capsule filled with evobrutinib solution, a capsule filled with evobrutinib powder or an evobrutinib-containing tablet.

Compositions of evobrutinib may further comprise one or more pharmaceutically acceptable additional ingredient(s) such as alum, stabilizers, antimicrobial agents, buffers, coloring agents, flavoring agents, adjuvants, and the like.

Compositions may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

Compositions may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The above-described information about compositions, formulations in any pharmaceutical preparation or preparations for oral administration applies accordingly to other BTK inhibitors.

### Post-Hoc Analysis of Clinical Study Data

In the Experimental Section herein a post hoc analysis of data obtained in two phase II clinical studies with a BTK inhibitor (evobrutinib) has been carried out.

In the context of treating different autoimmune diseases (SLE, MS) with this BTK inhibitor, it was surprisingly found that during treatment with the BTK inhibitor, different vaccinations (influenza, COVID-19) resulted in a humoral immune responses similar to patients treated with a placebo (SLE/influenza vaccine) or the antibody levels induced were in line with published data (MS/mRNA COVID-19 vaccine). In contrast to the general recommendations so far, it has thus been surprisingly found that subjects suffering from autoimmune disorders can be subjected to BTK inhibitor treatment, in particular evobrutinib treatment, and nevertheless can be safely vaccinated and/or immunized against infections at any time such vaccination is deemed necessary or desirable. The fact that such observations were made in the treatment of different autoimmune diseases and with different types of vaccines (an influenza vaccine presumed to reflect a recall response; and a COVID-19 vaccine, which is an mRNA vaccine and assumed to reflect the response to a novel antigen due to the recent emergence of this virus) suggests broader applicability of these findings also to other autoimmune diseases and possibly even other inflammatory diseases, and different types of vaccines and antigens.

As a result, it has been found that an immune response to vaccinations can be mounted in various contexts during BTK inhibitor treatment, despite the presumed negative impact of BTK inhibitor administration on the part of the immune system responsible for mounting such an immune response to vaccinations.

Advantageously, these findings allow for a much higher applicability of vaccinations in the context of BTK inhibitor treatment, specifically in the context of evobrutinib treatment, and a much greater flexibility when to vaccinate in the context of treatment with such a BTK inhibitor.

Thus, in various aspects the present disclosure further relates to the following: In an aspect, the present disclosure relates to a method for treating a disease in a patient in need thereof, the method comprising:
(a) administering a BTK inhibitor (Bruton's tyrosine kinase inhibitor) to said patient, and
(b) vaccinating the patient against an infection.

BTK inhibitors are known to a skilled person and further described in the present disclosure. Additional information about BTK inhibitors and their use in the treatment of diseases is available from the published, see e.g. Ringheim et al., Frontiers in Immunology (2021), vol. 66, p. 2223; Tasso et al., Molecules (2021), vol. 26, p. 7411; and cited references therein.

Appropriate dosages will depend on the specific patient and clinical situation and thus will be determined by the responsible doctor.

Vaccinating occurs by administering a vaccine to said patient. Vaccines and their use to prevent infections are part of the skilled person's expertise. The exact vaccination protocol will be determined by the responsible physician based on the clinical prescriptions of the vaccine used and the specifics of the case.

As described herein, the present disclosure is directed to BTK inhibitor treatment and vaccination in close temporal proximity or even simultaneously, without the need for a specific order of the BTK inhibitor treatment and vaccination steps. Hence, the lettering (a) and (b) in the above method (or similar lettering relating to BTK inhibitor treatment and vaccination in further methods below) are not to be understood in a chronological sense. Administering the BTK inhibitor and vaccinating said patient according to this method of treatment can occur in either order, at overlapping times or simultaneously, unless explicitly specified otherwise.

As used herein, the term “patient” refers to a human to be treated by the methods of the present invention.

As used herein, the term “treating” or “treatment” includes any effect, e.g., lessening, reducing, modulating, ameliorating or eliminating, that results in the improvement of the disease.

Corresponding considerations apply to the further aspects of the present disclosure below.

In another aspect, the present disclosure relates to a method for treating a disease in a patient in need thereof, the method comprising:
(a) identifying a patient in need of treatment of said disease who is at risk of contracting an infection,
(b) vaccinating said patient who is at risk of contracting said infection against said infection, and
(c) administering a BTK inhibitor to said patient.

The step of “identifying a patient who is at risk of contracting an infection” (or “identifying that a patient is at risk of contracting an infection”) is a routine step for the ones skilled in the art. Typically, it will be carried out by a skilled physician (e.g. a physician in general medicine, in the field of immunology and/or the field of autoimmune disorders) by employing standard laboratory procedures, including e.g. determining antibody titers. From such antibody titers, it can be assessed to which degree a patient is already immune against an infection, and accordingly whether said patient is at risk of contracting such an infection.

With regard to the step of identifying whether a patient is at risk of contracting an infection and the step of vaccinating said patient, it is understood that the identification step precedes the vaccination step, since through the identification step patients to be vaccinated are identified.

In another aspect, the present disclosure relates to a method for treating a disease in a patient in need thereof, the method comprising:
(a) identifying a patient in need of treatment of said disease who is at risk of contracting an infection,
(b) vaccinating said patient who is at risk of contracting said infection against said infection, and
(c) treating said disease by administering a BTK inhibitor to said patient.

In another aspect, the present disclosure relates to a method for treating a disease in a patient in need thereof, the method comprising:
(a) administering a BTK inhibitor to said patient,
(b) identifying that said patient is at risk of contracting an infection, and
(c) vaccinating said patient against said infection.

In another aspect, the present disclosure relates to a method for treating a disease in a patient in need thereof, the method comprising:
(a) administering said BTK inhibitor recurrently to a patient, thereby treating said patient for said disease,
(b) identifying that said patient is at risk of contracting an infection while on recurrent treatment with said BTK inhibitor, and
(c) vaccinating said patient against said infection while on recurrent treatment with said BTK inhibitor, and optionally
(d) continuing said recurrent treatment with said BTK inhibitor.

As used herein, “recurrent” administration of said BTK inhibitor or “recurrent” treatment of said BTK inhibitor means that said BTK inhibitor is administered in regular time intervals as prescribed by the treatment protocol for said BTK inhibitor and disease (as it may e.g. be defined in the drug label of said BTK inhibitor), e.g. every day.

In another aspect, the present disclosure relates to a method for treating a disease in a patient in need thereof, the method comprising:
(a) administering a BTK inhibitor to said patient,
(b) vaccinating said patient against an infection, and
(c) continuing to administer said BTK inhibitor to said patient.

As the skilled person understands, the step of “continuing to administer said BTK inhibitor” will be carried out after the step of “administering a BTK inhibitor”. There can either be a time where no BTK inhibitor is administered between completion of the step of administering a BTK inhibitor and the step of continuing to administer said BTK inhibitor (i.e. there is an interruption in the treatment with said BTK inhibitor; features defining the length of such an interruption are disclosed further below) or the step of continuing to administer said BTK inhibitor can start right at the time when the step of administering a BTK inhibitor is completed (i.e. the patient is continuously treated). The step of vaccinating said patient can either fully or partially overlap with the step of administering a BTK inhibitor, the step of continuing to administer said BTK inhibitor, or both.

In another aspect, the present disclosure relates to a method for treating a disease in a patient in need thereof, the method comprising:
(a) optionally administering a BTK inhibitor to said patient,
(b) identifying that said patient is at risk of contracting an infection,
(c) vaccinating said patient against said infection, and
(d) optionally administering said BTK inhibitor to said patient,
with the proviso that said method comprises one or more treatment periods in which said BTK inhibitor is administered,
thereby treating said patient against said disease and reducing the risk of said patient of contracting said infection.

As used herein, “reducing the risk of said patient of contracting said infection” can either be complete reduction (i.e. the patient becomes fully immune against said infection) or partial reduction.

In another aspect, the present disclosure relates to a method for treating a disease in a patient in need thereof, said treatment comprising administering a BTK inhibitor to said patient, wherein, while said patient is under ongoing treatment with said BTK inhibitor, said patient is vaccinated against an infection.

The route of administration of said BTK inhibitor is not particularly limited. Administration can occur by any route that allows for successful treatment of said disease. Typically said administration of said BTK inhibitor will be by oral administration, e.g. in the form of a tablet, or by injection.

If the present disclosure states that at a certain time a patient is “under ongoing treatment” with a BTK inhibitor, this means that said treatment with a BTK inhibitor has started before that certain time and continues, without interruption, until that certain time or longer. In some embodiments, it means that at that certain time, the most recent administration of said BTK inhibitor occurred not more than 72 hours ago. In some embodiments, it means that at that certain time, the most recent administration of said BTK inhibitor occurred not more than 48 hours ago. In some embodiments, it means that at that certain time, the most recent administration of said BTK inhibitor occurred not more than 36 hours ago. In some embodiments, it means that at that certain time, the most recent administration of said BTK inhibitor occurred not more than 24 hours ago.

Similarly, if the present disclosure states that a certain event (e.g. vaccination) occurs while a patient is “under ongoing treatment” with a BTK inhibitor, this means that said treatment with a BTK inhibitor has started before that event and continues, without interruption, to the day of said event or longer (e.g., said treatment of the patient with a BTK inhibitor has started and continues, without interruption, until the patient has received at least one dose of the respective vaccine). In some embodiments, it means that at the time of said event, the most recent administration of said BTK inhibitor occurred not more than 72 hours ago. In some embodiments, it means that at the time of said event, the most recent administration of said BTK inhibitor occurred not more than 48 hours ago. In some embodiments, it means that at the time of said event, the most recent administration of said BTK inhibitor occurred not more than 36 hours ago. In some embodiments, it means that at the time of said event, the most recent administration of said BTK inhibitor occurred not more than 24 hours ago. Treatment will typically involve the recurrent administration of said BTK inhibitor at regular time intervals according to a protocol, e.g. every day.

In another aspect, the present disclosure relates to a method for reducing the risk of a subject to contract an infection, the method comprising:
(a) administering a BTK inhibitor to said subject, and
(b) vaccinating said subject against said infection.

In some embodiments, said subject is a patient having a disease.

In some embodiments, said administering a BTK inhibitor to said subject is for treating a disease of said subject.

In some embodiments, said subject is a patient in need of such treatment.

In another aspect, the present disclosure relates to a method for reducing the risk of a subject to contract an infection, the method comprising vaccinating said subject against said infection, wherein said subject is vaccinated while under ongoing treatment with a BTK inhibitor.

In some embodiments, said subject is a patient having a disease.

In some embodiments, said treatment with said BTK inhibitor is for treating a disease in a patient in need thereof.

In some embodiments, said treatment involves administering said BTK inhibitor to said subject.

In another aspect, the present disclosure relates to a method for treating a disease in a patient in need thereof and reducing the risk of an infection in said patient, said method comprising
(a) administering a BTK inhibitor to said patient, and
(b) vaccinating said patient against said infection.

In another aspect, the present disclosure relates to a BTK inhibitor for use in the treatment of a disease by a method comprising:
(a) administering said BTK inhibitor to a patient having said disease, and
(b) vaccinating the patient against an infection.

In another aspect, the present disclosure relates to a BTK inhibitor for use in the treatment of a disease by a method comprising:
(a) identifying a patient who is at risk of contracting an infection,
(b) vaccinating said patient who is at risk of contracting said infection against said infection, and
(c) administering said BTK inhibitor to said patient.

In some embodiments, said patient is a patient having said disease.

In some embodiments, said BTK inhibitor is administered to treat said disease.

In another aspect, the present disclosure relates to a BTK inhibitor for use in the treatment of a disease, wherein said BTK inhibitor is administered as follows:
(a) administering said BTK inhibitor to a patient,
(b) identifying that said patient is at risk of contracting an infection, and
(c) vaccinating said patient against said infection.

In some embodiments, said patient is a patient having said disease.

In some embodiments, said BTK inhibitor is administered to treat said disease.

In another aspect, the present disclosure relates to a BTK inhibitor for use in the treatment of a disease, wherein said BTK inhibitor is administered as follows:
(a) administering said BTK inhibitor recurrently to a patient, thereby treating said patient for said disease,
(b) identifying that said patient is at risk of contracting an infection while on recurrent treatment with said BTK inhibitor, and
(c) vaccinating said patient against said infection while on recurrent treatment with said BTK inhibitor, and optionally
(d) continuing said recurrent treatment with said BTK inhibitor.

In some embodiments, said patient is a patient having said disease.

In another aspect, the present disclosure relates to a BTK inhibitor for use in the treatment of a disease, wherein said BTK inhibitor is administered as follows:
(a) administering said BTK inhibitor to a patient,
(b) vaccinating said patient against an infection, and
(c) continuing to administer said BTK inhibitor to said patient.

In some embodiments, said patient is a patient having said disease.

In some embodiments, said BTK inhibitor is administered to treat said disease.

In another aspect, the present disclosure relates to a BTK inhibitor for use in the treatment of a disease, wherein said BTK inhibitor is administered by the following method:
(a) optionally administering said BTK inhibitor to a patient,
(b) identifying that said patient is at risk of contracting an infection,
(c) vaccinating said patient against said infection, and
(d) optionally administering said BTK inhibitor to said patient,
with the proviso that said method comprises one or more treatment periods in which said BTK inhibitor is administered.

In some embodiments, said patient is a patient having said disease.

In some embodiments, said BTK inhibitor is administered to treat said disease.

In another aspect, the present disclosure relates to a BTK inhibitor for use in the treatment of a disease by a method that involves administering said BTK inhibitor to a patient having said disease, said method involving vaccinating said patient against an infection while said patient is under ongoing treatment with said BTK inhibitor.

In another aspect, the present disclosure relates to a BTK inhibitor for use in the treatment of a disease, wherein, while a patient having said disease is under ongoing treatment with said BTK inhibitor, said patient is vaccinated against an infection.

In some embodiments, said treatment with said BTK inhibitor involves administering said BTK inhibitor to said patient.

In some embodiments, said method involves administering said BTK inhibitor to a patient having said disease

In another aspect, the present disclosure relates to a BTK inhibitor for use in the treatment of a disease, wherein said BTK inhibitor is administered in combination with a vaccine.

In some embodiments, said treatment is by administration of said BTK inhibitor to a patient in need thereof.

In some embodiments, said vaccine is a vaccine against an infection.

In some embodiments, said patient is vaccinated with said vaccine against an infection.

In some embodiments, said vaccine is administered to said patient.

In some embodiments, said treatment with said BTK inhibitor and vaccination with said vaccine occur either simultaneously or sequentially.

In some embodiments, administration of said BTK inhibitor and vaccination with said vaccine occur either simultaneously or sequentially.

In another aspect, the present disclosure relates to a vaccine for use in the prevention of an infection by a method comprising:
(a) administering a BTK inhibitor to a subject, and
(b) vaccinating the subject against said infection.

As used herein, “prevention” of an infection can mean complete prevention of said infection or partial prevention of said infection. Thus, “prevention” of an infection can either mean that by said vaccination the subject becomes fully immune or partially immune against that infection.

Accordingly, “prevention” of an infection can either mean that upon vaccination the infection is avoided, the risk of contracting is reduced or, if the patient contracts an infection, said infection is ameliorated (compared to a situation where no such vaccination had taken place).

In some embodiments, said subject is a patient having a disease.

In some embodiments, said administering a BTK inhibitor to said subject is for treating said disease.

In some embodiments, said administering a BTK inhibitor to said subject is for treating a disease of said subject.

In some embodiments, said subject is a patient in need of such treatment.

In another aspect, the present disclosure relates to a vaccine for use in the prevention of an infection by a method comprising vaccinating said subject against said infection, wherein said subject is vaccinated while under ongoing treatment with a BTK inhibitor.

In some embodiments, said subject is a patient having a disease.

In some embodiments, said treatment with said BTK inhibitor is for treating a disease in a patient.

In some embodiments, said patient is a patient having said disease.

In some embodiments, said treatment involves administering said BTK inhibitor to said subject.

In another aspect, the present disclosure relates to a vaccine for use in the prevention of an infection by a method comprising:
(a) identifying a subject who is at risk of contracting said infection,
(b) vaccinating said subject who is at risk of contracting said infection with said vaccine against said infection, and
(c) administering a BTK inhibitor to said subject.

In some embodiments, said BTK inhibitor is administered to treat a disease.

In some embodiments, wherein said subject is a patient having a disease.

In some embodiments, said BTK inhibitor is administered to treat said disease.

In another aspect, the present disclosure relates to a vaccine for use in the prevention of an infection by a method comprising:
(a) administering a BTK inhibitor to a subject,
(b) vaccinating said subject with said vaccine against said infection, and
(c) continuing to administer said BTK inhibitor to said subject.

In some embodiments, said BTK inhibitor is administered to treat a disease.

In some embodiments, said subject is a patient having a disease.

In some embodiments, said BTK inhibitor is administered to treat said disease.

In another aspect, the present disclosure relates to the use of a BTK inhibitor for the manufacture of a medicament for the treatment of a disease by a method comprising:
(a) administering said BTK inhibitor to a patient having said disease, and
(b) vaccinating the patient against an infection.

In another aspect, the present disclosure relates to the use of a BTK inhibitor for the manufacture of a medicament for the treatment of a disease by a method comprising:
(a) identifying a patient who is at risk of contracting an infection,
(b) vaccinating said patient who is at risk of contracting said infection against said infection, and
(c) administering said BTK inhibitor to said patient.

In some embodiments, said patient is a patient having said disease.

In some embodiments, said BTK inhibitor is administered to treat said disease.

In another aspect, the present disclosure relates to the use of a BTK inhibitor for the manufacture of a medicament for the treatment of a disease, wherein said BTK inhibitor is administered as follows:
(a) administering said BTK inhibitor to a patient,
(b) identifying that said patient is at risk of contracting an infection, and
(c) vaccinating said patient against said infection.

In some embodiments, said patient is a patient having said disease.

In some embodiments, said BTK inhibitor is administered to treat said disease.

In another aspect, the present disclosure relates to the use of a BTK inhibitor for the manufacture of a medicament for the treatment of a disease, wherein said BTK inhibitor is administered as follows:
(a) administering said BTK inhibitor recurrently to a patient, thereby treating said patient for said disease,
(b) identifying that said patient is at risk of contracting an infection while on recurrent treatment with said BTK inhibitor, and
(c) vaccinating said patient against said infection while on recurrent treatment with said BTK inhibitor, and optionally
(d) continuing said recurrent treatment with said BTK inhibitor.

In some embodiments, said patient is a patient having said disease.

In another aspect, the present disclosure relates to the use of a BTK inhibitor for the manufacture of a medicament for the treatment of a disease, wherein said BTK inhibitor is administered as follows:
(a) administering said BTK inhibitor to a patient,
(b) vaccinating said patient against an infection, and
(c) continuing to administer said BTK inhibitor to said patient.

In some embodiments, said patient is a patient having said disease.

In some embodiments, said BTK inhibitor is administered to treat said disease.

In another aspect, the present disclosure relates to the use of a BTK inhibitor for the manufacture of a medicament for the treatment of a disease, wherein said BTK inhibitor is administered by the following method:
(a) optionally administering said BTK inhibitor to a patient,
(b) identifying that said patient is at risk of contracting an infection,
(c) vaccinating said patient against said infection, and
(d) optionally administering said BTK inhibitor to said patient,
with the proviso that said method comprises one or more treatment periods in which said BTK inhibitor is administered.

In some embodiments, said patient is a patient having said disease.

In some embodiments, said BTK inhibitor is administered to treat said disease.

In another aspect, the present disclosure relates to the use of a BTK inhibitor for the manufacture of a medicament for the treatment of a disease by a method wherein, while a patient having said disease is under ongoing treatment with said BTK inhibitor, said patient is vaccinated against an infection.

In some embodiments, said method involves administering said BTK inhibitor to a patient having said disease

In some embodiments, said treatment with said BTK inhibitor involves administering said BTK inhibitor to said patient.

In another aspect, the present disclosure relates to the use of a vaccine for the manufacture of a medicament for the prevention of an infection by a method comprising:
(a) administering a BTK inhibitor to a subject, and
(b) vaccinating the subject against said infection.

In some embodiments, said subject is a patient having a disease.

In some embodiments, said administering a BTK inhibitor to said subject is for treating said disease.

In some embodiments, said administering a BTK inhibitor to said subject is for treating a disease of said subject.

In some embodiments, said subject is a patient in need of such treatment.

In another aspect, the present disclosure relates to the use of a vaccine for the manufacture of a medicament for the prevention of an infection by a method comprising vaccinating said subject against said infection, wherein said subject is vaccinated while under ongoing treatment with a BTK inhibitor.

In some embodiments, said subject is a patient having a disease.

In some embodiments, said treatment with said BTK inhibitor is for treating said disease.

In some embodiments, said treatment involves administering said BTK inhibitor to said subject.

In another aspect, the present disclosure relates to the use of a vaccine for the manufacture of a medicament for the prevention of an infection by a method comprising:
(a) identifying a subject who is at risk of contracting an infection,
(b) vaccinating said subject who is at risk of contracting said infection with said vaccine against said infection, and
(c) administering a BTK inhibitor to said subject.

In some embodiments, said BTK inhibitor is administered to treat a disease.

In some embodiments, said subject is a patient having a disease.

In some embodiments, said BTK inhibitor is administered to treat said disease.

In another aspect, the present disclosure relates to the use of a vaccine for the manufacture of a medicament for the prevention of an infection by a method comprising:
(a) administering a BTK inhibitor to a subject,
(b) vaccinating said subject with said vaccine against said infection, and
(c) continuing to administer said BTK inhibitor to said subject.

In some embodiments, said subject is a human.

In some embodiments, said BTK inhibitor is administered to treat a disease.

In some embodiments, said subject is a patient having a disease.

In some embodiments, said BTK inhibitor is administered to treat said disease.

The following embodiments relate to any of the aspects disclosed above (i.e. to any method for treating a disease, method for reducing the risk of a subject to contract an infection, BTK inhibitor for use, vaccine for use, use of a BTK inhibitor for the manufacture of a medicament or use of a vaccine for the manufacture of a medicament described above) or any of their embodiments described above.

In some embodiments, said patient is vaccinated while said patient is under ongoing BTK inhibitor treatment.

In some embodiments, said vaccine is administered while said patient is under ongoing BTK inhibitor treatment.

In some embodiments, at least one dose of said vaccine is administered during the treatment with said BTK inhibitor.

In some embodiments, at least one dose of said vaccine is administered while said patient is under ongoing BTK inhibitor treatment.

In some embodiments, at least a part of the vaccination procedure is carried out while said patient/subjection is under treatment with said BTK inhibitor.

In some embodiments, said BTK inhibitor treatment starts before said vaccination starts and continues until after said vaccination has started.

As the skilled person understands, in this context the expression “said vaccination starts” or “said vaccination has been completed” refers to the complete vaccination procedure (which may include the administration of multiple individual doses to the patient). Thus, in such a context the term “vaccination” comprises the administration of all doses required for completion of the vaccination procedure.

In some embodiments, wherein said BTK inhibitor treatment starts before said vaccination starts and continues until after said vaccination has been completed.

In some embodiments, said BTK inhibitor treatment starts after said vaccination has started and before said vaccination has been completed, and continues until after said vaccination has been completed.

In some embodiments, treatment with said BTK inhibitor is started before administration of said vaccine and is continued at least until administration of said vaccine.

In this context, “administration of said vaccine” refers to the administration of an individual dose of said vaccine. Depending on the specific vaccine, in some cases administration of a single dose of vaccine may be sufficient to complete the vaccination procedure, while in other cases the administration of more than one dose of vaccine may be required to complete the vaccination procedure.

In some embodiments, treatment with said BTK inhibitor is started before administration of said vaccine and is continued until after administration of said vaccine.

In some embodiments, said BTK inhibitor is administered before, during and after administration of said vaccine.

In some embodiments, said vaccine is administered at a time when treatment with said BTK inhibitor has started and is still ongoing.

In some embodiments, there is a temporal overlap between said vaccination procedure and said BTK inhibitor treatment.

In some embodiments, said BTK inhibitor is administered during said vaccination.

In some embodiments, treatment with said BTK inhibitor is started before administration of said vaccine starts and said treatment with said BTK inhibitor either does not end before administration of said vaccine starts or ends less than 4 weeks before administration of said vaccine starts.

As used herein, that the treatment with said BTK inhibitor starts at a first time and “does not end before” a second time means that said treatment starts at said first time and is continued at least until said second time, i.e. from the first time on at least until the second time, there are recurrent administrations of said BTK inhibitor (wherein, preferably, recurrent administrations of said BTK inhibitor means one or more administrations essentially every day).

As used herein, administration “essentially every day” means that administration occurs every day or almost every day (i.e. only on a very exceptional basis, there may be a day where no administration occurs). Administration “essentially every day” is not limited with regard to the number of doses that are administered per day, i.e. it can be one or more doses per day.

In some embodiments, treatment with said BTK inhibitor is started before administration of said vaccine starts and said treatment with said BTK inhibitor either does not end before administration of said vaccine starts or ends less than 3 weeks before administration of said vaccine starts.

In some embodiments, treatment with said BTK inhibitor is started before administration of said vaccine starts and said treatment with said BTK inhibitor either does not end before administration of said vaccine starts or ends less than 2 weeks before administration of said vaccine starts.

In some embodiments, treatment with said BTK inhibitor is started before administration of said vaccine starts and said treatment with said BTK inhibitor either does not end before administration of said vaccine starts or ends less than 1 week before administration of said vaccine starts.

In some embodiments, treatment with said BTK inhibitor is started before administration of said vaccine starts, and from this time on said BTK inhibitor is administered essentially every day until less than 4 weeks before administration of said vaccine starts or later.

In some embodiments, treatment with said BTK inhibitor is started before administration of said vaccine starts, and from this time on said BTK inhibitor is administered essentially every day until less than 3 weeks before administration of said vaccine starts or later.

In some embodiments, treatment with said BTK inhibitor is started before administration of said vaccine starts, and from this time on said BTK inhibitor is administered essentially every day until less than 2 weeks before administration of said vaccine starts or later.

In some embodiments, treatment with said BTK inhibitor is started before administration of said vaccine starts, and from this time on said BTK inhibitor is administered essentially every day until less than 1 week before administration of said vaccine starts or later.

In some embodiments, treatment with said BTK inhibitor is started before administration of said vaccine starts, and from this time on said BTK inhibitor is administered essentially every day until administration of said vaccine starts or later.

In some embodiments, said administration essentially every day can be one or more than one doses of BTK inhibitor administered essentially every day.

In some embodiments, treatment with said BTK inhibitor is started before administration of said vaccine starts and said treatment with said BTK inhibitor ends less than 4 weeks before administration of said vaccine starts.

In some embodiments, treatment with said BTK inhibitor is started before administration of said vaccine starts and said treatment with said BTK inhibitor ends not more than 3 weeks before administration of said vaccine starts.

In some embodiments, treatment with said BTK inhibitor is started before administration of said vaccine starts and said treatment with said BTK inhibitor ends not more than 2 weeks before administration of said vaccine starts.

In some embodiments, treatment with said BTK inhibitor is started before administration of said vaccine starts and said treatment with said BTK inhibitor ends not more than 1 week before administration of said vaccine starts.

In some embodiments, treatment with said BTK inhibitor starts after said vaccination has started and said treatment with said BTK inhibitor starts less than 4 weeks after said vaccination has been completed.

In some embodiments, treatment with said BTK inhibitor starts after said vaccination has started and said treatment with said BTK inhibitor starts not more than 3 weeks after said vaccination has been completed.

In some embodiments, treatment with said BTK inhibitor starts after said vaccination has started and said treatment with said BTK inhibitor starts not more than 2 weeks after said vaccination has been completed.

In some embodiments, treatment with said BTK inhibitor starts after said vaccination has started and said treatment with said BTK inhibitor starts not more than 1 week after said vaccination has been completed.

In some embodiments, the time period between administration of said BTK inhibitor and vaccination is less than 4 weeks.

In some embodiments, the time period between administration of said BTK inhibitor and vaccination is less than 3 weeks.

In some embodiments, the time period between administration of said BTK inhibitor and vaccination is less than 2 weeks.

In some embodiments, the time period between administration of said BTK inhibitor and vaccination is less than 1 weeks.

In some embodiments, vaccination occurs while said patient is under BTK inhibitor treatment and treatment with said BTK inhibitor is not interrupted or is interrupted for less than 8 weeks for administration of said vaccine.

In some embodiments, vaccination occurs while said patient is under BTK inhibitor treatment and treatment with said BTK inhibitor is not interrupted or is interrupted for less than 6 weeks for administration of said vaccine.

In some embodiments, vaccination occurs while said patient is under BTK inhibitor treatment and treatment with said BTK inhibitor is not interrupted or is interrupted for less than 4 weeks for administration of said vaccine.

In some embodiments, vaccination occurs while said patient is under BTK inhibitor treatment and treatment with said BTK inhibitor is not interrupted or is interrupted for less than 2 weeks for administration of said vaccine.

In some embodiments, vaccination occurs while said patient is under BTK inhibitor treatment and treatment with said BTK inhibitor is not interrupted or is interrupted for less than 1 weeks for administration of said vaccine.

In some embodiments, vaccination occurs while said patient is under BTK inhibitor treatment and treatment with said BTK inhibitor is not interrupted for administration of said vaccine.

In some embodiments, during the term where said patient is under BTK inhibitor treatment said BTK inhibitor is administered to said patient essentially every day.

In some embodiments, during the term where said patient is under BTK inhibitor treatment said BTK inhibitor is administered to said patient every day.

In some embodiments, during the BTK inhibitor treatment the patient receives recurrent administrations of BTK inhibitor essentially every day.

In some embodiments, during the BTK inhibitor treatment the patient receives recurrent administrations of BTK inhibitor every day.

In some embodiments, recurrent treatment with a BTK inhibitor means administration of said BTK inhibitor essentially every day.

In some embodiments, recurrent administration of said BTK inhibitor means that BTK inhibitor administration occurs essentially every day.

In some embodiments, step (d) starts less than 8 weeks after step (a) ends.

In some embodiments, step (d) starts less than 6 weeks after step (a) ends.

In some embodiments, step (d) starts less than 4 weeks after step (a) ends.

In some embodiments, step (d) starts less than 2 weeks after step (a) ends.

In some embodiments, step (d) starts less than 1 week after step (a) ends.

In some embodiments, step (d) starts right when step (a) ends.

In some embodiments, said subject is a human.

In some embodiments, said patient is a human.

In some embodiments, the subject/patient is an adult.

In some embodiments, the subject/patient is 18 to 75 years old.

In some embodiments, said patient/subject is identified as being at risk of contracting said infection by testing said patient/subject for a history of infection.

In some embodiments, said patient/subject is identified as being at risk of contracting said infection by determining in blood, serum or plasma of said patient/subject one or more antibody titers that are indicative of immunity against said infection.

In some embodiments, said step of identifying a patient who is at risk of contracting an infection/identifying that a patient is at risk of contracting an infection involves the measurement of one or more antibody titers, preferably from a blood, serum or plasma sample.

In some embodiments, by said method said patient is treated for said disease.

In some embodiments, by said method the symptoms of said disease are reduced.

In some embodiments, said administration of said BTK inhibitor is for treating a disease in a patient in need thereof.

In some embodiments, by administering said BTK inhibitor to said patient, said patient is treated for said disease.

In some embodiments, by administering said BTK inhibitor to said patient the symptoms of said disease are reduced.

In some embodiments, by said treatment said patient is treated for said disease.

In some embodiments, by said treatment the symptoms of said disease are reduced.

In some embodiments, by said treatment the frequency of clinical exacerbations of said autoimmune disease are reduced.

In some embodiments, by said treatment progression of said autoimmune disease is slowed down or stopped.

In some embodiments, administration of said BTK inhibitor to said patient results in successful treatment of said patient.

In some embodiments, by said method the risk of said patient to contract said infection is reduced.

In some embodiments, by said method prophylaxis against said infection is achieved.

In some embodiments, by said method an immune response of said patient against the agent causing said infection is achieved.

In some embodiments, by administering said vaccine to said patient, the risk of said patient to contract said infection is reduced.

In some embodiments, by administering said vaccine to said patient prophylaxis against said infection is achieved.

In some embodiments, by administering said vaccine to said patient an immune response of said patient against the agent causing said infection is achieved.

In some embodiments, by vaccinating the patient against said infection, the risk of said patient to contract said infection is reduced.

In some embodiments, by vaccinating the patient against said infection prophylaxis against said infection is achieved.

In some embodiments, by vaccinating the patient against said infection an immune response of said patient against the agent causing said infection is achieved.

In some embodiments, by vaccinating the patient against said infection full or partial immunization against said infection is accomplished.

In some embodiments, by vaccinating the patient against said infection full immunization against said infection is accomplished.

In some embodiments, prevention of an infection means that said subject becomes fully immune or partially immune against said infection.

In some embodiments, prevention of an infection means that said subject becomes fully immune against said infection.

In some embodiments, prevention of an infection means that said infection is avoided, that the risk of contracting said infection is reduced or that, if the patient contracts said infection, said infection is ameliorated (compared to a situation where no such prevention has taken place).

In some embodiments, said method is a method for vaccinating said subject and not a method for treating a disease.

In some embodiments, said method is a method for vaccinating said subject and not a method for treating a disease.

In some embodiments, said BTK inhibitor is selected from the group consisting of evobrutinib, tolebrutinib, fenebrutinib and remibrutinib.

In some embodiments, said BTK inhibitor is an irreversible BTK inhibitor.

In some embodiments, said BTK inhibitor is a BTK inhibitor that binds covalently to cysteine 481 of BTK.

In some embodiments, said BTK inhibitor has been shown in clinical studies to allow for successful treatment of said disease.

In some embodiments, said marketing authorization for the use of said BTK inhibitor in the treatment of said disease has been granted by the US Food and Drug Administration (FDA) or the European Medicines Agency (EMA).

In some embodiments, said BTK inhibitor is
1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof, or
1-(4-(((6-amino-5-(4-phenoxyphenyl)pyrimidin-4-yl)amino)methyl)-4-fluoropiperidin-1-yl)prop-2-en-1-one or a pharmaceutically acceptable salt, tautomer, or stereoisomer thereof, or
a mixture of any of the above at any ratio.

The phrase “pharmaceutically acceptable” is used herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio.

In some embodiments, said BTK inhibitor is
1-[4-({[6-amino-5-(4-phenoxyphenyl)pyrimidin-4-yl]amino}methyl)piperidin-1-yl]prop-2-en-1-one or a pharmaceutically acceptable salt thereof, or
1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

In some embodiments, said BTK inhibitor is 1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof, or a mixture of any of these at any ratio.

In some embodiments, said BTK inhibitor is 1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof.

In some embodiments, said BTK inhibitor is 1-[4-({[6-amino-5-(4-phenoxyphenyl)pyrimidin-4-yl]amino}methyl)piperidin-1-yl]prop-2-en-1-one or a pharmaceutically acceptable salt thereof.

In some embodiments, said BTK inhibitor is 1-[4-({[6-amino-5-(4-phenoxyphenyl)pyrimidin-4-yl]amino}methyl)piperidin-1-yl]prop-2-en-1-one.

In some embodiments, said BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt, tautomer, or stereoisomer thereof, or a mixture of any of these at any ratio.

In some embodiments, said BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

In some embodiments, said BTK inhibitor is 1-(4-(((6-amino-5-(4-phenoxyphenyl)pyrimidin-4-yl)amino)methyl)-4-fluoropiperidin-1-yl)prop-2-en-1-one.

In some embodiments, said BTK inhibitor is evobrutinib or a pharmaceutically acceptable salt, tautomer, or stereoisomer thereof, or a mixture of any of these at any ratio.

In some embodiments, said BTK inhibitor is evobrutinib or a pharmaceutically acceptable salt thereof.

In some embodiments, said BTK inhibitor is evobrutinib.

In some embodiments, said BTK inhibitor is a compound according to formula (I):

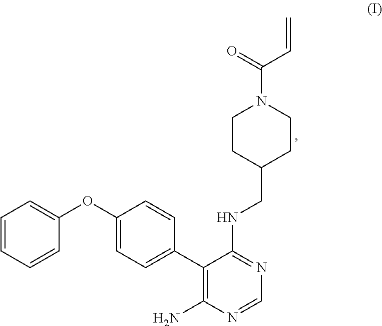 [SMILES: C=CC(=O)N1CCC(CNc2ncnc(N)c2-c2ccc(Oc3ccccc3)cc2)CC1]

or a pharmaceutically acceptable salt, tautomer, or stereoisomer thereof.

In some embodiments, said BTK inhibitor is a compound according to formula (I):

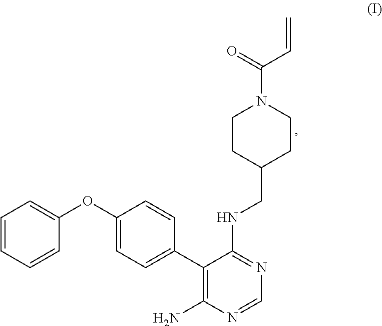 [SMILES: C=CC(=O)N1CCC(CNc2ncnc(N)c2-c2ccc(Oc3ccccc3)cc2)CC1]

or a pharmaceutically acceptable salt thereof.

In some embodiments, said BTK inhibitor is a compound according to formula (I):

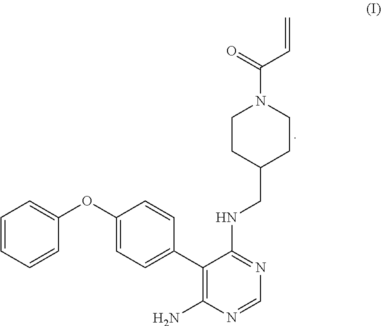 [SMILES: C=CC(=O)N1CCC(CNc2ncnc(N)c2-c2ccc(Oc3ccccc3)cc2)CC1]

In some embodiments, said disease is selected from the group consisting of systemic lupus erythematosus (SLE), multiple sclerosis (MS), rheumatoid arthritis (RA), pemphigus vulgaris (PV), Sjogren's syndrome (SJ), idiopathic thrombocytopenia (ITP), chronic spontaneous urticaria (CSU), graft versus host disease (GVHD), large vessel vasculitis (LVV) and asthma.

In some embodiments, said disease is selected from the group consisting of an autoimmune disease and an inflammatory disease.

In some embodiments, said disease is selected from the group consisting of an autoimmune disease, graft versus host disease (GVHD), large vessel vasculitis (LVV) and asthma.

In some embodiments, said inflammatory disease is selected from the group consisting of graft versus host disease (GVHD), large vessel vasculitis (LVV) and asthma.

In some embodiments, said disease is an autoimmune disease.

In some embodiments, the autoimmune disease is selected from the group consisting of systemic lupus erythematosus (SLE), multiple sclerosis (MS), rheumatoid arthritis (RA), pemphigus vulgaris (PV), Sjogren's syndrome (SJ), idiopathic thrombocytopenia (ITP) and chronic spontaneous urticaria (CSU).

In some embodiments, the autoimmune disease is selected from the group consisting of systemic lupus erythematosus (SLE), multiple sclerosis (MS), rheumatoid arthritis (RA), neuromyelitis optica spectrum disorders (NMOSD) and myasthenia gravis (MG), pemphigus vulgaris (PV), Sjogren's syndrome (SJ), idiopathic thrombocytopenia (ITP) and chronic spontaneous urticaria (CSU).

In some embodiments, the autoimmune disease is selected from the group consisting of systemic lupus erythematosus (SLE), multiple sclerosis (MS), rheumatoid arthritis (RA), neuromyelitis optica spectrum disorders (NMOSD) and myasthenia gravis (MG).

In some embodiments, said autoimmune disease is SLE (systemic lupus erythematosus) or MS (multiple sclerosis).

In some embodiments, said autoimmune disease is SLE (systemic lupus erythematosus).

In some embodiments, said autoimmune disease is MS (multiple sclerosis).

In some embodiments, said autoimmune disease is multiple sclerosis (MS), including one or more indications selected from the group consisting of relapsing multiple sclerosis (RMS), relapsing-remitting multiple sclerosis (RRMS), secondary progressive multiple sclerosis (SPMS) and primary progressive multiple sclerosis (PPMS).

In some embodiments, said autoimmune disease is selected from the group consisting of relapsing multiple sclerosis (RMS), relapsing-remitting multiple sclerosis (RRMS), secondary progressive multiple sclerosis (SPMS) and primary progressive multiple sclerosis (PPMS).

In some embodiments, said autoimmune disease is relapsing multiple sclerosis (RMS).

In some embodiments, said disease is a disease that can be treated with said BTK inhibitor.

In some embodiments, that treatment with said BTK inhibitor has been ongoing for a certain time means that at least for this time said BTK inhibitor has been administered essentially every day.

In some embodiments, being under ongoing treatment with said BTK inhibitor means that the most recent administration of said BTK inhibitor occurred not more than 72 hours ago.

In some embodiments, being under ongoing treatment with said BTK inhibitor means that the most recent administration of said BTK inhibitor occurred not more than 48 hours ago.

In some embodiments, being under ongoing treatment with said BTK inhibitor means that the most recent administration of said BTK inhibitor occurred not more than 36 hours ago.

In some embodiments, being under ongoing treatment with said BTK inhibitor means that the most recent administration of said BTK inhibitor occurred not more than 24 hours ago.

In some embodiments, an “ongoing treatment” with said BTK inhibitor means that the most recent administration of said BTK inhibitor occurred not more than 72 hours ago.

In some embodiments, an “ongoing treatment” with said BTK inhibitor means that the most recent administration of said BTK inhibitor occurred not more than 48 hours ago.

In some embodiments, an “ongoing treatment” with said BTK inhibitor means that the most recent administration of said BTK inhibitor occurred not more than 36 hours ago.

In some embodiments, an “ongoing treatment” with said BTK inhibitor means that the most recent administration of said BTK inhibitor occurred not more than 24 hours ago.

In some embodiments, said BTK inhibitor is administered at a daily amount ranging from 20 mg to 300 mg.

As the skilled person understands, said daily amount can be administered in a single administration or in multiple administrations that together result in the complete daily amount being administered. For example, a daily amount of 100 mg could either be administered by the patient taking one tablet of 100 mg in the morning or by the patient taking one table of 50 mg in the morning and one tablet of 50 mg in the evening, adding up to a total daily amount of 100 mg.

In some embodiments, said BTK inhibitor is administered at a daily amount ranging from 20 mg to 200 mg.

In some embodiments, said BTK inhibitor is administered at a daily amount ranging from 25 mg to 150 mg.

In some embodiments, said BTK inhibitor is administered at a daily amount ranging from 85 mg to 95 mg.

In some embodiments, said BTK inhibitor is administered at a daily amount ranging from 25 mg to 50 mg.

In some embodiments, said BTK inhibitor is administered at a daily amount ranging from 50 mg to 75 mg.

In some embodiments, said BTK inhibitor is administered at a daily amount ranging from 75 mg to 100 mg.

In some embodiments, said BTK inhibitor is administered at a daily amount ranging from 100 mg to 150 mg.

In some embodiments, said BTK inhibitor is administered at a daily amount ranging from 100 mg to 125 mg.

In some embodiments, said BTK inhibitor is administered at a daily amount ranging from 125 mg to 150 mg.

In some embodiments, said BTK inhibitor is administered at a daily amount ranging from 90 mg to 130 mg.

In some embodiments, said BTK inhibitor is administered at a daily amount of about 90 mg.

The term “about”, as used herein with respect to numbers, ranges or amounts, is used to refer to “circa” or “approximately” that number, range or amount. The meaning of those terms is well known in the art and indicates that a variance, deviation and/or variability is included in the respective number, range or amount, for example a variance, deviation and/or variability of up to plus/minus 15%. In some embodiments, the term “about” indicates a variance, deviation and/or variability of up to plus/minus 5%.

In some embodiments, said BTK inhibitor is administered at a daily amount of 90 mg.

In some embodiments, said daily amount of said BTK inhibitor is administered in a single unit formulation.

A unit formulation may for example be a tablet (in case of oral administration) or one injection shot (in case of administration by injection). Thus, if a patient is administered a daily amount of 100 mg by taking one tablet of 100 mg in the morning this would be administration of the daily amount in a single unit formulation. If, on the other hand, the patient is administered a daily amount of 100 mg by taking one tablet of 50 mg in the morning and one tablet of 50 mg in the evening, then this would be administration of the daily amount in two unit formulations.

In some embodiments, said daily amount of said BTK inhibitor is administered in two unit formulations that are administered independently and together make up that daily amount.

In some embodiments, said daily amount of said BTK inhibitor is administered in two or more unit formulations that are administered independently and together make up that daily amount.

In some embodiments, said daily amount of said BTK inhibitor is administered in a single unit formulation or in two unit formulations that are administered independently and together make up that daily amount.

In some embodiments, the individual unit formulations administered per day all contain the same amount of said BTK inhibitor.

In some embodiments, the unit formulation(s) are a tablet or capsule.

In some embodiments, 65 to 85 mg of said BTK inhibitor are administered once daily.

In some embodiments, 70 to 80 mg of said BTK inhibitor are administered once daily.

In some embodiments, about 75 mg of said BTK inhibitor are administered once daily.

In some embodiments, 75 mg of said BTK inhibitor are administered once daily.

In some embodiments, said once-daily administration of said BTK inhibitor is administered in a single unit dose.

In some embodiments, 40 to 85 mg of said BTK inhibitor are administered twice daily.

In some embodiments, 45 to 80 mg of said BTK inhibitor are administered twice daily.

In some embodiments, about 50 to about 75 mg of said BTK inhibitor are administered twice daily.

In some embodiments, 50 to 75 mg of said BTK inhibitor are administered twice daily.

In some embodiments, 40 to 60 mg of said BTK inhibitor are administered twice daily.

In some embodiments, 45 to 55 mg of said BTK inhibitor are administered twice daily.

In some embodiments, about 50 mg of said BTK inhibitor are administered twice daily.

In some embodiments, 50 mg of said BTK inhibitor are administered twice daily.

In some embodiments, 65 to 85 mg of said BTK inhibitor are administered twice daily.

In some embodiments, 70 to 80 mg of said BTK inhibitor are administered twice daily.

In some embodiments, about 75 mg of said BTK inhibitor are administered twice daily.

In some embodiments, 75 mg of said BTK inhibitor are administered twice daily.

In some embodiments, about 45 mg of said BTK inhibitor are administered twice daily.

In some embodiments, 45 mg of said BTK inhibitor are administered twice daily.

In some embodiments, administration of said BTK inhibitor twice daily means that two single unit doses of the same amount are administered per day.

In some embodiments, said BTK inhibitor is administered in such a manner that the resulting concentration of said BTK inhibitor in the plasma of said patient is similar to the concentration of said BTK inhibitor in the plasma achieved if said BTK inhibitor is administered by a dosing/administration regime as defined in any of the embodiments above by oral administration as a tablet.

In some embodiments, said BTK inhibitor is administered in such a manner that the resulting concentration of said BTK inhibitor in the plasma of said patient is similar to the concentration of said BTK inhibitor in the plasma achieved if said BTK inhibitor is administered at 40 mg to 85 mg twice daily by oral administration as a tablet.

In some embodiments, said BTK inhibitor is administered in such a manner that the resulting concentration of said BTK inhibitor in the plasma of said patient is similar to the concentration of said BTK inhibitor in the plasma achieved if said BTK inhibitor is administered at 40 mg to 85 mg twice daily under fed conditions by oral administration as a tablet.

In some embodiments, said BTK inhibitor is administered in such a manner that the therapeutic effect of said BTK inhibitor is similar to the therapeutic effect achieved if said BTK inhibitor is administered by a dosing/administration regime as defined in any of the embodiments above by oral administration as a tablet.

In some embodiments, administration “in such a manner” means administration “in such an amount, in such a formulation and by such an administration regime”.

In some embodiments, said BTK inhibitor is, at each occasion where said BTK inhibitor is administered, administered with a meal.

In some embodiments, said BTK inhibitor is, at each occasion where said BTK inhibitor is administered, administered without a meal.

In some embodiments, said BTK inhibitor is administered to a patient who has consumed food within 1 hour prior to receiving said BTK inhibitor.

In some embodiments, said BTK inhibitor is administered to a patient who has consumed food within 1 hour prior to being administered said BTK inhibitor and/or who does consume food within 1 hour after being administered said BTK inhibitor.

In some embodiments, said BTK inhibitor is administered to a patient who has not consumed food within 1 hour prior to being administered said BTK inhibitor and who does not consume food within 1 hour after being administered said BTK inhibitor.

In some embodiments, said BTK inhibitor is administered orally.

In some embodiments, said BTK inhibitor is administered as tablet(s) or capsule(s).

In some embodiments, said BTK inhibitor is administered as tablet(s).

In some embodiments, said vaccinating is performed by administering a vaccine.

In some embodiments, said vaccinating is performed by administering a vaccine to the subject/patient.

In some embodiments, said patient is vaccinated by administering a vaccine to said patient.

In some embodiments, said vaccine is selected from the group consisting of an inactivated vaccine, a live-attenuated vaccine, a live vaccine, a subunit vaccine, a recombinant vaccine, a polysaccharide vaccine, a conjugate vaccine, and a toxoid vaccine, or a combination thereof.

In some embodiments, said vaccine is selected from the group consisting of a live-attenuated vaccine and a live vaccine, or a combination thereof.

In some embodiments, said vaccine is an inactivated vaccine.

In some embodiments, said vaccination is performed with a vaccine selected from

In some embodiments, said vaccination is performed with a vaccine selected from the group consisting of an inactivated vaccine, a live-attenuated vaccine, a live vaccine, a subunit vaccine, a recombinant vaccine, a polysaccharide vaccine, a conjugate vaccine, and a toxoid vaccine, or a combination thereof.

In some embodiments, said vaccination is performed with a vaccine selected from the group consisting of a live-attenuated vaccine and a live vaccine, or a combination thereof.

In some embodiments, said vaccination is performed with a vaccine that is an inactivated vaccine.

In some embodiments, said vaccine is selected from the group consisting of anthrax prophylaxis vaccine, cholera prophylaxis vaccine, diphtheria prophylaxis vaccine, Haemophilus influenzae prophylaxis vaccine, meningococcal meningitis prophylaxis vaccine, pertussis prophylaxis vaccine, plague prophylaxis vaccine, pneumococcal disease prophylaxis vaccine, Streptococcus pneumoniae prophylaxis vaccine, tetanus prophylaxis vaccine, tuberculosis prophylaxis vaccine, typhoid prophylaxis vaccine.

In some embodiments, said vaccine is selected from the group consisting of: a varicella zoster virus vaccine, an attenuated live varicella zoster virus vaccine, an inactive varicella zoster virus vaccine, and an inactivated and/or recombinant varicella zoster virus vaccine.

In some embodiments, said vaccine is a recombinant, adjuvanted herpes zoster vaccine.

In some embodiments, said vaccine is a recombinant, adjuvanted herpes zoster vaccine that comprises recombinant varicella zoster virus glycoprotein E.

In some embodiments, said vaccine is SHINGRIX®.

In some embodiments, said patient is antibody-negative to varicella zoster virus, preferably antibody-negative to varicella zoster virus prior to said vaccination and/or prior to the start of said BTK inhibitor treatment.

In some embodiments, said vaccine is an influenza virus vaccine selected from the group consisting of a monovalent influenza virus vaccine, a divalent influenza virus vaccine, a trivalent influenza virus vaccine, a quadrivalent influenza virus vaccines and a polyvalent influenza virus vaccine.

In some embodiments, said vaccine is a quadrivalent influenza virus vaccines.

In some embodiments, said vaccine is selected from the group consisting of a corona vaccine, a COVID-19 vaccine, a SARS-COVID-19 vaccine and a SARS-CoV-2 vaccine.

In some embodiments, said vaccine is a corona vaccine.

In some embodiments, said vaccine is a COVID-19 vaccine.

In some embodiments, said vaccine is an mRNA vaccine.

In some embodiments, said vaccine is not an mRNA vaccine.

In some embodiments, said vaccine is a non-mRNA vaccine.

In some embodiments, said vaccine is a non-mRNA COVID-19 vaccine.

In some embodiments, said vaccine is an mRNA-based vaccine.

In some embodiments, said vaccine is a viral vector-based vaccine.

In some embodiments, said vaccine is a protein-based vaccine.

In some embodiments, said vaccine is not an mRNA-based vaccine.

In some embodiments, said vaccine is an mRNA-based COVID-19 vaccine.

In some embodiments, said vaccine is not an mRNA-based COVID-19 vaccine.

In some embodiments, said vaccine is an mRNA vaccine against COVID-19 of BioNTech/Pfizer or an mRNA vaccine against COVID-19 of Moderna.

In some embodiments, said vaccine is Comirnaty® by BioNTech/Pfizer or Spikevax® by Moderna or an identical vaccine distributed under a different name.

In some embodiments, said vaccine is Comirnaty® by BioNTech/Pfizer or Spikevax® by Moderna or an identical vaccine.

In some embodiments, said vaccine is mRNA vaccine against COVID-19 of BioNTech/Pfizer.

In some embodiments, said vaccine is Vaxzevria®/Covishield® (AstraZeneca) or an identical vaccine distributed under a different name.

In some embodiments, said vaccine is Vaxzevria® (AstraZeneca) or an identical vaccine.

In some embodiments, said vaccine is Jcovden® (Janssen/Johnson & Johnson) or an identical vaccine distributed under a different name.

In some embodiments, said vaccine is Jcovden® (Janssen/Johnson & Johnson) or an identical vaccine.

In some embodiments, said patient is, prior to said vaccination, antibody-negative for antibodies against the target antigen of said vaccination.

In some embodiments, said patient is, prior to said vaccination, seronegative for the antibodies that are generated in the immune response against said vaccine.

In some embodiments, the antigens to which said patient is exposed upon vaccination are novel antigens for the patient.

In some embodiments, the immune response of said patient to said vaccination is a de novo response.

In some embodiments, said patient is, prior to said vaccination, antibody-positive for antibodies against the target antigen of said vaccination.

In some embodiments, said patient is, prior to said vaccination, seropositive for the antibodies that are generated in the immune response against said vaccine.

In some embodiments, the antigens to which said patient is exposed upon vaccination are recall antigens for the patient.

In some embodiments, the immune response of said patient to said vaccination is a recall response.

In some embodiments, said vaccine is a vaccine against an infection.

In some embodiments, said infection is a viral infection.

In some embodiments, said viral infection is selected from the group consisting of hepatitis, chickenpox (varicella), shingles (herpes zoster), measles, influenza, poliovirus, a human papilloma virus (HPV)-related disease, SARS (severe acute respiratory syndrome), MERS (Middle East respiratory syndrome) and COVID-19 (Corona virus disease 2019).

In some embodiments, said hepatitis is hepatitis A and/or hepatitis B.

In some embodiments, said hepatitis is hepatitis B.

In some embodiments, said infection is a bacterial infection.

In some embodiments, said bacterial infection is selected from the group consisting of anthrax infection, cholera, diphtheria, Haemophilus influenzae infection, meningococcal meningitis, pertussis, plague, pneumococcal disease, Streptococcus pneumoniae infection, pneumococcal pneumonia, tetanus, tuberculosis, and typhus.

In some embodiments, said infection is a coronavirus infection.

In some embodiments, said infection is selected from the group consisting of SARS (severe acute respiratory syndrome), MERS (Middle East respiratory syndrome) and COVID-19 (Corona virus disease 2019).

In some embodiments, said infection is COVID-19.

In some embodiments, said infection is caused by a virus belonging to the realm of Riboviria.

In some embodiments, said infection is caused by a virus belonging to the realm of Riboviria and within that realm to the order of Nidovirales.

In some embodiments, said infection is caused by a virus belonging to the realm of Riboviria, within that realm to the order of Nidovirales, and within that order to the family of Coronaviridae.

In some embodiments, said infection is caused by a virus belonging to the realm of Riboviria, within that realm to the order of Nidovirales, within that order to the family of Coronaviridae, and within that family to the sub-family of Orthocoronavirinae.

In some embodiments, said infection is caused by a corona virus.

In some embodiments, said infection is caused by a virus selected from the group consisting of SARS-CoV-1 (severe acute respiratory syndrome coronavirus 1), MERS-CoV (Middle East respiratory syndrome-related coronavirus) and SARS-CoV-2 (severe acute respiratory syndrome coronavirus 2).

In some embodiments, said infection is caused by SARS-CoV-2 (severe acute respiratory syndrome coronavirus 2).

In some embodiments, said infection is influenza.

In some embodiments, said infection is seasonal flu.

In some embodiments, said infection is a varicella zoster infection.

In some embodiments, said infection is a herpes zoster infection.

In some embodiments, said infection is chickenpox.

In some embodiments, said infection is shingles.

In some embodiments, said vaccine is a vaccine against an infectious agent.

In some embodiments, said infection is an infection caused by an infectious agent.

In some embodiments, said infection is caused by an infectious agent.

In some embodiments, said infectious agent is a virus belonging to the realm of Riboviria.

In some embodiments, said infectious agent is a virus belonging to the realm of Riboviria and to the order of Nidovirales.

In some embodiments, said infectious agent is a virus belonging to the realm of Riboviria, to the order of Nidovirales, and to the family of Coronaviridae.

In some embodiments, said infectious agent is a virus belonging to the realm of Riboviria, to the order of Nidovirales, to the family of Coronaviridae, and to the sub-family of Orthocoronavirinae.

In some embodiments, said infectious agent is a corona virus.

In some embodiments, said infectious agent is a virus selected from the group consisting of SARS-CoV-1 (severe acute respiratory syndrome coronavirus 1), MERS-CoV (Middle East respiratory syndrome-related coronavirus) and SARS-CoV-2 (severe acute respiratory syndrome coronavirus 2).

In some embodiments, said infectious agent is SARS-CoV-2 (severe acute respiratory syndrome coronavirus 2).

In some embodiments, said vaccine is selected from the group consisting of: an mRNA-based SARS-CoV-2/COVID-19 virus vaccine, a vector-based SARS-CoV-2/COVID-19 virus vaccine, an adenovirus/vector-based SARS-CoV-2/COVID-19 virus vaccine, a virus-like particles (VLPs) based SARS-CoV-2/COVID-19 virus vaccine, a DNA-based SARS-CoV-2/COVID-19 virus vaccine, a protein-based SARS-CoV-2/COVID-19 virus vaccine, an inactivated virus-based SARS-CoV-2/COVID-19 virus vaccine, and a dead virus-based SARS-CoV-2/COVID-19 virus vaccine.

In some embodiments, said vaccine is selected from the group consisting of: an mRNA-based SARS-CoV-2/COVID-19 virus vaccine, a vector-based SARS-CoV-2/COVID-19 virus vaccine, and an adenovirus/vector-based SARS-CoV-2/COVID-19 virus vaccine.

In some embodiments, said vaccine is an mRNA-based SARS-CoV-2/COVID-19 virus vaccine.

In some embodiments, said vaccine is Comirnaty® (by BioNTech/Pfizer) or Spikevax® (by Moderna).

In some embodiments, said vaccine is Comirnaty®.

In some embodiments, said vaccine is Spikevax®.

In some embodiments, said infectious agent is an influenza virus.

In some embodiments, said infectious agent is an influenza virus of the strain HI A (human influenza A) or HI B (human influenza B).

In some embodiments, said infectious agent is an influenza virus of the strain HI A (human influenza A).

In some embodiments, said infectious agent is an influenza virus of the strain HI A (human influenza A), subtype A(H1N1).

In some embodiments, said infectious agent is an influenza virus of the strain HI A (human influenza A), subtype A(H3N2).

In some embodiments, said infectious agent is an influenza virus of the strain HI B (human influenza B).

In some embodiments, said infectious agent is an influenza virus of the strain HI B (human influenza B), lineage B/Yamagata.

In some embodiments, said infectious agent is an influenza virus of the strain HI B (human influenza B), lineage B/Victoria.

In some embodiments, said infectious agent is varicella zoster virus.

In some embodiments, said vaccine is administered by intramuscular injection.

In some embodiments, said vaccine is administered by oral administration.

In some embodiments, said vaccine is administered as a nasal spray.

In some embodiments, said vaccination consists of administering a single dose of vaccine.

In some embodiments, said vaccination consists of administering two separate doses of vaccine administered on different days.

In some embodiments, said vaccination consists of administering one dose of vaccine or two separate doses of vaccine administered on different days.

In some embodiments, said vaccination consists of administering a single dose of vaccine or 2 to 6 separate doses of vaccine administered on different days.

In some embodiments, said administering said dose(s) of vaccine is administering said dose(s) to the patient.

In some embodiments, said two doses resp. said 2 to 6 doses are administered on different days within 1 week to 12 months.

In some embodiments, said two doses resp. said 2 to 6 separate doses are administered on different days within 1 week to 6 months.

In some embodiments, said two doses resp. said 2 to 6 separate doses are administered on different days within 1 to 12 weeks.

In some embodiments, said two doses resp. said 2 to 6 separate doses are administered on different days within 1 to 6 weeks.

In some embodiments, said two doses resp. said 2 to 6 separate doses are administered on different days within 1 to 4 weeks.

In some embodiments, said two separate doses resp. said 2 to 6 separate doses are administered on different days separated by a time period of at least 2 weeks.

In some embodiments, said two separate doses resp. said 2 to 6 separate doses are administered on different days separated by a time period of at least 4 weeks.

In some embodiments, said two separate doses are administered to the patient on different days separated by a time period of at least 2 months.

In some embodiments, said vaccinating optionally involves administration of a booster vaccination.

In some embodiments, said vaccinating involves administration of a booster vaccination.

In some embodiments, said vaccinating does not involve administration of a booster vaccination.

In some embodiments, the administration of said vaccine optionally involves administration of a booster vaccination.

In some embodiments, the administration of said vaccine involves administration of a booster vaccination.

In some embodiments, the administration of said vaccine does not involve administration of a booster vaccination.

A booster vaccination is a dose of a vaccine that is administered to an individual that has already received a primary vaccination (or vaccination series). Booster vaccinations are recommended when protection from the primary vaccination decreases over time and help to maintain or increase the protective immune response achieved by the primary vaccination. For example, after primary vaccination against tetanus, a booster vaccination for tetanus vaccination is typically recommended every 10 years, by which point memory cells specific against tetanus lose their function or undergo apoptosis. The recommended time interval between the primary vaccination and the administration of the booster vaccination for a specific vaccine is known to physicians with expertise in the respective medical field and can also be obtained from the product label of the vaccine or from the company that produces and markets the vaccine. In the case of an mRNA-based COVID-19 vaccine like Comirnaty® by BioNTech/Pfizer or Spikevax® by Moderna, the booster vaccination may for example be administered about 6 months after administration of the previous COVID-19 vaccination dose.

In some embodiments, said booster vaccination is administered before said BTK inhibitor is administered.

In some embodiments, said booster vaccination is administered after said BTK inhibitor is administered.

In some embodiments, said booster vaccination is administered while said patient is on recurrent treatment with said BTK inhibitor.

In some embodiments, said booster vaccination is administered while said patient is under ongoing BTK inhibitor treatment.

In some embodiments, the treatment with said BTK inhibitor lasts for at least 2 weeks.

In some embodiments, the treatment with said BTK inhibitor lasts for at least 1 month.

In some embodiments, the treatment with said BTK inhibitor lasts for at least 2 months.

In some embodiments, the treatment with said BTK inhibitor lasts for at least 3 months.

In some embodiments, the treatment with said BTK inhibitor lasts for at least 6 months.

In some embodiments, the treatment with said BTK inhibitor lasts for at least 12 months.

Due to the fact that most of the autoimmune diseases and other inflammatory diseases discussed herein can rarely be finally cured and thus are of chronic nature, treatment durations with said BTK inhibitors may span one or more years, typically several years, in which said BTK inhibitor is administered to said patients, preferably as described herein above and/or below, without a significant pause or break, or essentially without a significant pause or break.

However, there may be exceptional cases where the treatment period is limited as defined in the embodiments below.

In some embodiments, the treatment with said BTK inhibitor lasts for not more than 12 months.

In some embodiments, the treatment with said BTK inhibitor lasts for not more than 6 months.

In some embodiments, the treatment with said BTK inhibitor lasts for not more than 3 months.

In some embodiments, the treatment with said BTK inhibitor lasts for not more than 2 months.

In some embodiments, the treatment with said BTK inhibitor lasts for not more than 1 month.

In some embodiments, the treatment with said BTK inhibitor lasts for not more than 2 weeks.

In some embodiments, during the time for which said treatment with said BTK inhibitor lasts, the patient is under continuous treatment with said BTK inhibitor, without intermittent periods in which no treatment with said BTK inhibitor takes place.

In some embodiments, by administering said BTK inhibitor to said patient or subject, said patient or subject is treated for said disease.

In some embodiments, by administering said BTK inhibitor to said patient or subject, said patient or subject is treated for said disease.

In some embodiments, by administering a BTK inhibitor, BTK inhibitor treatment is performed.

In some embodiments, by administering a BTK inhibitor said patient is treated with said BTK inhibitor.

In some embodiments, at the time when said vaccination starts the treatment with said BTK inhibitor has been ongoing for at least 1 week.

In some embodiments, at the time when said vaccination starts the treatment with said BTK inhibitor has been ongoing for at least 2 weeks.

In some embodiments, at the time when said vaccination starts the treatment with said BTK inhibitor has been ongoing for at least 1 month.

In some embodiments, at the time when said vaccination starts the treatment with said BTK inhibitor has been ongoing for at least 2 months.

In some embodiments, at the time when said vaccination starts the treatment with said BTK inhibitor has been ongoing for at least 3 months.

In some embodiments, at the time when said vaccination starts the treatment with said BTK inhibitor has been ongoing for at least 6 months.

In some embodiments, at the time when said vaccination starts the treatment with said BTK inhibitor has been ongoing for at least 12 months.

In some embodiments, at the time when said vaccination starts the treatment with said BTK inhibitor has been ongoing for not more than 12 months.

In some embodiments, at the time when said vaccination starts the treatment with said BTK inhibitor has been ongoing for not more than 6 months.

In some embodiments, at the time when said vaccination starts the treatment with said BTK inhibitor has been ongoing for not more than 3 months.

In some embodiments, at the time when said vaccination starts the treatment with said BTK inhibitor has been ongoing for not more than 2 months.

In some embodiments, at the time when said vaccination starts the treatment with said BTK inhibitor has been ongoing for not more than 1 month.

In some embodiments, at the time when said vaccination starts the treatment with said BTK inhibitor has been ongoing for not more than 2 weeks.

In some embodiments, at the time when said vaccination starts the treatment with said BTK inhibitor has been ongoing for not more than 1 week.

In some embodiments, the treatment with said BTK inhibitor continues for at least 1 week after the time when said vaccination is completed.

In some embodiments, the treatment with said BTK inhibitor continues for at least 2 weeks after the time when said vaccination is completed.

In some embodiments, the treatment with said BTK inhibitor continues for at least 1 month after the time when said vaccination is completed.

In some embodiments, the treatment with said BTK inhibitor continues for at least 2 months after the time when said vaccination is completed.

In some embodiments, the treatment with said BTK inhibitor continues for at least 3 months after the time when said vaccination is completed.

In some embodiments, the treatment with said BTK inhibitor continues for at least 6 months after the time when said vaccination is completed.

In some embodiments, the treatment with said BTK inhibitor continues for at least 12 months after the time when said vaccination is completed.

In some embodiments, the treatment with said BTK inhibitor continues for not more than 12 months after the time when said vaccination is completed.

In some embodiments, the treatment with said BTK inhibitor continues for not more than 6 months after the time when said vaccination is completed.

In some embodiments, the treatment with said BTK inhibitor continues for not more than 3 months after the time when said vaccination is completed.

In some embodiments, the treatment with said BTK inhibitor continues for not more than 2 months after the time when said vaccination is completed.

In some embodiments, the treatment with said BTK inhibitor continues for not more than 1 month after the time when said vaccination is completed.

In some embodiments, the treatment with said BTK inhibitor continues for not more than 2 weeks after the time when said vaccination is completed.

In some embodiments, the treatment with said BTK inhibitor continues for not more than 1 week after the time when said vaccination is completed.

Also disclosed with regard to the above-described subject matter is the following:
[1] A method for treating a disease in a patient in need thereof, the method comprising:
   (a) administering a BTK inhibitor (Bruton's tyrosine kinase inhibitor) to said patient, and
   (b) vaccinating the patient against an infection.
[2] A method for treating a disease in a patient in need thereof, the method comprising:
   (a) identifying a patient in need of treatment of said disease who is at risk of contracting an infection,
   (b) vaccinating said patient who is at risk of contracting said infection against said infection, and
   (c) administering a BTK inhibitor to said patient.
[3] A method for treating a disease in a patient in need thereof, the method comprising:
   (a) identifying a patient in need of treatment of said disease who is at risk of contracting an infection,
   (b) vaccinating said patient who is at risk of contracting said infection against said infection, and
   (c) treating said disease by administering a BTK inhibitor to said patient.
[4] A method for treating a disease in a patient in need thereof, the method comprising:
   (a) administering a BTK inhibitor to said patient,
   (b) identifying that said patient is at risk of contracting an infection, and
   (c) vaccinating said patient against said infection.
[5] A method for treating a disease in a patient in need thereof, the method comprising:
   (a) administering said BTK inhibitor recurrently to a patient, thereby treating said patient for said disease,
   (b) identifying that said patient is at risk of contracting an infection while on recurrent treatment with said BTK inhibitor, and
   (c) vaccinating said patient against said infection while on recurrent treatment with said BTK inhibitor, and optionally
   (d) continuing said recurrent treatment with said BTK inhibitor.
[6] A method for treating a disease in a patient in need thereof, the method comprising:
   (a) administering a BTK inhibitor to said patient,
   (b) vaccinating said patient against an infection, and
   (c) continuing to administer said BTK inhibitor to said patient.
[7] A method for treating a disease in a patient in need thereof, the method comprising:
   (a) optionally administering a BTK inhibitor to said patient,
   (b) identifying that said patient is at risk of contracting an infection,
   (c) vaccinating said patient against said infection, and
   (d) optionally administering said BTK inhibitor to said patient,
   with the proviso that said method comprises one or more treatment periods in which said BTK inhibitor is administered,
   thereby treating said patient against said disease and reducing the risk of said patient of contracting said infection.
[8] A method for treating a disease in a patient in need thereof, said treatment comprising administering a BTK inhibitor to said patient, wherein, while said patient is under ongoing treatment with said BTK inhibitor, said patient is vaccinated against an infection.
[9] A method for reducing the risk of a subject to contract an infection, the method comprising:
   (a) administering a BTK inhibitor to said subject, and
   (b) vaccinating said subject against said infection.
[10] The method according to item [9], wherein said subject is a patient having a disease.
[11] The method according to any of items [9] or [10], wherein said administering a BTK inhibitor to said subject is for treating a disease of said subject.
[12] The method according to any of items [9] to [11], wherein said subject is a patient in need of such treatment.
[13] A method for reducing the risk of a subject to contract an infection, the method comprising vaccinating said subject against said infection, wherein said subject is vaccinated while under ongoing treatment with a BTK inhibitor.
[14] The method according to item [13], wherein said subject is a patient having a disease.
[15] The method according to any of items [13] or [14], wherein said treatment with said BTK inhibitor is for treating a disease in a patient in need thereof.
[16] The method according to any of items [13] to [15], wherein said treatment involves administering said BTK inhibitor to said subject.
[17] A method for treating a disease in a patient in need thereof and reducing the risk of an infection in said patient, said method comprising
   (a) administering a BTK inhibitor to said patient, and
   (b) vaccinating said patient against said infection.
[18]A BTK inhibitor for use in the treatment of a disease by a method comprising:
   (a) administering said BTK inhibitor to a patient having said disease, and
   (b) vaccinating the patient against an infection.
[19]A BTK inhibitor for use in the treatment of a disease by a method comprising:
   (a) identifying a patient who is at risk of contracting an infection,
   (b) vaccinating said patient who is at risk of contracting said infection against said infection, and
   (c) administering said BTK inhibitor to said patient.
[20] The BTK inhibitor for use according to item [19], wherein said patient is a patient having said disease.
[21] The BTK inhibitor for use according to any of items [19] or [20], wherein said BTK inhibitor is administered to treat said disease.
[22]A BTK inhibitor for use in the treatment of a disease, wherein said BTK inhibitor is administered as follows:
   (a) administering said BTK inhibitor to a patient,
   (b) identifying that said patient is at risk of contracting an infection, and
   (c) vaccinating said patient against said infection.
[23] The BTK inhibitor for use according to item [22], wherein said patient is a patient having said disease.
[24] The BTK inhibitor for use according to any of items [22] or [23], wherein said BTK inhibitor is administered to treat said disease.
[25]A BTK inhibitor for use in the treatment of a disease, wherein said BTK inhibitor is administered as follows:
   (a) administering said BTK inhibitor recurrently to a patient, thereby treating said patient for said disease,
   (b) identifying that said patient is at risk of contracting an infection while on recurrent treatment with said BTK inhibitor, and
   (c) vaccinating said patient against said infection while on recurrent treatment with said BTK inhibitor, and optionally
   (d) continuing said recurrent treatment with said BTK inhibitor.
[26] The BTK inhibitor for use according to item [25], wherein said patient is a patient having said disease.
[27]A BTK inhibitor for use in the treatment of a disease, wherein said BTK inhibitor is administered as follows:
   (a) administering said BTK inhibitor to a patient,
   (b) vaccinating said patient against an infection, and
   (c) continuing to administer said BTK inhibitor to said patient.
[28] The BTK inhibitor for use according to item [27], wherein said patient is a patient having said disease.
[29] The BTK inhibitor for use according to any of items [27] or [28], wherein said BTK inhibitor is administered to treat said disease.
[30]A BTK inhibitor for use in the treatment of a disease, wherein said BTK inhibitor is administered by the following method:
   (a) optionally administering said BTK inhibitor to a patient,
   (b) identifying that said patient is at risk of contracting an infection,
   (c) vaccinating said patient against said infection, and
   (d) optionally administering said BTK inhibitor to said patient,
   with the proviso that said method comprises one or more treatment periods in which said BTK inhibitor is administered.
[31] The BTK inhibitor for use according to item [30], wherein said patient is a patient having said disease.
[32] The BTK inhibitor for use according to any of items [30] or [31], wherein said BTK inhibitor is administered to treat said disease.
[33]A BTK inhibitor for use in the treatment of a disease by a method that involves administering said BTK inhibitor to a patient having said disease, said method involving vaccinating said patient against an infection while said patient is under ongoing treatment with said BTK inhibitor.
[34]A BTK inhibitor for use in the treatment of a disease, wherein, while a patient having said disease is under ongoing treatment with said BTK inhibitor, said patient is vaccinated against an infection.
[35] The BTK inhibitor for use according to item [34], wherein said treatment with said BTK inhibitor involves administering said BTK inhibitor to said patient.
[36] The BTK inhibitor for use according to any of items [34] or [35], wherein said method involves administering said BTK inhibitor to a patient having said disease
[37]A BTK inhibitor for use in the treatment of a disease, wherein said BTK inhibitor is administered in combination with a vaccine.
[38] The BTK inhibitor for use according to item [37], wherein said treatment is by administration of said BTK inhibitor to a patient in need thereof.
[39] The BTK inhibitor for use according to any of items [37] of [38], wherein said vaccine is a vaccine against an infection.
[40] The BTK inhibitor for use according to any of items [37] to [39], wherein said patient is vaccinated with said vaccine against an infection.
[41] The BTK inhibitor for use according to any of items [37] to [40], wherein said vaccine is administered to said patient.
[42] The BTK inhibitor for use according to any of items [37] to [41], wherein said treatment with said BTK inhibitor and vaccination with said vaccine occur either simultaneously or sequentially.
[43] The BTK inhibitor for use according to any of items [37] to [42], wherein administration of said BTK inhibitor and vaccination with said vaccine occur either simultaneously or sequentially.
[44]A vaccine for use in the prevention of an infection by a method comprising:
   (a) administering a BTK inhibitor to a subject, and
   (b) vaccinating the subject against said infection.
[45] The vaccine for use according to item [44], wherein said subject is a patient having a disease.
[46] The vaccine for use according to item [45], wherein said administering a BTK inhibitor to said subject is for treating said disease.
[47] The vaccine for use according to any of items [44] to [46], wherein said administering a BTK inhibitor to said subject is for treating a disease of said subject.
[48] The vaccine for use according to any of items [44] to [47], wherein said subject is a patient in need of such treatment.
[49]A vaccine for use in the prevention of an infection by a method comprising vaccinating said subject against said infection, wherein said subject is vaccinated while under ongoing treatment with a BTK inhibitor.
[50] The vaccine for use according to item [49], wherein said subject is a patient having a disease.
[51] The vaccine for use according to any of items [49] or [50], wherein said treatment with said BTK inhibitor is for treating a disease in a patient.
[52] The vaccine for use according to item [51], wherein said patient is a patient having said disease.
[53] The vaccine for use according to any of items [49] to [52], wherein said treatment involves administering said BTK inhibitor to said subject.
[54]A vaccine for use in the prevention of an infection by a method comprising:
   (a) identifying a subject who is at risk of contracting said infection,
   (b) vaccinating said subject who is at risk of contracting said infection with said vaccine against said infection, and
   (c) administering a BTK inhibitor to said subject.
[55] The vaccine for use according to item [54], wherein said BTK inhibitor is administered to treat a disease.
[56] The vaccine for use according to any of items [54] or [55], wherein said subject is a patient having a disease.
[57] The vaccine for use according to any of items [54] to [56], wherein said BTK inhibitor is administered to treat said disease.
[58]A vaccine for use in the prevention of an infection by a method comprising:
   (a) administering a BTK inhibitor to a subject,
   (b) vaccinating said subject with said vaccine against said infection, and
   (c) continuing to administer said BTK inhibitor to said subject.
[59] The vaccine for use according to item [58], wherein said BTK inhibitor is administered to treat a disease.
[60] The vaccine for use according to any of items [58] or [59], wherein said subject is a patient having a disease.
[61] The vaccine for use according to any of items [58] to [60], wherein said BTK inhibitor is administered to treat said disease.
[62] Use of a BTK inhibitor for the manufacture of a medicament for the treatment of a disease by a method comprising:
   (a) administering said BTK inhibitor to a patient having said disease, and
   (b) vaccinating the patient against an infection.
[63] Use of a BTK inhibitor for the manufacture of a medicament for the treatment of a disease by a method comprising:
   (a) identifying a patient who is at risk of contracting an infection,
   (b) vaccinating said patient who is at risk of contracting said infection against said infection, and
   (c) administering said BTK inhibitor to said patient.
[64] The use according to item [63], wherein said patient is a patient having said disease.
[65] The use according to any of items [63] or [64], wherein said BTK inhibitor is administered to treat said disease.
[66] Use of a BTK inhibitor for the manufacture of a medicament for the treatment of a disease, wherein said BTK inhibitor is administered as follows:
   (a) administering said BTK inhibitor to a patient,
   (b) identifying that said patient is at risk of contracting an infection, and
   (c) vaccinating said patient against said infection.
[67] The use according to item [66], wherein said patient is a patient having said disease.
[68] The use according to any of items [66] or [67], wherein said BTK inhibitor is administered to treat said disease.
[69] Use of a BTK inhibitor for the manufacture of a medicament for the treatment of a disease, wherein said BTK inhibitor is administered as follows:
   (a) administering said BTK inhibitor recurrently to a patient, thereby treating said patient for said disease,
   (b) identifying that said patient is at risk of contracting an infection while on recurrent treatment with said BTK inhibitor, and
   (c) vaccinating said patient against said infection while on recurrent treatment with said BTK inhibitor, and optionally
   (d) continuing said recurrent treatment with said BTK inhibitor.
[70] The use according to item [69], wherein said patient is a patient having said disease.
[71] Use of a BTK inhibitor for the manufacture of a medicament for the treatment of a disease, wherein said BTK inhibitor is administered as follows:
   (a) administering said BTK inhibitor to a patient,
   (b) vaccinating said patient against an infection, and
   (c) continuing to administer said BTK inhibitor to said patient.
[72] The use according to item [71], wherein said patient is a patient having said disease.
[73] The use according to any of items [71] or [72], wherein said BTK inhibitor is administered to treat said disease.
[74] Use of a BTK inhibitor for the manufacture of a medicament for the treatment of a disease, wherein said BTK inhibitor is administered by the following method:
   (a) optionally administering said BTK inhibitor to a patient,
   (b) identifying that said patient is at risk of contracting an infection,
   (c) vaccinating said patient against said infection, and
   (d) optionally administering said BTK inhibitor to said patient,
   with the proviso that said method comprises one or more treatment periods in which said BTK inhibitor is administered.
[75] The use according to item [74], wherein said patient is a patient having said disease.
[76] The use according to any of items [74] or [75], wherein said BTK inhibitor is administered to treat said disease.
[77] Use of a BTK inhibitor for the manufacture of a medicament for the treatment of a disease by a method wherein, while a patient having said disease is under ongoing treatment with said BTK inhibitor, said patient is vaccinated against an infection.
[78] The use according to item [77], wherein said method involves administering said BTK inhibitor to a patient having said disease
[79] The use according to any of items [77] or [78], wherein said treatment with said BTK inhibitor involves administering said BTK inhibitor to said patient.
[80] Use of a vaccine for the manufacture of a medicament for the prevention of an infection by a method comprising:
   (a) administering a BTK inhibitor to a subject, and
   (b) vaccinating the subject against said infection.
[81] The use according to item [80], wherein said subject is a patient having a disease.
[82] The use according to any of items [80] or [81], wherein said administering a BTK inhibitor to said subject is for treating said disease.
[83] The use according to any of items [80] to [82], wherein said administering a BTK inhibitor to said subject is for treating a disease of said subject.
[84] The use according to any of items [80] to [83], wherein said subject is a patient in need of such treatment.
[85] Use of a vaccine for the manufacture of a medicament for the prevention of an infection by a method comprising vaccinating said subject against said infection, wherein said subject is vaccinated while under ongoing treatment with a BTK inhibitor.
[86] The use according to item [85], wherein said subject is a patient having a disease.
[87] The use according to item [86], wherein said treatment with said BTK inhibitor is for treating said disease.
[88] The use according to any of items [85] to [87], wherein said treatment involves administering said BTK inhibitor to said subject.
[89] Use of a vaccine for the manufacture of a medicament for the prevention of an infection by a method comprising:
   (a) identifying a subject who is at risk of contracting an infection,
   (b) vaccinating said subject who is at risk of contracting said infection with said vaccine against said infection, and
   (c) administering a BTK inhibitor to said subject.
[90] The use according to item [89], wherein said BTK inhibitor is administered to treat a disease.
[91] The use according to any of items [89] or [90], wherein said subject is a patient having a disease.
[92] The use according to any of items [89] to [91], wherein said BTK inhibitor is administered to treat said disease.
[93] Use of a vaccine for the manufacture of a medicament for the prevention of an infection by a method comprising:
   (a) administering a BTK inhibitor to a subject,
   (b) vaccinating said subject with said vaccine against said infection, and
   (c) continuing to administer said BTK inhibitor to said subject.
[94] The use according to item [93], wherein said subject is a human.
[95] The use according to any of items [93] or [94], wherein said BTK inhibitor is administered to treat a disease.
[96] The use according to any of items [93] to [95], wherein said subject is a patient having a disease.
[97] The use according to any of items [93] to [96], wherein said BTK inhibitor is administered to treat said disease.
[98] The method according to any of items [1] to [17] or the BTK inhibitor for use according to any of items [18] to [43] or the vaccine for use according to any of items [44] to [61] or the use according to any of items [62] to [97], wherein said patient is vaccinated while said patient is under ongoing BTK inhibitor treatment.
[99] The method according to any of items [1] to [17] or [98] or the BTK inhibitor for use according to any of items [18] to [43] or [98] or the vaccine for use according to any of items [44] to [61] or [98] or the use according to any of items [62] to [98], wherein said vaccine is administered while said patient is under ongoing BTK inhibitor treatment.
[100] The method according to any of items [1] to [17] or [98] to [99] or the BTK inhibitor for use according to any of items [18] to [43] or [98] to [99] or the vaccine for use according to any of items [44] to [61] or [98] to [99] or the use according to any of items [62] to [99], wherein at least one dose of said vaccine is administered during the treatment with said BTK inhibitor.
[101] The method according to any of items [1] to [17] or [98] to [100] or the BTK inhibitor for use according to any of items [18] to [43] or [98] to [100] or the vaccine for use according to any of items [44] to [61] or [98] to [100] or the use according to any of items [62] to [100], wherein at least one dose of said vaccine is administered while said patient is under ongoing BTK inhibitor treatment.
[102] The method according to any of items [1] to [17] or [98] to [101] or the BTK inhibitor for use according to any of items [18] to [43] or [98] to [101] or the vaccine for use according to any of items [44] to [61] or [98] to [101] or the use according to any of items [62] to [101], wherein at least a part of the vaccination procedure is carried out while said patient/subjection is under treatment with said BTK inhibitor.
[103] The method according to any of items [1] to [17] or [98] to [102] or the BTK inhibitor for use according to any of items [18] to [43] or [98] to [102] or the vaccine for use according to any of items [44] to [61] or [98] to [102] or the use according to any of items [62] to [102], wherein said BTK inhibitor treatment starts before said vaccination starts and continues until after said vaccination has started.
[104] The method according to any of items [1] to [17] or [98] to [103] or the BTK inhibitor for use according to any of items [18] to [43] or [98] to [103] or the vaccine for use according to any of items [44] to [61] or [98] to [103] or the use according to any of items [62] to [103], wherein said BTK inhibitor treatment starts before said vaccination starts and continues until after said vaccination has been completed.
[105] The method according to any of items [1] to [17] or [98] to [104] or the BTK inhibitor for use according to any of items [18] to [43] or [98] to [104] or the vaccine for use according to any of items [44] to [61] or [98] to [104] or the use according to any of items [62] to [104], wherein said BTK inhibitor treatment starts after said vaccination has started and before said vaccination has been completed, and continues until after said vaccination has been completed.
[106] The method according to any of items [1] to [17] or [98] to [105] or the BTK inhibitor for use according to any of items [18] to [43] or [98] to [105] or the vaccine for use according to any of items [44] to [61] or [98] to [105] or the use according to any of items [62] to [105], wherein treatment with said BTK inhibitor is started before administration of said vaccine and is continued at least until administration of said vaccine.
[107] The method according to any of items [1] to [17] or [98] to [106] or the BTK inhibitor for use according to any of items [18] to [43] or [98] to [106] or the vaccine for use according to any of items [44] to [61] or [98] to [106] or the use according to any of items [62] to [106], wherein treatment with said BTK inhibitor is started before administration of said vaccine and is continued until after administration of said vaccine.
[108] The method according to any of items [1] to [17] or [98] to [107] or the BTK inhibitor for use according to any of items [18] to [43] or [98] to [107] or the vaccine for use according to any of items [44] to [61] or [98] to [107] or the use according to any of items [62] to [107], wherein said BTK inhibitor is administered before, during and after administration of said vaccine.
[109] The method according to any of items [1] to [17] or [98] to [108] or the BTK inhibitor for use according to any of items [18] to [43] or [98] to [108] or the vaccine for use according to any of items [44] to [61] or [98] to [108] or the use according to any of items [62] to [108], wherein said vaccine is administered at a time when treatment with said BTK inhibitor has started and is still ongoing.
[110] The method according to any of items [1] to [17] or [98] to [109] or the BTK inhibitor for use according to any of items [18] to [43] or [98] to [109] or the vaccine for use according to any of items [44] to [61] or [98] to [109] or the use according to any of items [62] to [109], wherein there is a temporal overlap between said vaccination procedure and said BTK inhibitor treatment.
[111] The method according to any of items [1] to [17] or [98] to [110] or the BTK inhibitor for use according to any of items [18] to [43] or [98] to [110] or the vaccine for use according to any of items [44] to [61] or [98] to [110] or the use according to any of items [62] to [110], wherein said BTK inhibitor is administered during said vaccination.
[112] The method according to any of items [1] to [17] or [98] to [111] or the BTK inhibitor for use according to any of items [18] to [43] or [98] to [111] or the vaccine for use according to any of items [44] to [61] or [98] to [111] or the use according to any of items [62] to [111], wherein treatment with said BTK inhibitor is started before administration of said vaccine starts and wherein said treatment with said BTK inhibitor either does not end before administration of said vaccine starts or ends less than 4 weeks before administration of said vaccine starts.
[113] The method according to any of items [1] to [17] or [98] to [112] or the BTK inhibitor for use according to any of items [18] to [43] or [98] to [112] or the vaccine for use according to any of items [44] to [61] or [98] to [112] or the use according to any of items [62] to [112], wherein treatment with said BTK inhibitor is started before administration of said vaccine starts and wherein said treatment with said BTK inhibitor either does not end before administration of said vaccine starts or ends less than 3 weeks before administration of said vaccine starts.
[114] The method according to any of items [1] to [17] or [98] to [113] or the BTK inhibitor for use according to any of items [18] to [43] or [98] to [113] or the vaccine for use according to any of items [44] to [61] or [98] to [113] or the use according to any of items [62] to [113], wherein treatment with said BTK inhibitor is started before administration of said vaccine starts and wherein said treatment with said BTK inhibitor either does not end before administration of said vaccine starts or ends less than 2 weeks before administration of said vaccine starts.
[115] The method according to any of items [1] to [17] or [98] to [114] or the BTK inhibitor for use according to any of items [18] to [43] or [98] to [114] or the vaccine for use according to any of items [44] to [61] or [98] to [114] or the use according to any of items [62] to [114], wherein treatment with said BTK inhibitor is started before administration of said vaccine starts and wherein said treatment with said BTK inhibitor either does not end before administration of said vaccine starts or ends less than 1 week before administration of said vaccine starts.
[116] The method according to any of items [1] to [17] or [98] to [115] or the BTK inhibitor for use according to any of items [18] to [43] or [98] to [115] or the vaccine for use according to any of items [44] to [61] or [98] to [115] or the use according to any of items [62] to [115], wherein treatment with said BTK inhibitor is started before administration of said vaccine starts, and from this time on said BTK inhibitor is administered essentially every day until less than 4 weeks before administration of said vaccine starts or later.
[117] The method according to any of items [1] to [17] or [98] to [116] or the BTK inhibitor for use according to any of items [18] to [43] or [98] to [116] or the vaccine for use according to any of items [44] to [61] or [98] to [116] or the use according to any of items [62] to [116], wherein treatment with said BTK inhibitor is started before administration of said vaccine starts, and from this time on said BTK inhibitor is administered essentially every day until less than 3 weeks before administration of said vaccine starts or later.
[118] The method according to any of items [1] to [17] or [98] to [117] or the BTK inhibitor for use according to any of items [18] to [43] or [98] to [117] or the vaccine for use according to any of items [44] to [61] or [98] to [117] or the use according to any of items [62] to [117], wherein treatment with said BTK inhibitor is started before administration of said vaccine starts, and from this time on said BTK inhibitor is administered essentially every day until less than 2 weeks before administration of said vaccine starts or later.
[119] The method according to any of items [1] to [17] or [98] to [118] or the BTK inhibitor for use according to any of items [18] to [43] or [98] to [118] or the vaccine for use according to any of items [44] to [61] or [98] to [118] or the use according to any of items [62] to [118], wherein treatment with said BTK inhibitor is started before administration of said vaccine starts, and from this time on said BTK inhibitor is administered essentially every day until less than 1 week before administration of said vaccine starts or later.
[120] The method according to any of items [1] to [17] or [98] to [119] or the BTK inhibitor for use according to any of items [18] to [43] or [98] to [119] or the vaccine for use according to any of items [44] to [61] or [98] to [119] or the use according to any of items [62] to [119], wherein treatment with said BTK inhibitor is started before administration of said vaccine starts, and from this time on said BTK inhibitor is administered essentially every day until administration of said vaccine starts or later.
[121] The method according to any of items [1] to [17] or [98] to [120] or the BTK inhibitor for use according to any of items [18] to [43] or [98] to [120] or the vaccine for use according to any of items [44] to [61] or [98] to [120] or the use according to any of items [62] to [120], wherein said administration essentially every day can be one or more than one doses of BTK inhibitor administered essentially every day.
[122] The method according to any of items [1] to [17] or [98] to [121] or the BTK inhibitor for use according to any of items [18] to [43] or [98] to [121] or the vaccine for use according to any of items [44] to [61] or [98] to [121] or the use according to any of items [62] to [121], wherein treatment with said BTK inhibitor is started before administration of said vaccine starts and wherein said treatment with said BTK inhibitor ends less than 4 weeks before administration of said vaccine starts.
[123] The method according to any of items [1] to [17] or [98] to [122] or the BTK inhibitor for use according to any of items [18] to [43] or [98] to [122] or the vaccine for use according to any of items [44] to [61] or [98] to [122] or the use according to any of items [62] to [122], wherein treatment with said BTK inhibitor is started before administration of said vaccine starts and wherein said treatment with said BTK inhibitor ends not more than 3 weeks before administration of said vaccine starts.
[124] The method according to any of items [1] to [17] or [98] to [123] or the BTK inhibitor for use according to any of items [18] to [43] or [98] to [123] or the vaccine for use according to any of items [44] to [61] or [98] to [123] or the use according to any of items [62] to [123], wherein treatment with said BTK inhibitor is started before administration of said vaccine starts and wherein said treatment with said BTK inhibitor ends not more than 2 weeks before administration of said vaccine starts.
[125] The method according to any of items [1] to [17] or [98] to [124] or the BTK inhibitor for use according to any of items [18] to [43] or [98] to [124] or the vaccine for use according to any of items [44] to [61] or [98] to [124] or the use according to any of items [62] to [124], wherein treatment with said BTK inhibitor is started before administration of said vaccine starts and wherein said treatment with said BTK inhibitor ends not more than 1 week before administration of said vaccine starts.
[126] The method according to any of items [1] to [17] or [98] to [125] or the BTK inhibitor for use according to any of items [18] to [43] or [98] to [125] or the vaccine for use according to any of items [44] to [61] or [98] to [125] or the use according to any of items [62] to [125], wherein treatment with said BTK inhibitor starts after said vaccination has started and wherein treatment with said BTK inhibitor starts less than 4 weeks after said vaccination has been completed.
[127] The method according to any of items [1] to [17] or [98] to [126] or the BTK inhibitor for use according to any of items [18] to [43] or [98] to [126] or the vaccine for use according to any of items [44] to [61] or [98] to [126] or the use according to any of items [62] to [126], wherein treatment with said BTK inhibitor starts after said vaccination has started and wherein treatment with said BTK inhibitor starts not more than 3 weeks after said vaccination has been completed.
[128] The method according to any of items [1] to [17] or [98] to [127] or the BTK inhibitor for use according to any of items [18] to [43] or [98] to [127] or the vaccine for use according to any of items [44] to [61] or [98] to [127] or the use according to any of items [62] to [127], wherein treatment with said BTK inhibitor starts after said vaccination has started and wherein treatment with said BTK inhibitor starts not more than 2 weeks after said vaccination has been completed.
[129] The method according to any of items [1] to [17] or [98] to [128] or the BTK inhibitor for use according to any of items [18] to [43] or [98] to [128] or the vaccine for use according to any of items [44] to [61] or [98] to [128] or the use according to any of items [62] to [128], wherein treatment with said BTK inhibitor starts after said vaccination has started and wherein treatment with said BTK inhibitor starts not more than 1 week after said vaccination has been completed.
[130] The method according to any of items [1] to [17] or [98] to [129] or the BTK inhibitor for use according to any of items [18] to [43] or [98] to [129] or the vaccine for use according to any of items [44] to [61] or [98] to [129] or the use according to any of items [62] to [129], wherein the time period between administration of said BTK inhibitor and vaccination is less than 4 weeks.
[131] The method according to any of items [1] to [17] or [98] to [130] or the BTK inhibitor for use according to any of items [18] to [43] or [98] to [130] or the vaccine for use according to any of items [44] to [61] or [98] to [130] or the use according to any of items [62] to [130], wherein the time period between administration of said BTK inhibitor and vaccination is less than 3 weeks.
[132] The method according to any of items [1] to [17] or [98] to [131] or the BTK inhibitor for use according to any of items [18] to [43] or [98] to [131] or the vaccine for use according to any of items [44] to [61] or [98] to [131] or the use according to any of items [62] to [131], wherein the time period between administration of said BTK inhibitor and vaccination is less than 2 weeks.
[133] The method according to any of items [1] to [17] or [98] to [132] or the BTK inhibitor for use according to any of items [18] to [43] or [98] to [132] or the vaccine for use according to any of items [44] to [61] or [98] to [132] or the use according to any of items [62] to [132], wherein the time period between administration of said BTK inhibitor and vaccination is less than 1 weeks.
[134] The method according to any of items [1] to [17] or [98] to [133] or the BTK inhibitor for use according to any of items [18] to [43] or [98] to [133] or the vaccine for use according to any of items [44] to [61] or [98] to [133] or the use according to any of items [62] to [133], wherein vaccination occurs while said patient is under BTK inhibitor treatment and treatment with said BTK inhibitor is not interrupted or is interrupted for less than 8 weeks for administration of said vaccine.
[135] The method according to any of items [1] to [17] or [98] to [134] or the BTK inhibitor for use according to any of items [18] to [43] or [98] to [134] or the vaccine for use according to any of items [44] to [61] or [98] to [134] or the use according to any of items [62] to [134], wherein vaccination occurs while said patient is under BTK inhibitor treatment and treatment with said BTK inhibitor is not interrupted or is interrupted for less than 6 weeks for administration of said vaccine.
[136] The method according to any of items [1] to [17] or [98] to [135] or the BTK inhibitor for use according to any of items [18] to [43] or [98] to [135] or the vaccine for use according to any of items [44] to [61] or [98] to [135] or the use according to any of items [62] to [135], wherein vaccination occurs while said patient is under BTK inhibitor treatment and treatment with said BTK inhibitor is not interrupted or is interrupted for less than 4 weeks for administration of said vaccine.
[137] The method according to any of items [1] to [17] or [98] to [136] or the BTK inhibitor for use according to any of items [18] to [43] or [98] to [136] or the vaccine for use according to any of items [44] to [61] or [98] to [136] or the use according to any of items [62] to [136], wherein vaccination occurs while said patient is under BTK inhibitor treatment and treatment with said BTK inhibitor is not interrupted or is interrupted for less than 2 weeks for administration of said vaccine.
[138] The method according to any of items [1] to [17] or [98] to [137] or the BTK inhibitor for use according to any of items [18] to [43] or [98] to [137] or the vaccine for use according to any of items [44] to [61] or [98] to [137] or the use according to any of items [62] to [137], wherein vaccination occurs while said patient is under BTK inhibitor treatment and treatment with said BTK inhibitor is not interrupted or is interrupted for less than 1 weeks for administration of said vaccine.
[139] The method according to any of items [1] to [17] or [98] to [138] or the BTK inhibitor for use according to any of items [18] to [43] or [98] to [138] or the vaccine for use according to any of items [44] to [61] or [98] to [138] or the use according to any of items [62] to [138], wherein vaccination occurs while said patient is under BTK inhibitor treatment and treatment with said BTK inhibitor is not interrupted for administration of said vaccine.
[140] The method according to any of items [1] to [17] or [98] to [139] or the BTK inhibitor for use according to any of items [18] to [43] or [98] to [139] or the vaccine for use according to any of items [44] to [61] or [98] to [139] or the use according to any of items [62] to [139], wherein during the term where said patient is under BTK inhibitor treatment said BTK inhibitor is administered to said patient essentially every day.
[141] The method according to any of items [1] to [17] or [98] to [140] or the BTK inhibitor for use according to any of items [18] to [43] or [98] to [140] or the vaccine for use according to any of items [44] to [61] or [98] to [140] or the use according to any of items [62] to [140], wherein during the term where said patient is under BTK inhibitor treatment said BTK inhibitor is administered to said patient every day.
[142] The method according to any of items [1] to [17] or [98] to [141] or the BTK inhibitor for use according to any of items [18] to [43] or [98] to [141] or the vaccine for use according to any of items [44] to [61] or [98] to [141] or the use according to any of items [62] to [141], wherein during the BTK inhibitor treatment the patient receives recurrent administrations of BTK inhibitor essentially every day.
[143] The method according to any of items [1] to [17] or [98] to [142] or the BTK inhibitor for use according to any of items [18] to [43] or [98] to [142] or the vaccine for use according to any of items [44] to [61] or [98] to [142] or the use according to any of items [62] to [142], wherein during the BTK inhibitor treatment the patient receives recurrent administrations of BTK inhibitor every day.
[144] The method according to any of items [1] to [17] or [98] to [143] or the BTK inhibitor for use according to any of items [18] to [43] or [98] to [143] or the vaccine for use according to any of items [44] to [61] or [98] to [143] or the use according to any of items [62] to [143], wherein recurrent treatment with a BTK inhibitor means administration of said BTK inhibitor essentially every day.
[145] The method according to any of items [1] to [17] or [98] to [144] or the BTK inhibitor for use according to any of items [18] to [43] or [98] to [144] or the vaccine for use according to any of items [44] to [61] or [98] to [144] or the use according to any of items [62] to [144], wherein recurrent administration of said BTK inhibitor means that BTK inhibitor administration occurs essentially every day.
[146] The method according to any of items [5] or [7] or [98] to [145] or the BTK inhibitor for use according to any of items [25] to [26] or [30] to [32] or [98] to [145] or the use according to any of items [69] to [70] or [74] to [76] or [98] to [145], wherein step (d) starts less than 8 weeks after step (a) ends.
[147] The method according to any of items [5] or [7] or [98] to [146] or the BTK inhibitor for use according to any of items [25] to [26] or [30] to [32] or [98] to [146] or the use according to any of items [69] to [70] or [74] to [76] or [98] to [146], wherein step (d) starts less than 6 weeks after step (a) ends.
[148] The method according to any of items [5] or [7] or [98] to [147] or the BTK inhibitor for use according to any of items [25] to [26] or [30] to [32] or [98] to [147] or the use according to any of items [69] to [70] or [74] to [76] or [98] to [147], wherein step (d) starts less than 4 weeks after step (a) ends.
[149] The method according to any of items [5] or [7] or [98] to [148] or the BTK inhibitor for use according to any of items [25] to [26] or [30] to [32] or [98] to [148] or the use according to any of items [69] to [70] or [74] to [76] or [98] to [148], wherein step (d) starts less than 2 weeks after step (a) ends.
[150] The method according to any of items [5] or [7] or [98] to [149] or the BTK inhibitor for use according to any of items [25] to [26] or [30] to [32] or [98] to [149] or the use according to any of items [69] to [70] or [74] to [76] or [98] to [149], wherein step (d) starts less than 1 week after step (a) ends.
[151] The method according to any of items [5] or [7] or [98] to [150] or the BTK inhibitor for use according to any of items [25] to [26] or [30] to [32] or [98] to [150] or the use according to any of items [69] to [70] or [74] to [76] or [98] to [150], wherein step (d) starts right when step (a) ends.
[152] The method according to any of items [1] to [17] or [98] to [151] or the BTK inhibitor for use according to any of items [18] to [43] or [98] to [151] or the vaccine for use according to any of items [44] to [61] or [98] to [145] or the use according to any of items [62] to [151], wherein said subject is a human.
[153] The method according to any of items [1] to [17] or [98] to [152] or the BTK inhibitor for use according to any of items [18] to [43] or [98] to [152] or the vaccine for use according to any of items [44] to [61] or [98] to [145] or [152] or the use according to any of items [62] to [152], wherein said patient is a human.
[154] The method according to any of items [1] to [17] or [98] to [153] or the BTK inhibitor for use according to any of items [18] to [43] or [98] to [153] or the vaccine for use according to any of items [44] to [61] or [98] to [145] or [152] to [153] or the use according to any of items [62] to [153], wherein the subject/patient is an adult.
[155] The method according to any of items [1] to [17] or [98] to [154] or the BTK inhibitor for use according to any of items [18] to [43] or [98] to [154] or the vaccine for use according to any of items [44] to [61] or [98] to [145] or [152] to [154] or the use according to any of items [62] to [154], wherein the subject/patient is 18 to 75 years old.
[156] The method according to any of items [1] to [17] or [98] to [155] or the BTK inhibitor for use according to any of items [18] to [43] or [98] to [155] or the vaccine for use according to any of items [44] to [61] or [98] to [145] or [152] to [155] or the use according to any of items [62] to [155], wherein said patient/subject is identified as being at risk of contracting said infection by testing said patient/subject for a history of infection.
[157] The method according to any of items [1] to [17] or [98] to [156] or the BTK inhibitor for use according to any of items [18] to [43] or [98] to [156] or the vaccine for use according to any of items [44] to [61] or [98] to [145] or [152] to [156] or the use according to any of items [62] to [156], wherein said patient/subject is identified as being at risk of contracting said infection by determining in blood, serum or plasma of said patient/subject one or more antibody titers that are indicative of immunity against said infection.
[158] The method according to any of items [1] to [17] or [98] to [157] or the BTK inhibitor for use according to any of items [18] to [43] or [98] to [157] or the vaccine for use according to any of items [44] to [61] or [98] to [145] or [152] to [157] or the use according to any of items [62] to [157], wherein said step of identifying a patient who is at risk of contracting an infection/identifying that a patient is at risk of contracting an infection involves the measurement of one or more antibody titers, preferably from a blood, serum or plasma sample.
[159] The method according to any of items [1] to [17] or [98] to [158] or the BTK inhibitor for use according to any of items [18] to [43] or [98] to [158] or the vaccine for use according to any of items [44] to [61] or [98] to [145] or [152] to [158] or the use according to any of items [62] to [158], wherein by said method said patient is treated for said disease.
[160] The method according to any of items [1] to [17] or [98] to [159] or the BTK inhibitor for use according to any of items [18] to [43] or [98] to [159] or the vaccine for use according to any of items [44] to [61] or [98] to [145] or [152] to [159] or the use according to any of items [62] to [159], wherein by said method the symptoms of said disease are reduced.
[161] The method according to any of items [1] to [17] or [98] to [160] or the BTK inhibitor for use according to any of items [18] to [43] or [98] to [160] or the vaccine for use according to any of items [44] to [61] or [98] to [145] or [152] to [160] or the use according to any of items [62] to [160], wherein said administration of said BTK inhibitor is for treating a disease in a patient in need thereof.
[162] The method according to any of items [1] to [17] or [98] to [161] or the BTK inhibitor for use according to any of items [18] to [43] or [98] to [161] or the vaccine for use according to any of items [44] to [61] or [98] to [145] or [152] to [161] or the use according to any of items [62] to [161], wherein by administering said BTK inhibitor to said patient, said patient is treated for said disease.
[163] The method according to any of items [1] to [17] or [98] to [162] or the BTK inhibitor for use according to any of items [18] to [43] or [98] to [162] or the vaccine for use according to any of items [44] to [61] or [98] to [145] or [152] to [162] or the use according to any of items [62] to [162], wherein by administering said BTK inhibitor to said patient the symptoms of said disease are reduced.
[164] The method according to any of items [1] to [17] or [98] to [163] or the BTK inhibitor for use according to any of items [18] to [43] or [98] to [163] or the vaccine for use according to any of items [44] to [61] or [98] to [145] or [152] to [163] or the use according to any of items [62] to [163], wherein by said treatment said patient is treated for said disease.
[165] The method according to any of items [1] to [17] or [98] to [164] or the BTK inhibitor for use according to any of items [18] to [43] or [98] to [164] or the vaccine for use according to any of items [44] to [61] or [98] to [145] or [152] to [164] or the use according to any of items [62] to [164], wherein by said treatment the symptoms of said disease are reduced.
[166] The method according to any of items [1] to [17] or [98] to [165] or the BTK inhibitor for use according to any of items [18] to [43] or [98] to [165] or the vaccine for use according to any of items [44] to [61] or [98] to [145] or [152] to [165] or the use according to any of items [62] to [165], wherein by said treatment the frequency of clinical exacerbations of said autoimmune disease are reduced.
[167] The method according to any of items [1] to [17] or [98] to [166] or the BTK inhibitor for use according to any of items [18] to [43] or [98] to [166] or the vaccine for use according to any of items [44] to [61] or [98] to [145] or [152] to [166] or the use according to any of items [62] to [166], wherein by said treatment progression of said autoimmune disease is slowed down or stopped.
[168] The method according to any of items [1] to [17] or [98] to [167] or the BTK inhibitor for use according to any of items [18] to [43] or [98] to [167] or the vaccine for use according to any of items [44] to [61] or [98] to [145] or [152] to [167] or the use according to any of items [62] to [167], wherein administration of said BTK inhibitor to said patient results in successful treatment of said patient.
[169] The method according to any of items [1] to [17] or [98] to [168] or the BTK inhibitor for use according to any of items [18] to [43] or [98] to [168] or the vaccine for use according to any of items [44] to [61] or [98] to [145] or [152] to [168] or the use according to any of items [62] to [168], wherein by said method the risk of said patient to contract said infection is reduced.
[170] The method according to any of items [1] to [17] or [98] to [169] or the BTK inhibitor for use according to any of items [18] to [43] or [98] to [169] or the vaccine for use according to any of items [44] to [61] or [98] to [145] or [152] to [169] or the use according to any of items [62] to [169], wherein by said method prophylaxis against said infection is achieved.
[171] The method according to any of items [1] to [17] or [98] to [170] or the BTK inhibitor for use according to any of items [18] to [43] or [98] to [170] or the vaccine for use according to any of items [44] to [61] or [98] to [145] or [152] to [170] or the use according to any of items [62] to [170], wherein by said method an immune response of said patient against the agent causing said infection is achieved.
[172] The method according to any of items [1] to [17] or [98] to [171] or the BTK inhibitor for use according to any of items [18] to [43] or [98] to [171] or the vaccine for use according to any of items [44] to [61] or [98] to [145] or [152] to [171] or the use according to any of items [62] to [171], wherein by administering said vaccine to said patient, the risk of said patient to contract said infection is reduced.
[173] The method according to any of items [1] to [17] or [98] to [172] or the BTK inhibitor for use according to any of items [18] to [43] or [98] to [172] or the vaccine for use according to any of items [44] to [61] or [98] to [145] or [152] to [172] or the use according to any of items [62] to [172], wherein by administering said vaccine to said patient prophylaxis against said infection is achieved.
[174] The method according to any of items [1] to [17] or [98] to [173] or the BTK inhibitor for use according to any of items [18] to [43] or [98] to [173] or the vaccine for use according to any of items [44] to [61] or [98] to [145] or [152] to [173] or the use according to any of items [62] to [173], wherein by administering said vaccine to said patient an immune response of said patient against the agent causing said infection is achieved.
[175] The method according to any of items [1] to [17] or [98] to [174] or the BTK inhibitor for use according to any of items [18] to [43] or [98] to [174] or the vaccine for use according to any of items [44] to [61] or [98] to [145] or [152] to [174] or the use according to any of items [62] to [174], wherein by vaccinating the patient against said infection, the risk of said patient to contract said infection is reduced.
[176] The method according to any of items [1] to [17] or [98] to [175] or the BTK inhibitor for use according to any of items [18] to [43] or [98] to [175] or the vaccine for use according to any of items [44] to [61] or [98] to [145] or [152] to [175] or the use according to any of items [62] to [175], wherein by vaccinating the patient against said infection prophylaxis against said infection is achieved.
[177] The method according to any of items [1] to [17] or [98] to [176] or the BTK inhibitor for use according to any of items [18] to [43] or [98] to [176] or the vaccine for use according to any of items [44] to [61] or [98] to [145] or [152] to [176] or the use according to any of items [62] to [176], wherein by vaccinating the patient against said infection an immune response of said patient against the agent causing said infection is achieved.
[178] The method according to any of items [1] to [17] or [98] to [177] or the BTK inhibitor for use according to any of items [18] to [43] or [98] to [177] or the vaccine for use according to any of items [44] to [61] or [98] to [145] or [152] to [177] or the use according to any of items [62] to [177], wherein by vaccinating the patient against said infection full or partial immunization against said infection is accomplished.
[179] The method according to any of items [1] to [17] or [98] to [178] or the BTK inhibitor for use according to any of items [18] to [43] or [98] to [178] or the vaccine for use according to any of items [44] to [61] or [98] to [145] or [152] to [178] or the use according to any of items [62] to [178], wherein by vaccinating the patient against said infection full immunization against said infection is accomplished.
[180] The method according to any of items [1] to [17] or [98] to [179] or the BTK inhibitor for use according to any of items [18] to [43] or [98] to [179] or the vaccine for use according to any of items [44] to [61] or [98] to [145] or [152] to [179] or the use according to any of items [62] to [179], wherein prevention of an infection means that said subject becomes fully immune or partially immune against said infection.
[181] The method according to any of items [1] to [17] or [98] to [180] or the BTK inhibitor for use according to any of items [18] to [43] or [98] to [180] or the vaccine for use according to any of items [44] to [61] or [98] to [145] or [152] to [180] or the use according to any of items [62] to [180], wherein prevention of an infection means that said subject becomes fully immune against said infection.
[182] The method according to any of items [1] to [17] or [98] to [181] or the BTK inhibitor for use according to any of items [18] to [43] or [98] to [181] or the vaccine for use according to any of items [44] to [61] or [98] to [145] or [152] to [181] or the use according to any of items [62] to [181], wherein prevention of an infection means that said infection is avoided, that the risk of contracting said infection is reduced or that, if the patient contracts said infection, said infection is ameliorated (compared to a situation where no such prevention has taken place).
[183] The method according to any of items [1] to [17] or [98] to [182] or the BTK inhibitor for use according to any of items [18] to [43] or [98] to [182] or the vaccine for use according to any of items [44] to [61] or [98] to [145] or [152] to [182] or the use according to any of items [62] to [182], wherein said method is a method for vaccinating said subject and not a method for treating a disease.
[184] The method according to any of items [1] to [17] or [98] to [183] or the BTK inhibitor for use according to any of items [18] to [43] or [98] to [183] or the vaccine for use according to any of items [44] to [61] or [98] to [145] or [152] to [183] or the use according to any of items [62] to [183], wherein said BTK inhibitor is selected from the group consisting of evobrutinib, tolebrutinib, fenebrutinib and remibrutinib.
[185] The method according to any of items [1] to [17] or [98] to [184] or the BTK inhibitor for use according to any of items [18] to [43] or [98] to [184] or the vaccine for use according to any of items [44] to [61] or [98] to [145] or [152] to [184] or the use according to any of items [62] to [184], wherein said BTK inhibitor is an irreversible BTK inhibitor.
[186] The method according to any of items [1] to [17] or [98] to [185] or the BTK inhibitor for use according to any of items [18] to [43] or [98] to [185] or the vaccine for use according to any of items [44] to [61] or [98] to [145] or [152] to [185] or the use according to any of items [62] to [185], wherein said BTK inhibitor is a BTK inhibitor that binds covalently to cysteine 481 of BTK.
[187] The method according to any of items [1] to [17] or [98] to [186] or the BTK inhibitor for use according to any of items [18] to [43] or [98] to [186] or the vaccine for use according to any of items [44] to [61] or [98] to [145] or [152] to [186] or the use according to any of items [62] to [186], wherein said BTK inhibitor has been shown in clinical studies to allow for successful treatment of said disease.
[188] The method according to any of items [1] to [17] or [98] to [187] or the BTK inhibitor for use according to any of items [18] to [43] or [98] to [187] or the vaccine for use according to any of items [44] to [61] or [98] to [145] or [152] to [187] or the use according to any of items [62] to [187], wherein marketing authorization for the use of said BTK inhibitor in the treatment of said disease has been granted by the US Food and Drug Administration (FDA) or the European Medicines Agency (EMA).
[189] The method according to any of items [1] to [17] or [98] to [188] or the BTK inhibitor for use according to any of items [18] to [43] or [98] to [188] or the vaccine for use according to any of items [44] to [61] or [98] to [145] or [152] to [188] or the use according to any of items [62] to [188], wherein said BTK inhibitor is
   1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof, or
   1-(4-(((6-amino-5-(4-phenoxyphenyl)pyrimidin-4-yl)amino)methyl)-4-fluoropiperidin-1-yl)prop-2-en-1-one or a pharmaceutically acceptable salt, tautomer, or stereoisomer thereof, or
   a mixture of any of the above at any ratio.
[190] The method according to any of items [1] to [17] or [98] to [189] or the BTK inhibitor for use according to any of items [18] to [43] or [98] to [189] or the vaccine for use according to any of items [44] to [61] or [98] to [145] or [152] to [189] or the use according to any of items [62] to [189], wherein said BTK inhibitor is
   1-[4-({[6-amino-5-(4-phenoxyphenyl)pyrimidin-4-yl]amino}methyl)piperidin-1-yl]prop-2-en-1-one or a pharmaceutically acceptable salt thereof, or
   1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.
[191] The method according to any of items [1] to [17] or [98] to [190] or the BTK inhibitor for use according to any of items [18] to [43] or [98] to [190] or the vaccine for use according to any of items [44] to [61] or [98] to [145] or [152] to [190] or the use according to any of items [62] to [190], wherein said BTK inhibitor is 1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof, or a mixture of any of these at any ratio.
[192] The method according to any of items [1] to [17] or [98] to [191] or the BTK inhibitor for use according to any of items [18] to [43] or [98] to [191] or the vaccine for use according to any of items [44] to [61] or [98] to [145] or [152] to [191] or the use according to any of items [62] to [191], wherein said BTK inhibitor is 1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof.
[193] The method according to any of items [1] to [17] or [98] to [192] or the BTK inhibitor for use according to any of items [18] to [43] or [98] to [192] or the vaccine for use according to any of items [44] to [61] or [98] to [145] or [152] to [192] or the use according to any of items [62] to [192], wherein said BTK inhibitor is 1-[4-({[6-amino-5-(4-phenoxyphenyl)pyrimidin-4-yl]amino}methyl)piperidin-1-yl]prop-2-en-1-one or a pharmaceutically acceptable salt thereof.
[194] The method according to any of items [1] to [17] or [98] to [193] or the BTK inhibitor for use according to any of items [18] to [43] or [98] to [193] or the vaccine for use according to any of items [44] to [61] or [98] to [145] or [152] to [193] or the use according to any of items [62] to [193], wherein said BTK inhibitor is 1-[4-({[6-amino-5-(4-phenoxyphenyl)pyrimidin-4-yl]amino}methyl)piperidin-1-yl]prop-2-en-1-one.
[195] The method according to any of items [1] to [17] or [98] to [194] or the BTK inhibitor for use according to any of items [18] to [43] or [98] to [194] or the vaccine for use according to any of items [44] to [61] or [98] to [145] or [152] to [194] or the use according to any of items [62] to [194], wherein said BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt, tautomer, or stereoisomer thereof, or a mixture of any of these at any ratio.
[196] The method according to any of items [1] to [17] or [98] to [195] or the BTK inhibitor for use according to any of items [18] to [43] or [98] to [195] or the vaccine for use according to any of items [44] to [61] or [98] to [145] or [152] to [195] or the use according to any of items [62] to [195], wherein said BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.
[197] The method according to any of items [1] to [17] or [98] to [196] or the BTK inhibitor for use according to any of items [18] to [43] or [98] to [196] or the vaccine for use according to any of items [44] to [61] or [98] to [145] or [152] to [196] or the use according to any of items [62] to [196], wherein said BTK inhibitor is 1-(4-(((6-amino-5-(4-phenoxyphenyl)pyrimidin-4-yl)amino)methyl)-4-fluoropiperidin-1-yl)prop-2-en-1-one.
[198] The method according to any of items [1] to [17] or [98] to [197] or the BTK inhibitor for use according to any of items [18] to [43] or [98] to [197] or the vaccine for use according to any of items [44] to [61] or [98] to [145] or [152] to [197] or the use according to any of items [62] to [197], wherein said BTK inhibitor is evobrutinib or a pharmaceutically acceptable salt, tautomer, or stereoisomer thereof, or a mixture of any of these at any ratio.
[199] The method according to any of items [1] to [17] or [98] to [198] or the BTK inhibitor for use according to any of items [18] to [43] or [98] to [198] or the vaccine for use according to any of items [44] to [61] or [98] to [145] or [152] to [198] or the use according to any of items [62] to [198], wherein said BTK inhibitor is evobrutinib or a pharmaceutically acceptable salt thereof.
[200] The method according to any of items [1] to [17] or [98] to [199] or the BTK inhibitor for use according to any of items [18] to [43] or [98] to [199] or the vaccine for use according to any of items [44] to [61] or [98] to [145] or [152] to [199] or the use according to any of items [62] to [199], wherein said BTK inhibitor is evobrutinib.
[201] The method according to any of items [1] to [17] or [98] to [199] or the BTK inhibitor for use according to any of items [18] to [43] or [98] to [199] or the vaccine for use according to any of items [44] to [61] or [98] to [145] or [152] to [199] or the use according to any of items [62] to [199], wherein said BTK inhibitor is a compound according to formula (I):

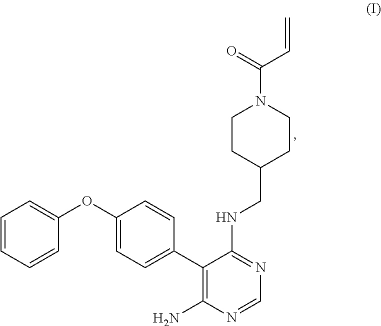 [SMILES: C=CC(=O)N1CCC(CNc2ncnc(N)c2-c2ccc(Oc3ccccc3)cc2)CC1]

or a pharmaceutically acceptable salt, tautomer, or stereoisomer thereof.
[202] The method according to any of items [1] to [17] or [98] to [201] or the BTK inhibitor for use according to any of items [18] to [43] or [98] to [201] or the vaccine for use according to any of items [44] to [61] or [98] to [145] or [152] to [201] or the use according to any of items [62] to [201], wherein said BTK inhibitor is a compound according to formula (I):

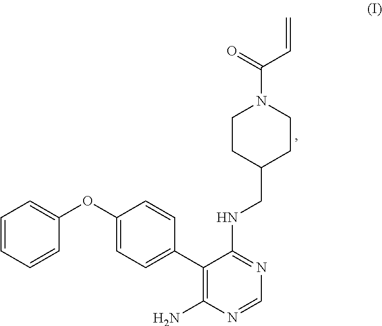 [SMILES: C=CC(=O)N1CCC(CNc2ncnc(N)c2-c2ccc(Oc3ccccc3)cc2)CC1]

or a pharmaceutically acceptable salt thereof.
[203] The method according to any of items [1] to [17] or [98] to [202] or the BTK inhibitor for use according to any of items [18] to [43] or [98] to [202] or the vaccine for use according to any of items [44] to [61] or [98] to [145] or [152] to [202] or the use according to any of items [62] to [202], wherein said BTK inhibitor is a compound according to formula (I):

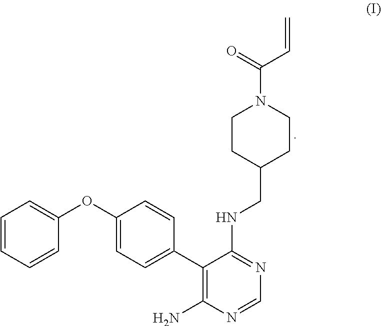 [SMILES: C=CC(=O)N1CCC(CNc2ncnc(N)c2-c2ccc(Oc3ccccc3)cc2)CC1]

[204] The method according to any of items [1] to [17] or [98] to [203] or the BTK inhibitor for use according to any of items [18] to [43] or [98] to [203] or the vaccine for use according to any of items [44] to [61] or [98] to [145] or [152] to [203] or the use according to any of items [62] to [203], wherein said disease is selected from the group consisting of systemic lupus erythematosus (SLE), multiple sclerosis (MS), rheumatoid arthritis (RA), pemphigus vulgaris (PV), Sjogren's syndrome (SJ), idiopathic thrombocytopenia (ITP), chronic spontaneous urticaria (CSU), graft versus host disease (GVHD), large vessel vasculitis (LVV) and asthma.
[205] The method according to any of items [1] to [17] or [98] to [204] or the BTK inhibitor for use according to any of items [18] to [43] or [98] to [204] or the vaccine for use according to any of items [44] to [61] or [98] to [145] or [152] to [204] or the use according to any of items [62] to [204], wherein said disease is selected from the group consisting of an autoimmune disease and an inflammatory disease.
[206] The method according to any of items [1] to [17] or [98] to [205] or the BTK inhibitor for use according to any of items [18] to [43] or [98] to [205] or the vaccine for use according to any of items [44] to [61] or [98] to [145] or [152] to [205] or the use according to any of items [62] to [205], wherein said disease is selected from the group consisting of an autoimmune disease, graft versus host disease (GVHD), large vessel vasculitis (LVV) and asthma.
[207] The method according to any of items [1] to [17] or [98] to [206] or the BTK inhibitor for use according to any of items [18] to [43] or [98] to [206] or the vaccine for use according to any of items [44] to [61] or [98] to [145] or [152] to [206] or the use according to any of items [62] to [206], wherein said inflammatory disease is selected from the group consisting of graft versus host disease (GVHD), large vessel vasculitis (LVV) and asthma.
[208] The method according to any of items [1] to [17] or [98] to [207] or the BTK inhibitor for use according to any of items [18] to [43] or [98] to [207] or the vaccine for use according to any of items [44] to [61] or [98] to [145] or [152] to [207] or the use according to any of items [62] to [207], wherein said disease is an autoimmune disease.
[209] The method according to any of items [1] to [17] or [98] to [208] or the BTK inhibitor for use according to any of items [18] to [43] or [98] to [208] or the vaccine for use according to any of items [44] to [61] or [98] to [145] or [152] to [208] or the use according to any of items [62] to [208], wherein the autoimmune disease is selected from the group consisting of systemic lupus erythematosus (SLE), multiple sclerosis (MS), rheumatoid arthritis (RA), pemphigus vulgaris (PV), Sjogren's syndrome (SJ), idiopathic thrombocytopenia (ITP) and chronic spontaneous urticaria (CSU).
[210] The method according to any of items [1] to [17] or [98] to [209] or the BTK inhibitor for use according to any of items [18] to [43] or [98] to [209] or the vaccine for use according to any of items [44] to [61] or [98] to [145] or [152] to [209] or the use according to any of items [62] to [209], wherein the autoimmune disease is selected from the group consisting of systemic lupus erythematosus (SLE), multiple sclerosis (MS), rheumatoid arthritis (RA), neuromyelitis optica spectrum disorders (NMOSD) and myasthenia gravis (MG), pemphigus vulgaris (PV), Sjogren's syndrome (SJ), idiopathic thrombocytopenia (ITP) and chronic spontaneous urticaria (CSU).
[211] The method according to any of items [1] to [17] or [98] to [210] or the BTK inhibitor for use according to any of items [18] to [43] or [98] to [210] or the vaccine for use according to any of items [44] to [61] or [98] to [145] or [152] to [210] or the use according to any of items [62] to [210], wherein the autoimmune disease is selected from the group consisting of systemic lupus erythematosus (SLE), multiple sclerosis (MS), rheumatoid arthritis (RA), neuromyelitis optica spectrum disorders (NMOSD) and myasthenia gravis (MG).
[212] The method according to any of items [1] to [17] or [98] to [211] or the BTK inhibitor for use according to any of items [18] to [43] or [98] to [211] or the vaccine for use according to any of items [44] to [61] or [98] to [145] or [152] to [211] or the use according to any of items [62] to [211], wherein said autoimmune disease is SLE (systemic lupus erythematosus) or MS (multiple sclerosis).
[213] The method according to any of items [1] to [17] or [98] to [212] or the BTK inhibitor for use according to any of items [18] to [43] or [98] to [212] or the vaccine for use according to any of items [44] to [61] or [98] to [145] or [152] to [212] or the use according to any of items [62] to [212], wherein said autoimmune disease is SLE (systemic lupus erythematosus).
[214] The method according to any of items [1] to [17] or [98] to [213] or the BTK inhibitor for use according to any of items [18] to [43] or [98] to [213] or the vaccine for use according to any of items [44] to [61] or [98] to [145] or [152] to [213] or the use according to any of items [62] to [213], wherein said autoimmune disease is MS (multiple sclerosis).
[215] The method according to any of items [1] to [17] or [98] to [214] or the BTK inhibitor for use according to any of items [18] to [43] or [98] to [214] or the vaccine for use according to any of items [44] to [61] or [98] to [145] or [152] to [214] or the use according to any of items [62] to [214], wherein said autoimmune disease is multiple sclerosis (MS), including one or more indications selected from the group consisting of relapsing multiple sclerosis (RMS), relapsing-remitting multiple sclerosis (RRMS), secondary progressive multiple sclerosis (SPMS) and primary progressive multiple sclerosis (PPMS).
[216] The method according to any of items [1] to [17] or [98] to [215] or the BTK inhibitor for use according to any of items [18] to [43] or [98] to [215] or the vaccine for use according to any of items [44] to [61] or [98] to [145] or [152] to [215] or the use according to any of items [62] to [215], wherein said autoimmune disease is selected from the group consisting of relapsing multiple sclerosis (RMS), relapsing-remitting multiple sclerosis (RRMS), secondary progressive multiple sclerosis (SPMS) and primary progressive multiple sclerosis (PPMS).
[217] The method according to any of items [1] to [17] or [98] to [216] or the BTK inhibitor for use according to any of items [18] to [43] or [98] to [216] or the vaccine for use according to any of items [44] to [61] or [98] to [145] or [152] to [216] or the use according to any of items [62] to [216], wherein said autoimmune disease is relapsing multiple sclerosis (RMS).
[218] The method according to any of items [1] to [17] or [98] to [217] or the BTK inhibitor for use according to any of items [18] to [43] or [98] to [217] or the vaccine for use according to any of items [44] to [61] or [98] to [145] or [152] to [217] or the use according to any of items [62] to [217], wherein said disease is a disease that can be treated with said BTK inhibitor.
[219] The method according to any of items [1] to [17] or [98] to [218] or the BTK inhibitor for use according to any of items [18] to [43] or [98] to [218] or the vaccine for use according to any of items [44] to [61] or [98] to [145] or [152] to [218] or the use according to any of items [62] to [218], wherein that treatment with said BTK inhibitor has been ongoing for a certain time means that at least for this time said BTK inhibitor has been administered essentially every day.
[220] The method according to any of items [1] to [17] or [98] to [219] or the BTK inhibitor for use according to any of items [18] to [43] or [98] to [219] or the vaccine for use according to any of items [44] to [61] or [98] to [145] or [152] to [219] or the use according to any of items [62] to [219], wherein being under ongoing treatment with said BTK inhibitor means that the most recent administration of said BTK inhibitor occurred not more than 72 hours ago.
[221] The method according to any of items [1] to [17] or [98] to [220] or the BTK inhibitor for use according to any of items [18] to [43] or [98] to [220] or the vaccine for use according to any of items [44] to [61] or [98] to [145] or [152] to [220] or the use according to any of items [62] to [220], wherein being under ongoing treatment with said BTK inhibitor means that the most recent administration of said BTK inhibitor occurred not more than 48 hours ago.
[222] The method according to any of items [1] to [17] or [98] to [221] or the BTK inhibitor for use according to any of items [18] to [43] or [98] to [221] or the vaccine for use according to any of items [44] to [61] or [98] to [145] or [152] to [221] or the use according to any of items [62] to [221], wherein being under ongoing treatment with said BTK inhibitor means that the most recent administration of said BTK inhibitor occurred not more than 36 hours ago.
[223] The method according to any of items [1] to [17] or [98] to [222] or the BTK inhibitor for use according to any of items [18] to [43] or [98] to [222] or the vaccine for use according to any of items [44] to [61] or [98] to [145] or [152] to [222] or the use according to any of items [62] to [222], wherein being under ongoing treatment with said BTK inhibitor means that the most recent administration of said BTK inhibitor occurred not more than 24 hours ago.
[224] The method according to any of items [1] to [17] or [98] to [223] or the BTK inhibitor for use according to any of items [18] to [43] or [98] to [223] or the vaccine for use according to any of items [44] to [61] or [98] to [145] or [152] to [223] or the use according to any of items [62] to [223], wherein an “ongoing treatment” with said BTK inhibitor means that the most recent administration of said BTK inhibitor occurred not more than 72 hours ago.
[225] The method according to any of items [1] to [17] or [98] to [224] or the BTK inhibitor for use according to any of items [18] to [43] or [98] to [224] or the vaccine for use according to any of items [44] to [61] or [98] to [145] or [152] to [224] or the use according to any of items [62] to [224], wherein an “ongoing treatment” with said BTK inhibitor means that the most recent administration of said BTK inhibitor occurred not more than 48 hours ago.
[226] The method according to any of items [1] to [17] or [98] to [225] or the BTK inhibitor for use according to any of items [18] to [43] or [98] to [225] or the vaccine for use according to any of items [44] to [61] or [98] to [145] or [152] to [225] or the use according to any of items [62] to [225], wherein an “ongoing treatment” with said BTK inhibitor means that the most recent administration of said BTK inhibitor occurred not more than 36 hours ago.
[227] The method according to any of items [1] to [17] or [98] to [226] or the BTK inhibitor for use according to any of items [18] to [43] or [98] to [226] or the vaccine for use according to any of items [44] to [61] or [98] to [145] or [152] to [226] or the use according to any of items [62] to [226], wherein an “ongoing treatment” with said BTK inhibitor means that the most recent administration of said BTK inhibitor occurred not more than 24 hours ago.
[228] The method according to any of items [1] to [17] or [98] to [227] or the BTK inhibitor for use according to any of items [18] to [43] or [98] to [227] or the vaccine for use according to any of items [44] to [61] or [98] to [145] or [152] to [227] or the use according to any of items [62] to [227], wherein said BTK inhibitor is administered at a daily amount ranging from 20 mg to 300 mg.
[229] The method according to any of items [1] to [17] or [98] to [228] or the BTK inhibitor for use according to any of items [18] to [43] or [98] to [228] or the vaccine for use according to any of items [44] to [61] or [98] to [145] or [152] to [228] or the use according to any of items [62] to [228], wherein said BTK inhibitor is administered at a daily amount ranging from 20 mg to 200 mg.
[230] The method according to any of items [1] to [17] or [98] to [229] or the BTK inhibitor for use according to any of items [18] to [43] or [98] to [229] or the vaccine for use according to any of items [44] to [61] or [98] to [145] or [152] to [229] or the use according to any of items [62] to [229], wherein said BTK inhibitor is administered at a daily amount ranging from 25 mg to 150 mg.
[231] The method according to any of items [1] to [17] or [98] to [230] or the BTK inhibitor for use according to any of items [18] to [43] or [98] to [230] or the vaccine for use according to any of items [44] to [61] or [98] to [145] or [152] to [230] or the use according to any of items [62] to [230], wherein said BTK inhibitor is administered at a daily amount ranging from 85 mg to 95 mg.
[232] The method according to any of items [1] to [17] or [98] to [231] or the BTK inhibitor for use according to any of items [18] to [43] or [98] to [231] or the vaccine for use according to any of items [44] to [61] or [98] to [145] or [152] to [231] or the use according to any of items [62] to [231], wherein said BTK inhibitor is administered at a daily amount ranging from 25 mg to 50 mg.
[233] The method according to any of items [1] to [17] or [98] to [232] or the BTK inhibitor for use according to any of items [18] to [43] or [98] to [232] or the vaccine for use according to any of items [44] to [61] or [98] to [145] or [152] to [232] or the use according to any of items [62] to [232], wherein said BTK inhibitor is administered at a daily amount ranging from 50 mg to 75 mg.
[234] The method according to any of items [1] to [17] or [98] to [233] or the BTK inhibitor for use according to any of items [18] to [43] or [98] to [233] or the vaccine for use according to any of items [44] to [61] or [98] to [145] or [152] to [233] or the use according to any of items [62] to [233], wherein said BTK inhibitor is administered at a daily amount ranging from 75 mg to 100 mg.
[235] The method according to any of items [1] to [17] or [98] to [234] or the BTK inhibitor for use according to any of items [18] to [43] or [98] to [234] or the vaccine for use according to any of items [44] to [61] or [98] to [145] or [152] to [234] or the use according to any of items [62] to [234], wherein said BTK inhibitor is administered at a daily amount ranging from 100 mg to 150 mg.
[236] The method according to any of items [1] to [17] or [98] to [235] or the BTK inhibitor for use according to any of items [18] to [43] or [98] to [235] or the vaccine for use according to any of items [44] to [61] or [98] to [145] or [152] to [235] or the use according to any of items [62] to [235], wherein said BTK inhibitor is administered at a daily amount ranging from 100 mg to 125 mg.
[237] The method according to any of items [1] to [17] or [98] to [236] or the BTK inhibitor for use according to any of items [18] to [43] or [98] to [236] or the vaccine for use according to any of items [44] to [61] or [98] to [145] or [152] to [236] or the use according to any of items [62] to [236], wherein said BTK inhibitor is administered at a daily amount ranging from 125 mg to 150 mg.
[238] The method according to any of items [1] to [17] or [98] to [237] or the BTK inhibitor for use according to any of items [18] to [43] or [98] to [237] or the vaccine for use according to any of items [44] to [61] or [98] to [145] or [152] to [237] or the use according to any of items [62] to [237], wherein said BTK inhibitor is administered at a daily amount ranging from 90 mg to 130 mg.
[239] The method according to any of items [1] to [17] or [98] to [238] or the BTK inhibitor for use according to any of items [18] to [43] or [98] to [238] or the vaccine for use according to any of items [44] to [61] or [98] to [145] or [152] to [238] or the use according to any of items [62] to [238], wherein said BTK inhibitor is administered at a daily amount of about 90 mg.
[240] The method according to any of items [1] to [17] or [98] to [239] or the BTK inhibitor for use according to any of items [18] to [43] or [98] to [239] or the vaccine for use according to any of items [44] to [61] or [98] to [145] or [152] to [239] or the use according to any of items [62] to [239], wherein said BTK inhibitor is administered at a daily amount of 90 mg.
[241] The method according to any of items [1] to [17] or [98] to [240] or the BTK inhibitor for use according to any of items [18] to [43] or [98] to [240] or the vaccine for use according to any of items [44] to [61] or [98] to [145] or [152] to [240] or the use according to any of items [62] to [240], wherein said daily amount of said BTK inhibitor is administered in a single unit formulation.
[242] The method according to any of items [1] to [17] or [98] to [241] or the BTK inhibitor for use according to any of items [18] to [43] or [98] to [241] or the vaccine for use according to any of items [44] to [61] or [98] to [145] or [152] to [241] or the use according to any of items [62] to [241], wherein said daily amount of said BTK inhibitor is administered in two unit formulations that are administered independently and together make up that daily amount.
[243] The method according to any of items [1] to [17] or [98] to [242] or the BTK inhibitor for use according to any of items [18] to [43] or [98] to [242] or the vaccine for use according to any of items [44] to [61] or [98] to [145] or [152] to [242] or the use according to any of items [62] to [242], wherein said daily amount of said BTK inhibitor is administered in two or more unit formulations that are administered independently and together make up that daily amount.
[244] The method according to any of items [1] to [17] or [98] to [243] or the BTK inhibitor for use according to any of items [18] to [43] or [98] to [243] or the vaccine for use according to any of items [44] to [61] or [98] to [145] or [152] to [243] or the use according to any of items [62] to [243], wherein said daily amount of said BTK inhibitor is administered in a single unit formulation or in two unit formulations that are administered independently and together make up that daily amount.
[245] The method according to any of items [1] to [17] or [98] to [244] or the BTK inhibitor for use according to any of items [18] to [43] or [98] to [244] or the vaccine for use according to any of items [44] to [61] or [98] to [145] or [152] to [244] or the use according to any of items [62] to [244], wherein the individual unit formulations administered per day all contain the same amount of said BTK inhibitor.
[246] The method according to any of items [1] to [17] or [98] to [245] or the BTK inhibitor for use according to any of items [18] to [43] or [98] to [245] or the vaccine for use according to any of items [44] to [61] or [98] to [145] or [152] to [245] or the use according to any of items [62] to [245], wherein the unit formulation(s) are a tablet or capsule.
[247] The method according to any of items [1] to [17] or [98] to [246] or the BTK inhibitor for use according to any of items [18] to [43] or [98] to [246] or the vaccine for use according to any of items [44] to [61] or [98] to [145] or [152] to [246] or the use according to any of items [62] to [246], wherein 65 to 85 mg of said BTK inhibitor are administered once daily.
[248] The method according to any of items [1] to [17] or [98] to [247] or the BTK inhibitor for use according to any of items [18] to [43] or [98] to [247] or the vaccine for use according to any of items [44] to [61] or [98] to [145] or [152] to [247] or the use according to any of items [62] to [247], wherein 70 to 80 mg of said BTK inhibitor are administered once daily.
[249] The method according to any of items [1] to [17] or [98] to [248] or the BTK inhibitor for use according to any of items [18] to [43] or [98] to [248] or the vaccine for use according to any of items [44] to [61] or [98] to [145] or [152] to [248] or the use according to any of items [62] to [248], wherein about 75 mg of said BTK inhibitor are administered once daily.
[250] The method according to any of items [1] to [17] or [98] to [249] or the BTK inhibitor for use according to any of items [18] to [43] or [98] to [249] or the vaccine for use according to any of items [44] to [61] or [98] to [145] or [152] to [249] or the use according to any of items [62] to [249], wherein 75 mg of said BTK inhibitor are administered once daily.
[251] The method according to any of items [1] to [17] or [98] to [250] or the BTK inhibitor for use according to any of items [18] to [43] or [98] to [250] or the vaccine for use according to any of items [44] to [61] or [98] to [145] or [152] to [250] or the use according to any of items [62] to [250], wherein said once-daily administration of said BTK inhibitor is administered in a single unit dose.
[252] The method according to any of items [1] to [17] or [98] to [251] or the BTK inhibitor for use according to any of items [18] to [43] or [98] to [251] or the vaccine for use according to any of items [44] to [61] or [98] to [145] or [152] to [251] or the use according to any of items [62] to [251], wherein 40 to 85 mg of said BTK inhibitor are administered twice daily.
[253] The method according to any of items [1] to [17] or [98] to [252] or the BTK inhibitor for use according to any of items [18] to [43] or [98] to [252] or the vaccine for use according to any of items [44] to [61] or [98] to [145] or [152] to [252] or the use according to any of items [62] to [252], wherein 45 to 80 mg of said BTK inhibitor are administered twice daily.
[254] The method according to any of items [1] to [17] or [98] to [253] or the BTK inhibitor for use according to any of items [18] to [43] or [98] to [253] or the vaccine for use according to any of items [44] to [61] or [98] to [145] or [152] to [253] or the use according to any of items [62] to [253], wherein about 50 to about 75 mg of said BTK inhibitor are administered twice daily.
[255] The method according to any of items [1] to [17] or [98] to [254] or the BTK inhibitor for use according to any of items [18] to [43] or [98] to [254] or the vaccine for use according to any of items [44] to [61] or [98] to [145] or [152] to [254] or the use according to any of items [62] to [254], wherein 50 to 75 mg of said BTK inhibitor are administered twice daily.
[256] The method according to any of items [1] to [17] or [98] to [255] or the BTK inhibitor for use according to any of items [18] to [43] or [98] to [255] or the vaccine for use according to any of items [44] to [61] or [98] to [145] or [152] to [255] or the use according to any of items [62] to [255], wherein 40 to 60 mg of said BTK inhibitor are administered twice daily.
[257] The method according to any of items [1] to [17] or [98] to [256] or the BTK inhibitor for use according to any of items [18] to [43] or [98] to [256] or the vaccine for use according to any of items [44] to [61] or [98] to [145] or [152] to [256] or the use according to any of items [62] to [256], wherein 45 to 55 mg of said BTK inhibitor are administered twice daily.
[258] The method according to any of items [1] to [17] or [98] to [257] or the BTK inhibitor for use according to any of items [18] to [43] or [98] to [257] or the vaccine for use according to any of items [44] to [61] or [98] to [145] or [152] to [257] or the use according to any of items [62] to [257], wherein about 50 mg of said BTK inhibitor are administered twice daily.
[259] The method according to any of items [1] to [17] or [98] to [258] or the BTK inhibitor for use according to any of items [18] to [43] or [98] to [258] or the vaccine for use according to any of items [44] to [61] or [98] to [145] or [152] to [258] or the use according to any of items [62] to [258], wherein 50 mg of said BTK inhibitor are administered twice daily.
[260] The method according to any of items [1] to [17] or [98] to [259] or the BTK inhibitor for use according to any of items [18] to [43] or [98] to [259] or the vaccine for use according to any of items [44] to [61] or [98] to [145] or [152] to [259] or the use according to any of items [62] to [259], wherein 65 to 85 mg of said BTK inhibitor are administered twice daily.
[261] The method according to any of items [1] to [17] or [98] to [260] or the BTK inhibitor for use according to any of items [18] to [43] or [98] to [260] or the vaccine for use according to any of items [44] to [61] or [98] to [145] or [152] to [260] or the use according to any of items [62] to [260], wherein 70 to 80 mg of said BTK inhibitor are administered twice daily.
[262] The method according to any of items [1] to [17] or [98] to [261] or the BTK inhibitor for use according to any of items [18] to [43] or [98] to [261] or the vaccine for use according to any of items [44] to [61] or [98] to [145] or [152] to [261] or the use according to any of items [62] to [261], wherein about 75 mg of said BTK inhibitor are administered twice daily.
[263] The method according to any of items [1] to [17] or [98] to [262] or the BTK inhibitor for use according to any of items [18] to [43] or [98] to [262] or the vaccine for use according to any of items [44] to [61] or [98] to [145] or [152] to [262] or the use according to any of items [62] to [262], wherein 75 mg of said BTK inhibitor are administered twice daily.
[264] The method according to any of items [1] to [17] or [98] to [263] or the BTK inhibitor for use according to any of items [18] to [43] or [98] to [263] or the vaccine for use according to any of items [44] to [61] or [98] to [145] or [152] to [263] or the use according to any of items [62] to [263], wherein about 45 mg of said BTK inhibitor are administered twice daily.
[265] The method according to any of items [1] to [17] or [98] to [264] or the BTK inhibitor for use according to any of items [18] to [43] or [98] to [264] or the vaccine for use according to any of items [44] to [61] or [98] to [145] or [152] to [264] or the use according to any of items [62] to [264], wherein 45 mg of said BTK inhibitor are administered twice daily.
[266] The method according to any of items [1] to [17] or [98] to [265] or the BTK inhibitor for use according to any of items [18] to [43] or [98] to [265] or the vaccine for use according to any of items [44] to [61] or [98] to [145] or [152] to [265] or the use according to any of items [62] to [265], wherein administration of said BTK inhibitor twice daily means that two single unit doses of the same amount are administered per day.
[267] The method according to any of items [1] to [17] or [98] to [266] or the BTK inhibitor for use according to any of items [18] to [43] or [98] to [266] or the vaccine for use according to any of items [44] to [61] or [98] to [145] or [152] to [266] or the use according to any of items [62] to [266], wherein said BTK inhibitor is administered in such a manner that the resulting concentration of said BTK inhibitor in the plasma of said patient is similar to the concentration of said BTK inhibitor in the plasma achieved if said BTK inhibitor is administered by a dosing/administration regime as defined in any of items [247] to [266] above by oral administration as a tablet.
[268] The method according to any of items [1] to [17] or [98] to [267] or the BTK inhibitor for use according to any of items [18] to [43] or [98] to [267] or the vaccine for use according to any of items [44] to [61] or [98] to [145] or [152] to [267] or the use according to any of items [62] to [267], wherein said BTK inhibitor is administered in such a manner that the resulting concentration of said BTK inhibitor in the plasma of said patient is similar to the concentration of said BTK inhibitor in the plasma achieved if said BTK inhibitor is administered at 40 mg to 85 mg twice daily by oral administration as a tablet.
[269] The method according to any of items [1] to [17] or [98] to [268] or the BTK inhibitor for use according to any of items [18] to [43] or [98] to [268] or the vaccine for use according to any of items [44] to [61] or [98] to [145] or [152] to [268] or the use according to any of items [62] to [268], wherein said BTK inhibitor is administered in such a manner that the resulting concentration of said BTK inhibitor in the plasma of said patient is similar to the concentration of said BTK inhibitor in the plasma achieved if said BTK inhibitor is administered at 40 mg to 85 mg twice daily under fed conditions by oral administration as a tablet.
[270] The method according to any of items [1] to [17] or [98] to [269] or the BTK inhibitor for use according to any of items [18] to [43] or [98] to [269] or the vaccine for use according to any of items [44] to [61] or [98] to [145] or [152] to [269] or the use according to any of items [62] to [269], wherein said BTK inhibitor is administered in such a manner that the therapeutic effect of said BTK inhibitor is similar to the therapeutic effect achieved if said BTK inhibitor is administered by a dosing/administration regime as defined in any of items [247] to [266] above by oral administration as a tablet.
[271] The method according to any of items [1] to [17] or [98] to [270] or the BTK inhibitor for use according to any of items [18] to [43] or [98] to [270] or the vaccine for use according to any of items [44] to [61] or [98] to [145] or [152] to [270] or the use according to any of items [62] to [270], wherein administration “in such a manner” means administration “in such an amount, in such a formulation and by such an administration regime”.
[272] The method according to any of items [1] to [17] or [98] to [271] or the BTK inhibitor for use according to any of items [18] to [43] or [98] to [271] or the vaccine for use according to any of items [44] to [61] or [98] to [145] or [152] to [271] or the use according to any of items [62] to [271], wherein said BTK inhibitor is, at each occasion where said BTK inhibitor is administered, administered with a meal.
[273] The method according to any of items [1] to [17] or [98] to [272] or the BTK inhibitor for use according to any of items [18] to [43] or [98] to [272] or the vaccine for use according to any of items [44] to [61] or [98] to [145] or [152] to [272] or the use according to any of items [62] to [272], wherein said BTK inhibitor is, at each occasion where said BTK inhibitor is administered, administered without a meal.
[274] The method according to any of items [1] to [17] or [98] to [273] or the BTK inhibitor for use according to any of items [18] to [43] or [98] to [273] or the vaccine for use according to any of items [44] to [61] or [98] to [145] or [152] to [273] or the use according to any of items [62] to [273], wherein said BTK inhibitor is administered to a patient who has consumed food within 1 hour prior to receiving said BTK inhibitor.
[275] The method according to any of items [1] to [17] or [98] to [274] or the BTK inhibitor for use according to any of items [18] to [43] or [98] to [274] or the vaccine for use according to any of items [44] to [61] or [98] to [145] or [152] to [274] or the use according to any of items [62] to [274], wherein said BTK inhibitor is administered to a patient who has consumed food within 1 hour prior to being administered said BTK inhibitor and/or who does consume food within 1 hour after being administered said BTK inhibitor.
[276] The method according to any of items [1] to [17] or [98] to [275] or the BTK inhibitor for use according to any of items [18] to [43] or [98] to [275] or the vaccine for use according to any of items [44] to [61] or [98] to [145] or [152] to [275] or the use according to any of items [62] to [275], wherein said BTK inhibitor is administered to a patient who has not consumed food within 1 hour prior to being administered said BTK inhibitor and who does not consume food within 1 hour after being administered said BTK inhibitor.
[277] The method according to any of items [1] to [17] or [98] to [276] or the BTK inhibitor for use according to any of items [18] to [43] or [98] to [276] or the vaccine for use according to any of items [44] to [61] or [98] to [145] or [152] to [276] or the use according to any of items [62] to [276], wherein said BTK inhibitor is administered orally.
[278] The method according to any of items [1] to [17] or [98] to [277] or the BTK inhibitor for use according to any of items [18] to [43] or [98] to [277] or the vaccine for use according to any of items [44] to [61] or [98] to [145] or [152] to [277] or the use according to any of items [62] to [277], wherein said BTK inhibitor is administered as tablet(s) or capsule(s).
[279] The method according to any of items [1] to [17] or [98] to [278] or the BTK inhibitor for use according to any of items [18] to [43] or [98] to [278] or the vaccine for use according to any of items [44] to [61] or [98] to [145] or [152] to [278] or the use according to any of items [62] to [278], wherein said BTK inhibitor is administered as tablet(s).
[280] The method according to any of items [1] to [17] or [98] to [279] or the BTK inhibitor for use according to any of items [18] to [43] or [98] to [279] or the vaccine for use according to any of items [44] to [61] or [98] to [145] or [152] to [279] or the use according to any of items [62] to [279], wherein said vaccinating is performed by administering a vaccine.
[281] The method according to any of items [1] to [17] or [98] to [280] or the BTK inhibitor for use according to any of items [18] to [43] or [98] to [280] or the vaccine for use according to any of items [44] to [61] or [98] to [145] or [152] to [280] or the use according to any of items [62] to [280], wherein said vaccinating is performed by administering a vaccine to the subject/patient.
[282] The method according to any of items [1] to [17] or [98] to [281] or the BTK inhibitor for use according to any of items [18] to [43] or [98] to [281] or the vaccine for use according to any of items [44] to [61] or [98] to [145] or [152] to [281] or the use according to any of items [62] to [281], wherein said patient is vaccinated by administering a vaccine to said patient.
[283] The method according to any of items [1] to [17] or [98] to [282] or the BTK inhibitor for use according to any of items [18] to [43] or [98] to [282] or the vaccine for use according to any of items [44] to [61] or [98] to [145] or [152] to [282] or the use according to any of items [62] to [282], wherein said vaccine is selected from the group consisting of an inactivated vaccine, a live-attenuated vaccine, a live vaccine, a subunit vaccine, a recombinant vaccine, a polysaccharide vaccine, a conjugate vaccine, and a toxoid vaccine, or a combination thereof.
[284] The method according to any of items [1] to [17] or [98] to [283] or the BTK inhibitor for use according to any of items [18] to [43] or [98] to [283] or the vaccine for use according to any of items [44] to [61] or [98] to [145] or [152] to [283] or the use according to any of items [62] to [283], wherein said vaccine is selected from the group consisting of a live-attenuated vaccine and a live vaccine, or a combination thereof.
[285] The method according to any of items [1] to [17] or [98] to [284] or the BTK inhibitor for use according to any of items [18] to [43] or [98] to [284] or the vaccine for use according to any of items [44] to [61] or [98] to [145] or [152] to [284] or the use according to any of items [62] to [284], wherein said vaccine is an inactivated vaccine.
[286] The method according to any of items [1] to [17] or [98] to [285] or the BTK inhibitor for use according to any of items [18] to [43] or [98] to [285] or the vaccine for use according to any of items [44] to [61] or [98] to [145] or [152] to [285] or the use according to any of items [62] to [285], wherein said vaccination is performed with a vaccine selected from
[287] The method according to any of items [1] to [17] or [98] to [286] or the BTK inhibitor for use according to any of items [18] to [43] or [98] to [286] or the vaccine for use according to any of items [44] to [61] or [98] to [145] or [152] to [286] or the use according to any of items [62] to [286], wherein said vaccination is performed with a vaccine selected from the group consisting of an inactivated vaccine, a live-attenuated vaccine, a live vaccine, a subunit vaccine, a recombinant vaccine, a polysaccharide vaccine, a conjugate vaccine, and a toxoid vaccine, or a combination thereof.
[288] The method according to any of items [1] to [17] or [98] to [287] or the BTK inhibitor for use according to any of items [18] to [43] or [98] to [287] or the vaccine for use according to any of items [44] to [61] or [98] to [145] or [152] to [287] or the use according to any of items [62] to [287], wherein said vaccination is performed with a vaccine selected from the group consisting of a live-attenuated vaccine and a live vaccine, or a combination thereof.
[289] The method according to any of items [1] to [17] or [98] to [288] or the BTK inhibitor for use according to any of items [18] to [43] or [98] to [288] or the vaccine for use according to any of items [44] to [61] or [98] to [145] or [152] to [288] or the use according to any of items [62] to [288], wherein said vaccination is performed with a vaccine that is an inactivated vaccine.
[290] The method according to any of items [1] to [17] or [98] to [289] or the BTK inhibitor for use according to any of items [18] to [43] or [98] to [289] or the vaccine for use according to any of items [44] to [61] or [98] to [145] or [152] to [289] or the use according to any of items [62] to [289], wherein said vaccine is selected from the group consisting of anthrax prophylaxis vaccine, cholera prophylaxis vaccine, diphtheria prophylaxis vaccine, Haemophilus influenzae prophylaxis vaccine, meningococcal meningitis prophylaxis vaccine, pertussis prophylaxis vaccine, plague prophylaxis vaccine, pneumococcal disease prophylaxis vaccine, Streptococcus pneumoniae prophylaxis vaccine, tetanus prophylaxis vaccine, tuberculosis prophylaxis vaccine, typhoid prophylaxis vaccine.
[291] The method according to any of items [1] to [17] or [98] to [290] or the BTK inhibitor for use according to any of items [18] to [43] or [98] to [290] or the vaccine for use according to any of items [44] to [61] or [98] to [145] or [152] to [290] or the use according to any of items [62] to [290], wherein said vaccine is selected from the group consisting of: a varicella zoster virus vaccine, an attenuated live varicella zoster virus vaccine, an inactive varicella zoster virus vaccine, and an inactivated and/or recombinant varicella zoster virus vaccine.
[292] The method according to any of items [1] to [17] or [98] to [291] or the BTK inhibitor for use according to any of items [18] to [43] or [98] to [291] or the vaccine for use according to any of items [44] to [61] or [98] to [145] or [152] to [291] or the use according to any of items [62] to [291], wherein said vaccine is a recombinant, adjuvanted herpes zoster vaccine.
[293] The method according to any of items [1] to [17] or [98] to [292] or the BTK inhibitor for use according to any of items [18] to [43] or [98] to [292] or the vaccine for use according to any of items [44] to [61] or [98] to [145] or [152] to [292] or the use according to any of items [62] to [292], wherein said vaccine is a recombinant, adjuvanted herpes zoster vaccine that comprises recombinant varicella zoster virus glycoprotein E.
[294] The method according to any of items [1] to [17] or [98] to [293] or the BTK inhibitor for use according to any of items [18] to [43] or [98] to [293] or the vaccine for use according to any of items [44] to [61] or [98] to [145] or [152] to [293] or the use according to any of items [62] to [293], wherein said vaccine is SHINGRIX®.
[295] The method according to any of items [1] to [17] or [98] to [294] or the BTK inhibitor for use according to any of items [18] to [43] or [98] to [294] or the vaccine for use according to any of items [44] to [61] or [98] to [145] or [152] to [294] or the use according to any of items [62] to [294], wherein said patient is antibody-negative to varicella zoster virus, preferably antibody-negative to varicella zoster virus prior to said vaccination and/or prior to the start of said BTK inhibitor treatment.
[296] The method according to any of items [1] to [17] or [98] to [295] or the BTK inhibitor for use according to any of items [18] to [43] or [98] to [295] or the vaccine for use according to any of items [44] to [61] or [98] to [145] or [152] to [295] or the use according to any of items [62] to [295], wherein said vaccine is an influenza virus vaccine selected from the group consisting of a monovalent influenza virus vaccine, a divalent influenza virus vaccine, a trivalent influenza virus vaccine, a quadrivalent influenza virus vaccines and a polyvalent influenza virus vaccine.
[297] The method according to any of items [1] to [17] or [98] to [296] or the BTK inhibitor for use according to any of items [18] to [43] or [98] to [296] or the vaccine for use according to any of items [44] to [61] or [98] to [145] or [152] to [296] or the use according to any of items [62] to [296], wherein said vaccine is a quadrivalent influenza virus vaccines.
[298] The method according to any of items [1] to [17] or [98] to [297] or the BTK inhibitor for use according to any of items [18] to [43] or [98] to [297] or the vaccine for use according to any of items [44] to [61] or [98] to [145] or [152] to [297] or the use according to any of items [62] to [297], wherein said vaccine is selected from the group consisting of a corona vaccine, a COVID-19 vaccine, a SARS-COVID-19 vaccine and a SARS-CoV-2 vaccine.
[299] The method according to any of items [1] to [17] or [98] to [298] or the BTK inhibitor for use according to any of items [18] to [43] or [98] to [298] or the vaccine for use according to any of items [44] to [61] or [98] to [145] or [152] to [298] or the use according to any of items [62] to [298], wherein said vaccine is a corona vaccine.
[300] The method according to any of items [1] to [17] or [98] to [299] or the BTK inhibitor for use according to any of items [18] to [43] or [98] to [299] or the vaccine for use according to any of items [44] to [61] or [98] to [145] or [152] to [299] or the use according to any of items [62] to [299], wherein said vaccine is a COVID-19 vaccine.
[301] The method according to any of items [1] to [17] or [98] to [300] or the BTK inhibitor for use according to any of items [18] to [43] or [98] to [300] or the vaccine for use according to any of items [44] to [61] or [98] to [145] or [152] to [300] or the use according to any of items [62] to [300], wherein said vaccine is an mRNA-based COVID-19 vaccine.
[302] The method according to any of items [1] to [17] or [98] to [301] or the BTK inhibitor for use according to any of items [18] to [43] or [98] to [301] or the vaccine for use according to any of items [44] to [61] or [98] to [145] or [152] to [301] or the use according to any of items [62] to [301], wherein said vaccine is not an mRNA-based COVID-19 vaccine.
[303] The method according to any of items [1] to [17] or [98] to [302] or the BTK inhibitor for use according to any of items [18] to [43] or [98] to [302] or the vaccine for use according to any of items [44] to [61] or [98] to [145] or [152] to [302] or the use according to any of items [62] to [302], wherein said vaccine is an mRNA vaccine against COVID-19 of BioNTech/Pfizer or an mRNA vaccine against COVID-19 of Moderna.
[304] The method according to any of items [1] to [17] or [98] to [303] or the BTK inhibitor for use according to any of items [18] to [43] or [98] to [303] or the vaccine for use according to any of items [44] to [61] or [98] to [145] or [152] to [303] or the use according to any of items [62] to [303], wherein said vaccine is Comirnaty® by BioNTech/Pfizer or Spikevax® by Moderna or an identical vaccine distributed under a different name.
[305] The method according to any of items [1] to [17] or [98] to [304] or the BTK inhibitor for use according to any of items [18] to [43] or [98] to [304] or the vaccine for use according to any of items [44] to [61] or [98] to [145] or [152] to [304] or the use according to any of items [62] to [304], wherein said vaccine is Comirnaty® by BioNTech/Pfizer or Spikevax® by Moderna or an identical vaccine.
[306] The method according to any of items [1] to [17] or [98] to [305] or the BTK inhibitor for use according to any of items [18] to [43] or [98] to [305] or the vaccine for use according to any of items [44] to [61] or [98] to [145] or [152] to [305] or the use according to any of items [62] to [305], wherein said vaccine is mRNA vaccine against COVID-19 of BioNTech/Pfizer.
[307] The method according to any of items [1] to [17] or [98] to [306] or the BTK inhibitor for use according to any of items [18] to [43] or [98] to [306] or the vaccine for use according to any of items [44] to [61] or [98] to [145] or [152] to [306] or the use according to any of items [62] to [306], wherein said patient is, prior to said vaccination, antibody-negative for antibodies against the target antigen of said vaccination.
[308] The method according to any of items [1] to [17] or [98] to [307] or the BTK inhibitor for use according to any of items [18] to [43] or [98] to [307] or the vaccine for use according to any of items [44] to [61] or [98] to [145] or [152] to [307] or the use according to any of items [62] to [307], wherein said patient is, prior to said vaccination, seronegative for the antibodies that are generated in the immune response against said vaccine.
[309] The method according to any of items [1] to [17] or [98] to [308] or the BTK inhibitor for use according to any of items [18] to [43] or [98] to [308] or the vaccine for use according to any of items [44] to [61] or [98] to [145] or [152] to [308] or the use according to any of items [62] to [308], wherein the antigens to which said patient is exposed upon vaccination are novel antigens for the patient.
[310] The method according to any of items [1] to [17] or [98] to [309] or the BTK inhibitor for use according to any of items [18] to [43] or [98] to [309] or the vaccine for use according to any of items [44] to [61] or [98] to [145] or [152] to [309] or the use according to any of items [62] to [309], wherein the immune response of said patient to said vaccination is a de novo response.
[311] The method according to any of items [1] to [17] or [98] to [310] or the BTK inhibitor for use according to any of items [18] to [43] or [98] to [310] or the vaccine for use according to any of items [44] to [61] or [98] to [145] or [152] to [310] or the use according to any of items [62] to [310], wherein said patient is, prior to said vaccination, antibody-positive for antibodies against the target antigen of said vaccination.
[312] The method according to any of items [1] to [17] or [98] to [311] or the BTK inhibitor for use according to any of items [18] to [43] or [98] to [311] or the vaccine for use according to any of items [44] to [61] or [98] to [145] or [152] to [311] or the use according to any of items [62] to [311], wherein said patient is, prior to said vaccination, seropositive for the antibodies that are generated in the immune response against said vaccine.
[313] The method according to any of items [1] to [17] or [98] to [312] or the BTK inhibitor for use according to any of items [18] to [43] or [98] to [312] or the vaccine for use according to any of items [44] to [61] or [98] to [145] or [152] to [312] or the use according to any of items [62] to [312], wherein the antigens to which said patient is exposed upon vaccination are recall antigens for the patient.
[314] The method according to any of items [1] to [17] or [98] to [313] or the BTK inhibitor for use according to any of items [18] to [43] or [98] to [313] or the vaccine for use according to any of items [44] to [61] or [98] to [145] or [152] to [313] or the use according to any of items [62] to [313], wherein the immune response of said patient to said vaccination is a recall response.
[315] The method according to any of items [1] to [17] or [98] to [314] or the BTK inhibitor for use according to any of items [18] to [43] or [98] to [314] or the vaccine for use according to any of items [44] to [61] or [98] to [145] or [152] to [314] or the use according to any of items [62] to [314], wherein said vaccine is a vaccine against an infection.
[316] The method according to any of items [1] to [17] or [98] to [315] or the BTK inhibitor for use according to any of items [18] to [43] or [98] to [315] or the vaccine for use according to any of items [44] to [61] or [98] to [145] or [152] to [315] or the use according to any of items [62] to [315], wherein said infection is a viral infection.
[317] The method according to any of items [1] to [17] or [98] to [316] or the BTK inhibitor for use according to any of items [18] to [43] or [98] to [316] or the vaccine for use according to any of items [44] to [61] or [98] to [145] or [152] to [316] or the use according to any of items [62] to [316], wherein said viral infection is selected from the group consisting of hepatitis, chickenpox (varicella), shingles (herpes zoster), measles, influenza, poliovirus, a human papilloma virus (HPV)-related disease, SARS (severe acute respiratory syndrome), MERS (Middle East respiratory syndrome) and COVID-19 (Corona virus disease 2019).
[318] The method according to any of items [1] to [17] or [98] to [317] or the BTK inhibitor for use according to any of items [18] to [43] or [98] to [317] or the vaccine for use according to any of items [44] to [61] or [98] to [145] or [152] to [317] or the use according to any of items [62] to [317], wherein said hepatitis is hepatitis A and/or hepatitis B.
[319] The method according to any of items [1] to [17] or [98] to [318] or the BTK inhibitor for use according to any of items [18] to [43] or [98] to [318] or the vaccine for use according to any of items [44] to [61] or [98] to [145] or [152] to [318] or the use according to any of items [62] to [318], wherein said hepatitis is hepatitis B.
[320] The method according to any of items [1] to [17] or [98] to [319] or the BTK inhibitor for use according to any of items [18] to [43] or [98] to [319] or the vaccine for use according to any of items [44] to [61] or [98] to [145] or [152] to [319] or the use according to any of items [62] to [319], wherein said infection is a bacterial infection.
[321] The method according to any of items [1] to [17] or [98] to [320] or the BTK inhibitor for use according to any of items [18] to [43] or [98] to [320] or the vaccine for use according to any of items [44] to [61] or [98] to [145] or [152] to [320] or the use according to any of items [62] to [320], wherein said bacterial infection is selected from the group consisting of anthrax infection, cholera, diphtheria, Haemophilus influenzae infection, meningococcal meningitis, pertussis, plague, pneumococcal disease, Streptococcus pneumoniae infection, pneumococcal pneumonia, tetanus, tuberculosis, and typhus.
[322] The method according to any of items [1] to [17] or [98] to [321] or the BTK inhibitor for use according to any of items [18] to [43] or [98] to [321] or the vaccine for use according to any of items [44] to [61] or [98] to [145] or [152] to [321] or the use according to any of items [62] to [321], wherein said infection is a coronavirus infection.
[323] The method according to any of items [1] to [17] or [98] to [322] or the BTK inhibitor for use according to any of items [18] to [43] or [98] to [322] or the vaccine for use according to any of items [44] to [61] or [98] to [145] or [152] to [322] or the use according to any of items [62] to [322], wherein said infection is selected from the group consisting of SARS (severe acute respiratory syndrome), MERS (Middle East respiratory syndrome) and COVID-19 (Corona virus disease 2019).
[324] The method according to any of items [1] to [17] or [98] to [323] or the BTK inhibitor for use according to any of items [18] to [43] or [98] to [323] or the vaccine for use according to any of items [44] to [61] or [98] to [145] or [152] to [323] or the use according to any of items [62] to [323], wherein said infection is COVID-19.
[325] The method according to any of items [1] to [17] or [98] to [324] or the BTK inhibitor for use according to any of items [18] to [43] or [98] to [324] or the vaccine for use according to any of items [44] to [61] or [98] to [145] or [152] to [324] or the use according to any of items [62] to [324], wherein said infection is caused by a virus belonging to the realm of Riboviria.
[326] The method according to any of items [1] to [17] or [98] to [325] or the BTK inhibitor for use according to any of items [18] to [43] or [98] to [325] or the vaccine for use according to any of items [44] to [61] or [98] to [145] or [152] to [325] or the use according to any of items [62] to [325], wherein said infection is caused by a virus belonging to the realm of Riboviria and within that realm to the order of Nidovirales.
[327] The method according to any of items [1] to [17] or [98] to [326] or the BTK inhibitor for use according to any of items [18] to [43] or [98] to [326] or the vaccine for use according to any of items [44] to [61] or [98] to [145] or [152] to [326] or the use according to any of items [62] to [326], wherein said infection is caused by a virus belonging to the realm of Riboviria, within that realm to the order of Nidovirales, and within that order to the family of Coronaviridae.
[328] The method according to any of items [1] to [17] or [98] to [327] or the BTK inhibitor for use according to any of items [18] to [43] or [98] to [327] or the vaccine for use according to any of items [44] to [61] or [98] to [145] or [152] to [327] or the use according to any of items [62] to [327], wherein said infection is caused by a virus belonging to the realm of Riboviria, within that realm to the order of Nidovirales, within that order to the family of Coronaviridae, and within that family to the sub-family of Orthocoronavirinae.
[329] The method according to any of items [1] to [17] or [98] to [328] or the BTK inhibitor for use according to any of items [18] to [43] or [98] to [328] or the vaccine for use according to any of items [44] to [61] or [98] to [145] or [152] to [328] or the use according to any of items [62] to [328], wherein said infection is caused by a corona virus.
[330] The method according to any of items [1] to [17] or [98] to [329] or the BTK inhibitor for use according to any of items [18] to [43] or [98] to [329] or the vaccine for use according to any of items [44] to [61] or [98] to [145] or [152] to [329] or the use according to any of items [62] to [329], wherein said infection is caused by a virus selected from the group consisting of SARS-CoV-1 (severe acute respiratory syndrome coronavirus 1), MERS-CoV (Middle East respiratory syndrome-related coronavirus) and SARS-CoV-2 (severe acute respiratory syndrome coronavirus 2).
[331] The method according to any of items [1] to [17] or [98] to [330] or the BTK inhibitor for use according to any of items [18] to [43] or [98] to [330] or the vaccine for use according to any of items [44] to [61] or [98] to [145] or [152] to [330] or the use according to any of items [62] to [330], wherein said infection is caused by SARS-CoV-2 (severe acute respiratory syndrome coronavirus 2).
[332] The method according to any of items [1] to [17] or [98] to [331] or the BTK inhibitor for use according to any of items [18] to [43] or [98] to [331] or the vaccine for use according to any of items [44] to [61] or [98] to [145] or [152] to [331] or the use according to any of items [62] to [331], wherein said infection is influenza.
[333] The method according to any of items [1] to [17] or [98] to [332] or the BTK inhibitor for use according to any of items [18] to [43] or [98] to [332] or the vaccine for use according to any of items [44] to [61] or [98] to [145] or [152] to [332] or the use according to any of items [62] to [332], wherein said infection is seasonal flu.
[334] The method according to any of items [1] to [17] or [98] to [333] or the BTK inhibitor for use according to any of items [18] to [43] or [98] to [333] or the vaccine for use according to any of items [44] to [61] or [98] to [145] or [152] to [333] or the use according to any of items [62] to [333], wherein said infection is a varicella zoster infection.
[335] The method according to any of items [1] to [17] or [98] to [334] or the BTK inhibitor for use according to any of items [18] to [43] or [98] to [334] or the vaccine for use according to any of items [44] to [61] or [98] to [145] or [152] to [334] or the use according to any of items [62] to [334], wherein said infection is a herpes zoster infection.
[336] The method according to any of items [1] to [17] or [98] to [335] or the BTK inhibitor for use according to any of items [18] to [43] or [98] to [335] or the vaccine for use according to any of items [44] to [61] or [98] to [145] or [152] to [335] or the use according to any of items [62] to [335], wherein said infection is chickenpox.
[337] The method according to any of items [1] to [17] or [98] to [336] or the BTK inhibitor for use according to any of items [18] to [43] or [98] to [336] or the vaccine for use according to any of items [44] to [61] or [98] to [145] or [152] to [336] or the use according to any of items [62] to [336], wherein said infection is shingles.
[338] The method according to any of items [1] to [17] or [98] to [337] or the BTK inhibitor for use according to any of items [18] to [43] or [98] to [337] or the vaccine for use according to any of items [44] to [61] or [98] to [145] or [152] to [337] or the use according to any of items [62] to [337], wherein said vaccine is a vaccine against an infectious agent.
[339] The method according to any of items [1] to [17] or [98] to [338] or the BTK inhibitor for use according to any of items [18] to [43] or [98] to [338] or the vaccine for use according to any of items [44] to [61] or [98] to [145] or [152] to [338] or the use according to any of items [62] to [338], wherein said infection is an infection caused by an infectious agent.
[340] The method according to any of items [1] to [17] or [98] to [339] or the BTK inhibitor for use according to any of items [18] to [43] or [98] to [339] or the vaccine for use according to any of items [44] to [61] or [98] to [145] or [152] to [339] or the use according to any of items [62] to [339], wherein said infection is caused by an infectious agent.
[341] The method according to any of items [1] to [17] or [98] to [340] or the BTK inhibitor for use according to any of items [18] to [43] or [98] to [340] or the vaccine for use according to any of items [44] to [61] or [98] to [145] or [152] to [340] or the use according to any of items [62] to [340], wherein said infectious agent is a virus belonging to the realm of Riboviria.
[342] The method according to any of items [1] to [17] or [98] to [341] or the BTK inhibitor for use according to any of items [18] to [43] or [98] to [341] or the vaccine for use according to any of items [44] to [61] or [98] to [145] or [152] to [341] or the use according to any of items [62] to [341], wherein said infectious agent is a virus belonging to the realm of Riboviria and to the order of Nidovirales.
[343] The method according to any of items [1] to [17] or [98] to [342] or the BTK inhibitor for use according to any of items [18] to [43] or [98] to [342] or the vaccine for use according to any of items [44] to [61] or [98] to [145] or [152] to [342] or the use according to any of items [62] to [342], wherein said infectious agent is a virus belonging to the realm of Riboviria, to the order of Nidovirales, and to the family of Coronaviridae.
[344] The method according to any of items [1] to [17] or [98] to [343] or the BTK inhibitor for use according to any of items [18] to [43] or [98] to [343] or the vaccine for use according to any of items [44] to [61] or [98] to [145] or [152] to [343] or the use according to any of items [62] to [343], wherein said infectious agent is a virus belonging to the realm of Riboviria, to the order of Nidovirales, to the family of Coronaviridae, and to the sub-family of Orthocoronavirinae.
[345] The method according to any of items [1] to [17] or [98] to [344] or the BTK inhibitor for use according to any of items [18] to [43] or [98] to [344] or the vaccine for use according to any of items [44] to [61] or [98] to [145] or [152] to [344] or the use according to any of items [62] to [344], wherein said infectious agent is a corona virus.
[346] The method according to any of items [1] to [17] or [98] to [345] or the BTK inhibitor for use according to any of items [18] to [43] or [98] to [345] or the vaccine for use according to any of items [44] to [61] or [98] to [145] or [152] to [345] or the use according to any of items [62] to [345], wherein said infectious agent is a virus selected from the group consisting of SARS-CoV-1 (severe acute respiratory syndrome coronavirus 1), MERS-CoV (Middle East respiratory syndrome-related coronavirus) and SARS-CoV-2 (severe acute respiratory syndrome coronavirus 2).
[347] The method according to any of items [1] to [17] or [98] to [346] or the BTK inhibitor for use according to any of items [18] to [43] or [98] to [346] or the vaccine for use according to any of items [44] to [61] or [98] to [145] or [152] to [346] or the use according to any of items [62] to [346], wherein said infectious agent is SARS-CoV-2 (severe acute respiratory syndrome coronavirus 2).
[348] The method according to any of items [1] to [17] or [98] to [347] or the BTK inhibitor for use according to any of items [18] to [43] or [98] to [347] or the vaccine for use according to any of items [44] to [61] or [98] to [145] or [152] to [347] or the use according to any of items [62] to [347], wherein said vaccine is selected from the group consisting of: an mRNA-based SARS-CoV-2/COVID-19 virus vaccine, a vector-based SARS-CoV-2/COVID-19 virus vaccine, an adenovirus/vector-based SARS-CoV-2/COVID-19 virus vaccine, a virus-like particles (VLPs) based SARS-CoV-2/COVID-19 virus vaccine, a DNA-based SARS-CoV-2/COVID-19 virus vaccine, a protein-based SARS-CoV-2/COVID-19 virus vaccine, an inactivated virus-based SARS-CoV-2/COVID-19 virus vaccine, and a dead virus-based SARS-CoV-2/COVID-19 virus vaccine.
[349] The method according to any of items [1] to [17] or [98] to [348] or the BTK inhibitor for use according to any of items [18] to [43] or [98] to [348] or the vaccine for use according to any of items [44] to [61] or [98] to [145] or [152] to [348] or the use according to any of items [62] to [348], wherein said vaccine is selected from the group consisting of: an mRNA-based SARS-CoV-2/COVID-19 virus vaccine, a vector-based SARS-CoV-2/COVID-19 virus vaccine, and an adenovirus/vector-based SARS-CoV-2/COVID-19 virus vaccine.
[350] The method according to any of items [1] to [17] or [98] to [349] or the BTK inhibitor for use according to any of items [18] to [43] or [98] to [349] or the vaccine for use according to any of items [44] to [61] or [98] to [145] or [152] to [349] or the use according to any of items [62] to [349], wherein said vaccine is an mRNA-based SARS-CoV-2/COVID-19 virus vaccine.
[351] The method according to any of items [1] to [17] or [98] to [350] or the BTK inhibitor for use according to any of items [18] to [43] or [98] to [350] or the vaccine for use according to any of items [44] to [61] or [98] to [145] or [152] to [350] or the use according to any of items [62] to [350], wherein said vaccine is Comirnaty® (by BioNTech/Pfizer) or Spikevax® (by Moderna).
[352] The method according to any of items [1] to [17] or [98] to [351] or the BTK inhibitor for use according to any of items [18] to [43] or [98] to [351] or the vaccine for use according to any of items [44] to [61] or [98] to [145] or [152] to [351] or the use according to any of items [62] to [351], wherein said vaccine is Comirnaty®.
[353] The method according to any of items [1] to [17] or [98] to [352] or the BTK inhibitor for use according to any of items [18] to [43] or [98] to [352] or the vaccine for use according to any of items [44] to [61] or [98] to [145] or [152] to [352] or the use according to any of items [62] to [352], wherein said vaccine is Spikevax®.
[354] The method according to any of items [1] to [17] or [98] to [353] or the BTK inhibitor for use according to any of items [18] to [43] or [98] to [353] or the vaccine for use according to any of items [44] to [61] or [98] to [145] or [152] to [353] or the use according to any of items [62] to [353], wherein said infectious agent is an influenza virus.
[355] The method according to any of items [1] to [17] or [98] to [354] or the BTK inhibitor for use according to any of items [18] to [43] or [98] to [354] or the vaccine for use according to any of items [44] to [61] or [98] to [145] or [152] to [354] or the use according to any of items [62] to [354], wherein said infectious agent is an influenza virus of the strain HI A (human influenza A) or HI B (human influenza B).
[356] The method according to any of items [1] to [17] or [98] to [355] or the BTK inhibitor for use according to any of items [18] to [43] or [98] to [355] or the vaccine for use according to any of items [44] to [61] or [98] to [145] or [152] to [355] or the use according to any of items [62] to [355], wherein said infectious agent is an influenza virus of the strain HI A (human influenza A).
[357] The method according to any of items [1] to [17] or [98] to [356] or the BTK inhibitor for use according to any of items [18] to [43] or [98] to [356] or the vaccine for use according to any of items [44] to [61] or [98] to [145] or [152] to [356] or the use according to any of items [62] to [356], wherein said infectious agent is an influenza virus of the strain HI A (human influenza A), subtype A(H1N1).
[358] The method according to any of items [1] to [17] or [98] to [357] or the BTK inhibitor for use according to any of items [18] to [43] or [98] to [357] or the vaccine for use according to any of items [44] to [61] or [98] to [145] or [152] to [357] or the use according to any of items [62] to [357], wherein said infectious agent is an influenza virus of the strain HI A (human influenza A), subtype A(H3N2).
[359] The method according to any of items [1] to [17] or [98] to [358] or the BTK inhibitor for use according to any of items [18] to [43] or [98] to [358] or the vaccine for use according to any of items [44] to [61] or [98] to [145] or [152] to [358] or the use according to any of items [62] to [358], wherein said infectious agent is an influenza virus of the strain HI B (human influenza B).
[360] The method according to any of items [1] to [17] or [98] to [359] or the BTK inhibitor for use according to any of items [18] to [43] or [98] to [359] or the vaccine for use according to any of items [44] to [61] or [98] to [145] or [152] to [359] or the use according to any of items [62] to [359], wherein said infectious agent is an influenza virus of the strain HI B (human influenza B), lineage B/Yamagata.
[361] The method according to any of items [1] to [17] or [98] to [360] or the BTK inhibitor for use according to any of items [18] to [43] or [98] to [360] or the vaccine for use according to any of items [44] to [61] or [98] to [145] or [152] to [360] or the use according to any of items [62] to [360], wherein said infectious agent is an influenza virus of the strain HI B (human influenza B), lineage B/Victoria.
[362] The method according to any of items [1] to [17] or [98] to [361] or the BTK inhibitor for use according to any of items [18] to [43] or [98] to [361] or the vaccine for use according to any of items [44] to [61] or [98] to [145] or [152] to [361] or the use according to any of items [62] to [361], wherein said infectious agent is varicella zoster virus.
[363] The method according to any of items [1] to [17] or [98] to [362] or the BTK inhibitor for use according to any of items [18] to [43] or [98] to [362] or the vaccine for use according to any of items [44] to [61] or [98] to [145] or [152] to [362] or the use according to any of items [62] to [362], wherein said vaccine is administered by intramuscular injection.
[364] The method according to any of items [1] to [17] or [98] to [363] or the BTK inhibitor for use according to any of items [18] to [43] or [98] to [363] or the vaccine for use according to any of items [44] to [61] or [98] to [145] or [152] to [363] or the use according to any of items [62] to [363], wherein said vaccine is administered by oral administration.
[365] The method according to any of items [1] to [17] or [98] to [364] or the BTK inhibitor for use according to any of items [18] to [43] or [98] to [364] or the vaccine for use according to any of items [44] to [61] or [98] to [145] or [152] to [364] or the use according to any of items [62] to [364], wherein said vaccine is administered as a nasal spray.
[366] The method according to any of items [1] to [17] or [98] to [365] or the BTK inhibitor for use according to any of items [18] to [43] or [98] to [365] or the vaccine for use according to any of items [44] to [61] or [98] to [145] or [152] to [365] or the use according to any of items [62] to [365], wherein said vaccination consists of administering a single dose of vaccine.
[367] The method according to any of items [1] to [17] or [98] to [366] or the BTK inhibitor for use according to any of items [18] to [43] or [98] to [366] or the vaccine for use according to any of items [44] to [61] or [98] to [145] or [152] to [366] or the use according to any of items [62] to [366], wherein said vaccination consists of administering two separate doses of vaccine administered on different days.
[368] The method according to any of items [1] to [17] or [98] to [367] or the BTK inhibitor for use according to any of items [18] to [43] or [98] to [367] or the vaccine for use according to any of items [44] to [61] or [98] to [145] or [152] to [367] or the use according to any of items [62] to [367], wherein said vaccination consists of administering one dose of vaccine or two separate doses of vaccine administered on different days.
[369] The method according to any of items [1] to [17] or [98] to [368] or the BTK inhibitor for use according to any of items [18] to [43] or [98] to [368] or the vaccine for use according to any of items [44] to [61] or [98] to [145] or [152] to [368] or the use according to any of items [62] to [368], wherein said vaccination consists of administering a single dose of vaccine or 2 to 6 separate doses of vaccine administered on different days.
[370] The method according to any of items [1] to [17] or [98] to [369] or the BTK inhibitor for use according to any of items [18] to [43] or [98] to [369] or the vaccine for use according to any of items [44] to [61] or [98] to [145] or [152] to [369] or the use according to any of items [62] to [369], wherein said administering said dose(s) of vaccine is administering said dose(s) to the patient.
[371] The method according to any of items [1] to [17] or [98] to [370] or the BTK inhibitor for use according to any of items [18] to [43] or [98] to [370] or the vaccine for use according to any of items [44] to [61] or [98] to [145] or [152] to [370] or the use according to any of items [62] to [370], wherein said two doses resp. said 2 to 6 doses are administered on different days within 1 week to 12 months.
[372] The method according to any of items [1] to [17] or [98] to [371] or the BTK inhibitor for use according to any of items [18] to [43] or [98] to [371] or the vaccine for use according to any of items [44] to [61] or [98] to [145] or [152] to [371] or the use according to any of items [62] to [371], wherein said two doses resp. said 2 to 6 separate doses are administered on different days within 1 week to 6 months.
[373] The method according to any of items [1] to [17] or [98] to [372] or the BTK inhibitor for use according to any of items [18] to [43] or [98] to [372] or the vaccine for use according to any of items [44] to [61] or [98] to [145] or [152] to [372] or the use according to any of items [62] to [372], wherein said two doses resp. said 2 to 6 separate doses are administered on different days within 1 to 12 weeks.
[374] The method according to any of items [1] to [17] or [98] to [373] or the BTK inhibitor for use according to any of items [18] to [43] or [98] to [373] or the vaccine for use according to any of items [44] to [61] or [98] to [145] or [152] to [373] or the use according to any of items [62] to [373], wherein said two doses resp. said 2 to 6 separate doses are administered on different days within 1 to 6 weeks.
[375] The method according to any of items [1] to [17] or [98] to [374] or the BTK inhibitor for use according to any of items [18] to [43] or [98] to [374] or the vaccine for use according to any of items [44] to [61] or [98] to [145] or [152] to [374] or the use according to any of items [62] to [374], wherein said two doses resp. said 2 to 6 separate doses are administered on different days within 1 to 4 weeks.
[376] The method according to any of items [1] to [17] or [98] to [375] or the BTK inhibitor for use according to any of items [18] to [43] or [98] to [375] or the vaccine for use according to any of items [44] to [61] or [98] to [145] or [152] to [375] or the use according to any of items [62] to [375], wherein said two separate doses resp. said 2 to 6 separate doses are administered on different days separated by a time period of at least 2 weeks.
[377] The method according to any of items [1] to [17] or [98] to [376] or the BTK inhibitor for use according to any of items [18] to [43] or [98] to [376] or the vaccine for use according to any of items [44] to [61] or [98] to [145] or [152] to [376] or the use according to any of items [62] to [376], wherein said two separate doses resp. said 2 to 6 separate doses are administered on different days separated by a time period of at least 4 weeks.
[378] The method according to any of items [1] to [17] or [98] to [377] or the BTK inhibitor for use according to any of items [18] to [43] or [98] to [377] or the vaccine for use according to any of items [44] to [61] or [98] to [145] or [152] to [377] or the use according to any of items [62] to [377], wherein said two separate doses are administered to the patient on different days separated by a time period of at least 2 months.
[379] The method according to any of items [1] to [17] or [98] to [378] or the BTK inhibitor for use according to any of items [18] to [43] or [98] to [378] or the vaccine for use according to any of items [44] to [61] or [98] to [145] or [152] to [378] or the use according to any of items [62] to [378], wherein the treatment with said BTK inhibitor lasts for at least 2 weeks.
[380] The method according to any of items [1] to [17] or [98] to [379] or the BTK inhibitor for use according to any of items [18] to [43] or [98] to [379] or the vaccine for use according to any of items [44] to [61] or [98] to [145] or [152] to [379] or the use according to any of items [62] to [379], wherein the treatment with said BTK inhibitor lasts for at least 1 month.
[381] The method according to any of items [1] to [17] or [98] to [380] or the BTK inhibitor for use according to any of items [18] to [43] or [98] to [380] or the vaccine for use according to any of items [44] to [61] or [98] to [145] or [152] to [380] or the use according to any of items [62] to [380], wherein the treatment with said BTK inhibitor lasts for at least 2 months.
[382] The method according to any of items [1] to [17] or [98] to [381] or the BTK inhibitor for use according to any of items [18] to [43] or [98] to [381] or the vaccine for use according to any of items [44] to [61] or [98] to [145] or [152] to [381] or the use according to any of items [62] to [381], wherein the treatment with said BTK inhibitor lasts for at least 3 months.
[383] The method according to any of items [1] to [17] or [98] to [382] or the BTK inhibitor for use according to any of items [18] to [43] or [98] to [382] or the vaccine for use according to any of items [44] to [61] or [98] to [145] or [152] to [382] or the use according to any of items [62] to [382], wherein the treatment with said BTK inhibitor lasts for at least 6 months.
[384] The method according to any of items [1] to [17] or [98] to [383] or the BTK inhibitor for use according to any of items [18] to [43] or [98] to [383] or the vaccine for use according to any of items [44] to [61] or [98] to [145] or [152] to [383] or the use according to any of items [62] to [383], wherein the treatment with said BTK inhibitor lasts for at least 12 months.
[385] The method according to any of items [1] to [17] or [98] to [384] or the BTK inhibitor for use according to any of items [18] to [43] or [98] to [384] or the vaccine for use according to any of items [44] to [61] or [98] to [145] or [152] to [384] or the use according to any of items [62] to [384], wherein the treatment with said BTK inhibitor lasts for not more than 12 months.
[386] The method according to any of items [1] to [17] or [98] to [385] or the BTK inhibitor for use according to any of items [18] to [43] or [98] to [385] or the vaccine for use according to any of items [44] to [61] or [98] to [145] or [152] to [385] or the use according to any of items [62] to [385], wherein the treatment with said BTK inhibitor lasts for not more than 6 months.
[387] The method according to any of items [1] to [17] or [98] to [386] or the BTK inhibitor for use according to any of items [18] to [43] or [98] to [386] or the vaccine for use according to any of items [44] to [61] or [98] to [145] or [152] to [386] or the use according to any of items [62] to [386], wherein the treatment with said BTK inhibitor lasts for not more than 3 months.
[388] The method according to any of items [1] to [17] or [98] to [387] or the BTK inhibitor for use according to any of items [18] to [43] or [98] to [387] or the vaccine for use according to any of items [44] to [61] or [98] to [145] or [152] to [387] or the use according to any of items [62] to [387], wherein the treatment with said BTK inhibitor lasts for not more than 2 months.
[389] The method according to any of items [1] to [17] or [98] to [388] or the BTK inhibitor for use according to any of items [18] to [43] or [98] to [388] or the vaccine for use according to any of items [44] to [61] or [98] to [145] or [152] to [388] or the use according to any of items [62] to [388], wherein the treatment with said BTK inhibitor lasts for not more than 1 month.
[390] The method according to any of items [1] to [17] or [98] to [389] or the BTK inhibitor for use according to any of items [18] to [43] or [98] to [389] or the vaccine for use according to any of items [44] to [61] or [98] to [145] or [152] to [389] or the use according to any of items [62] to [389], wherein the treatment with said BTK inhibitor lasts for not more than 2 weeks.
[391] The method according to any of items [1] to [17] or [98] to [390] or the BTK inhibitor for use according to any of items [18] to [43] or [98] to [390] or the vaccine for use according to any of items [44] to [61] or [98] to [145] or [152] to [390] or the use according to any of items [62] to [390], wherein during the time for which said treatment with said BTK inhibitor lasts, the patient is under continuous treatment with said BTK inhibitor, without intermittent periods in which no treatment with said BTK inhibitor takes place.
[392] The method according to any of items [1] to [17] or [98] to [391] or the BTK inhibitor for use according to any of items [18] to [43] or [98] to [391] or the vaccine for use according to any of items [44] to [61] or [98] to [145] or [152] to [391] or the use according to any of items [62] to [391], wherein by administering said BTK inhibitor to said patient or subject, said patient or subject is treated for said disease.
[393] The method according to any of items [1] to [17] or [98] to [392] or the BTK inhibitor for use according to any of items [18] to [43] or [98] to [392] or the vaccine for use according to any of items [44] to [61] or [98] to [145] or [152] to [392] or the use according to any of items [62] to [392], wherein by administering said BTK inhibitor to said patient or subject, said patient or subject is treated for said disease.
[394] The method according to any of items [1] to [17] or [98] to [393] or the BTK inhibitor for use according to any of items [18] to [43] or [98] to [393] or the vaccine for use according to any of items [44] to [61] or [98] to [145] or [152] to [393] or the use according to any of items [62] to [393], wherein by administering a BTK inhibitor, BTK inhibitor treatment is performed.
[395] The method according to any of items [1] to [17] or [98] to [394] or the BTK inhibitor for use according to any of items [18] to [43] or [98] to [394] or the vaccine for use according to any of items [44] to [61] or [98] to [145] or [152] to [394] or the use according to any of items [62] to [394], wherein by administering a BTK inhibitor said patient is treated with said BTK inhibitor.
[396] The method according to any of items [1] to [17] or [98] to [395] or the BTK inhibitor for use according to any of items [18] to [43] or [98] to [395] or the vaccine for use according to any of items [44] to [61] or [98] to [145] or [152] to [395] or the use according to any of items [62] to [395], wherein at the time when said vaccination starts the treatment with said BTK inhibitor has been ongoing for at least 1 week.
[397] The method according to any of items [1] to [17] or [98] to [396] or the BTK inhibitor for use according to any of items [18] to [43] or [98] to [396] or the vaccine for use according to any of items [44] to [61] or [98] to [145] or [152] to [396] or the use according to any of items [62] to [396], wherein at the time when said vaccination starts the treatment with said BTK inhibitor has been ongoing for at least 2 weeks.
[398] The method according to any of items [1] to [17] or [98] to [397] or the BTK inhibitor for use according to any of items [18] to [43] or [98] to [397] or the vaccine for use according to any of items [44] to [61] or [98] to [145] or [152] to [397] or the use according to any of items [62] to [397], wherein at the time when said vaccination starts the treatment with said BTK inhibitor has been ongoing for at least 1 month.
[399] The method according to any of items [1] to [17] or [98] to [398] or the BTK inhibitor for use according to any of items [18] to [43] or [98] to [398] or the vaccine for use according to any of items [44] to [61] or [98] to [145] or [152] to [398] or the use according to any of items [62] to [398], wherein at the time when said vaccination starts the treatment with said BTK inhibitor has been ongoing for at least 2 months.
[400] The method according to any of items [1] to [17] or [98] to [399] or the BTK inhibitor for use according to any of items [18] to [43] or [98] to [399] or the vaccine for use according to any of items [44] to [61] or [98] to [145] or [152] to [399] or the use according to any of items [62] to [399], wherein at the time when said vaccination starts the treatment with said BTK inhibitor has been ongoing for at least 3 months.
[401] The method according to any of items [1] to [17] or [98] to [400] or the BTK inhibitor for use according to any of items [18] to [43] or [98] to [400] or the vaccine for use according to any of items [44] to [61] or [98] to [145] or [152] to [400] or the use according to any of items [62] to [400], wherein at the time when said vaccination starts the treatment with said BTK inhibitor has been ongoing for at least 6 months.
[402] The method according to any of items [1] to [17] or [98] to [401] or the BTK inhibitor for use according to any of items [18] to [43] or [98] to [401] or the vaccine for use according to any of items [44] to [61] or [98] to [145] or [152] to [401] or the use according to any of items [62] to [401], wherein at the time when said vaccination starts the treatment with said BTK inhibitor has been ongoing for at least 12 months.
[403] The method according to any of items [1] to [17] or [98] to [402] or the BTK inhibitor for use according to any of items [18] to [43] or [98] to [402] or the vaccine for use according to any of items [44] to [61] or [98] to [145] or [152] to [402] or the use according to any of items [62] to [402], wherein at the time when said vaccination starts the treatment with said BTK inhibitor has been ongoing for not more than 12 months.
[404] The method according to any of items [1] to [17] or [98] to [403] or the BTK inhibitor for use according to any of items [18] to [43] or [98] to [403] or the vaccine for use according to any of items [44] to [61] or [98] to [145] or [152] to [403] or the use according to any of items [62] to [403], wherein at the time when said vaccination starts the treatment with said BTK inhibitor has been ongoing for not more than 6 months.
[405] The method according to any of items [1] to [17] or [98] to [404] or the BTK inhibitor for use according to any of items [18] to [43] or [98] to [404] or the vaccine for use according to any of items [44] to [61] or [98] to [145] or [152] to [404] or the use according to any of items [62] to [404], wherein at the time when said vaccination starts the treatment with said BTK inhibitor has been ongoing for not more than 3 months.
[406] The method according to any of items [1] to [17] or [98] to [405] or the BTK inhibitor for use according to any of items [18] to [43] or [98] to [405] or the vaccine for use according to any of items [44] to [61] or [98] to [145] or [152] to [405] or the use according to any of items [62] to [405], wherein at the time when said vaccination starts the treatment with said BTK inhibitor has been ongoing for not more than 2 months.
[407] The method according to any of items [1] to [17] or [98] to [406] or the BTK inhibitor for use according to any of items [18] to [43] or [98] to [406] or the vaccine for use according to any of items [44] to [61] or [98] to [145] or [152] to [406] or the use according to any of items [62] to [406], wherein at the time when said vaccination starts the treatment with said BTK inhibitor has been ongoing for not more than 1 month.
[408] The method according to any of items [1] to [17] or [98] to [407] or the BTK inhibitor for use according to any of items [18] to [43] or [98] to [407] or the vaccine for use according to any of items [44] to [61] or [98] to [145] or [152] to [407] or the use according to any of items [62] to [407], wherein at the time when said vaccination starts the treatment with said BTK inhibitor has been ongoing for not more than 2 weeks.
[409] The method according to any of items [1] to [17] or [98] to [408] or the BTK inhibitor for use according to any of items [18] to [43] or [98] to [408] or the vaccine for use according to any of items [44] to [61] or [98] to [145] or [152] to [408] or the use according to any of items [62] to [408], wherein at the time when said vaccination starts the treatment with said BTK inhibitor has been ongoing for not more than 1 week.
[410] The method according to any of items [1] to [17] or [98] to [409] or the BTK inhibitor for use according to any of items [18] to [43] or [98] to [409] or the vaccine for use according to any of items [44] to [61] or [98] to [145] or [152] to [409] or the use according to any of items [62] to [409], wherein the treatment with said BTK inhibitor continues for at least 1 week after the time when said vaccination is completed.
[411] The method according to any of items [1] to [17] or [98] to [410] or the BTK inhibitor for use according to any of items [18] to [43] or [98] to [410] or the vaccine for use according to any of items [44] to [61] or [98] to [145] or [152] to [410] or the use according to any of items [62] to [410], wherein the treatment with said BTK inhibitor continues for at least 2 weeks after the time when said vaccination is completed.
[412] The method according to any of items [1] to [17] or [98] to [411] or the BTK inhibitor for use according to any of items [18] to [43] or [98] to [411] or the vaccine for use according to any of items [44] to [61] or [98] to [145] or [152] to [411] or the use according to any of items [62] to [411], wherein the treatment with said BTK inhibitor continues for at least 1 month after the time when said vaccination is completed.
[413] The method according to any of items [1] to [17] or [98] to [412] or the BTK inhibitor for use according to any of items [18] to [43] or [98] to [412] or the vaccine for use according to any of items [44] to [61] or [98] to [145] or [152] to [412] or the use according to any of items [62] to [412], wherein the treatment with said BTK inhibitor continues for at least 2 months after the time when said vaccination is completed.
[414] The method according to any of items [1] to [17] or [98] to [413] or the BTK inhibitor for use according to any of items [18] to [43] or [98] to [413] or the vaccine for use according to any of items [44] to [61] or [98] to [145] or [152] to [413] or the use according to any of items [62] to [413], wherein the treatment with said BTK inhibitor continues for at least 3 months after the time when said vaccination is completed.
[415] The method according to any of items [1] to [17] or [98] to [414] or the BTK inhibitor for use according to any of items [18] to [43] or [98] to [414] or the vaccine for use according to any of items [44] to [61] or [98] to [145] or [152] to [414] or the use according to any of items [62] to [414], wherein the treatment with said BTK inhibitor continues for at least 6 months after the time when said vaccination is completed.
[416] The method according to any of items [1] to [17] or [98] to [415] or the BTK inhibitor for use according to any of items [18] to [43] or [98] to [415] or the vaccine for use according to any of items [44] to [61] or [98] to [145] or [152] to [415] or the use according to any of items [62] to [415], wherein the treatment with said BTK inhibitor continues for at least 12 months after the time when said vaccination is completed.
[417] The method according to any of items [1] to [17] or [98] to [416] or the BTK inhibitor for use according to any of items [18] to [43] or [98] to [416] or the vaccine for use according to any of items [44] to [61] or [98] to [145] or [152] to [416] or the use according to any of items [62] to [416], wherein the treatment with said BTK inhibitor continues for not more than 12 months after the time when said vaccination is completed.
[418] The method according to any of items [1] to [17] or [98] to [417] or the BTK inhibitor for use according to any of items [18] to [43] or [98] to [417] or the vaccine for use according to any of items [44] to [61] or [98] to [145] or [152] to [417] or the use according to any of items [62] to [417], wherein the treatment with said BTK inhibitor continues for not more than 6 months after the time when said vaccination is completed.
[419] The method according to any of items [1] to [17] or [98] to [418] or the BTK inhibitor for use according to any of items [18] to [43] or [98] to [418] or the vaccine for use according to any of items [44] to [61] or [98] to [145] or [152] to [418] or the use according to any of items [62] to [418], wherein the treatment with said BTK inhibitor continues for not more than 3 months after the time when said vaccination is completed.
[420] The method according to any of items [1] to [17] or [98] to [419] or the BTK inhibitor for use according to any of items [18] to [43] or [98] to [419] or the vaccine for use according to any of items [44] to [61] or [98] to [145] or [152] to [419] or the use according to any of items [62] to [419], wherein the treatment with said BTK inhibitor continues for not more than 2 months after the time when said vaccination is completed.
[421] The method according to any of items [1] to [17] or [98] to [420] or the BTK inhibitor for use according to any of items [18] to [43] or [98] to [420] or the vaccine for use according to any of items [44] to [61] or [98] to [145] or [152] to [420] or the use according to any of items [62] to [420], wherein the treatment with said BTK inhibitor continues for not more than 1 month after the time when said vaccination is completed.
[422] The method according to any of items [1] to [17] or [98] to [421] or the BTK inhibitor for use according to any of items [18] to [43] or [98] to [421] or the vaccine for use according to any of items [44] to [61] or [98] to [145] or [152] to [421] or the use according to any of items [62] to [421], wherein the treatment with said BTK inhibitor continues for not more than 2 weeks after the time when said vaccination is completed.
[423] The method according to any of items [1] to [17] or [98] to [422] or the BTK inhibitor for use according to any of items [18] to [43] or [98] to [422] or the vaccine for use according to any of items [44] to [61] or [98] to [145] or [152] to [422] or the use according to any of items [62] to [422], wherein the treatment with said BTK inhibitor continues for not more than 1 week after the time when said vaccination is completed.
[424] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is an mRNA vaccine.
[425] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is not an mRNA vaccine.
[426] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is a non-mRNA vaccine.
[427] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is a non-mRNA COVID-19 vaccine.
[428] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is an mRNA-based vaccine.
[429] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is a viral vector-based vaccine.
[430] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is a protein-based vaccine.
[431] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is not an mRNA-based vaccine.
[432] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is Vaxzevria®/Covishield® (AstraZeneca) or an identical vaccine distributed under a different name.
[433] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is Vaxzevria® (AstraZeneca) or an identical vaccine.
[434] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is Jcovden® (Janssen/Johnson & Johnson) or an identical vaccine distributed under a different name.
[435] The method according to any of items [1] to [17] or [98] to [423] or the BTK inhibitor for use according to any of items [18] to [43] or [98] to [423] or the vaccine for use according to any of items [44] to [61] or [98] to [145] or [152] to [423] or the use according to any of items [62] to [423], wherein said vaccine is Jcovden® (Janssen/Johnson & Johnson) or an identical vaccine.
[436] The method according to any of items [1] to [17] or [98] to [435] or the BTK inhibitor for use according to any of items [18] to [43] or [98] to [435] or the vaccine for use according to any of items [44] to [61] or [98] to [145] or [152] to [435] or the use according to any of items [62] to [435], wherein said vaccinating optionally involves administration of a booster vaccination.
[437] The method according to any of items [1] to [17] or [98] to [435] or the BTK inhibitor for use according to any of items [18] to [43] or [98] to [435] or the vaccine for use according to any of items [44] to [61] or [98] to [145] or [152] to [435] or the use according to any of items [62] to [435], wherein said vaccinating involves administration of a booster vaccination.
[438] The method according to any of items [1] to [17] or [98] to [435] or the BTK inhibitor for use according to any of items [18] to [43] or [98] to [435] or the vaccine for use according to any of items [44] to [61] or [98] to [145] or [152] to [435] or the use according to any of items [62] to [435], wherein said vaccinating does not involve administration of a booster vaccination.
[439] The method according to any of items [1] to [17] or [98] to [435] or the BTK inhibitor for use according to any of items [18] to [43] or [98] to [435] or the vaccine for use according to any of items [44] to [61] or [98] to [145] or [152] to [435] or the use according to any of items [62] to [435], wherein the administration of said vaccine optionally involves administration of a booster vaccination.
[440] The method according to any of items [1] to [17] or [98] to [435] or the BTK inhibitor for use according to any of items [18] to [43] or [98] to [435] or the vaccine for use according to any of items [44] to [61] or [98] to [145] or [152] to [435] or the use according to any of items [62] to [435], wherein the administration of said vaccine involves administration of a booster vaccination.
[441] The method according to any of items [1] to [17] or [98] to [435] or the BTK inhibitor for use according to any of items [18] to [43] or [98] to [435] or the vaccine for use according to any of items [44] to [61] or [98] to [145] or [152] to [435] or the use according to any of items [62] to [435], wherein the administration of said vaccine does not involve administration of a booster vaccination.
[442] The method according to any of items [1] to [17] or [98] to [441] or the BTK inhibitor for use according to any of items [18] to [43] or [98] to [441] or the vaccine for use according to any of items [44] to [61] or [98] to [145] or [152] to [441] or the use according to any of items [62] to [441], wherein said booster vaccination is administered before said BTK inhibitor is administered.
[443] The method according to any of items [1] to [17] or [98] to [442] or the BTK inhibitor for use according to any of items [18] to [43] or [98] to [442] or the vaccine for use according to any of items [44] to [61] or [98] to [145] or [152] to [442] or the use according to any of items [62] to [442], wherein said booster vaccination is administered after said BTK inhibitor is administered.
[444] The method according to any of items [1] to [17] or [98] to [443] or the BTK inhibitor for use according to any of items [18] to [43] or [98] to [443] or the vaccine for use according to any of items [44] to [61] or [98] to [145] or [152] to [443] or the use according to any of items [62] to [443], wherein said booster vaccination is administered while said patient is on recurrent treatment with said BTK inhibitor.
[445] The method according to any of items [1] to [17] or [98] to [444] or the BTK inhibitor for use according to any of items [18] to [43] or [98] to [444] or the vaccine for use according to any of items [44] to [61] or [98] to [145] or [152] to [444] or the use according to any of items [62] to [444], wherein said booster vaccination is administered while said patient is under ongoing BTK inhibitor treatment.

The invention is explained in greater detail below by means of examples. The invention preferably can be carried out throughout the range claimed and is not restricted to the examples given here.

Moreover, the following examples are given in order to assist the skilled artisan to better understand the present disclosure by way of exemplification. The examples are not intended to limit the scope of protection conferred by the claims. The features, properties and advantages exemplified for the processes, compounds, compositions and/or uses defined in the examples may be assigned to other processes, compounds, compositions and/or uses not specifically described and/or defined in the examples, but falling under the scope of what is defined in the claims.

Thus, the following examples describe the invention in more detail, but do not limit the invention and its scope as claimed.

### EXPERIMENTAL SECTION

The following examples describe clinical studies and post-hoc analyses in which vaccinal responses to influenza and SARS-CoV-2 mRNA vaccines in patients under treatment with the BTK inhibitor evobrutinib were studied. It is understood that various embodiments of the disclosure reflected in the examples may be practiced, given the general description provided above. Although the disclosure is described in some detail by way of illustration and example for purposes of clarity of understanding, the description and examples should not be construed as limiting the scope of the invention.

### Example 1

In the clinical study NCT02975336, subjects suffering from systemic lupus erythematosus (SLE) were treated with evobrutinib (M2951), as described below. In the course of the study, a number of patients were administered an influenza vaccine and the antibody response to this vaccine was examined in a post-hoc analysis, as described below.

### Clinical Study Design

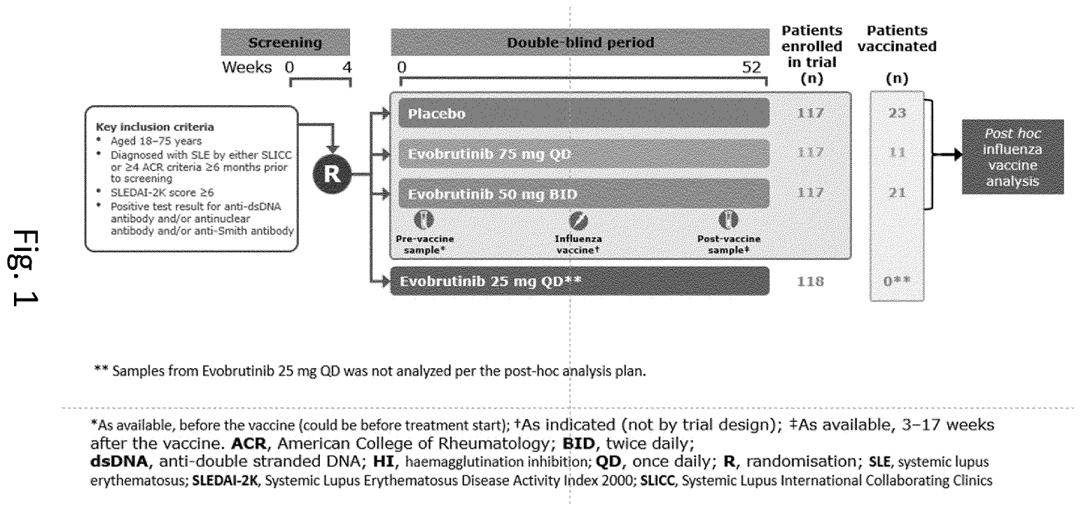

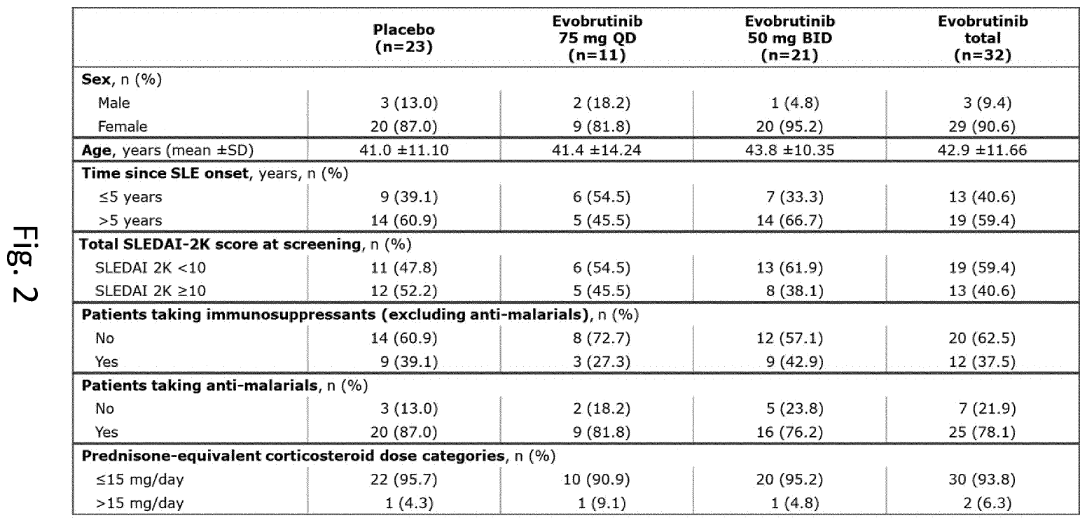

The clinical study NCT02975336 was a phase II, randomized, double-blind, placebo-controlled trial designed to evaluate the safety and efficacy of evobrutinib (M2951) in subjects with SLE (see FIG. 1). Details on this study have been published previously (Wallace et al., Lupus Science & Medicine (2019), vol 6(1):e000349 (doi: 10.1136/lupus-2019-lsm.212); Wallace et al. Arthritis Rheumatol. (2020), vol. 72 (suppl. 10); see also clinical study posting for NCT02975336 at www.clinicaltrials.gov). Briefly, patients were randomized 1:1:1:1 to receive oral evobrutinib 25 mg once-daily (QD), 75 mg QD, 50 mg twice-daily (BID), or placebo. The study was composed of a Screening Period of 4 weeks, a double blinded treatment period of 52 weeks, with an optional open-label Long-term Extension (LTE) Period of 104 weeks, and a Safety Follow-up Period of 4 weeks post-treatment.

For the post-hoc sub-study and analysis of the present example, only available samples from evobrutinib 75 mg once daily (QD), 50 mg twice-daily (BID) or placebo were evaluated.

Subjects were included in this sub-study who received at least 1 dose of study drug (evobrutinib or placebo) and who:
received an influenza vaccine while on treatment with evobrutinib 75 mg QD or 50 mg BID (excluding the vaccines that were received on the day of the treatment start), and
had a pre-vaccination blood sample, and
had a post-vaccination blood sample collected within 3-17 weeks after receiving the vaccine.

### Inclusion Criteria

Patients were enrolled i.a. based on the following inclusion criteria:
Aged 18-75 years
Diagnosed with SLE by either SLICC or ≥4 ACR ( ) criteria ≥6 months prior to screening (SLICC: Systemic Lupus International Collaborating Clinics; ACR: American College of Rheumatology)
SLEDAI-2K score ≥6 (SLEDAI-2K: Systemic Lupus Erythematosus Disease Activity Index 2000)
Positive test result for anti-dsDNA antibody and/or antinuclear antibody and/or anti-Smith antibody

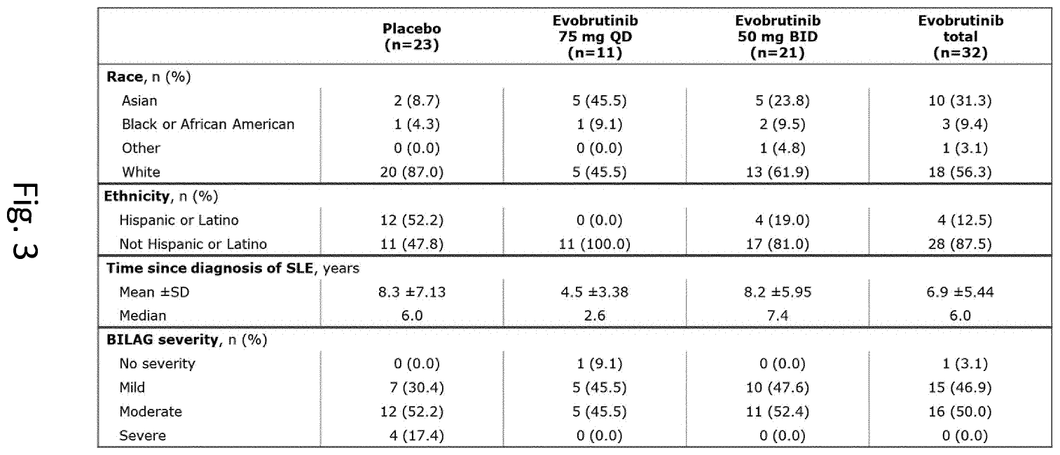

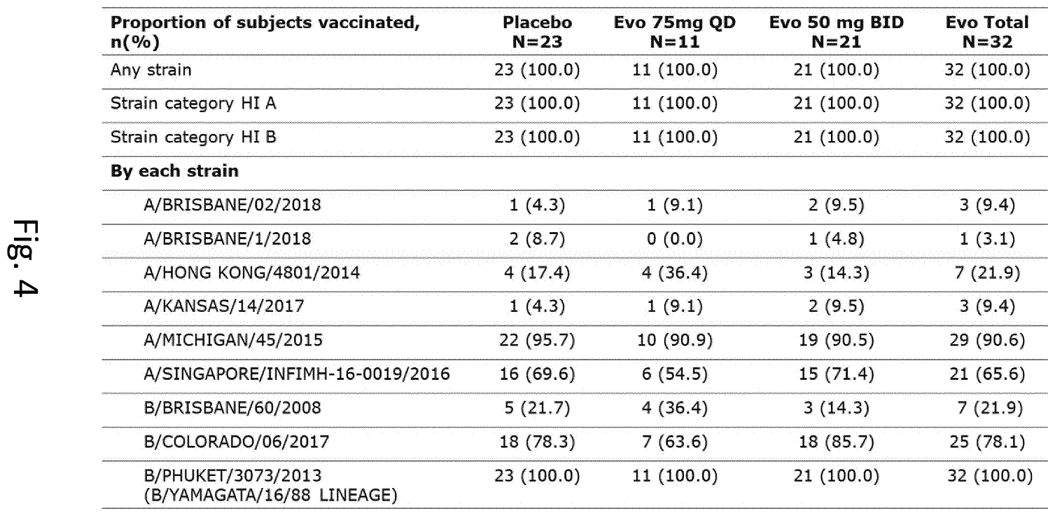

The baseline characteristics of the patients enrolled in the post-hoc influenza vaccine analysis are provided in FIGS. 2 and 3.

### Vaccination

Only patients that received the influenza vaccine were considered in this sub-study. Subjects were administered vaccine during the course of the study based on investigator's judgement.

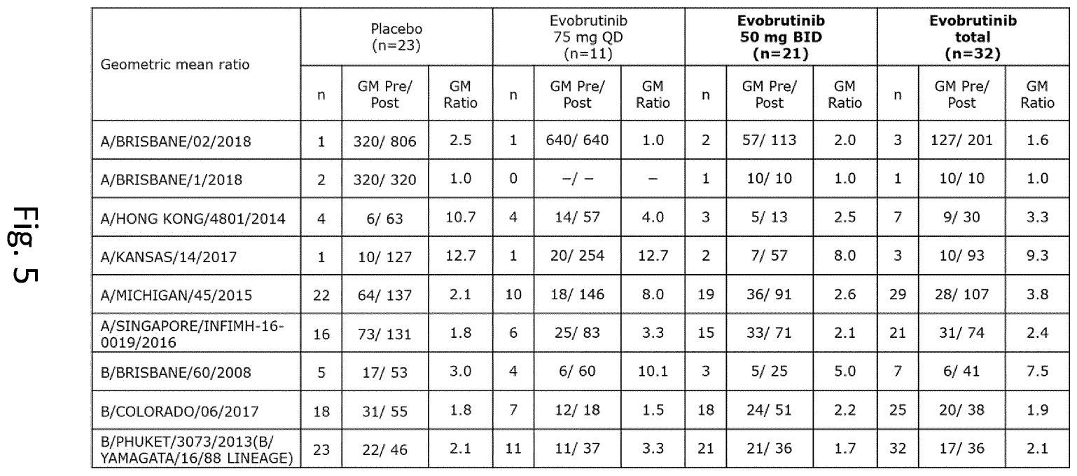

A summary of the proportion of subjects in the different treatment groups vaccinated with specific strains by category and type is provided in FIG. 4. (Of note, such influenza vaccines are quadrivalent vaccines, i.e., they are composed of four different flu viruses: an influenza A(H1N1) virus, an influenza A(H3N2) virus, and two influenza B viruses.)

### Measurement of the Influenza Vaccinal Response—Haemagglutination Inhibition (HI) Serum Antibody Titer Assay

The HI serum antibody titers were determined as follows: Patients' serum samples were serially diluted into a 96-well plate and incubated with a constant amount of specific influenza virus strains (strains were tested individually and matched the strains used in the vaccine administered to the patient which was based upon year of administration and geographical location). Subsequently, red blood cells (RBCs) were added to each well. After incubation, the plate was tilted by 90 degrees to evaluate the level of hemagglutination inhibition. If anti-influenza antibodies were present in the patient's serum sample that cross-react with the virus strain, haemagglutination of the RBCs was inhibited (i.e., virus could not bind RBCs) and the RBCs appeared as a uniformed pellet in the well. If anti-influenza antibodies were not present (or sufficiently diluted) the virus particles were free to bind HA (haemagglutinin) binding sites on RBCs, resulting in cross-linking of the cells and inhibition of pellet formation in the well (i.e., cells appeared dispersed). The well with the highest dilution factor associated with inhibition of haemagglutination was defined as the anti-influenza haemagglutination inhibition antibody titer.

The median time of assessment of HI serum antibody titers post-vaccination in the different treatment groups was 8.1-11.6 weeks.

### Response Definitions

a. Seroconversion

The following definition for seroconversion was used: pre-vaccination titers of <1:10 rising to ≥1:40 or ≥1:10 increasing >4-fold post-vaccination. The seroconversion rate was calculated as percentage of patients who met this definition for seroconversion.

b. Seroprotection

The following definition for seroprotection was used: serum HI titer of ≥1:40 post-vaccination. The seroprotection rate was calculated as percentage of patients who met this definition for seroprotection.

### Results

The median treatment exposure prior to vaccination in the different treatment groups was 16.4-36.4 weeks. The median time of assessment of HI serum antibody titers post-vaccination in the different treatment groups was 8.1-11.6 weeks.

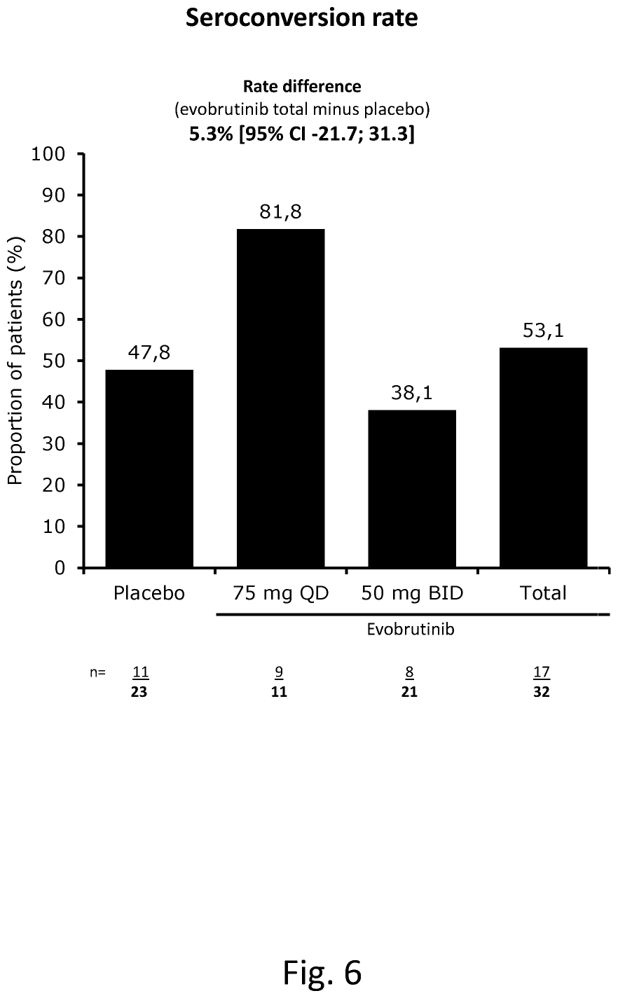

Measurements of the haemagglutination inhibition serum antibody titers in the pre- and post-vaccination samples obtained during this study to different viral strains are summarized in FIG. 5 (geometric mean values). The figure also presents the ratio of the geometric mean pre- and post-vaccine HI titer levels.

As can be seen from these data, the geometric mean ratios in the placebo and the evobrutinib groups are generally comparable and similar.

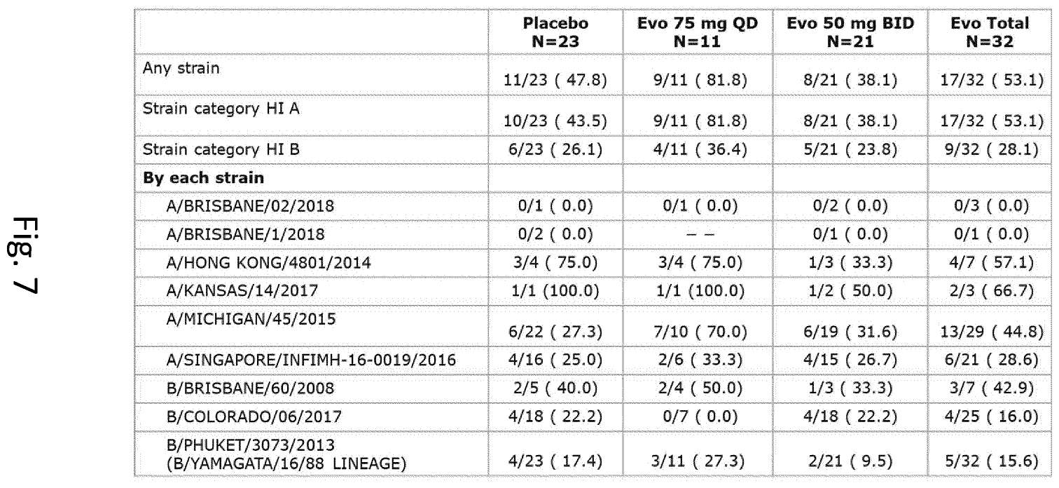

The serological analysis led to the seroconversion summarized in FIGS. 6 and 7.

As can be seen from FIG. 6, the seroconversion rate in the placebo and evobrutinib [total] groups were 47.8% and 53.1%, respectively, with a difference of 5.3% in favor of evobrutinib [total]. Thus, the seroconversion rate was comparable between placebo- and evobrutinib-treated patients.

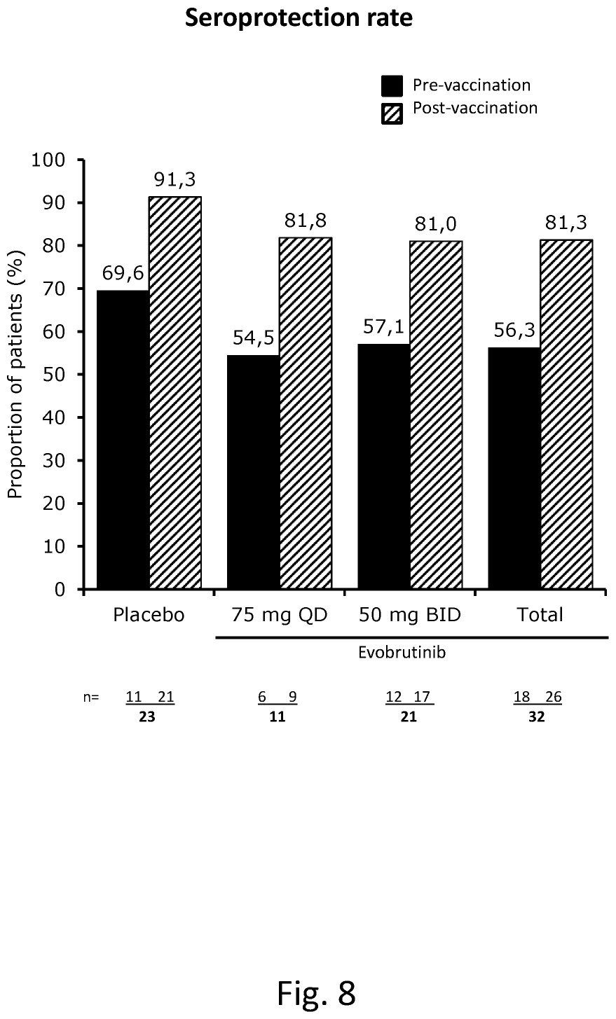

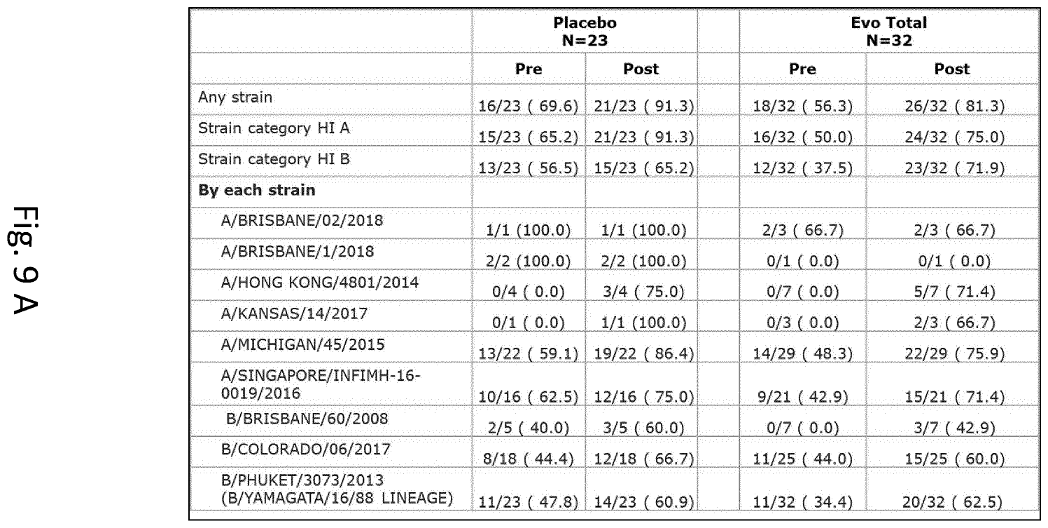

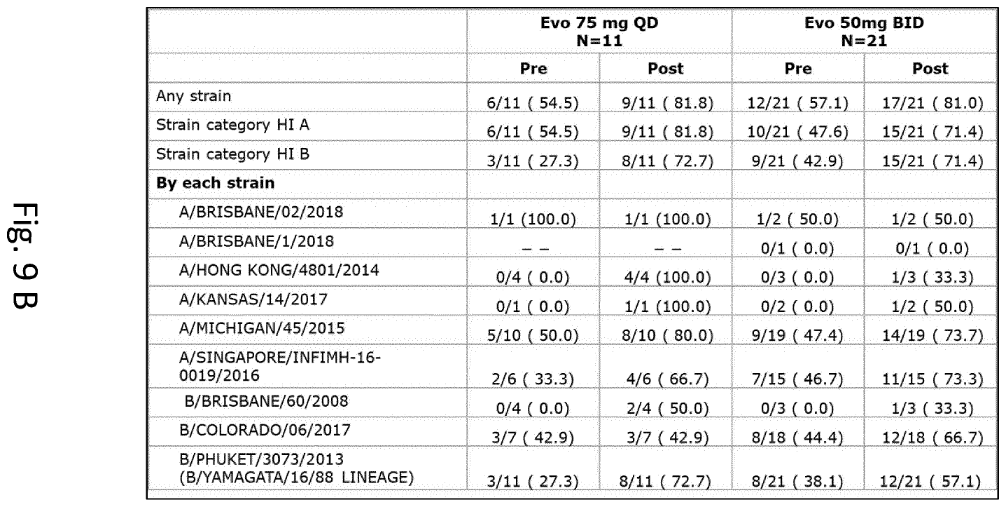

The seroprotection data obtained in this study is summarized in FIGS. 8 and 9.

As can be seen from FIG. 8, seroprotection rate in the pre- vs post-vaccination in the placebo and evobrutinib [total] groups were 69.6% (pre-vaccination) vs 91.3% (post-vaccination), and 56.3% (pre-vaccination) vs 81.3% (post-vaccination), respectively. Based on these data, it is evident that there was comparable and similar seroprotection rates post-vaccination, both in placebo and evobrutinib [total] groups.

In sum, the overall humoral immune response to the influenza vaccine was comparable between placebo- and evobrutinib-treated patients in this phase II trial with SLE patients.

It is expected that the large proportion of the patients in this study subjected to influenza vaccination were previously exposed to influenza strains underlying the present analysis due to infections and/or previous influenza vaccinations. Thus, the response to the vaccination observed in this study was for many patients a recall response against an antigen their immune system had previously encountered. The present study, therefore, shows a recall response can be successfully elicited upon vaccination of subjects that are at the same time undergoing treatment with evobrutinib.

This is the first evidence demonstrating that patients treated with evobrutinib or any BTK inhibitor for an autoimmune disease (such as SLE) can mount a humoral response to seasonal influenza vaccination.

### Example 2

In the clinical study NCT02975349, subjects suffering from relapsing multiple sclerosis (RMS) were treated with evobrutinib (M2951). Details of this study have been published in Montalban et al., N Engl J Med (2019), vol. 380(25), p. 2406-2417. See also clinical study posting for NCT02975349 at www.clinicaltrials.gov.

In the course of the study, a number of subjects were administered mRNA and non-mRNA COVID-19 vaccines. The antibody response to these vaccinations was examined in a post-hoc analysis, as described below.

The goal of this vaccination analysis was to assess if an immune response can be induced in subjects under treatment with evobrutinib. The analysis was carried out as a retrospective analysis of SARS-CoV-2 IgG anti-S1/S2 antibodies (also referred to as S1/S2 IgG antibody) in subjects treated with evobrutinib for RMS.

### Clinical Study Design

The clinical study NCT02975349 is a phase II, randomized, placebo-controlled trial to evaluate the safety and efficacy of evobrutinib (M2951) in subjects with relapsing multiple sclerosis (RMS). Details on this study have been published previously (refer to reference provided above Montalban et al., 2019 N Engl J Med). The study was composed of a screening period of 4 weeks, a double blinded treatment period of 48 weeks, with an optional open-label extension (OLE) period of 312 weeks, and a safety follow-up period of 4 weeks post-treatment.

Patients were enrolled i.a. based on the following inclusion criteria:
Aged 18 to 65 years
Had relapsing-remitting multiple sclerosis or secondary progressive multiple sclerosis with superimposed relapses revised McDonald 2010 criteria for MS and Lublin and Reingold (Polman et al., Ann Neurol. (2011), vol. 69, p. 292-302; Lublin et al., Neurology (2014), vol. 83(3), p. 278-286)
One or more documented relapses within the 2 years before Screening with either:
   a) One relapse which occurred within the last year prior to randomization, or
   b) the presence of at least 1 Gadolinium-enhancing T1 lesion within 6 months prior to randomization
Had a score of no more than 6 on the EDSS (Expanded Disability Status Scale which ranges from 0 [no disability] to 10 [death])

Patients were randomized 1:1:1:1: to receive placebo, oral evobrutinib 25 mg once-daily (QD), 75 mg QD, 75 mg twice-daily (BID), or open-label dimethyl fumarate. Patients who received placebo during the first 24 weeks of the DBP, were switched to evobrutinib 25 mg QD until the end of the double-blind period (i.e., 48 weeks). Patients participating in the OLE after the DBP received EVO 75 mg QD and then, after a mean of 49.8 weeks, switched to EVO 75 mg BID.

For the analysis of pre-vaccination, post-vaccination and post-booster vaccination S1/S2 IgG antibody levels, plasma samples were selected according to the following criteria:
Pre-vaccination: sample taken at the last doctor's visit of the patient before the date when the first COVID-19 vaccine dose was administered
post-vaccination: sample taken at the first doctor's visit of the patient that took place at least 28 days after a complete vaccination cycle (as defined by the manufacturer) was administered (for example, in case of the mRNA COVID vaccine of VAC-OLE I, at least 28 days after the date when the second dose of the mRNA COVID vaccine was administered)
post-booster vaccination: sample taken in the first doctor's visit of the patients that took place at least 7 days after the date when the booster vaccination was administered

### Vaccination

An initial analysis was performed on patients (N=24) that received only mRNA SARS-CoV-2 vaccines (referred to as VAC-OLE I). A subsequent analysis was performed on patients (N=45), which include 24 patients from VAC-OLE I and additional patients (N=21) that received either an mRNA or a non-mRNA SARS-CoV-2 vaccine (referred to as VAC-OLE II). Within VAC-OLE II, a subset of these patients (N=14) also received a booster vaccine.

### VAC-OLE I Analysis

Only SARS-CoV-2 mRNA vaccines against COVID-19 were considered for this vaccination analysis. Eligible patients were selected based on the information in the “Concomitant Medications” eCRF form in the study provided by the investigators.

A total of 24 patients, fulfilling the following criteria, were included in the analysis and constituted the VAC-OLE I analysis set:
Subjects who were in the OLE and received evobrutinib 75 mg BID, and
had 2 doses of mRNA vaccine against COVID reported as concomitant medication, and
signed the Informed Consent for the vaccination analysis.

Subjects were administered the two doses of vaccine during the course of the study based on investigator's judgement. Of the 24 vaccinated patients:
20 received the mRNA vaccine by BioNTech/Pfizer
4 patients received the mRNA vaccine by Moderna.

### VAC-OLE II Analysis

SARS-CoV-2 mRNA and non-mRNA vaccines were considered for this analysis. Eligible patients were selected based upon the information in the “Concomitant Medications” eCRF form in the study provided by the investigators.

A total of 45 participants (24 from the VAC-OLE I study and 21 additional participants from the VAC-OLE II study) fulfilling the following criteria were identified:
Subjects who were in the OLE and received evobrutinib 75 mg BID, and
2 doses of BioNTech/Pfizer/Comirnaty®, Moderna/Spikevax® or Vaxzevria®/Covishield® or 1 dose of Jcovden® vaccine against COVID reported as concomitant medication, and
signed the Informed Consent for the vaccination analysis.

Subjects were administered a complete COVID-19 vaccination cycle (i.e., one or two doses of a vaccine depending on the manufacturer's instructions) during the course of the study based on investigator's judgement. For the 45 fully vaccinated patients:
31 received the mRNA vaccine by BioNTech/Pfizer/Comirnaty®
6 received the mRNA vaccine by Moderna/Spikevax®
4 received the non-mRNA vaccine by Vaxzevria®/Covishield®
4 received the non-mRNA vaccine by Jcovden.

Additionally, a sub-group of these patients (N=14) were also administered a booster vaccination by BioNTech/Pfizer/Comirnaty® or Moderna/Spikevax® based on the local guidance and reported as concomitant medication.

Of the 14 patients that received booster vaccinations:
13 received the mRNA vaccine by BioNTech/Pfizer/Comirnaty®
1 received the mRNA vaccine by Moderna/Spikevax®.

### Measurement of S1/S2 IgG Antibody Levels

SARS-CoV-2 antibody levels were determined using the LIAISON® SARS-CoV-2 S1/S2 IgG assay (DiaSorin, Inc); an indirect sandwich chemiluminescent immunoassay that qualitatively detects IgG antibodies to SARS-CoV-2. The assay is described at https://www.fda.gov/media/137359/download. Briefly, magnetic particles coated with recombinant S1 and S2 SARS-CoV-2 proteins were incubated with patients' plasma samples, calibrator samples or control samples. After washing away unbound material, a mouse monoclonal anti-human IgG antibody linked to an isoluminol derivative was added. Following another incubation and wash step, the starter reagents were added triggering a flash chemiluminescence reaction. The resulting light signal, representative of the amount of bound isoluminol-antibody conjugate, was measured by a photo multiplier as relative light units (RLU) and was indicative of the presence of IgG S1/S2 antibodies to SARS-CoV-2 virus. Test results are assessed qualitatively as positive or negative. The lower limit of quantification (LLoQ) in this assay was 3.8 AU/mL. In line with FDA guidance, values below a cutoff of 15 AU/mL (i.e. about 4× above the LLoQ for the assay) were considered as a negative readout.

The primary endpoint of this study was defined as fold changes in levels of S1/S2 IgG antibodies from pre- to post-vaccination.

### Results

### VAC-OLE I

In the VAC-OLE I analysis set, the proportion of female (79.2%) was higher than in the mITT (modified intention-to-treat) group (69.3%). The mean (SD) age was comparable: 44.6 years (10.07) in the VAC-OLE subgroup compared to 42.4 years (10.67) in the mITT.

The median treatment exposure with evobrutinib 75 mg BID in the OLE prior to vaccination was 106 weeks. The median time of assessment of S1/S2 IgG antibody levels post-vaccination (second dose) was 8.7 weeks.

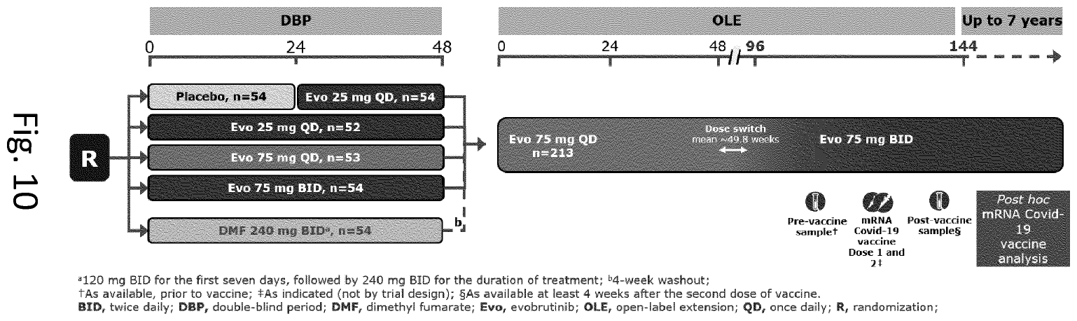

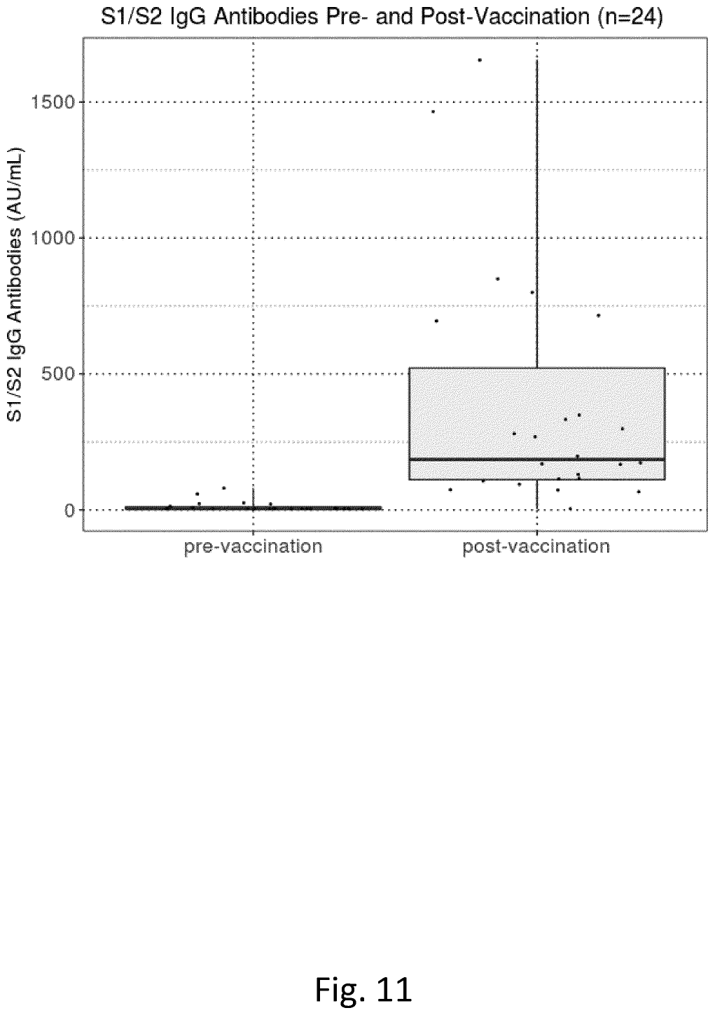

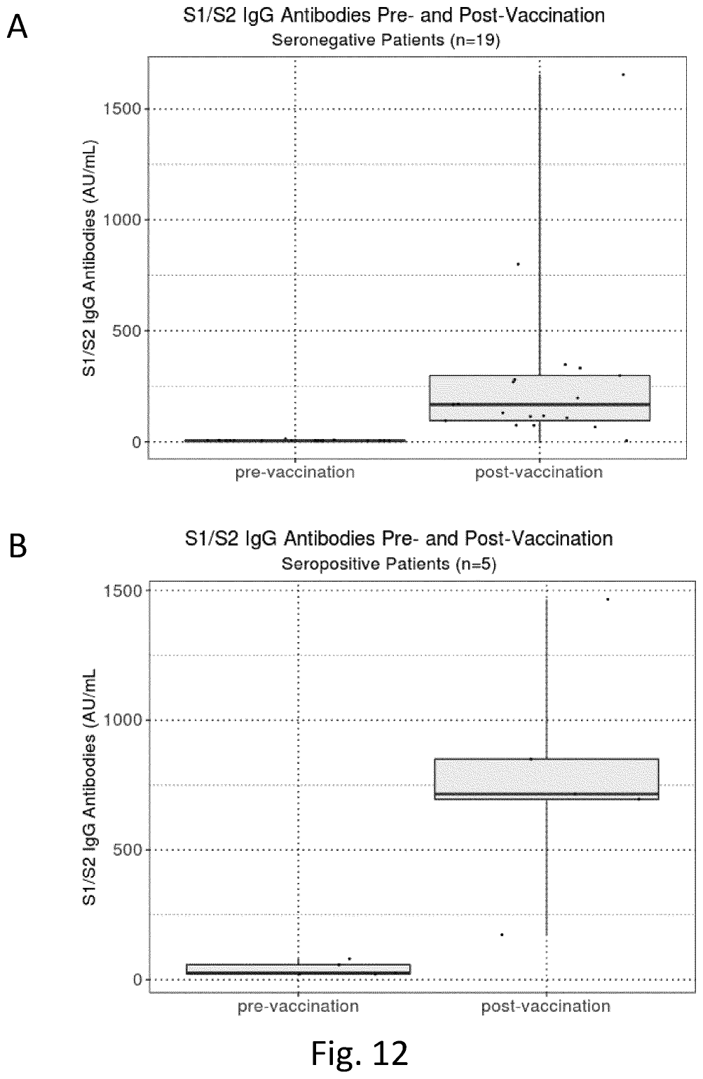

Results of the S1/S2 IgG antibody levels in pre- and post-vaccination samples in patients who received two doses of an mRNA vaccine against COVID-19 while on treatment with 75 mg evobrutinib twice a day are shown in FIGS. 11-13.

Out of 24 patients of the VAC-OLE I study, 5 patients were seropositive before the first mRNA vaccine administration (S1/S2 IgG antibody level ≥15 AU/ml), whereas 19 patients were seronegative before vaccination. The majority of patients in the seronegative subgroup had a value at the lower limit of quantification (i.e., around 3.8 AU/ml) prior to vaccination and a geometric mean post-vaccination level of 155.8 AU/mL. Patients with seropositive pre-vaccination samples had a geometric mean level of 35.5 AU/mL which increased to 639.6 AU/mL after the second vaccination. In the overall VAC-OLE population, S1/S2 IgG antibody levels were low before the first vaccination (geometric mean of 6.7 AU/mL) and increased to a geometric mean level of 209.1 AU/mL after the second vaccination. Subjects who had been seropositive before the first vaccine dose achieved visibly higher antibody levels after the second vaccine dose as compared to subjects who had been seronegative before the first vaccine dose.

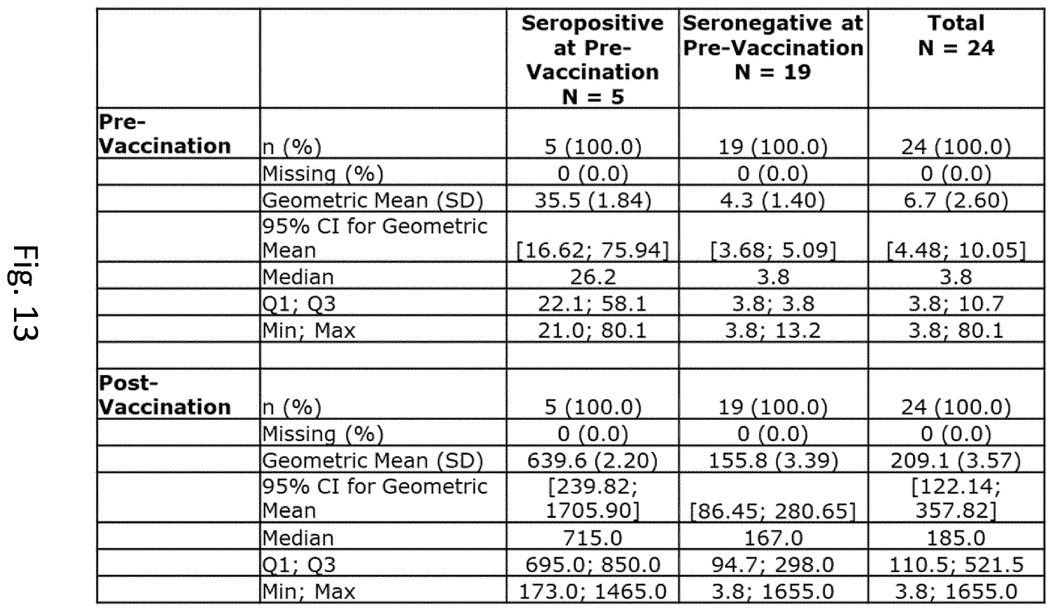

Fold changes were calculated as the ratio between the post- and pre-vaccination S1/S2 IgG antibody levels. The results are summarized in FIG. 14.

As can be seen from this figure, the majority of patients had at least a 10- to 60-fold induction of S1/S2 IgG antibody levels from pre-vaccination to post-vaccination. The geometric mean fold change in seropositive patients (18.0) was lower as compared to seronegative patients (36.0), since S1/S2 IgG antibody levels were already increased pre-vaccination in seropositive patients.

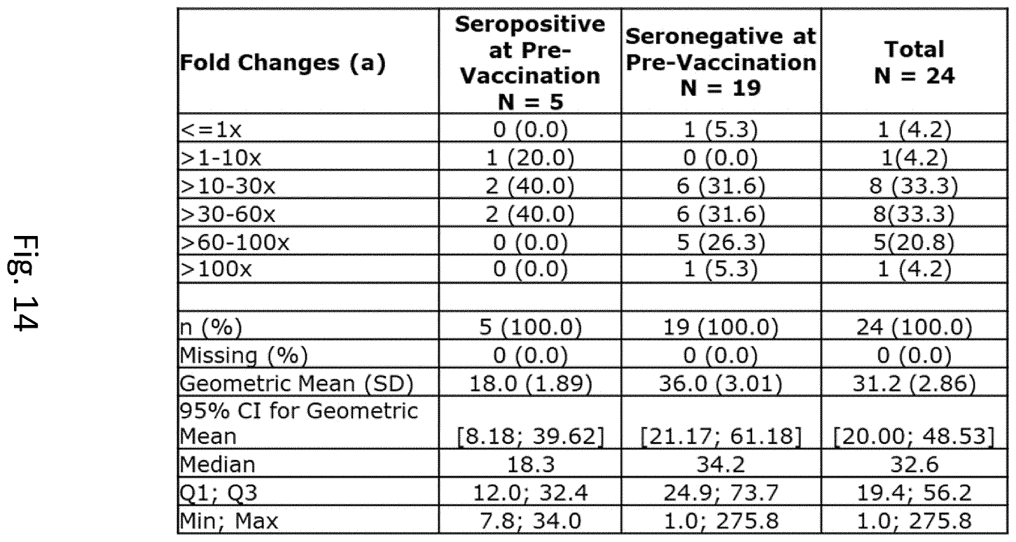

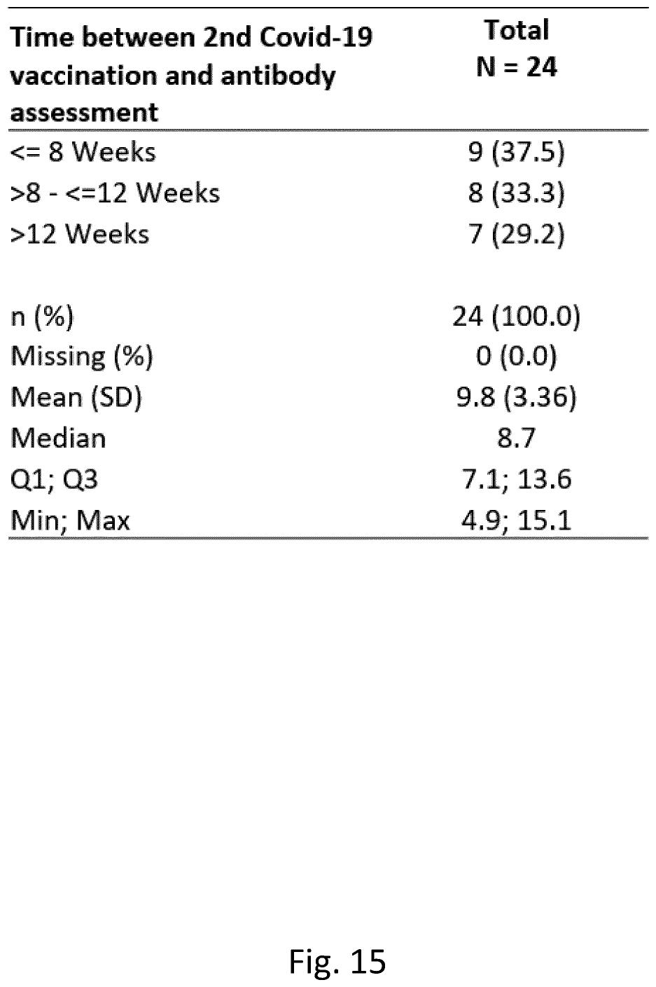

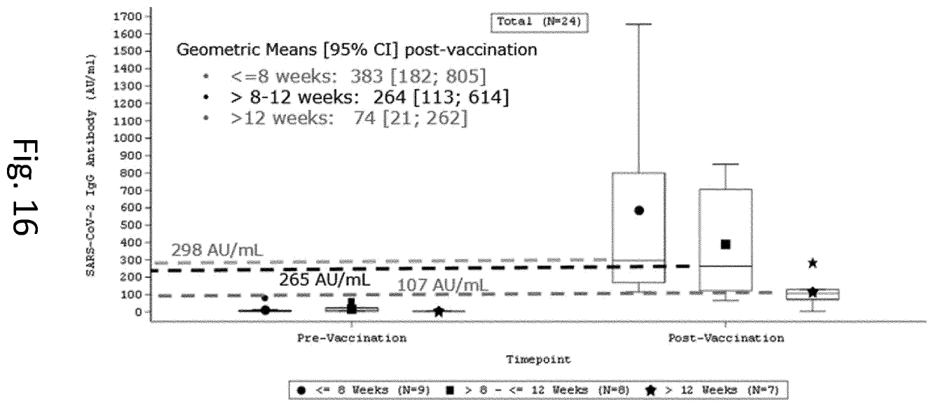

In a next step, the effect of the time elapsed between the date of second mRNA vaccine against COVID-19 and antibody assessment (i.e. the date of post-vaccination sample collection to be analyzed by antibody assessment) was examined. Results are summarized in FIGS. 15 and 16. For the majority of patients, the time between the second mRNA vaccine against COVID-19 and the sample for antibody assessment was >8 weeks (62.5%), as can be seen from FIG. 15. FIG. 16 shows that, with increasing time between the second COVID-19 vaccination and antibody assessment, a decrease in S1/S2 IgG antibody levels was observed. Patients who had a time interval between the second COVID-19 vaccination and post-vaccination sample collection which was less or equal to 8 weeks had the highest level (geometric mean of 383 AU/mL) compared to those with a duration >12 weeks (geometric mean of 74). Patients with post-vaccination samples collected between 8 and 12 weeks after receiving the second dose of mRNA Covid-19 vaccine had a geometric mean antibody level of 264.

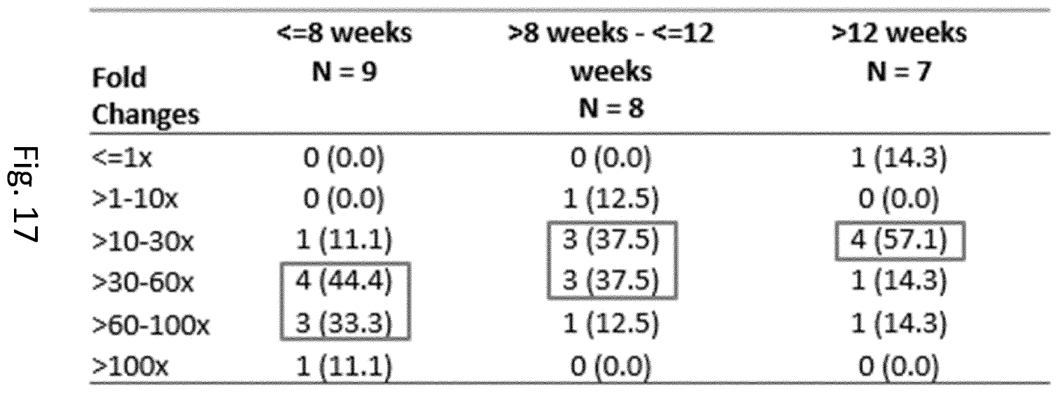

In a further step, the frequency was examined at which certain fold-changes of the S1/S2 IgG antibody level were observed in dependence on the time elapsed between the second COVID-19 vaccination and antibody assessment for the group of patients in the VAC-OLE. Results are summarized in FIG. 17.

As can be seen from this figure, the majority of patients with a time period ≤8 weeks between vaccination and S1/S2 IgG antibody level assessment had fold changes between 30 and 100. In patients with longer time periods between vaccination and antibody assessment, the frequencies shift towards smaller fold change categories (i.e., 10- to 60-fold for patients in the 8-12 weeks category and 10- to 30-fold for patients in the >12 weeks category).

### VAC-OLE II

In the VAC-OLE II study (N=45), the proportion of female (68.9%) was similar to the mean in the mITT group (69.3%); the mean (SD) age was comparable: 46.0 years (9.62) versus 42.4 years (10.67) in the mITT group; the median treatment exposure with evobrutinib 75 mg BID during the OLE prior to vaccination was 104 weeks; the median time of S1/S2 IgG antibody levels assessments after receiving a complete vaccination cycle was 10.6 weeks; mRNA vaccines were administered to 37 of 45 (82.2%) participants, non-mRNA vaccines were administered to 8 of 45 (17.9%) participants. For participants who also received a booster vaccination (N=14), the median time between booster vaccination and post-booster sample collection was 7.6 weeks and all received mRNA vaccines.

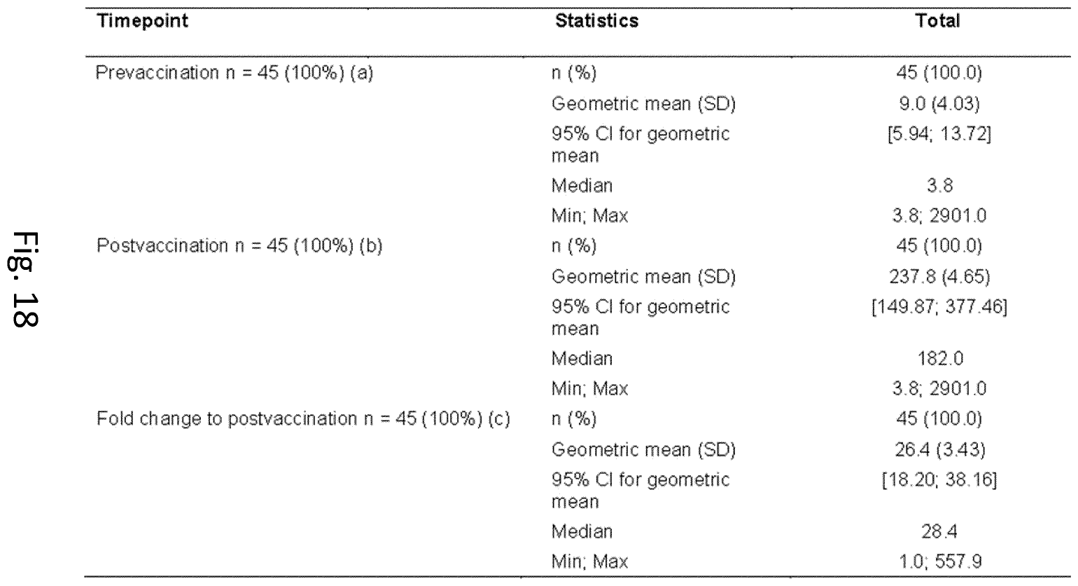

Results of the S1/S2 IgG antibody levels in pre-vaccination and post-vaccination samples for participants receiving a complete vaccination cycle of either an mRNA or a non-mRNA COVID-19 vaccine while on treatment with 75 mg evobrutinib twice a day were assessed. The results are summarized in FIGS. 18-22.

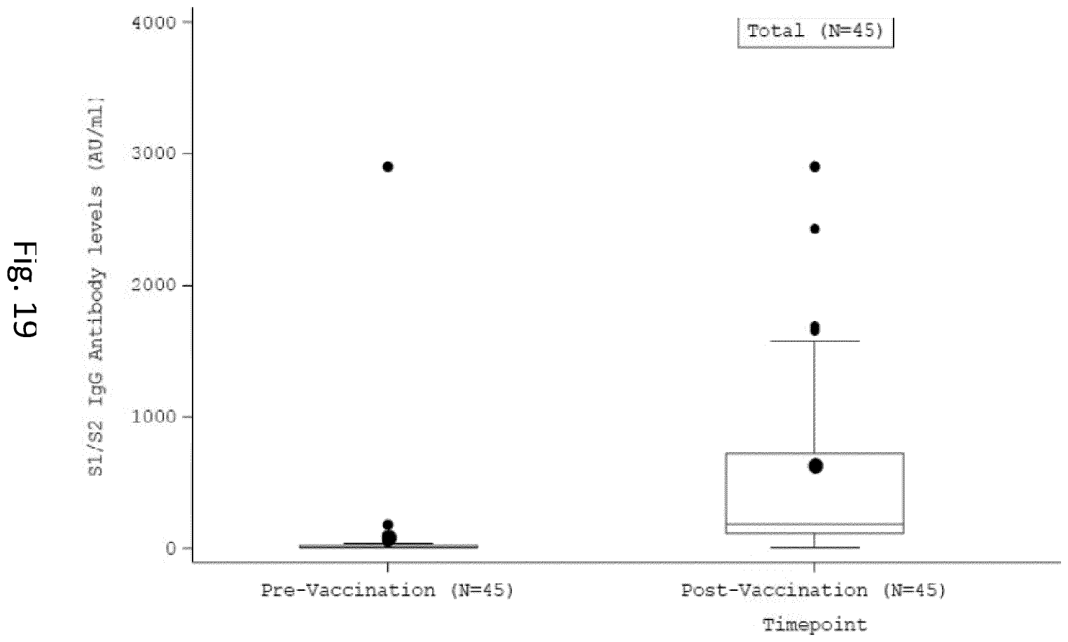

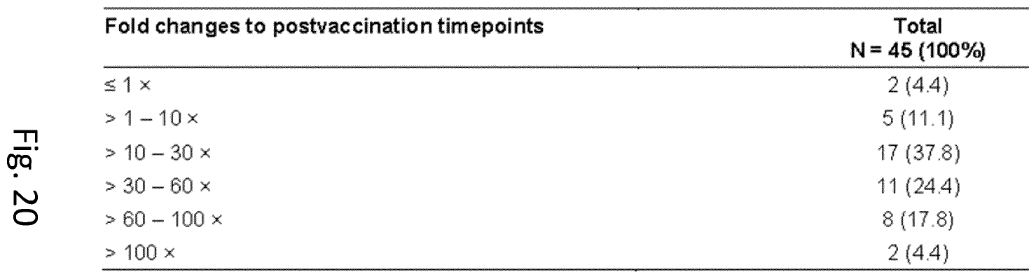

S1/S2 IgG antibody levels were relatively low pre-vaccination with a geometric mean of 9.0 AU/mL and increased to 237.8 AU/mL after a complete vaccination cycle resulting in a 45-fold increase in anti-COVID-19 antibody levels (FIGS. 18 and 19). The majority of participants (38 of 45 or 84%) had a >10-fold increase of S1/S2 IgG antibody levels from pre-vaccination to post-vaccination, and almost half (21 of 45 or 47%) had a >30-fold induction of S1/S2 IgG antibody levels (FIG. 20).

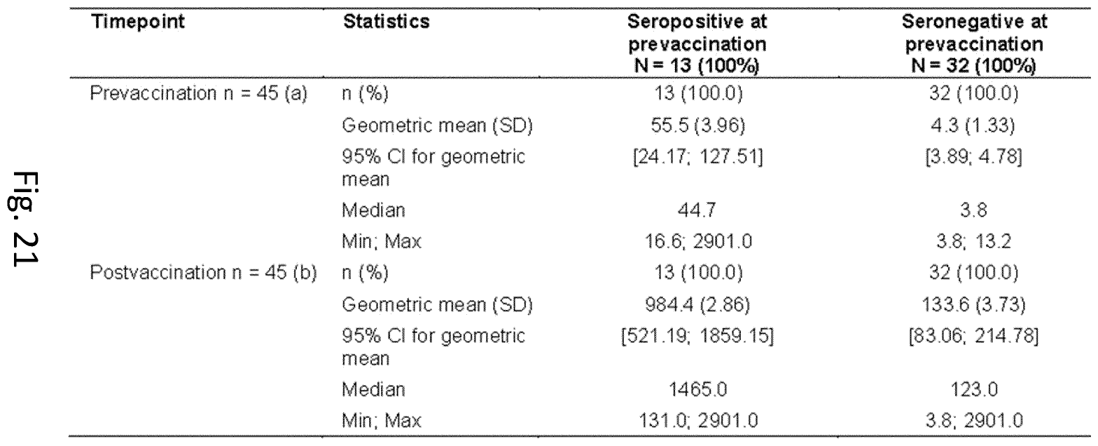

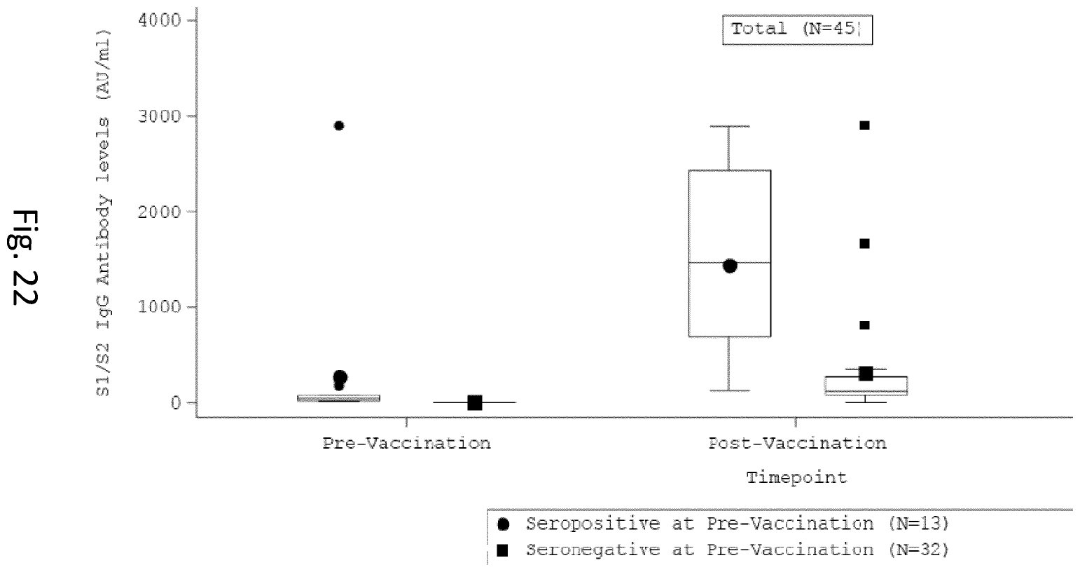

Of the 45 participants in this study, 32 were seronegative before vaccination (S1/S2 antibody levels <15 AU/mL) and 13 participants were seropositive (S1/S2 IgG antibody levels ≥15 AU/mL) (FIG. 21). For the majority of participants in the seronegative subgroup, their antibody values were at the lower limit of quantification for the assay (3.8 AU/mL) prior to vaccination and their post-vaccination antibody geometric mean level increased to 133.6 AU/mL. Participants with seropositive pre-vaccination samples had an antibody geometric mean level of 55.5 AU/mL which increased to 984.4 AU/mL after a complete vaccination cycle (FIGS. 21 and 22). Participants who were seropositive before vaccination achieved noticeably higher antibody levels after vaccination compared to participants who were seronegative before vaccination.

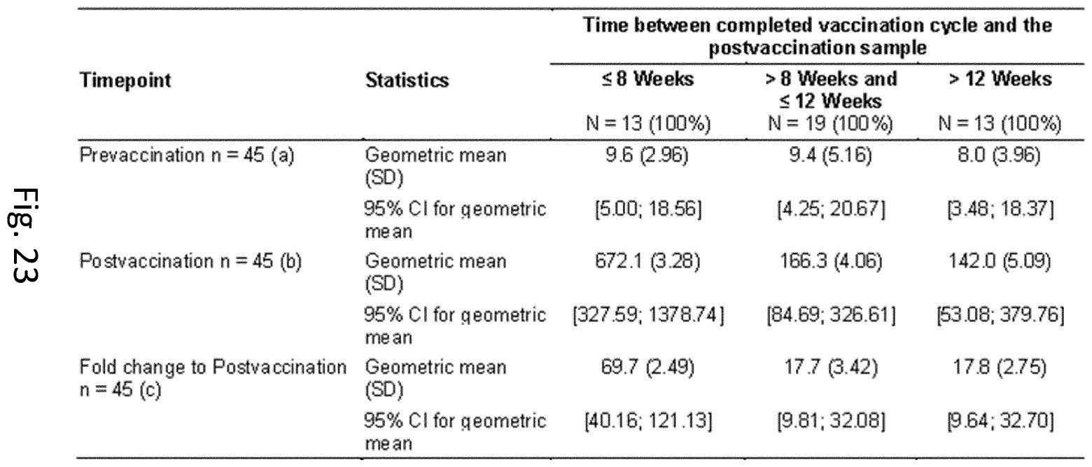

The elapsed time between the date of the complete COVID-19 vaccination cycle and date of the post-vaccination S1/S2 IgG antibody assessment was evaluated. Results are summarized in FIGS. 23-25.

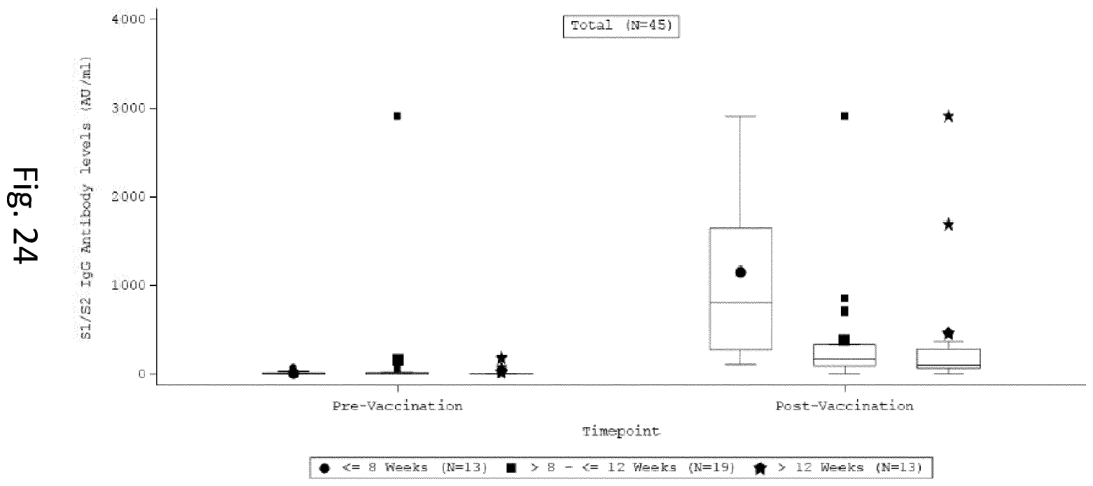

The elapsed time between the completion of the vaccination cycle and post-vaccination sample collection ranged from 4.9 to 19.1 weeks. As discussed below, others have demonstrated that antibody responses to COVID-19 vaccines tend to peak 2 to 4 weeks after vaccination and then decline over time (Favresse et al. (2021); Naaber et al. (2021)). In the VAC-OLE II study, the pre-vaccination antibody levels were similar when participants' antibody levels were sub-grouped as follows: ≤8 weeks, >8 week to ≤12 weeks or >12 weeks after a complete vaccination cycle, i.e., values ranged from 8.0 to 9.6 AU/mL (FIGS. 23 and 24). In contrast, the geometric mean post-vaccination was substantially higher for the <8 weeks subgroup (672 AU/mL) compared to 166 and 142 AU/mL for the >8 week to ≤12 weeks and >12 weeks subgroups, respectively. This difference was reflected in the fold change in the geometric mean, i.e., 69.7, 17.7 and 17.8 for participants whose blood samples were assessed ≤8 weeks, >8 week to ≤12 weeks, or >12 weeks, respectively, after a complete vaccination cycle.

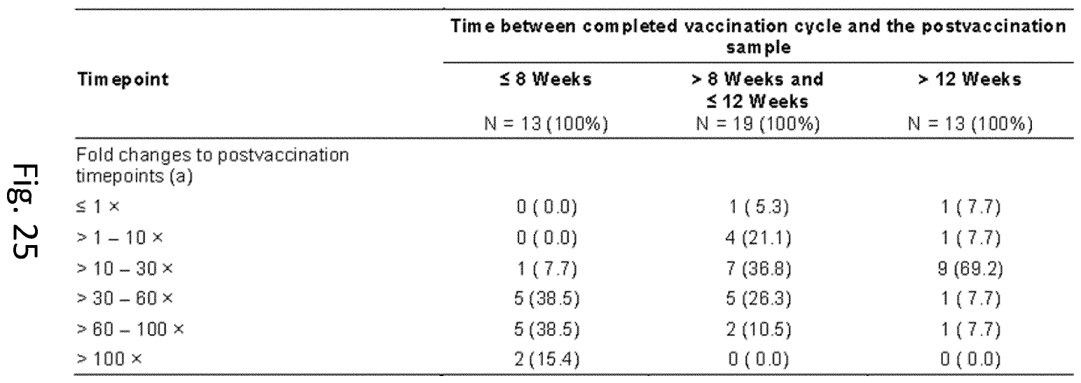

Although the overall fold change for the >8 week to ≤12 weeks and >12 weeks subgroup were very similar, a closer evaluation of the data showed that the number of participants with a >30-fold change in antibody levels after vaccination were notably higher in the >8 and ≤12 weeks subgroup (7 of 19 or 37%) than the >12 weeks group (2/13 or 15%) (FIG. 25).

Of the 45 participants included in the VAC-OLE II study, 37 participants (82.2%) received mRNA vaccines and 8 participants (17.8%) received non-mRNA vaccines. The pre-vaccination S1/S2 IgG antibody geometric means were similar at 8.5 AU/mL (95% CI 5.23; 13.72) and 12.1 AU/mL (95% CI 4.62; 31.75) for participants that received mRNA and non-mRNA vaccines, respectively. The post-vaccination antibody geometric means were 247.7 AU/mL (95% CI 150.45; 407.78) and 197.1 AU/mL (95% CI 43.09; 901.78) for participants that received mRNA and non-mRNA vaccines, respectively. These data provide evidence that participants with MS treated with evobrutinib can mount an antibody response to both mRNA and non-mRNA COVID-19 vaccines.

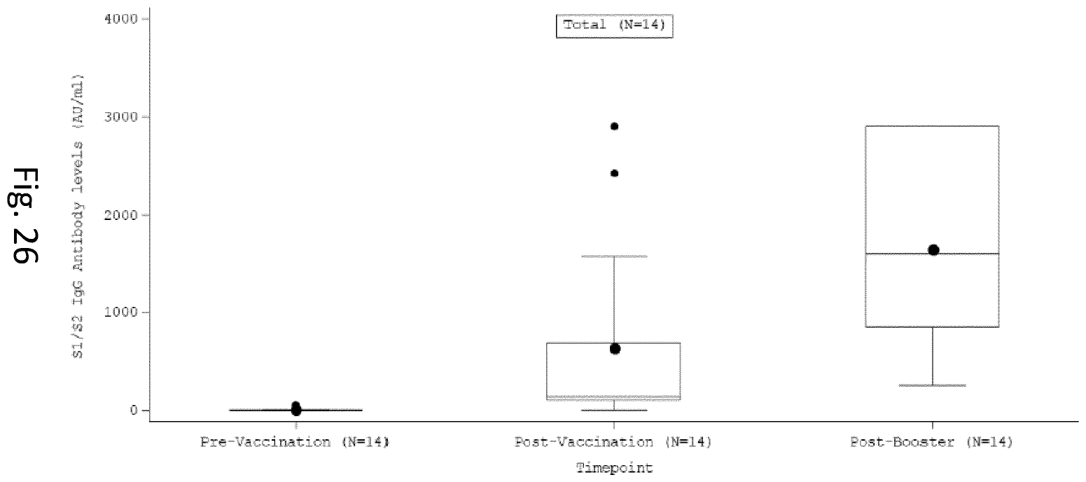

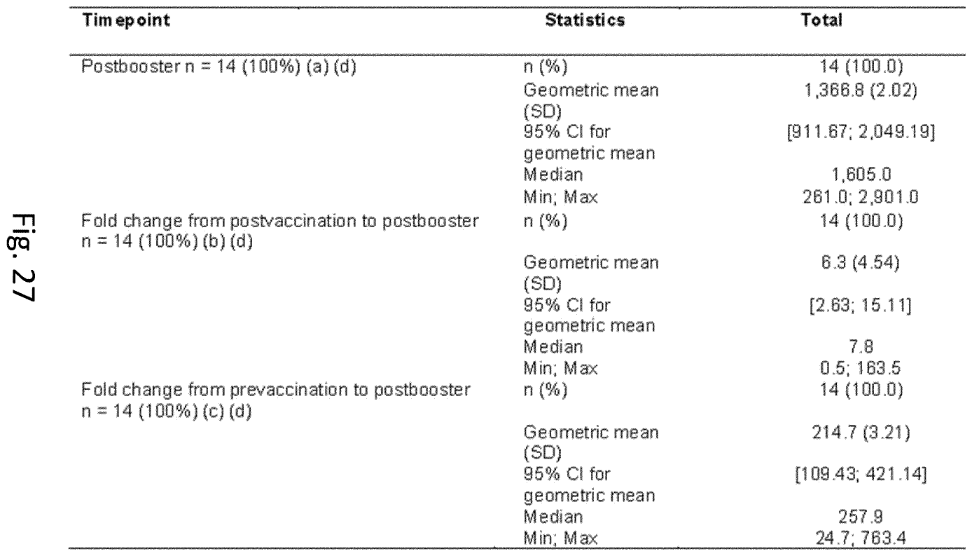

Results of the S1/S2 IgG antibody levels in participants that also received a booster vaccination were assessed. Results are summarized in FIGS. 26 and 27.

Of the 45 participants included in the VAC-OLE II study, 14 participants (31.1%) received a booster vaccination; all participants received mRNA vaccines as booster vaccine. Higher S1/S2 IgG antibody levels after booster vaccination were observed compared to levels after the complete vaccination cycle (FIG. 26). The geometric mean fold change from post-vaccination to post-booster was 6.3 and from pre-vaccination to post-booster was 214 (FIG. 27). Others have shown that in healthy individuals, COVID-19 booster vaccinations result in higher antibody levels compared to the complete vaccination (M. Gilboa et al., JAMA Netw Open (2022), 5(9):e2231778).

In Brill at al., JAMA Neurology (2021), vol. 78(12), p. 1510-1514, S1/S2 IgG antibody levels were measured in healthy control, untreated MS patients and ocrelizumab-treated MS patients (a disease modifying treatment for multiple sclerosis) 2 to 4 weeks after receiving the second dose (i.e., a complete cycle) of mRNA COVID-19 vaccines. The authors stated that vaccinated untreated MS patients reported similar levels of S1/S2 IgG antibody levels compared to vaccinated healthy control, whereas patients with MS treated with ocrelizumab had very low S1/S2 IgG antibody levels.

In Naaber et al., The Lancet Regional Health—Europe (2021), vol. 10:100208, the antibody levels of 122 subjects were monitored over time. The authors reported a robust antibody response to the corona virus spike protein after a complete vaccination cycle. Antibody levels declined at 12 weeks and 6 months post-vaccination. At 6 months after a complete vaccination cycle, the spike antibody levels were similar to the levels in subjects vaccinated with one dose or in COVID-19 convalescent subjects.

In Favresse et al., Emerging Microbes and Infections (2021), vol. 10(1), p. 1495-1498, the authors showed based on antibody kinetic data that 3 months post vaccination the maximum antibody concentration was reached 28-42 days after vaccination in subject who were seronegative at baseline, compared to 14 to 42 days in subjects who were seropositive at baseline. The estimated half-life of antibodies was determined to be 55 days (95% CI: 37-107 days) for seronegative subjects and 80 days (95% CI, 46-303 days) for seropositive subjects.

Thus, from this example it can be concluded that subjects treated with evobrutinib respond to COVID vaccination comparably to healthy individuals who are not under treatment with a BTK inhibitor.

In Example 2, the large majority of the subjects in these analyses were seronegative at baseline and did not report having previously received a SARS-CoV-19 vaccine before the start of the study (SARS-CoV-2 appeared for the first time while the study was ongoing, and vaccination occurred worldwide mainly in 2021)); only 13 out of 45 participants were identified as seropositive. Thus, the results suggest that vaccination under evobrutinib treatment may also be carried out successfully for novel antigens (i.e., for antigens that an individual is exposed to for the first time at the vaccination in question). Additionally, this study demonstrated participants treated with evobrutinib could mount an antibody response when administered either the mRNA or non-mRNA COVID-19 vaccines and could mount a response to booster vaccination.

Whereas in Example 1 successful vaccination under evobrutinib treatment was shown for SLE patients, in Example 2 successful vaccination under evobrutinib treatment was shown for MS patients. This implies that successful vaccination under evobrutinib treatment (and likely under treatment with closely related BTK inhibitors) may not be limited to the specific autoimmune diseases of the present examples, but may occur for other autoimmune diseases as well, and feasibly also for other inflammatory diseases.

### REFERENCES

Bogers et al., ECTRIMS (2022), Abstract
Bullock et al., Med. Princ. Pract. (2018), vol. 27, p. 501-507
Brill at al., JAMA Neurology (2021), vol. 78(12), p. 1510-1514
Choi et al., Immunol. Allergy Clin. North Am. (2010), vol. 30(1), p. 75-101
Douglas et. al., Haematologica (2017), 102, p. e398
Favresse et al., Emerging Microbes and Infections (2021), vol. 10(1), p. 1495-1498
Filippi et al., Nature Reviews Disease Primers (2018), vol. 4, art. 43
Gilboa et al., JAMA Netw Open (2022), 5(9):e2231778. doi: 10.1001/jamanetworkopen.2022.31778
Gilhus et al., Nature Reviews Disease Primers (2019), vol. 5, art. 30
Holgate et al., Nature Reviews Disease Primers (2015), vol. 25(1), art. 15025
Horwood et al., J. Exp. Med. (2003), vol. 197, p. 1603
Iwaki et al., J. Biol. Chem. (2005), vol. 280, p. 40261
Jansson and Holmdahl, Clin. Exp. Immunol. (1993), vol. 94, p. 459
Jarius et al., Nature Reviews Disease Primers (2020), vol 6, art. 85
Kaul et al., Nature Reviews Disease Primers (2016), vol. 2(1), art. 16039
Li et al., Acta Neuropathologica (2022), vol. 143, p. 505
Lindvall et al., Immunol. Rev. (2005), vol. 203, p. 200
Lleo et al., Autoimmunity Reviews (2010), vol. 9, p. A259-266
Loebermann et al., Nat. Rev. Neurol. (2012), vol. 8, p. 143-151
Lublin et al., Neurology (2014), vol. 83(3), p. 278-286
Mathian et al., Curr. Opin. Rheumatol. (2018), vol. 30, p. 465-470
Matthews et al., Neurology (2013), vol. 80, p. 1330
Min et al., Allergy Asthma Immunol. Res. (2019), vol. 11, p. 470
Montalban et al., New Engl. J. of Medicine (2019), vol. 380, p. 2406
Naaber et al., The Lancet Regional Health—Europe (2021), vol. 10:100208, doi:10.1016/j.lanepe.2021.100208
Pan et al., Chem. Med Chem. (2007), vol. 2, p. 58-61
Papp et al., J. of Cutaneous Medicine and Surgery (2019), vol. 32, p. 50-74
Pleyer et al., Blood (2021), vol. 137(2), p. 185-189
Pleyer et al., Blood Advances (2022), vol. 6, p. 1733-1740
Polman et al., Ann Neurol. (2011), vol. 69, p. 292-302
Pugh et al., Nature Reviews Disease Primers (2021), vol. 7(1), art. 93
Reich et al., Lancet Neurology (2021), vol. 20, p. 729
Rijvers et al., AAN (2021), Abstract
Rijvers et al., JCI Insight (2022, in press)
Ringheim et al., Frontiers in Immunology (2021), vol. 66, p. 2223
Rosen et al., New Eng. J. Med. (1995), vol. 333, p. 431
Samson et al., Cureus (2019), vol. 11(10), p. e5849 (doi:10.7759/cureus.5849)
Scully & Challacombe, Crit. Rev. Oral. Biol. Med. (2002), vol. 13, p. 397
Siegrist, Vaccines—Vaccine Immunology, book chapter in: “Vaccines”, editors: Plotkin, Orenstein and Offit, published by Saunders/Elsevier, 5th ed. (2008), p. 16-34
Sun et al., JAMA Oncology (2016), vol. 2(12), p. 1656-1657
Tasso et al., Molecules (2021), vol. 26, p. 7411
Venables, Best. Pract. Res. Clin. Rheumatol. (2004), vol. 18(3), p. 313-329
Wallace et al., Lupus Science & Medicine (2019); vol. 6, abstract 212, doi: 10.1136/lupus-2019-lsm.212
Wallace et al. Arthritis Rheumatol. (2020), vol. 72 (suppl. 10), abstract 0865
Zrzavry et al., Frontiers in Immunology (2019), vol. 10, p. 1883

1. A method of treating an autoimmune disease or inflammatory disease in a patient, the following method comprising:
(a) optionally administering a BTK inhibitor to a patient in need thereof,
(b) identifying that said patient is at risk of contracting an infection,
(c) vaccinating said patient against said infection, and
(d) optionally administering said BTK inhibitor to said patient,
with the proviso that said method comprises one or more treatment periods in which said BTK inhibitor is administered,
wherein said inflammatory disease is selected from the group consisting of graft versus host disease (GVHD), large vessel vasculitis (LVV) and asthma,
and wherein said patient is vaccinated while said patient is under ongoing BTK inhibitor treatment or wherein a time period between administration of said BTK inhibitor and vaccination is less than 4 weeks.

2. The method according to claim 1, wherein the patient is a patient in need of treatment of an autoimmune disease.

3. The method according to claim 2, wherein the autoimmune disease is selected from the group consisting of systemic lupus erythematosus (SLE), multiple sclerosis (MS), rheumatoid arthritis (RA), neuromyelitis optica spectrum disorders (NMOSD), myasthenia gravis (MG), pemphigus vulgaris (PV), Sjogren's syndrome (SJ), chronic spontaneous urticaria (CSU) and idiopathic thrombocytopenia (ITP).

4. The method according to claim 2, wherein said autoimmune disease is SLE or MS.

5. The method according to claim 1, wherein said BTK inhibitor is
1-[4-({[6-amino-5-(4-phenoxyphenyl)pyrimidin-4-yl]amino}methyl)piperidin-1-yl]prop-2-en-1-one or a pharmaceutically acceptable salt, tautomer, or stereoisomer thereof, or
1-(4-({[6-amino-5-(4-phenoxyphenyl)pyrimidin-4-yl]amino}methyl)-4-fluoropiperidin-1-yl)prop-2-en-1-one or a pharmaceutically acceptable salt, tautomer, or stereoisomer thereof, or
a mixture of any of the above at any ratio.

6. The method according to claim 1, wherein said BTK inhibitor is evobrutinib.

7. The method according to claim 1, wherein said BTK inhibitor is administered at a daily amount ranging from 20 mg to 300 mg.

8. The method according to claim 1, wherein 40 to 85 mg of said BTK inhibitor are administered twice daily,
or wherein said BTK inhibitor is administered in such a manner that a resulting concentration of said BTK inhibitor in a plasma of said patient is similar toa concentration of said BTK inhibitor in a plasma achieved if said BTK inhibitor is administered at 40 to 85 ng administered twice daily by oral administration as a tablet.

9. The method according to claim 1, wherein said vaccination is performed with a vaccine selected from the group consisting of an inactivated vaccine, live-attenuated vaccine, a live vaccine, a subunit vaccine, a recombinant vaccine, a polysaccharide vaccine, a conjugate vaccine, and a toxoid vaccine, and a combination thereof.

10. The method according to claim 1, wherein said infection is a viral infection.

11. The method according to claim 1, wherein said infection is a bacterial infection.

12. The method according to claim 1, wherein said infection is influenza or COVID-19.
